SEQUENCE LISTING (771 sequences)

SEQ ID NO: 1 - DNA, 13 bp, synthetic construct
  source 1..13: mol_type = other DNA
gccgccaccatgg

SEQ ID NO: 2 - RNA, 13 nt, synthetic construct
  source 1..13: mol_type = other RNA
gccgccaccatgg

SEQ ID NO: 3 - DNA, 24 bp, synthetic construct
  source 1..24: mol_type = other DNA
caaaggctcttttcagagccacca

SEQ ID NO: 4 - RNA, 24 nt, synthetic construct
  source 1..24: mol_type = other RNA
caaaggctcttttcagagccacca

SEQ ID NO: 5 - RNA, 124 nt, synthetic construct
  source 1..124: mol_type = other RNA
caaaggctcttttcagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 6 - RNA, 100 nt, synthetic construct
  source 1..100: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 7 - RNA, 129 nt, synthetic construct
  source 1..129: mol_type = other RNA
caaaggctcttttcagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaatt

SEQ ID NO: 8 - RNA, 93 nt, synthetic construct
  source 1..93: mol_type = other RNA
caaaggctcttttcagagccaccaaaaaaaaaaaaaaaaaaaaaaaaaaaaaaaaaaaaaaaaaaaaaaaaaaaaaaaaaaaaaaaaagaatt

SEQ ID NO: 9 - RNA, 105 nt, synthetic construct
  source 1..105: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaatt

SEQ ID NO: 10 - RNA, 105 nt, synthetic construct
  source 1..105: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 11 - RNA, 110 nt, synthetic construct
  source 1..110: mol_type = other RNA
aaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 12 - DNA, 62 bp, Homo sapiens
  source 1..62: mol_type = genomic DNA
gtcccgcagtcggcgtccagcggctctgcttgttcgtgtgtgtgtcgttgcaggccttattc

SEQ ID NO: 13 - RNA, 62 nt, Homo sapiens
  source 1..62: mol_type = mRNA
gtcccgcagtcggcgtccagcggctctgcttgttcgtgtgtgtgtcgttgcaggccttattc

SEQ ID NO: 14 - DNA, 42 bp, Homo sapiens
  source 1..42: mol_type = genomic DNA
ggcgctgcctacggaggtggcagccatctccttctcggcatc

SEQ ID NO: 15 - RNA, 42 nt, Homo sapiens
  source 1..42: mol_type = mRNA
ggcgctgcctacggaggtggcagccatctccttctcggcatc

SEQ ID NO: 16 - DNA, 31 bp, Homo sapiens
  source 1..31: mol_type = genomic DNA
gccgcccagccggtccaggcctctggcgaac

SEQ ID NO: 17 - RNA, 31 nt, Homo sapiens
  source 1..31: mol_type = mRNA
gccgcccagccggtccaggcctctggcgaac

SEQ ID NO: 18 - DNA, 42 bp, Homo sapiens
  source 1..42: mol_type = genomic DNA
gcctctgctggagtccggggagtggcgttggctgctagagcg

SEQ ID NO: 19 - RNA, 42 nt, Homo sapiens
  source 1..42: mol_type = mRNA
gcctctgctggagtccggggagtggcgttggctgctagagcg

SEQ ID NO: 20 - DNA, 75 bp, Homo sapiens
  source 1..75: mol_type = genomic DNA
gcggctcggccattttgtcccagtcagtccggaggctgcggctgcagaagtaccgcctgcggagtaactgcaaag

SEQ ID NO: 21 - RNA, 75 nt, Homo sapiens
  source 1..75: mol_type = mRNA
gcggctcggccattttgtcccagtcagtccggaggctgcggctgcagaagtaccgcctgcggagtaactgcaaag

SEQ ID NO: 22 - DNA, 56 bp, Homo sapiens
  source 1..56: mol_type = genomic DNA
agtcaggaaggacgttggtgttgaggttagcatacgtatcaaggacagtaactacc

SEQ ID NO: 23 - RNA, 56 nt, Homo sapiens
  source 1..56: mol_type = mRNA
agtcaggaaggacgttggtgttgaggttagcatacgtatcaaggacagtaactacc

SEQ ID NO: 24 - DNA, 232 bp, Mus musculus
  source 1..232: mol_type = genomic DNA
ctctctccctctttcttttttccgccctagctggatctgtgttggaggagaggaaaagagacagaggattgcatctgttttgaaatctcctaatagcaagaccagttaagggattgtacctttttcctacaaatataaatatatatatattttaaaccaagtctttttttttccggctatctttgctttaaagctgtcctcttgagattacttcctcccgccccccggagag

SEQ ID NO: 25 - RNA, 232 nt, Mus musculus
  source 1..232: mol_type = mRNA
ctctctccctctttcttttttccgccctagctggatctgtgttggaggagaggaaaagagacagaggattgcatctgttttgaaatctcctaatagcaagaccagttaagggattgtacctttttcctacaaatataaatatatatatattttaaaccaagtctttttttttccggctatctttgctttaaagctgtcctcttgagattacttcctcccgccccccggagag

SEQ ID NO: 26 - DNA, 22 bp, Homo sapiens
  source 1..22: mol_type = genomic DNA
ggttcctctttcctcgctcaag

SEQ ID NO: 27 - RNA, 22 nt, Homo sapiens
  source 1..22: mol_type = mRNA
ggttcctctttcctcgctcaag

SEQ ID NO: 28 - DNA, 54 bp, Mus musculus
  source 1..54: mol_type = genomic DNA
ctttcccattctgtagcagaatttggtgttgcctgtggtcttggtcccgcggag

SEQ ID NO: 29 - RNA, 54 nt, Mus musculus
  source 1..54: mol_type = mRNA
ctttcccattctgtagcagaatttggtgttgcctgtggtcttggtcccgcggag

SEQ ID NO: 30 - DNA, 81 bp, Mus musculus
  source 1..81: mol_type = genomic DNA
gtccgctcagccaggttgcagaagcggcttagcgtgtgtcctaatcttctctctgcgtgtaggtaggcctgtgccgcaaac

SEQ ID NO: 31 - RNA, 81 nt, Mus musculus
  source 1..81: mol_type = mRNA
gtccgctcagccaggttgcagaagcggcttagcgtgtgtcctaatcttctctctgcgtgtaggtaggcctgtgccgcaaac

SEQ ID NO: 32 - DNA, 108 bp, Mus musculus
  source 1..108: mol_type = genomic DNA
ctcctgtcgggcggaagtaggaggagtagagtttaaaaacagtactctttttccggttcgggacgtagttgaagcaacgacaagccggataaccgctcttgagacagg

SEQ ID NO: 33 - RNA, 108 nt, Mus musculus
  source 1..108: mol_type = mRNA
ctcctgtcgggcggaagtaggaggagtagagtttaaaaacagtactctttttccggttcgggacgtagttgaagcaacgacaagccggataaccgctcttgagacagg

SEQ ID NO: 34 - DNA, 84 bp, Mus musculus
  source 1..84: mol_type = genomic DNA
cccgtgacccggaagttgtacggctacgcgactttccctcccacaaaccctcgcgcccttcctttcctacttgggcccggcaga

SEQ ID NO: 35 - RNA, 84 nt, Mus musculus
  source 1..84: mol_type = mRNA
cccgtgacccggaagttgtacggctacgcgactttccctcccacaaaccctcgcgcccttcctttcctacttgggcccggcaga

SEQ ID NO: 36 - DNA, 56 bp, Homo sapiens
  source 1..56: mol_type = genomic DNA
accgccatcttggctcctgtggaggcctgctgggaacgggacttctaaaaggaact

SEQ ID NO: 37 - RNA, 56 nt, Homo sapiens
  source 1..56: mol_type = mRNA
accgccatcttggctcctgtggaggcctgctgggaacgggacttctaaaaggaact

SEQ ID NO: 38 - DNA, 159 bp, Mus musculus
  source 1..159: mol_type = genomic DNA
gggcgcttggcttgcaaggaccctgagctgcggcattgaagcacacccaacccaactcgactgaagtcagcctcactgaaccggatctgagaatcttctctctctgggcttgccagggctctccgaacctagctagcatcctcttcaattccaactaga

SEQ ID NO: 39 - RNA, 159 nt, Mus musculus
  source 1..159: mol_type = mRNA
gggcgcttggcttgcaaggaccctgagctgcggcattgaagcacacccaacccaactcgactgaagtcagcctcactgaaccggatctgagaatcttctctctctgggcttgccagggctctccgaacctagctagcatcctcttcaattccaactaga

SEQ ID NO: 40 - DNA, 102 bp, Homo sapiens
  source 1..102: mol_type = genomic DNA
atataagcgttggcggagcgtcggttgtagcactctgcgcgcccgctcttctgctgctgtttgtctacttcctcctgcttccccgccgccgccgccgccatc

SEQ ID NO: 41 - RNA, 102 nt, Homo sapiens
  source 1..102: mol_type = mRNA
atataagcgttggcggagcgtcggttgtagcactctgcgcgcccgctcttctgctgctgtttgtctacttcctcctgcttccccgccgccgccgccgccatc

SEQ ID NO: 42 - DNA, 222 bp, Mus musculus
  source 1..222: mol_type = genomic DNA
cggagacggcctgcaggacctgctctctcagccctcagccgaggcctacgccgagccgagtgcgcagccgacgaccgggaggagccgcagccttcaactctgaggtactgtgatccgcgctgcccgccgggccgccccagtccgctgctgcggcacctccttccctcgcgccctcttcgctcgccagcgccttccctgtgagcctgcgtcaccgcggccgcc

SEQ ID NO: 43 - RNA, 222 nt, Mus musculus
  source 1..222: mol_type = mRNA
cggagacggcctgcaggacctgctctctcagccctcagccgaggcctacgccgagccgagtgcgcagccgacgaccgggaggagccgcagccttcaactctgaggtactgtgatccgcgctgcccgccgggccgccccagtccgctgctgcggcacctccttccctcgcgccctcttcgctcgccagcgccttccctgtgagcctgcgtcaccgcggccgcc

SEQ ID NO: 44 - DNA, 34 bp, Homo sapiens
  source 1..34: mol_type = genomic DNA
actcttctggtccccacagactcagagagaaccc

SEQ ID NO: 45 - RNA, 34 nt, Homo sapiens
  source 1..34: mol_type = mRNA
actcttctggtccccacagactcagagagaaccc

SEQ ID NO: 46 - DNA, 53 bp, synthetic construct
  source 1..53: mol_type = other DNA
agaataaactagtattcttctggtccccacagactcagagagaacccgccacc

SEQ ID NO: 47 - RNA, 53 nt, synthetic construct
  source 1..53: mol_type = other RNA
agaataaactagtattcttctggtccccacagactcagagagaacccgccacc

SEQ ID NO: 48 - DNA, 53 bp, synthetic construct
  source 1..53: mol_type = other DNA
agattaaactagtattcttctggtccccacagactcagagagaacccgccacc

SEQ ID NO: 49 - RNA, 53 nt, synthetic construct
  source 1..53: mol_type = other RNA
agattaaactagtattcttctggtccccacagactcagagagaacccgccacc

SEQ ID NO: 50 - DNA, 52 bp, synthetic construct
  source 1..52: mol_type = other DNA
agacgaactagtattcttctggtccccacagactcagagagaacccgccacc

SEQ ID NO: 51 - RNA, 52 nt, synthetic construct
  source 1..52: mol_type = other RNA
agacgaactagtattcttctggtccccacagactcagagagaacccgccacc

SEQ ID NO: 52 - DNA, 52 bp, synthetic construct
  source 1..52: mol_type = other DNA
gggcgaactagtattcttctggtccccacagactcagagagaacccgccacc

SEQ ID NO: 53 - RNA, 52 nt, synthetic construct
  source 1..52: mol_type = other RNA
gggcgaactagtattcttctggtccccacagactcagagagaacccgccacc

SEQ ID NO: 54 - DNA, 57 bp, synthetic construct
  source 1..57: mol_type = other DNA
gggaaataagagagaaaagaagagtaagaagaaatataagaccccggcgccgccacc

SEQ ID NO: 55 - RNA, 57 nt, synthetic construct
  source 1..57: mol_type = other RNA
gggaaataagagagaaaagaagagtaagaagaaatataagaccccggcgccgccacc

SEQ ID NO: 56 - DNA, 47 bp, synthetic construct
  source 1..47: mol_type = other DNA
gggaaataagagagaaaagaagagtaagaagaaatataagagccacc

SEQ ID NO: 57 - RNA, 47 nt, synthetic construct
  source 1..47: mol_type = other RNA
gggaaataagagagaaaagaagagtaagaagaaatataagagccacc

SEQ ID NO: 58 - DNA, 54 bp, synthetic construct
  source 1..54: mol_type = other DNA
gggaaataagagagaaaagaagagtaagaagaaatataagaccccggcgccacc

SEQ ID NO: 59 - RNA, 54 nt, synthetic construct
  source 1..54: mol_type = other RNA
gggaaataagagagaaaagaagagtaagaagaaatataagaccccggcgccacc

SEQ ID NO: 60 - DNA, 67 bp, synthetic construct
  source 1..67: mol_type = other DNA
gggaaataagagagaaaagaagagtaagaagaaatataagagcgccccgcggcgccccgcggccacc

SEQ ID NO: 61 - RNA, 67 nt, synthetic construct
  source 1..67: mol_type = other RNA
gggaaataagagagaaaagaagagtaagaagaaatataagagcgccccgcggcgccccgcggccacc

SEQ ID NO: 62 - DNA, 140 bp, synthetic construct
  source 1..140: mol_type = other DNA
ggacagatcgcctggagacgccatccacgctgttttgacctccatagaagacaccgggaccgatccagcctccgcggccgggaacggtgcattggaacgcggattccccgtgccaagagtgactcaccgtccttgacacg

SEQ ID NO: 63 - RNA, 140 nt, synthetic construct
  source 1..140: mol_type = other RNA
ggacagatcgcctggagacgccatccacgctgttttgacctccatagaagacaccgggaccgatccagcctccgcggccgggaacggtgcattggaacgcggattccccgtgccaagagtgactcaccgtccttgacacg

SEQ ID NO: 64 - DNA, 77 bp, synthetic construct
  source 1..77: mol_type = other DNA
aggagagtcccgcagtcggcgtccagcggctctgcttgttcgtgtgtgtgtcgttgcaggccttattcaagcttacc

SEQ ID NO: 65 - RNA, 77 nt, synthetic construct
  source 1..77: mol_type = other RNA
aggagagtcccgcagtcggcgtccagcggctctgcttgttcgtgtgtgtgtcgttgcaggccttattcaagcttacc

SEQ ID NO: 66 - DNA, 57 bp, Homo sapiens
  source 1..57: mol_type = genomic DNA
ccctgttcccagagcccacttttttttctttttttgaaataaaatagcctgtctttc

SEQ ID NO: 67 - RNA, 57 nt, Homo sapiens
  source 1..57: mol_type = mRNA
ccctgttcccagagcccacttttttttctttttttgaaataaaatagcctgtctttc

SEQ ID NO: 68 - DNA, 44 bp, Homo sapiens
  source 1..44: mol_type = genomic DNA
gcccgatgggcctcccaacgggccctcctcccctccttgcaccg

SEQ ID NO: 69 - RNA, 44 nt, Homo sapiens
  source 1..44: mol_type = mRNA
gcccgatgggcctcccaacgggccctcctcccctccttgcaccg

SEQ ID NO: 70 - DNA, 187 bp, Homo sapiens
  source 1..187: mol_type = genomic DNA
gcatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacct

SEQ ID NO: 71 - RNA, 187 nt, Homo sapiens
  source 1..187: mol_type = mRNA
gcatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacct

SEQ ID NO: 72 - DNA, 266 bp, Homo sapiens
  source 1..266: mol_type = genomic DNA
aataaaaatcccagaaggacaggaggattctcaacactttgaatttttttaacttcatttttctacactgctattatcattatctcagaatgcttatttccaattaaaacgcctacagctgcctcctagaatatagactgtctgtattattattcacctataattagtcattatgatgctttaaagctgtacttgcatttcaaagcttataagatataaatggagattttaaagtagaaataaatatgtattccatgtttttaaaa

SEQ ID NO: 73 - RNA, 266 nt, Homo sapiens
  source 1..266: mol_type = mRNA
aataaaaatcccagaaggacaggaggattctcaacactttgaatttttttaacttcatttttctacactgctattatcattatctcagaatgcttatttccaattaaaacgcctacagctgcctcctagaatatagactgtctgtattattattcacctataattagtcattatgatgctttaaagctgtacttgcatttcaaagcttataagatataaatggagattttaaagtagaaataaatatgtattccatgtttttaaaa

SEQ ID NO: 74 - DNA, 121 bp, Homo sapiens
  source 1..121: mol_type = genomic DNA
aataaggaaactgtatgaatgtctgtgggcaggaagtgaagagatccttctgtaaaggtttttggaattatgtctgctgaataataaacttttttgaaataataaatctggtagaaaaatg

SEQ ID NO: 75 - RNA, 121 nt, Homo sapiens
  source 1..121: mol_type = mRNA
aataaggaaactgtatgaatgtctgtgggcaggaagtgaagagatccttctgtaaaggtttttggaattatgtctgctgaataataaacttttttgaaataataaatctggtagaaaaatg

SEQ ID NO: 76 - DNA, 137 bp, Homo sapiens
  source 1..137: mol_type = genomic DNA
actggctgcatctccctttcctctgtcctccatccttctcccaggatggtgaagggggacctggtacccagtgatccccaccccaggatcctaaatcatgacttacctgctaataaaaactcattggaaaagtgaga

SEQ ID NO: 77 - RNA, 137 nt, Homo sapiens
  source 1..137: mol_type = mRNA
actggctgcatctccctttcctctgtcctccatccttctcccaggatggtgaagggggacctggtacccagtgatccccaccccaggatcctaaatcatgacttacctgctaataaaaactcattggaaaagtgaga

SEQ ID NO: 78 - DNA, 166 bp, Homo sapiens
  source 1..166: mol_type = genomic DNA
aaagagcgcaggtccacctggtggacacgtctgattagcttagaacctgtcttgtctcatcttcaaaaggtaacttattaaaagtcctttgcgtctgaagcctttctccttttctgtcacttgcaaattccaaattatagctaataaagatgactagataatttgc

SEQ ID NO: 79 - RNA, 166 nt, Homo sapiens
  source 1..166: mol_type = mRNA
aaagagcgcaggtccacctggtggacacgtctgattagcttagaacctgtcttgtctcatcttcaaaaggtaacttattaaaagtcctttgcgtctgaagcctttctccttttctgtcacttgcaaattccaaattatagctaataaagatgactagataatttgc

SEQ ID NO: 80 - DNA, 353 bp, Mus musculus
  source 1..353: mol_type = genomic DNA
gaagggaacacccaaatttaattcagccttaagcacaattaattaagagtgaaacgtaattgtacaagcagttggtcacccaccatagggcatgatcaacaccgcaacctttcctttttcccccagtgattctgaaaaacccctcttcccttcagcttgcttagatgttccaaatttagtaagcttaaggcggcctacagaagaaaaagaaaaaaaaggccacaaaagttccctctcactttcagtaaataaaataaaagcagcaacagaaataaagaaataaatgaaattcaaaatgaaataaatattgtgttgtgcagcattaaaaaatcaataaaaattaaaaatgagca

SEQ ID NO: 81 - RNA, 353 nt, Mus musculus
  source 1..353: mol_type = mRNA
gaagggaacacccaaatttaattcagccttaagcacaattaattaagagtgaaacgtaattgtacaagcagttggtcacccaccatagggcatgatcaacaccgcaacctttcctttttcccccagtgattctgaaaaacccctcttcccttcagcttgcttagatgttccaaatttagtaagcttaaggcggcctacagaagaaaaagaaaaaaaaggccacaaaagttccctctcactttcagtaaataaaataaaagcagcaacagaaataaagaaataaatgaaattcaaaatgaaataaatattgtgttgtgcagcattaaaaaatcaataaaaattaaaaatgagca

SEQ ID NO: 82 - DNA, 133 bp, Mus musculus
  source 1..133: mol_type = genomic DNA
ggaagcattttcctggctgattaaaagaaattactcagctatggtcatctgttcctgttagaaggctatgcagcatattatatactatgcgcatgttatgaaatgcataataaaaaattttaaaaaatctaaa

SEQ ID NO: 83 - RNA, 133 nt, Mus musculus
  source 1..133: mol_type = mRNA
ggaagcattttcctggctgattaaaagaaattactcagctatggtcatctgttcctgttagaaggctatgcagcatattatatactatgcgcatgttatgaaatgcataataaaaaattttaaaaaatctaaa

SEQ ID NO: 84 - DNA, 70 bp, Homo sapiens
  source 1..70: mol_type = genomic DNA
gtccacctgtccctcctgggctgctggattgtctcgttttcctgccaaataaacaggatcagcgctttac

SEQ ID NO: 85 - RNA, 70 nt, Homo sapiens
  source 1..70: mol_type = mRNA
gtccacctgtccctcctgggctgctggattgtctcgttttcctgccaaataaacaggatcagcgctttac

SEQ ID NO: 86 - DNA, 240 bp, Mus musculus
  source 1..240: mol_type = genomic DNA
cctaaccatacctaattgttgtcttgttctcctatacaataatggagagtactcttgaccccacgaagagctgggcctctcatgtggtgttagtgacggatatgaaataatgctctgtatttcttgtgaaatgatggctatgtctttgctgtttctcatttgtttttaacttgtatactttgaaatagtctctgtagttgtttcctggctttgaaaaaaatgttattaaaggaaaattgt

SEQ ID NO: 87 - RNA, 240 nt, Mus musculus
  source 1..240: mol_type = mRNA
cctaaccatacctaattgttgtcttgttctcctatacaataatggagagtactcttgaccccacgaagagctgggcctctcatgtggtgttagtgacggatatgaaataatgctctgtatttcttgtgaaatgatggctatgtctttgctgtttctcatttgtttttaacttgtatactttgaaatagtctctgtagttgtttcctggctttgaaaaaaatgttattaaaggaaaattgt

SEQ ID NO: 88 - DNA, 151 bp, Homo sapiens
  source 1..151: mol_type = genomic DNA
agccttcagtcactggggaaagcagggaagcagtgtgaactctttattcactcccagcctgtcctgtggcctgtcccactgtgtgcacttgctgttttccctgtccacatccatgctgtacagacaccaccattaaagcattttcatagtg

SEQ ID NO: 89 - RNA, 151 nt, Homo sapiens
  source 1..151: mol_type = mRNA
agccttcagtcactggggaaagcagggaagcagtgtgaactctttattcactcccagcctgtcctgtggcctgtcccactgtgtgcacttgctgttttccctgtccacatccatgctgtacagacaccaccattaaagcattttcatagtg

SEQ ID NO: 90 - DNA, 110 bp, Homo sapiens
  source 1..110: mol_type = genomic DNA
gctggagcctcggtggccatgcttcttgccccttgggcctccccccagcccctcctccccttcctgcacccgtacccccgtggtctttgaataaagtctgagtgggcggc

SEQ ID NO: 91 - RNA, 110 nt, Homo sapiens
  source 1..110: mol_type = mRNA
gctggagcctcggtggccatgcttcttgccccttgggcctccccccagcccctcctccccttcctgcacccgtacccccgtggtctttgaataaagtctgagtgggcggc

SEQ ID NO: 92 - DNA, 108 bp, Homo sapiens
  source 1..108: mol_type = genomic DNA
gctggagcctcggtagccgttcctcctgcccgctgggcctcccaacgggccctcctcccctccttgcaccggcccttcctggtctttgaataaagtctgagtgggcag

SEQ ID NO: 93 - RNA, 108 nt, Homo sapiens
  source 1..108: mol_type = mRNA
gctggagcctcggtagccgttcctcctgcccgctgggcctcccaacgggccctcctcccctccttgcaccggcccttcctggtctttgaataaagtctgagtgggcag

SEQ ID NO: 94 - DNA, 132 bp, Homo sapiens
  source 1..132: mol_type = genomic DNA
gctcgctttcttgctgtccaatttctattaaaggttcctttgttccctaagtccaactactaaactgggggatattatgaagggccttgagcatctggattctgcctaataaaaaacatttattttcattgc

SEQ ID NO: 95 - RNA, 132 nt, Homo sapiens
  source 1..132: mol_type = mRNA
gctcgctttcttgctgtccaatttctattaaaggttcctttgttccctaagtccaactactaaactgggggatattatgaagggccttgagcatctggattctgcctaataaaaaacatttattttcattgc

SEQ ID NO: 96 - DNA, 119 bp, synthetic construct
  source 1..119: mol_type = other DNA
tgataataggctggagcctcggtggcctagcttcttgccccttgggcctccccccagcccctcctccccttcctgcacccgtacccccgtggtctttgaataaagtctgagtgggcggc

SEQ ID NO: 97 - RNA, 119 nt, synthetic construct
  source 1..119: mol_type = other RNA
tgataataggctggagcctcggtggcctagcttcttgccccttgggcctccccccagcccctcctccccttcctgcacccgtacccccgtggtctttgaataaagtctgagtgggcggc

SEQ ID NO: 98 - DNA, 122 bp, synthetic construct
  source 1..122: mol_type = other DNA
tgataataggctggagcctcggtggcctagcttcttgccccttgggcctccccccagcccctcctccccttcctgcaggagagatagtggagtggtctttgaataaagtctgagtgggcggc

SEQ ID NO: 99 - RNA, 122 nt, synthetic construct
  source 1..122: mol_type = other RNA
tgataataggctggagcctcggtggcctagcttcttgccccttgggcctccccccagcccctcctccccttcctgcaggagagatagtggagtggtctttgaataaagtctgagtgggcggc

SEQ ID NO: 100 - DNA, 113 bp, synthetic construct
  source 1..113: mol_type = other DNA
tgataataggctggagcctcggtggcctagcttcttgccccttgggcctccccccagcccctcctccccttcctgcaggttagtggtctttgaataaagtctgagtgggcggc

SEQ ID NO: 101 - RNA, 113 nt, synthetic construct
  source 1..113: mol_type = other RNA
tgataataggctggagcctcggtggcctagcttcttgccccttgggcctccccccagcccctcctccccttcctgcaggttagtggtctttgaataaagtctgagtgggcggc

SEQ ID NO: 102 - DNA, 119 bp, synthetic construct
  source 1..119: mol_type = other DNA
tgataataggctggagcctcggtggccatgcttcttgccccttgggcctccccccagcccctcctccccttcctgcacccgtacccccgtggtctttgaataaagtctgagtgggcggc

SEQ ID NO: 103 - RNA, 119 nt, synthetic construct
  source 1..119: mol_type = other RNA
tgataataggctggagcctcggtggccatgcttcttgccccttgggcctccccccagcccctcctccccttcctgcacccgtacccccgtggtctttgaataaagtctgagtgggcggc

SEQ ID NO: 104 - DNA, 311 bp, synthetic construct
  source 1..311: mol_type = other DNA
ctcgagagctcgctttcttgctgtccaatttctattaaaggttcctttgttccctaagtccaactactaaactgggggatattatgaagggccttgagcatctggattctgcctaataaaaaacatttattttcattgctgcgtcgagagctcgctttcttgctgtccaatttctattaaaggttcctttgttccctaagtccaactactaaactgggggatattatgaagggccttgagcatctggattctgcctaataaaaaacatttattttcattgctgcgtcgagacctggtccagagtcgctagc

SEQ ID NO: 105 - RNA, 311 nt, synthetic construct
  source 1..311: mol_type = other RNA
ctcgagagctcgctttcttgctgtccaatttctattaaaggttcctttgttccctaagtccaactactaaactgggggatattatgaagggccttgagcatctggattctgcctaataaaaaacatttattttcattgctgcgtcgagagctcgctttcttgctgtccaatttctattaaaggttcctttgttccctaagtccaactactaaactgggggatattatgaagggccttgagcatctggattctgcctaataaaaaacatttattttcattgctgcgtcgagacctggtccagagtcgctagc

SEQ ID NO: 106 - DNA, 295 bp, synthetic construct
  source 1..295: mol_type = other DNA
ctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagc

SEQ ID NO: 107 - RNA, 295 nt, synthetic construct
  source 1..295: mol_type = other RNA
ctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagc

SEQ ID NO: 108 - DNA, 317 bp, synthetic construct
  source 1..317: mol_type = other DNA
ctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgct

SEQ ID NO: 109 - RNA, 317 nt, synthetic construct
  source 1..317: mol_type = other RNA
ctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgct

SEQ ID NO: 110 - DNA, 105 bp, synthetic construct
  source 1..105: mol_type = other DNA
cgggtggcatccctgtgacccctccccagtgcctctcctggccctggaagttgccactccagtgcccaccagccttgtcctaataaaattaagttgcatcaagct

SEQ ID NO: 111 - RNA, 105 nt, synthetic construct
  source 1..105: mol_type = other RNA
cgggtggcatccctgtgacccctccccagtgcctctcctggccctggaagttgccactccagtgcccaccagccttgtcctaataaaattaagttgcatcaagct

SEQ ID NO: 112 - DNA, 71 bp, synthetic construct
  source 1..71: mol_type = other DNA
ggactagtccctgttcccagagcccacttttttttctttttttgaaataaaatagcctgtctttcagatct

SEQ ID NO: 113 - RNA, 71 nt, synthetic construct
  source 1..71: mol_type = other RNA
ggactagtccctgttcccagagcccacttttttttctttttttgaaataaaatagcctgtctttcagatct

SEQ ID NO: 114 - DNA, 77 bp, synthetic construct
  source 1..77: mol_type = other DNA
tgatgaggactagtccctgttcccagagcccacttttttttctttttttgaaataaaatagcctgtctttcagatct

SEQ ID NO: 115 - RNA, 77 nt, synthetic construct
  source 1..77: mol_type = other RNA
tgatgaggactagtccctgttcccagagcccacttttttttctttttttgaaataaaatagcctgtctttcagatct

SEQ ID NO: 116 - DNA, 195 bp, synthetic construct
  source 1..195: mol_type = other DNA
ggactagtccctgttcccagagcccacttttttttctttttttgaaataaaatagcctgtctttcagatctcaaaggctcttttcagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 117 - RNA, 195 nt, synthetic construct
  source 1..195: mol_type = other RNA
ggactagtccctgttcccagagcccacttttttttctttttttgaaataaaatagcctgtctttcagatctcaaaggctcttttcagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 118 - DNA, 201 bp, synthetic construct
  source 1..201: mol_type = other DNA
tgatgaggactagtccctgttcccagagcccacttttttttctttttttgaaataaaatagcctgtctttcagatctcaaaggctcttttcagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 119 - RNA, 201 nt, synthetic construct
  source 1..201: mol_type = other RNA
tgatgaggactagtccctgttcccagagcccacttttttttctttttttgaaataaaatagcctgtctttcagatctcaaaggctcttttcagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 120 - DNA, 171 bp, synthetic construct
  source 1..171: mol_type = other DNA
ggactagtccctgttcccagagcccacttttttttctttttttgaaataaaatagcctgtctttcagatctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 121 - RNA, 171 nt, synthetic construct
  source 1..171: mol_type = other RNA
ggactagtccctgttcccagagcccacttttttttctttttttgaaataaaatagcctgtctttcagatctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 122 - DNA, 177 bp, synthetic construct
  source 1..177: mol_type = other DNA
tgatgaggactagtccctgttcccagagcccacttttttttctttttttgaaataaaatagcctgtctttcagatctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 123 - RNA, 177 nt, synthetic construct
  source 1..177: mol_type = other RNA
tgatgaggactagtccctgttcccagagcccacttttttttctttttttgaaataaaatagcctgtctttcagatctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 124 - protein, 43 aa, Homo sapiens [sequence omitted]

SEQ ID NO: 125 - protein, 15 aa, Homo sapiens
REVQVTSIKETFLSM

SEQ ID NO: 126 - protein, 15 aa, Homo sapiens
MYVSNREVQVTSIKE

SEQ ID NO: 127 - protein, 15 aa, Homo sapiens
TFLSMGPASGLMLGC

SEQ ID NO: 128 - protein, 17 aa, Homo sapiens
IKETFLSMGPASGLMLG

SEQ ID NO: 129 - protein, 15 aa, Homo sapiens
MLGCSRQEMGKLEKR

SEQ ID NO: 130 - protein, 15 aa, Homo sapiens
GCSRQEMGKLEKRTV

SEQ ID NO: 131 - protein, 14 aa, Homo sapiens
EVQVTSIKETFLSM

SEQ ID NO: 132 - protein, 8 aa, Homo sapiens
MYVSNREV

SEQ ID NO: 133 - protein, 10 aa, Homo sapiens
MYVSNREVQV

SEQ ID NO: 134 - protein, 10 aa, Homo sapiens
TSIKETFLSM

SEQ ID NO: 135 - protein, 10 aa, Homo sapiens
LSMGPASGLM

SEQ ID NO: 136 - protein, 10 aa, Homo sapiens
SMGPASGLML

SEQ ID NO: 137 - protein, 11 aa, Homo sapiens
SNREVQVTSIK

SEQ ID NO: 138 - protein, 11 aa, Homo sapiens
EVQVTSIKETF

SEQ ID NO: 139 - protein, 11 aa, Homo sapiens
VTSIKETFLSM

SEQ ID NO: 140 - protein, 11 aa, Homo sapiens
TFLSMGPASGL

SEQ ID NO: 141 - protein, 11 aa, Homo sapiens
FLSMGPASGLM

SEQ ID NO: 142 - protein, 11 aa, Homo sapiens
LSMGPASGLML

SEQ ID NO: 143 - protein, 11 aa, Homo sapiens
SMGPASGLMLG

SEQ ID NO: 144 - protein, 17 aa, Homo sapiens
MLGCSRQEMGKLEKRTV

SEQ ID NO: 145 - protein, 15 aa, Homo sapiens
MLGCSRQEMGKLEKR

SEQ ID NO: 146 - protein, 15 aa, Homo sapiens
GCSRQEMGKLEKRTV

SEQ ID NO: 147 - protein, 39 aa, Homo sapiens
MALCRCDPELCWISCEENSKEWMNAWKLTAGSHKAMTRK

SEQ ID NO: 148 - protein, 15 aa, Homo sapiens
KEWMNAWKLTAGSHK

SEQ ID NO: 149 - protein, 15 aa, Homo sapiens
AWKLTAGSHKAMTRK

SEQ ID NO: 150 - protein, 15 aa, Homo sapiens
RCDPELCWISCEENS

SEQ ID NO: 151 - protein, 15 aa, Homo sapiens
WISCEENSKEWMNAW

SEQ ID NO: 152 - protein, 8 aa, Homo sapiens
EWMNAWKL

SEQ ID NO: 153 - protein, 8 aa, Homo sapiens
MNAWKLTA

SEQ ID NO: 154 - protein, 9 aa, Homo sapiens
RCDPELCWI

SEQ ID NO: 155 - protein, 9 aa, Homo sapiens
WMNAWKLTA

SEQ ID NO: 156 - protein, 10 aa, Homo sapiens
RCDPELCWIS

SEQ ID NO: 157 - protein, 10 aa, Homo sapiens
SCEENSKEWM

SEQ ID NO: 158 - protein, 11 aa, Homo sapiens
RCDPELCWISC

SEQ ID NO: 159 - protein, 11 aa, Homo sapiens
KEWMNAWKLTA

SEQ ID NO: 160 - protein, 11 aa, Homo sapiens
NAWKLTAGSHK

SEQ ID NO: 161 - protein, 11 aa, Homo sapiens
LTAGSHKAMTR

SEQ ID NO: 162 - protein, 26 aa, Homo sapiens
MCVHAYLSGQPLEATTLCQLHSTCHL

SEQ ID NO: 163 - protein, 16 aa, Homo sapiens
AYLSGQPLEATTLCQL

SEQ ID NO: 164 - protein, 15 aa, Homo sapiens
CVHAYLSGQPLEATT

SEQ ID NO: 165 - protein, 16 aa, Homo sapiens
HAYLSGQPLEATTLCQ

SEQ ID NO: 166 - protein, 10 aa, Homo sapiens
CVHAYLSGQP

SEQ ID NO: 167 - protein, 10 aa, Homo sapiens
YLSGQPLEAT

SEQ ID NO: 168 - protein, 11 aa, Homo sapiens
CVHAYLSGQPL

SEQ ID NO: 169 - protein, 73 aa, Homo sapiens [sequence omitted]

SEQ ID NO: 170 - protein, 16 aa, Homo sapiens
SITALSYHFYKNHIHF

SEQ ID NO: 171 - protein, 17 aa, Homo sapiens
SLSGTQALVLYIQVISK

SEQ ID NO: 172 - protein, 17 aa, Homo sapiens
VLNSDQNSGEMLRRNSA

SEQ ID NO: 173 - protein, 15 aa, Homo sapiens
ASTERVSEGFKDSIT

SEQ ID NO: 174 - protein, 17 aa, Homo sapiens
ALSYHFYKNHIHFFPPT

SEQ ID NO: 175 - protein, 17 aa, Homo sapiens
HFFPPTCPFSLSGTQAL

SEQ ID NO: 176 - protein, 11 aa, Homo sapiens
ALVLYIQVISK

SEQ ID NO: 177 - protein, 8 aa, Homo sapiens
FKDSITAL

SEQ ID NO: 178 - protein, 8 aa, Homo sapiens
ALSYHFYK

SEQ ID NO: 179 - protein, 8 aa, Homo sapiens
FYKNHIHF

SEQ ID NO: 180 - protein, 8 aa, Homo sapiens
YKNHIHFF

SEQ ID NO: 181 - protein, 8 aa, Homo sapiens
TQALVLYI

SEQ ID NO: 182 - protein, 8 aa, Homo sapiens
ALVLYIQV

SEQ ID NO: 183 - protein, 9 aa, Homo sapiens
NSDQNSGEM

SEQ ID NO: 184 - protein, 9 aa, Homo sapiens
GFKDSITAL

SEQ ID NO: 185 - protein, 9 aa, Homo sapiens
TALSYHFYK

SEQ ID NO: 186 - protein, 9 aa, Homo sapiens
SYHFYKNHI

SEQ ID NO: 187 - protein, 9 aa, Homo sapiens
HFYKNHIHF

SEQ ID NO: 188 - protein, 9 aa, Homo sapiens
FYKNHIHFF

SEQ ID NO: 189 - protein, 9 aa, Homo sapiens
FPPTCPFSL

SEQ ID NO: 190 - protein, 9 aa, Homo sapiens
GTQALVLYI

SEQ ID NO: 191 - protein, 9 aa, Homo sapiens
QALVLYIQV

SEQ ID NO: 192 - protein, 9 aa, Homo sapiens
ALVLYIQVI

SEQ ID NO: 193 - protein, 10 aa, Homo sapiens
NSDQNSGEML

SEQ ID NO: 194 - protein, 10 aa, Homo sapiens
SGEMLRRNSA

SEQ ID NO: 195 - protein, 10 aa, Homo sapiens
STERVSEGFK

SEQ ID NO: 196 - protein, 10 aa, Homo sapiens
SITALSYHFY

SEQ ID NO: 197 - protein, 10 aa, Homo sapiens
ITALSYHFYK

SEQ ID NO: 198 - protein, 10 aa, Homo sapiens
LSYHFYKNHI

SEQ ID NO: 199 - protein, 10 aa, Homo sapiens
SYHFYKNHIH

SEQ ID NO: 200 - protein, 10 aa, Homo sapiens
YHFYKNHIHF

SEQ ID NO: 201 - protein, 10 aa, Homo sapiens
HFYKNHIHFF

SEQ ID NO: 202 - protein, 10 aa, Homo sapiens
YKNHIHFFPP

SEQ ID NO: 203 - protein, 10 aa, Homo sapiens
FPPTCPFSLS

SEQ ID NO: 204 - protein, 10 aa, Homo sapiens
FSLSGTQALV

SEQ ID NO: 205 - protein, 10 aa, Homo sapiens
SGTQALVLYI

SEQ ID NO: 206 - protein, 10 aa, Homo sapiens
TQALVLYIQV

SEQ ID NO: 207 - protein, 10 aa, Homo sapiens
QALVLYIQVI

SEQ ID NO: 208 - protein, 10 aa, Homo sapiens
LVLYIQVISK

SEQ ID NO: 209 - protein, 10 aa, Homo sapiens
VLYIQVISKG

SEQ ID NO: 210 - protein, 11 aa, Homo sapiens
VLNSDQNSGEM

SEQ ID NO: 211 - protein, 11 aa, Homo sapiens
NSGEMLRRNSA

SEQ ID NO: 212 - protein, 11 aa, Homo sapiens
ASTERVSEGFK

SEQ ID NO: 213 - protein, 15 aa, Homo sapiens
GFKDSITALSYHFYK

SEQ ID NO: 214 - protein, 11 aa, Homo sapiens
GFKDSITALSY

SEQ ID NO: 215 - protein, 11 aa, Homo sapiens
DSITALSYHFY

SEQ ID NO: 216 - protein, 11 aa, Homo sapiens
SITALSYHFYK

SEQ ID NO: 217 - protein, 11 aa, Homo sapiens
ALSYHFYKNHI

SEQ ID NO: 218 - protein, 11 aa, Homo sapiens
SYHFYKNHIHF

SEQ ID NO: 219 - protein, 11 aa, Homo sapiens
YHFYKNHIHFF

SEQ ID NO: 220 - protein, 11 aa, Homo sapiens
YKNHIHFFPPT

SEQ ID NO: 221 - protein, 11 aa, Homo sapiens
HFFPPTCPFSL

SEQ ID NO: 222 - protein, 11 aa, Homo sapiens
FPPTCPFSLSG

SEQ ID NO: 223 - protein, 11 aa, Homo sapiens
CPFSLSGTQAL

SEQ ID NO: 224 - protein, 11 aa, Homo sapiens
FSLSGTQALVL

SEQ ID NO: 225 - protein, 11 aa, Homo sapiens
GTQALVLYIQV

SEQ ID NO: 226 - protein, 11 aa, Homo sapiens
TQALVLYIQVI

SEQ ID NO: 227 - protein, 65 aa, Homo sapiens
MNVPKGQTGNSSRGPGDGGNKDHWKESDRKDGKKDQDSRSAPEPKKPEENPASKFSSASNYAALS

SEQ ID NO: 228 - protein, 15 aa, Homo sapiens
NSSRGPGDGGNKDHW

SEQ ID NO: 229 - protein, 15 aa, Homo sapiens
DQDSRSAPEPKKPEE

SEQ ID NO: 230 - protein, 15 aa, Homo sapiens
PASKFSSASNYAALS

SEQ ID NO: 231 - protein, 11 aa, Homo sapiens
SSRGPGDGGNK

SEQ ID NO: 232 - protein, 11 aa, Homo sapiens
GPGDGGNKDHW

SEQ ID NO: 233 - protein, 11 aa, Homo sapiens
DQDSRSAPEPK

SEQ ID NO: 234 - protein, 11 aa, Homo sapiens
SRSAPEPKKPE

SEQ ID NO: 235 - protein, 11 aa, Homo sapiens
KFSSASNYAAL

SEQ ID NO: 236 - protein, 8 aa, Homo sapiens
SASNYAAL

SEQ ID NO: 237 - protein, 9 aa, Homo sapiens
SSASNYAAL

SEQ ID NO: 238 - protein, 67 aa, Homo sapiens
MKTEVKNDHVLFYLENGFGRADSFPFSVEQSNLVFNIQPAPAMVYDYYEKEEYALAFYNIDSSSVSQ

SEQ ID NO: 239 - protein, 15 aa, Homo sapiens
SFPFSVEQSNLVFNI

SEQ ID NO: 240 - protein, 16 aa, Homo sapiens
KEEYALAFYNIDSSSV

SEQ ID NO: 241 - protein, 9 aa, Homo sapiens
FYNIDSSSV

SEQ ID NO: 242 - protein, 11 aa, Homo sapiens
KEEYALAFYNI

SEQ ID NO: 243 - protein, 11 aa, Homo sapiens
FPFSVEQSNLV

SEQ ID NO: 244 - protein, 65 aa, Homo sapiens
MILRIAGLFHLIILPLRSMKTLRHVEGQMENSMPTCFALWMCWMFPRAPCLTNRALSKPYSCWKR

SEQ ID NO: 245 - protein, 15 aa, Homo sapiens
MILRIAGLFHLIILP

SEQ ID NO: 246 - protein, 15 aa, Homo sapiens
RIAGLFHLIILPLRS

SEQ ID NO: 247 - protein, 15 aa, Homo sapiens
FHLIILPLRSMKTLR

SEQ ID NO: 248 - protein, 15 aa, Homo sapiens
MFPRAPCLTNRALSK

SEQ ID NO: 249 - protein, 11 aa, Homo sapiens
MILRIAGLFHL

SEQ ID NO: 250 - protein, 11 aa, Homo sapiens
RIAGLFHLIIL

SEQ ID NO: 251 - protein, 8 aa, Homo sapiens
GLFHLIIL

SEQ ID NO: 252 - protein, 11 aa, Homo sapiens
GLFHLIILPLR

SEQ ID NO: 253 - protein, 11 aa, Homo sapiens
IILPLRSMKTL

SEQ ID NO: 254 - protein, 11 aa, Homo sapiens
FPRAPCLTNRA

SEQ ID NO: 255 - protein, 11 aa, Homo sapiens
ITDFGRAKLLG

SEQ ID NO: 256 - protein, 11 aa, Homo sapiens
KITDFGRAKLL

SEQ ID NO: 257 - protein, 12 aa, Homo sapiens
KITDFGRAKLLG

SEQ ID NO: 258 - protein, 11 aa, Homo sapiens
VDSTPPPGTRF

SEQ ID NO: 259 - protein, 11 aa, Homo sapiens
PPGTRFRAMAI

SEQ ID NO: 260 - protein, 11 aa, Homo sapiens
TPPPGTRFRAM

SEQ ID NO: 261 - protein, 16 aa, Homo sapiens
VDSTPPPGTRFRAMAI

SEQ ID NO: 262 - protein, 11 aa, Homo sapiens
KIGDFGLATEK

SEQ ID NO: 263 - protein, 16 aa, Homo sapiens
KIGDFGLATEKSRWSG

SEQ ID NO: 264 - protein, 10 aa, Homo sapiens
LATEKSRWSG

SEQ ID NO: 265 - protein, 11 aa, Homo sapiens
KIGDFGLATKK

SEQ ID NO: 266 - protein, 11 aa, Homo sapiens
KIGDFGLATMK

SEQ ID NO: 267 - protein, 11 aa, Homo sapiens
CLLDILDTAGK

SEQ ID NO: 268 - protein, 15 aa, Homo sapiens
CLLDILDTAGKEEYS

SEQ ID NO: 269 - protein, 11 aa, Homo sapiens
ILDTAGLEEYS

SEQ ID NO: 270 - protein, 11 aa, Homo sapiens
ILDTAGKEEYS

SEQ ID NO: 271 - protein, 10 aa, Homo sapiens
ILDTAGKEEY

SEQ ID NO: 272 - protein, 10 aa, Homo sapiens
ILDTAGREEY

SEQ ID NO: 273 - protein, 11 aa, Homo sapiens
FLDLPVAIINL

SEQ ID NO: 274 - protein, 15 aa, Homo sapiens
VTFLDLPVAIINLKE

SEQ ID NO: 275 - protein, 10 aa, Homo sapiens
YQDPAAVFFV

SEQ ID NO: 276 - protein, 11 aa, Homo sapiens
VLHECNSSYIV

SEQ ID NO: 277 - protein, 15 aa, Homo sapiens
LQVLHECNSSYIVGF

SEQ ID NO: 278 - protein, 9 aa, Homo sapiens
ATFDLFLFV

SEQ ID NO: 279 - protein, 15 aa, Homo sapiens
AGATFDLFLFVAQKM

SEQ ID NO: 280 - protein, 9 aa, Homo sapiens
ILTDWTIFL

SEQ ID NO: 281 - protein, 15 aa, Homo sapiens
SSNILTDWTIFLLDT

SEQ ID NO: 282 - protein, 9 aa, Homo sapiens
KVAELVYFL

SEQ ID NO: 283 - protein, 11 aa, Homo sapiens
KVAELVYFLLL

SEQ ID NO: 284 - protein, 9 aa, Homo sapiens
ALKEMVVFL

SEQ ID NO: 285 - protein, 11 aa, Homo sapiens
ALKEMVVFLLL

SEQ ID NO: 286 - protein, 15 aa, Homo sapiens
HIAALKEMVVFLLLY

SEQ ID NO: 287 - protein, 9 aa, Homo sapiens
FLADQLDLI

SEQ ID NO: 288 - protein, 11 aa, Homo sapiens
FLADQLDLIYV

SEQ ID NO: 289 - protein, 15 aa, Homo sapiens
DSFLADQLDLIYVAY

SEQ ID NO: 290 - protein, 10 aa, Homo sapiens
FLFAAENEQM

SEQ ID NO: 291 - protein, 11 aa, Homo sapiens
FLFAAENEQML

SEQ ID NO: 292 - protein, 9 aa, Homo sapiens
KLVFGSVFL

SEQ ID NO: 293 - protein, 9 aa, Homo sapiens
LVFGSVFLF

SEQ ID NO: 294 - protein, 11 aa, Homo sapiens
KLVFGSVFLFA

SEQ ID NO: 295 - protein, 15 aa, Homo sapiens
LFKLVFGSVFLFAAE

SEQ ID NO: 296 - protein, 9 aa, Homo sapiens
FSGEYIPTV

SEQ ID NO: 297 - protein, 11 aa, Homo sapiens
NAFSGEYIPTV

SEQ ID NO: 298 - protein, 15 aa, Homo sapiens
TTNAFSGEYIPTVFD

SEQ ID NO: 299 - protein, 10 aa, Homo sapiens
NAFSGEYIPT

SEQ ID NO: 300 - protein, 11 aa, Homo sapiens
GLFYYRLLLVV

SEQ ID NO: 301 - protein, 15 aa, Homo sapiens
DRGLFYYRLLLVVID

SEQ ID NO: 302 - protein, 9 aa, Homo sapiens
KVSLEAFFV

SEQ ID NO: 303 - protein, 9 aa, Homo sapiens
LIDQVVQTV

SEQ ID NO: 304 - protein, 15 aa, Homo sapiens
RRPLIDQVVQTVLSE

SEQ ID NO: 305 - protein, 9 aa, Homo sapiens
RLDVLYLHL

SEQ ID NO: 306 - protein, 9 aa, Homo sapiens
VLDILAVLV

SEQ ID NO: 307 - protein, 11 aa, Homo sapiens
VLDILAVLVRL

SEQ ID NO: 308 - protein, 15 aa, Homo sapiens
PAVVLDILAVLVRLA

SEQ ID NO: 309 - protein, 9 aa, Homo sapiens
YLFDGQALL

SEQ ID NO: 310 - protein, 11 aa, Homo sapiens
YLFDGQALLHK

SEQ ID NO: 311 - protein, 15 aa, Homo sapiens
EPFYLFDGQALLHKF

SEQ ID NO: 312 - protein, 11 aa, Homo sapiens
FLLESFGFQIV

SEQ ID NO: 313 - protein, 15 aa, Homo sapiens
NMFLLESFGFQIVQS

SEQ ID NO: 314 - protein, 11 aa, Homo sapiens
STRDPLSEITK

SEQ ID NO: 315 - protein, 11 aa, Homo sapiens
LQLDEQTGEFL

SEQ ID NO: 316 - protein, 11 aa, Homo sapiens
RLFEYLRLTSL

SEQ ID NO: 317 - protein, 13 aa, Homo sapiens
TRLFEYLRLTSLG

SEQ ID NO: 318 - RNA, 132 nt, synthetic construct
  source 1..132: mol_type = other RNA
atgtacgtgagcaaccgcgaggtccaggtgacctccatcaaggagaccttcctgagcatgggccccgcctccgggctcatgctgggctgcagccggcaggagatggggaagctggagaagcgcacggtgtga

SEQ ID NO: 319 - RNA, 45 nt, synthetic construct
  source 1..45: mol_type = other RNA
cgcgaggtgcaggtcaccagcatcaaggagaccttcctgtccatg

SEQ ID NO: 320 - RNA, 45 nt, synthetic construct
  source 1..45: mol_type = other RNA
atgtacgtgagcaaccgcgaggtccaggtgacctccatcaaggag

SEQ ID NO: 321 - RNA, 45 nt, synthetic construct
  source 1..45: mol_type = other RNA
accttcctgagcatgggccccgcctccgggctcatgctgggctgc

SEQ ID NO: 322 - RNA, 51 nt, synthetic construct
  source 1..51: mol_type = other RNA
atcaaggagaccttcctgagcatgggccccgcctccgggctcatgctgggc

SEQ ID NO: 323 - RNA, 45 nt, synthetic construct
  source 1..45: mol_type = other RNA
atgctgggctgcagccgccaggagatggggaagctcgagaagcgg

SEQ ID NO: 324 - RNA, 45 nt, synthetic construct
  source 1..45: mol_type = other RNA
ggctgcagccgccaggagatggggaagctggagaagcggaccgtg

SEQ ID NO: 325 - RNA, 42 nt, synthetic construct
  source 1..42: mol_type = other RNA
gaggtgcaggtcaccagcatcaaggagaccttcctgtccatg

SEQ ID NO: 326 - RNA, 24 nt, synthetic construct
  source 1..24: mol_type = other RNA
atgtacgtgagcaaccgcgaggtc

SEQ ID NO: 327 - RNA, 30 nt, synthetic construct
  source 1..30: mol_type = other RNA
atgtacgtgagcaaccgcgaggtccaggtg

SEQ ID NO: 328 - RNA, 30 nt, synthetic construct
  source 1..30: mol_type = other RNA
accagcatcaaggagaccttcctgtccatg

SEQ ID NO: 329 - RNA, 30 nt, synthetic construct
  source 1..30: mol_type = other RNA
ctgagcatgggccccgcctccgggctcatg

SEQ ID NO: 330 - RNA, 30 nt, synthetic construct
  source 1..30: mol_type = other RNA
agcatgggccccgcctccgggctgatgctc

SEQ ID NO: 331 - RNA, 33 nt, synthetic construct
  source 1..33: mol_type = other RNA
agcaaccgcgaggtgcaggtcacctccatcaag

SEQ ID NO: 332 - RNA, 33 nt, synthetic construct
  source 1..33: mol_type = other RNA
gaggtgcaggtcaccagcatcaaggagaccttc

SEQ ID NO: 333 - RNA, 33 nt, synthetic construct
  source 1..33: mol_type = other RNA
gtgaccagcatcaaggagaccttcctgtccatg

SEQ ID NO: 334 - RNA, 33 nt, synthetic construct
  source 1..33: mol_type = other RNA
accttcctgagcatgggccccgcctccgggctc

SEQ ID NO: 335 - RNA, 33 nt, synthetic construct
  source 1..33: mol_type = other RNA
ttcctgagcatgggccccgcctccgggctcatg

SEQ ID NO: 336 - RNA, 33 nt, synthetic construct
  source 1..33: mol_type = other RNA
ctgagcatgggccccgcctccgggctcatgctg

SEQ ID NO: 337 - RNA, 33 nt, synthetic construct
  source 1..33: mol_type = other RNA
agcatgggccccgcctccgggctgatgctcggc

SEQ ID NO: 338 - RNA, 54 nt, synthetic construct
  source 1..54: mol_type = other RNA
atgctgggctgctcccgccaggagatggggaagctggagaagcggaccgtctga

SEQ ID NO: 339 - RNA, 45 nt, synthetic construct; identical to SEQ ID NO: 323
  source 1..45: mol_type = other RNA

SEQ ID NO: 340 - RNA, 45 nt, synthetic construct; identical to SEQ ID NO: 324
  source 1..45: mol_type = other RNA

SEQ ID NO: 341 - RNA, 120 nt, synthetic construct
  source 1..120: mol_type = other RNA
atggccctgtgccgctgcgaccccgagctctgctggatcagctgcgaggagaactccaaggagtggatgaacgcctggaagctgaccgcgggcagccacaaggccatgacccggaagtga

SEQ ID NO: 342 - RNA, 45 nt, synthetic construct
  source 1..45: mol_type = other RNA
aaggagtggatgaacgcctggaagctgaccgccggcagccacaag

SEQ ID NO: 343 - RNA, 45 nt, synthetic construct
  source 1..45: mol_type = other RNA
gcctggaagctgaccgccggcagccacaaggcgatgacccgcaag

SEQ ID NO: 344 - RNA, 45 nt, synthetic construct
  source 1..45: mol_type = other RNA
cgctgcgaccccgagctgtgctggatcagctgcgaggagaactcc

SEQ ID NO: 345 - RNA, 45 nt, synthetic construct
  source 1..45: mol_type = other RNA
tggatcagctgcgaggagaactccaaggagtggatgaacgcctgg

SEQ ID NO: 346 - RNA, 24 nt, synthetic construct
  source 1..24: mol_type = other RNA
gagtggatgaacgcctggaagctg

SEQ ID NO: 347 - RNA, 24 nt, synthetic construct
  source 1..24: mol_type = other RNA
atgaacgcctggaagctgaccgcc

SEQ ID NO: 348 - RNA, 27 nt, synthetic construct
  source 1..27: mol_type = other RNA
cgctgcgaccccgagctgtgctggatc

SEQ ID NO: 349 - RNA, 27 nt, synthetic construct
  source 1..27: mol_type = other RNA
tggatgaacgcctggaagctgaccgcc

SEQ ID NO: 350 - RNA, 30 nt, synthetic construct
  source 1..30: mol_type = other RNA
cgctgcgaccccgagctgtgctggatcagc

SEQ ID NO: 351 - RNA, 30 nt, synthetic construct
  source 1..30: mol_type = other RNA
agctgcgaggagaactccaaggagtggatg

SEQ ID NO: 352 - RNA, 33 nt, synthetic construct
  source 1..33: mol_type = other RNA
cgctgcgaccccgagctgtgctggatcagctgc

SEQ ID NO: 353 - RNA, 33 nt, synthetic construct
  source 1..33: mol_type = other RNA
aaggagtggatgaacgcctggaagctgaccgcc

SEQ ID NO: 354 - RNA, 33 nt, synthetic construct
  source 1..33: mol_type = other RNA
aacgcctggaagctgaccgccggcagccacaag

SEQ ID NO: 355 - RNA, 33 nt, synthetic construct
  source 1..33: mol_type = other RNA
ctgaccgccggcagccacaaggccatgacccgc

SEQ ID NO: 356 - RNA, 81 nt, synthetic construct
  source 1..81: mol_type = other RNA
atgtgcgtgcacgcctacctgagcggccagcccctcgaggccaccaccctgtgccagctgcactccacgtgccacctctga

SEQ ID NO: 357 - RNA, 48 nt, synthetic construct
  source 1..48: mol_type = other RNA
gcctacctgagcggccagcccctcgaggccaccaccctgtgccagctg

SEQ ID NO: 358 - RNA, 45 nt, synthetic construct
  source 1..45: mol_type = other RNA
tgcgtgcacgcctacctgagcggccagcccctcgaggccaccacc

SEQ ID NO: 359 - RNA, 48 nt, synthetic construct
  source 1..48: mol_type = other RNA
cacgcctacctgagcggccagcccctcgaggccaccaccctgtgccag

SEQ ID NO: 360 - RNA, 30 nt, synthetic construct
  source 1..30: mol_type = other RNA
tgcgtgcacgcctacctgagcggccagccc

SEQ ID NO: 361 - RNA, 30 nt, synthetic construct
  source 1..30: mol_type = other RNA
tacctgagcggccagcccctcgaggccacc

SEQ ID NO: 362 - RNA, 33 nt, synthetic construct
  source 1..33: mol_type = other RNA
tgcgtgcacgcctacctgagcggccagcccctc

SEQ ID NO: 363 - RNA, 222 nt, synthetic construct
  source 1..222: mol_type = other RNA
atggacgccaccgtgctgaacagcgaccagaactccggcgagatgctccgccggaacagcgcctccaccgagcgcgtcagcgaggggttcaaggactccatcaccgcgctgagctaccacttctacaagaaccacatccacttcttcccgcccacgtgccccttctccctgagcggcacccaggccctcgtgctgtacatccaggtgatctccaagggctga

SEQ ID NO: 364 - RNA, 48 nt, synthetic construct
  source 1..48: mol_type = other RNA
agcatcaccgccctgtcctaccacttctacaagaaccacatccacttc

SEQ ID NO: 365 - RNA, 51 nt, synthetic construct
  source 1..51: mol_type = other RNA
agcctgtccggcacccaggccctcgtgctgtacatccaggtcatcagcaag

SEQ ID NO: 366 - RNA, 51 nt, synthetic construct
  source 1..51: mol_type = other RNA
gtgctgaacagcgaccagaactccggcgagatgctccgccggaacagcgcc

SEQ ID NO: 367 - RNA, 45 nt, synthetic construct
  source 1..45: mol_type = other RNA
gccagcaccgagcgcgtgtccgagggcttcaaggacagcatcacc

SEQ ID NO: 368 - RNA, 51 nt, synthetic construct
  source 1..51: mol_type = other RNA
gccctgagctaccacttctacaagaaccacatccacttcttcccgcccacc

SEQ ID NO: 369 - RNA, 51 nt, synthetic construct
  source 1..51: mol_type = other RNA
cacttcttccccccgacctgccccttcagcctgtccggcacccaggccctc

SEQ ID NO: 370 - RNA, 33 nt, synthetic construct
  source 1..33: mol_type = other RNA
gccctggtgctctacatccaggtcatcagcaag

SEQ ID NO: 371 - RNA, 24 nt, synthetic construct
  source 1..24: mol_type = other RNA
ttcaaggacagcatcaccgccctg

SEQ ID NO: 372 - RNA, 24 nt, synthetic construct
  source 1..24: mol_type = other RNA
gccctgagctaccacttctacaag

SEQ ID NO: 373 - RNA, 24 nt, synthetic construct
  source 1..24: mol_type = other RNA
ttctacaagaaccacatccacttc

SEQ ID NO: 374 - RNA, 24 nt, synthetic construct
  source 1..24: mol_type = other RNA
tacaagaaccacatccacttcttc

SEQ ID NO: 375 - RNA, 24 nt, synthetic construct
  source 1..24: mol_type = other RNA
acccaggccctggtgctctacatc

SEQ ID NO: 376 - RNA, 24 nt, synthetic construct
  source 1..24: mol_type = other RNA
gccctggtgctctacatccaggtc

SEQ ID NO: 377 - RNA, 27 nt, synthetic construct
  source 1..27: mol_type = other RNA
aacagcgaccagaactccggcgagatg

SEQ ID NO: 378 - RNA, 27 nt, synthetic construct
  source 1..27: mol_type = other RNA
ggcttcaaggacagcatcaccgccctg

SEQ ID NO: 379 - RNA, 27 nt, synthetic construct
  source 1..27: mol_type = other RNA
accgccctgagctaccacttctacaag

SEQ ID NO: 380 - RNA, 27 nt, synthetic construct
  source 1..27: mol_type = other RNA
agctaccacttctacaagaaccacatc

SEQ ID NO: 381 - RNA, 27 nt, synthetic construct
  source 1..27: mol_type = other RNA
cacttctacaagaaccacatccacttc

SEQ ID NO: 382 - RNA, 27 nt, synthetic construct
  source 1..27: mol_type = other RNA
ttctacaagaaccacatccacttcttc

SEQ ID NO: 383 - RNA, 27 nt, synthetic construct
  source 1..27: mol_type = other RNA
ttccccccgacctgccccttcagcctg

SEQ ID NO: 384 - RNA, 27 nt, synthetic construct
  source 1..27: mol_type = other RNA
ggcacccaggccctggtgctctacatc

SEQ ID NO: 385 - RNA, 27 nt, synthetic construct
  source 1..27: mol_type = other RNA
caggccctggtgctctacatccaggtc

SEQ ID NO: 386 - RNA, 27 nt, synthetic construct
  source 1..27: mol_type = other RNA
gccctggtgctctacatccaggtcatc

SEQ ID NO: 387 - RNA, 30 nt, synthetic construct
  source 1..30: mol_type = other RNA
aacagcgaccagaactccggcgagatgctg

SEQ ID NO: 388 - RNA, 30 nt, synthetic construct
  source 1..30: mol_type = other RNA
agcggcgagatgctgcgccggaactccgcc

SEQ ID NO: 389 - RNA, 30 nt, synthetic construct
  source 1..30: mol_type = other RNA
agcaccgagcgcgtgtccgagggcttcaag

SEQ ID NO: 390 - RNA, 30 nt, synthetic construct
  source 1..30: mol_type = other RNA
agcatcaccgccctgtcctaccacttctac

SEQ ID NO: 391 - RNA, 30 nt, synthetic construct
  source 1..30: mol_type = other RNA
atcaccgccctgagctaccacttctacaag

SEQ ID NO: 392 - RNA, 30 nt, synthetic construct
  source 1..30: mol_type = other RNA
ctgagctaccacttctacaagaaccacatc

SEQ ID NO: 393 - RNA, 30 nt, synthetic construct
  source 1..30: mol_type = other RNA
agctaccacttctacaagaaccacatccac

SEQ ID NO: 394 - RNA, 30 nt, synthetic construct
  source 1..30: mol_type = other RNA
taccacttctacaagaaccacatccacttc

SEQ ID NO: 395 - RNA, 30 nt, synthetic construct
  source 1..30: mol_type = other RNA
cacttctacaagaaccacatccacttcttc

SEQ ID NO: 396 - RNA, 30 nt, synthetic construct
  source 1..30: mol_type = other RNA
tacaagaaccacatccacttcttcccgccc

SEQ ID NO: 397 - RNA, 30 nt, synthetic construct
  source 1..30: mol_type = other RNA
ttccccccgacctgccccttcagcctgtcc

SEQ ID NO: 398 - RNA, 30 nt, synthetic construct
  source 1..30: mol_type = other RNA
ttcagcctgtccggcacccaggccctcgtg

SEQ ID NO: 399 - RNA, 30 nt, synthetic construct
  source 1..30: mol_type = other RNA
agcggcacccaggccctggtgctctacatc

SEQ ID NO: 400 - RNA, 30 nt, synthetic construct
  source 1..30: mol_type = other RNA
acccaggccctggtgctctacatccaggtc

SEQ ID NO: 401 - RNA, 30 nt, synthetic construct
  source 1..30: mol_type = other RNA
caggccctggtgctctacatccaggtcatc

SEQ ID NO: 402 - RNA, 30 nt, synthetic construct
  source 1..30: mol_type = other RNA
ctggtgctctacatccaggtcatcagcaag

SEQ ID NO: 403 - RNA, 30 nt, synthetic construct
  source 1..30: mol_type = other RNA
gtgctgtacatccaggtcatcagcaagggc

SEQ ID NO: 404 - RNA, 33 nt, synthetic construct
  source 1..33: mol_type = other RNA
gtgctgaacagcgaccagaactccggcgagatg

SEQ ID NO: 405 - RNA, 33 nt, synthetic construct
  source 1..33: mol_type = other RNA
aacagcggcgagatgctgcgccggaactccgcc

SEQ ID NO: 406 - RNA, 33 nt, synthetic construct
  source 1..33: mol_type = other RNA
gccagcaccgagcgcgtgtccgagggcttcaag

SEQ ID NO: 407 - RNA, 45 nt, synthetic construct
  source 1..45: mol_type = other RNA
ggcttcaaggacagcatcaccgccctgtcctaccacttctacaag

SEQ ID NO: 408 - RNA, 33 nt, synthetic construct
  source 1..33: mol_type = other RNA
ggcttcaaggacagcatcaccgccctgtcctac

SEQ ID NO: 409 - RNA, 33 nt, synthetic construct
  source 1..33: mol_type = other RNA
gacagcatcaccgccctgtcctaccacttctac

SEQ ID NO: 410 - RNA, 33 nt, synthetic construct
  source 1..33: mol_type = other RNA
agcatcaccgccctgtcctaccacttctacaag

SEQ ID NO: 411 - RNA, 33 nt, synthetic construct
  source 1..33: mol_type = other RNA
gccctgagctaccacttctacaagaaccacatc

SEQ ID NO: 412 - RNA, 33 nt, synthetic construct
  source 1..33: mol_type = other RNA
agctaccacttctacaagaaccacatccacttc

SEQ ID NO: 413 - RNA, 33 nt, synthetic construct
  source 1..33: mol_type = other RNA
taccacttctacaagaaccacatccacttcttc

SEQ ID NO: 414 - RNA, 33 nt, synthetic construct
  source 1..33: mol_type = other RNA
tacaagaaccacatccacttcttcccgcccacc

SEQ ID NO: 415 - RNA, 33 nt, synthetic construct
  source 1..33: mol_type = other RNA
cacttcttccccccgacctgccccttcagcctg

SEQ ID NO: 416 - RNA, 33 nt, synthetic construct
  source 1..33: mol_type = other RNA
ttccccccgacctgccccttcagcctgtccggc

SEQ ID NO: 417 - RNA, 33 nt, synthetic construct
  source 1..33: mol_type = other RNA
tgccccttcagcctgtccggcacccaggccctc

SEQ ID NO: 418 - RNA, 33 nt, synthetic construct
  source 1..33: mol_type = other RNA
ttcagcctgtccggcacccaggccctcgtgctg

SEQ ID NO: 419 - RNA, 33 nt, synthetic construct
  source 1..33: mol_type = other RNA
ggcacccaggccctggtgctctacatccaggtc

SEQ ID NO: 420 - RNA, 33 nt, synthetic construct
  source 1..33: mol_type = other RNA
acccaggccctggtgctctacatccaggtcatc

SEQ ID NO: 421 - RNA, 198 nt, synthetic construct
  source 1..198: mol_type = other RNA
atgaacgtgcccaagggccagaccgggaacagctcccgcggcccgggggacggcgggaacaaggaccactggaaggagagcgaccggaaggacggcaagaaggaccaggactcccgcagcgcccccgagcccaagaagcccgaggagaacccggcctccaagttcagctccgcgagcaactacgccgccctgtcctga

SEQ ID NO: 422 - RNA, 45 nt, synthetic construct
  source 1..45: mol_type = other RNA
aacagctcccgcggccccggggacggcgggaacaaggaccactgg

SEQ ID NO: 423 - RNA, 45 nt, synthetic construct
  source 1..45: mol_type = other RNA
gaccaggacagccgctccgcccccgagccgaagaagcccgaggag

SEQ ID NO: 424 - RNA, 45 nt, synthetic construct
  source 1..45: mol_type = other RNA
cccgccagcaagttctccagcgcctccaactacgcggccctgagc

SEQ ID NO: 425 - RNA, 33 nt, synthetic construct
  source 1..33: mol_type = other RNA
agctcccgcggccccggggacggcgggaacaag

SEQ ID NO: 426 - RNA, 33 nt, synthetic construct
  source 1..33: mol_type = other RNA
ggccccggggacggcgggaacaaggaccactgg

SEQ ID NO: 427 - RNA, 33 nt, synthetic construct
  source 1..33: mol_type = other RNA
gaccaggacagccgctccgcccccgagccgaag

SEQ ID NO: 428 - RNA, 33 nt, synthetic construct
  source 1..33: mol_type = other RNA
agccgctccgcccccgagccgaagaagcccgag

SEQ ID NO: 429 - RNA, 33 nt, synthetic construct
  source 1..33: mol_type = other RNA
aagttcagctccgccagcaactacgccgcgctg

SEQ ID NO: 430 - RNA, 24 nt, synthetic construct
  source 1..24: mol_type = other RNA
agcgcctccaactacgccgcgctg

SEQ ID NO: 431 - RNA, 27 nt, synthetic construct
  source 1..27: mol_type = other RNA
agctccgccagcaactacgccgcgctg

SEQ ID NO: 432 - RNA, 204 nt, synthetic construct
  source 1..204: mol_type = other RNA
atgaagaccgaggtgaagaacgaccacgtcctgttctacctcgagaacggcttcgggcgcgccgacagcttccccttctccgtggagcagagcaacctggtgttcaacatccagccggcccccgcgatggtctacgactactacgagaaggaggagtacgccctggccttctacaacatcgactccagctccgtgagccagtga

SEQ ID NO: 433 - RNA, 45 nt, synthetic construct
  source 1..45: mol_type = other RNA
agcttccccttctccgtggagcagagcaacctggtcttcaacatc

SEQ ID NO: 434 - RNA, 48 nt, synthetic construct
  source 1..48: mol_type = other RNA
aaggaggagtacgccctggccttctacaacatcgacagctccagcgtg

SEQ ID NO: 435 - RNA, 27 nt, synthetic construct
  source 1..27: mol_type = other RNA
ttctacaacatcgacagctccagcgtg

SEQ ID NO: 436 - RNA, 33 nt, synthetic construct
  source 1..33: mol_type = other RNA
aaggaggagtacgccctggccttctacaacatc

SEQ ID NO: 437 - RNA, 33 nt, synthetic construct
  source 1..33: mol_type = other RNA
ttccccttcagcgtggagcagtccaacctggtc

SEQ ID NO: 438 - RNA, 198 nt, synthetic construct
  source 1..198: mol_type = other RNA
atgatcctgcgcatcgccggcctcttccacctgatcatcctgcccctccggagcatgaagaccctgcgccacgtggaggggcagatggagaactccatgccgacctgcttcgccctgtggatgtgctggatgttcccccgggcgccctgcctcacgaaccgcgccctgagcaagccctactcctgctggaagcggtga

SEQ ID NO: 439 - RNA, 45 nt, synthetic construct
  source 1..45: mol_type = other RNA
atgatcctgcgcatcgccggcctcttccacctgatcatcctgccc

SEQ ID NO: 440 - RNA, 45 nt, synthetic construct
  source 1..45: mol_type = other RNA
cgcatcgccggcctgttccacctcatcatcctgcccctgcggagc

SEQ ID NO: 441 - RNA, 45 nt, synthetic construct
  source 1..45: mol_type = other RNA
ttccacctgatcatcctccccctgcgcagcatgaagaccctgcgg

SEQ ID NO: 442 - RNA, 45 nt, synthetic construct
  source 1..45: mol_type = other RNA
atgttcccccgcgccccgtgcctgaccaaccgggccctcagcaag

SEQ ID NO: 443 - RNA, 33 nt, synthetic construct
  source 1..33: mol_type = other RNA
atgatcctgcgcatcgccggcctcttccacctg

SEQ ID NO: 444 - RNA, 33 nt, synthetic construct
  source 1..33: mol_type = other RNA
cgcatcgccggcctgttccacctcatcatcctg

SEQ ID NO: 445 - RNA, 24 nt, synthetic construct
  source 1..24: mol_type = other RNA
ggcctgttccacctcatcatcctg

SEQ ID NO: 446 - RNA, 33 nt, synthetic construct
  source 1..33: mol_type = other RNA
ggcctgttccacctcatcatcctgcccctgcgc

SEQ ID NO: 447 - RNA, 33 nt, synthetic construct
  source 1..33: mol_type = other RNA
atcatcctgcccctccgcagcatgaagaccctg

SEQ ID NO: 448 - RNA, 33 nt, synthetic construct
  source 1..33: mol_type = other RNA
ttcccccgcgccccgtgcctgaccaaccgggcc

SEQ ID NO: 449 - RNA, 33 nt, synthetic construct
  source 1..33: mol_type = other RNA
atcaccgacttcggccgcgccaagctgctcggg

SEQ ID NO: 450 - RNA, 33 nt, synthetic construct
  source 1..33: mol_type = other RNA
aagatcaccgacttcggccgcgccaagctgctc

SEQ ID NO: 451 - RNA, 36 nt, synthetic construct
  source 1..36: mol_type = other RNA
aagatcaccgacttcggccgcgccaagctgctcggg

SEQ ID NO: 452 - RNA, 33 nt, synthetic construct
  source 1..33: mol_type = other RNA
gtggacagcacccccccgcccggcacccgcttc

SEQ ID NO: 453 - RNA, 33 nt, synthetic construct
  source 1..33: mol_type = other RNA
cccccgggcacccgcttccgggccatggccatc

SEQ ID NO: 454 - RNA, 33 nt, synthetic construct
  source 1..33: mol_type = other RNA
acccccccgcccggcacccgcttccgggccatg

SEQ ID NO: 455 - RNA, 48 nt, synthetic construct
  source 1..48: mol_type = other RNA
gtggacagcacccccccgcccggcacccgcttccgggccatggccatc

SEQ ID NO: 456 - RNA, 33 nt, synthetic construct
  source 1..33: mol_type = other RNA
aagatcggcgacttcgggctggccaccgagaag

SEQ ID NO: 457 - RNA, 48 nt, synthetic construct
  source 1..48: mol_type = other RNA
aagatcggcgacttcgggctggccaccgagaagtcccgctggagcggc

SEQ ID NO: 458 - RNA, 30 nt, synthetic construct
  source 1..30: mol_type = other RNA
ctggccaccgagaagtcccgctggagcggc

SEQ ID NO: 459 - RNA, 33 nt, synthetic construct
  source 1..33: mol_type = other RNA
aagatcggcgacttcgggctggccaccaagaag

SEQ ID NO: 460 - RNA, 33 nt, synthetic construct
  source 1..33: mol_type = other RNA
aagatcggcgacttcgggctggccaccatgaag

SEQ ID NO: 461 - RNA, 33 nt, synthetic construct
  source 1..33: mol_type = other RNA
tgcctgctcgacatcctggacaccgccggcaag

SEQ ID NO: 462 - RNA, 45 nt, synthetic construct
  source 1..45: mol_type = other RNA
tgcctgctcgacatcctggacaccgccggcaaggaggagtacagc

SEQ ID NO: 463 - RNA, 33 nt, synthetic construct
  source 1..33: mol_type = other RNA
atcctggacaccgccggcctcgaggagtacagc

SEQ ID NO: 464 - RNA, 33 nt, synthetic construct
  source 1..33: mol_type = other RNA
atcctggacaccgccggcaaggaggagtacagc

SEQ ID NO: 465 - RNA, 30 nt, synthetic construct
  source 1..30: mol_type = other RNA
atcctggacaccgccggcaaggaggagtac

SEQ ID NO: 466 - RNA, 30 nt, synthetic construct
  source 1..30: mol_type = other RNA
atcctggacaccgccggccgcgaggagtac

SEQ ID NO: 467 - RNA, 33 nt, synthetic construct
  source 1..33: mol_type = other RNA
ttcctggacctccccgtggccatcatcaacctg

SEQ ID NO: 468 - RNA, 45 nt, synthetic construct
  source 1..45: mol_type = other RNA
gtgaccttcctggacctccccgtcgccatcatcaacctgaaggag

SEQ ID NO: 469 - RNA, 30 nt, synthetic construct
  source 1..30: mol_type = other RNA
taccaggaccccgccgccgtgttcttcgtc

SEQ ID NO: 470 - RNA, 33 nt, synthetic construct
  source 1..33: mol_type = other RNA
gtgctgcacgagtgcaacagctcctacatcgtc

SEQ ID NO: 471 - RNA, 45 nt, synthetic construct
  source 1..45: mol_type = other RNA
ctccaggtgctgcacgagtgcaacagctcctacatcgtcggcttc

SEQ ID NO: 472 - RNA, 27 nt, synthetic construct
  source 1..27: mol_type = other RNA
gccaccttcgacctgttcctcttcgtg

SEQ ID NO: 473 - RNA, 45 nt, synthetic construct
  source 1..45: mol_type = other RNA
gccggcgccaccttcgacctgttcctcttcgtggcgcagaagatg

SEQ ID NO: 474 - RNA, 27 nt, synthetic construct
  source 1..27: mol_type = other RNA
atcctgaccgactggaccatcttcctc

SEQ ID NO: 475 - RNA, 45 nt, synthetic construct
  source 1..45: mol_type = other RNA
agctccaacatcctgaccgactggaccatcttcctcctggacacg

SEQ ID NO: 476 - RNA, 27 nt, synthetic construct
  source 1..27: mol_type = other RNA
aaggtggccgagctggtctacttcctc

SEQ ID NO: 477 - RNA, 33 nt, synthetic construct
  source 1..33: mol_type = other RNA
aaggtggccgagctggtctacttcctcctgctg

SEQ ID NO: 478 - RNA, 27 nt, synthetic construct
  source 1..27: mol_type = other RNA
gccctgaaggagatggtggtcttcctc

SEQ ID NO: 479 - RNA, 33 nt, synthetic construct
  source 1..33: mol_type = other RNA
gccctgaaggagatggtggtcttcctcctgctg

SEQ ID NO: 480 - RNA, 45 nt, synthetic construct
  source 1..45: mol_type = other RNA
cacatcgccgccctgaaggagatggtggtcttcctcctgctgtac

SEQ ID NO: 481 - RNA, 27 nt, synthetic construct
  source 1..27: mol_type = other RNA
ttcctggccgaccagctcgacctgatc

SEQ ID NO: 482 - RNA, 33 nt, synthetic construct
  source 1..33: mol_type = other RNA
ttcctggccgaccagctcgacctgatctacgtg

SEQ ID NO: 483 - RNA, 45 nt, synthetic construct
  source 1..45: mol_type = other RNA
gacagcttcctggccgaccagctcgacctgatctacgtggcctac

SEQ ID NO: 484 - RNA, 30 nt, synthetic construct
  source 1..30: mol_type = other RNA
ttcctgttcgccgccgagaacgagcagatg

SEQ ID NO: 485 - RNA, 33 nt, synthetic construct
  source 1..33: mol_type = other RNA
ttcctgttcgccgccgagaacgagcagatgctc

SEQ ID NO: 486 - RNA, 27 nt, synthetic construct
  source 1..27: mol_type = other RNA
aagctggtgttcggcagcgtcttcctc

SEQ ID NO: 487 - RNA, 27 nt, synthetic construct
  source 1..27: mol_type = other RNA
ctggtgttcggcagcgtcttcctcttc

SEQ ID NO: 488 - RNA, 33 nt, synthetic construct
  source 1..33: mol_type = other RNA
aagctggtgttcggcagcgtcttcctcttcgcc

SEQ ID NO: 489 - RNA, 45 nt, synthetic construct
  source 1..45: mol_type = other RNA
ctgttcaagctcgtgttcggcagcgtcttcctgttcgccgccgag

SEQ ID NO: 490 - RNA, 27 nt, synthetic construct
  source 1..27: mol_type = other RNA
ttcagcggcgagtacatccccaccgtg

SEQ ID NO: 491 - RNA, 33 nt, synthetic construct
  source 1..33: mol_type = other RNA
aacgccttcagcggcgagtacatccccaccgtg

SEQ ID NO: 492 - RNA, 45 nt, synthetic construct
  source 1..45: mol_type = other RNA
accaccaacgccttcagcggcgagtacatccccacggtgttcgac

SEQ ID NO: 493 - RNA, 30 nt, synthetic construct
  source 1..30: mol_type = other RNA
aacgccttcagcggcgagtacatccccacc

SEQ ID NO: 494 - RNA, 33 nt, synthetic construct
  source 1..33: mol_type = other RNA
ggcctgttctactaccgcctcctgctggtggtc

SEQ ID NO: 495 - RNA, 45 nt, synthetic construct
  source 1..45: mol_type = other RNA
gaccgcggcctgttctactaccggctcctgctggtggtcatcgac

SEQ ID NO: 496 - RNA, 27 nt, synthetic construct
  source 1..27: mol_type = other RNA
aaggtgagcctggaggccttcttcgtc

SEQ ID NO: 497 - RNA, 27 nt, synthetic construct
  source 1..27: mol_type = other RNA
ctgatcgaccaggtggtccagaccgtg

SEQ ID NO: 498 - RNA, 45 nt, synthetic construct
  source 1..45: mol_type = other RNA
cgccggcccctgatcgaccaggtggtccagaccgtgctcagcgag

SEQ ID NO: 499 - RNA, 27 nt, synthetic construct
  source 1..27: mol_type = other RNA
cgcctggacgtgctctacctgcacctg

SEQ ID NO: 500 - RNA, 27 nt, synthetic construct
  source 1..27: mol_type = other RNA
gtgctggacatcctcgccgtcctggtg

SEQ ID NO: 501 - RNA, 33 nt, synthetic construct
  source 1..33: mol_type = other RNA
gtgctggacatcctcgccgtcctggtgcgcctg

SEQ ID NO: 502 - RNA, 45 nt, synthetic construct
  source 1..45: mol_type = other RNA
cccgccgtggtcctggacatcctcgccgtgctggtgcgcctggcg

SEQ ID NO: 503 - RNA, 27 nt, synthetic construct
  source 1..27: mol_type = other RNA
tacctgttcgacggccaggccctcctg

SEQ ID NO: 504 - RNA, 33 nt, synthetic construct
  source 1..33: mol_type = other RNA
tacctgttcgacggccaggccctcctgcacaag

SEQ ID NO: 505 - RNA, 45 nt, synthetic construct
  source 1..45: mol_type = other RNA
gagcccttctacctgttcgacggccaggccctcctgcacaagttc

SEQ ID NO: 506 - RNA, 33 nt, synthetic construct
  source 1..33: mol_type = other RNA
ttcctgctcgagagcttcggcttccagatcgtg

SEQ ID NO: 507 - RNA, 45 nt, synthetic construct
  source 1..45: mol_type = other RNA
aacatgttcctgctcgagagcttcggcttccagatcgtgcagtcc

SEQ ID NO: 508 - RNA, 33 nt, synthetic construct
  source 1..33: mol_type = other RNA
agcacccgcgaccccctgtccgagatcaccaag

SEQ ID NO: 509 - RNA, 33 nt, synthetic construct
  source 1..33: mol_type = other RNA
ctgcagctcgacgagcagaccggcgagttcctg

SEQ ID NO: 510 - RNA, 33 nt, synthetic construct
  source 1..33: mol_type = other RNA
cgcctgttcgagtacctccggctgaccagcctg

SEQ ID NO: 511 - RNA, 39 nt, synthetic construct
  source 1..39: mol_type = other RNA
acccgcctgttcgagtacctccggctgaccagcctgggc

SEQ ID NO: 512 - RNA, 4310 nt, Homo sapiens
  source 1..4310: mol_type = other RNA
  ncRNA 1..4310: ncRNA_class = lncRNA
actccctgaccgttgggcttcccaagtgaggcaatgcctcgccctgcttaggctcgcgcacggtgcgcgcacccactgacctgcgcccactgtctggcactccctagtgagatgaacccggtacctcagatggaagtgcagaaatcacccgtcttctgcgtcgctcacgctgggagctgtagaccggagctctccagtcagcccggtcaagcatgacagcaactcaaactcggcaaacttccaagacgtggaggacatgcctgacagatgtgccttggaagagtctgagagtccaggtgagccaaggcatcattcatggatagcaaaggtctgcccctgcaaggtgtcttaaatggaatctcatcagtaggagctgaatttggacaaattaagaaatccaaaagatgccatttgtttattactgtataaaagcattgttgttattggtcaagttattaggctgtagtggatttgctaatactttagccaacatgtattaaagtgattttaatacatgctgattacaatgcaatacatactgattgaaaatattcatattcatctaattttagaaaaatattgcctagatcattctctattcctgtttcttactttttctgttaatatttccaacagggaatgccagtccacagacacaaatttaatactgctttaaattttctcctatccttttagtccctgaattatataataaacaatgttaaaaccaatgtagtacacaatacttacttacaaatttaatactgcttcaaggtatttaatctaaaattttaccaactttgatttgtctggttaggatattttgttttagtggatatgctttaattcggatcaattactgcagtaaatctcatccctaagcatgaaatgttgtcaacaaatacccagttccatttagttatcaattagcccaaataagagatacaaagtataacagtgaccaaccttgtactgttgagttaatttgaacttctaatgacattgaggctaatgtctttagctcaaggttgatcttgttggccatatagatgtgaactagggaaggggaatcaacttacagcatatcacaattgatccttattaagtataaactcttgtaggtcttttcccagaaagaagcttgactagcaggaattctaaaactgaaatatatcaaacagcataaataggaatagacataaagtgctcttctattaaagcctttggtgatctatttactatgatttatattgtacagttcctcgatttacagaaaatcatcaaaattattaatctacatatcttatgtatataaatattgcctaatccatagaaaaaaggatataaagtattaaatatgtgatatatagctatatctatctatctatgtatctaatagggaagtccaagtcacttcaattgaagaaacatatctctgagcataggagcagcctcaggtcctatggtgggatgcagtggacaggagagggggaaattagaaaagagaactatataattgaaaaagggatataaagcattaaatatatgatatatagctatatctatgtatgtatctaacagagaagttcaagtcacttcaattaaagaaacatttttgagcatgggaccagcctcaggtcttatgctgggatgcagtagacaggagatggggaaattagaaaagagaactgtgtaattgaaatgacgtgggctgcacccttaaggaacttataattaatgatgatctgaataaacataccaggataaagatgtcaaatgagtgtgactcccttaaagtagattaaagtgtgcattctttgtttcctaaaatatgattttactgcttgaaattacatttgagttgaagtttagaaactaacatagcattaatatgaataatcatggaaaattattatcctttgaaaactgattgataaatatattccccctcctttagaaacagtcaaaagccacttcaaacaagtttcaaaataaaggaaggtagcaagttaggcgatggattatattttcttgcttgttgtataccagttgtcaaggacattataaggactcccaaaagcattttgaaggatggcaatatcaaataagtgtatgtcctctcaaatgaggcatttttaattgttaaaatctatttggacgctcaggttatgatatgtttatgaaaaataagcttcattatttttatagctacatcctattattcccttttagaaacaagaataacaataggttttaatagttgccatacttagcatttatcagttctaatgaaaccaatattgaatctctgataaatattttctgatgttactagctatgggaaattagaactggcacaaccctgacattactaagtggaaatgttaggatttttcggcatcgcatgttagaatctctaaaatttaaacattcctgttaaatgactaaggtttgcttttatcaatatgaattctgaaggccaatatcataccattaactatgaaagcttttaattcctaaaaatagttttagagatattcaagcaatgctctcctaatatccatacgcaagtgtgtttatgacacaaattcactagtctgtttaaaaatgaattctttatattgactggtgttccacatatttcagtaatttctgttatgagaggacttgaaatagcaaattgccacacagttaactggatagaccacgtacgtggtgatcataaccacttggtactacacccagaaactcaaaattgtctttctcctgatgagatatgggtgtccttttgtacgtctaggcctaggtaaccagtggagtgattatattagcaaatgtgtttgtatccggagtcttcctgtcattgtaataaaaaatttatttaaaaattttgtcttgtgtttcttttttggtaatcatcttggtgatatttatgaatgtatttgtgtgtgtgtgtgtgtgtgtatacatacatatatatgtgtgtatatatatacacacatatatatacacatatatatgtgtgtatatatatatatatatggcatatcccccaaaggaatatcatattttcatttgcaagccttgattctttgacttgctgttgagtttcacattggctccattgtatattgcaggaaaaagtacctggaaagcctggagggttaaaagcagaggctcctcggcagcgtgtccaggccgtgtgtgcttgctgtgcttgtcagagcctcattccctcccctgtgttggagagctttctcacagggctcatccagaggtgctagaaagcaatgtgagcactattaaacacttcaaatataaggaaaaatgtataatgttagaaacacacagttaaaaagtagccccaggtatcaaacattctgttactattttagaaactaaattaaggctgctttgaacatgatggtattagaaaaatggtagaatacagtttgttaaatattaatattctatctttagatgatagcatttggtgacttttatttcttttctcatacattttttccagtgttctgtatttttaaaatcctttcataattattataatgaacactttagaaaagatactatttctcttccatccttagagggcacattataataattgagtattttaaaattaaattttatgccatgtatttaaaatgaaagttcaggttacattaattccaactgattttaaagccttattttcacagagtggactcattcaattttttaaattttatattcatacattttgattgctcagttatataaaagcggtgttatagactcctttattctccacaaaatgttgtcttatatgtaaagtgccaaaagttgtgtttaatttcctaagaaagtcattgctttgtaaacatttgcataatacttagtgttttttgtaattttaaaaaattagcttaaccaaaagagttgcaaaattaaaacttacttgagtaggtatctttcagacaaagctgcatggcttcatgtacaatagtccccagatcacctcgcactgtagtttgctcaagaaggagcctacagatggcaggtaggagaaggtaaactggaaattaacaggtctgtctgtaagagcatgccactggaaacacccatccaatttgggacccctggaagacagcatgtgtctaatgggaggcctgtcctagtacaactcctttactcttttcaccttcatcttgaccttctcagataccatctttatgtaaaatgttgatgttgtggagtttttgcagtaatttaatttttataaatattgtattaaaatatatttatcttga

SEQ ID NO: 513 - RNA, 1768 nt, Homo sapiens
  source 1..1768: mol_type = other RNA
  ncRNA 1..1768: ncRNA_class = lncRNA
ggcgggcgcccctccccccgcctcgctgccgccttgcagtttgatctcagactgctgtgctagcaatcagcgagactccgtgggcgtaggaccctccgagccagagatgggatcttactacattgcccaggctgaagtgcagtggctattaacagctgtaatcataatgcattgcagctttgaattcctgggcttgagcgacctccccaccttagcctcctgagtagctgagactacaggcactgtcctaggtactgtggggaccaaaacattcagacctgcttctaaggagtgcagtccatggaagagtcagaataacaaatcaacatttgtactacaccagaccagctctcttctaaggttgctatgacttcccagcaggcattctaatatatgcattcttcataacaactgtgtgaaacatattaatatcctcattgttaacgatgaagaaactaaatctcaggaagaaaaactaagtgaagacaaaagaaggatttgaactgaggtttgtcagactctcgggaccatgctgttgaaaccactaaaccacgctgcctctgggtcacttgattattctgggagattctgggagtcttttctccaacactccagtggcatctgactatactgatggccctttgcaggtgtgaccctgagctctgctggatttcttgtgaagaaaactccaaggaatggatgaatgcctggaaacttactgctggaagtcataaggcaatgacaagaaaatagcatatcattggaagacttgacccatgggatctataaattcctagggaagctccttactataatttgtttggtttctatggaatccaacaagaaaagaaatagcgaaacatgtgggttgtctcctctttggcactaagcctgggagaatatgaagatatctgaaaacaaagaggcttccttcaggtgttcaggagggggctaacactgcctacttgtatgtcaagggcccatgggagtagacactcccagaatgctaccacttggattctccagggatcatatgtgataccaatcagctgacaaagaaaataacaacccatctaaggggattcagcagctagaaagaaaatgagtatgccaagcaaactgaatacacgaccagcaaaatagaacttcaaaaggagacaaaatacaacattctctctctctctgtgtgtgtgtgtgtgtgtgtgtgtgtgtgtgtttgcatgtaaataacaaaattcaagttctggacacaatttttagagctaaacacatgaccttccaactaaactttttattagatatcaaagaagctggtcactgttgaggtgaagatcaagtgcaagatacattttgaggcacagagcaaaaatgtaaagaatcagaaaaagacaagagacttgaggatttaatccaaacaactgaacagctaaaaacaagagctacaggggcagaagaagaaaaagtttaagaaagagacagtatattattttctctgagaagaggtaaagctggaaacctgttgtaagcaagatttattgaattttctcatcatagtcttaaaagagcagtactccaataggtcttgtttactaaaggaagatttgttgaaagaatcaaacaactgatggaggctgatgaaaagaatacatgacgatcaatgacacttggtacacacgttcacactcacagacacatacacaaacgttggataggaaaaggctggctggcaacatgtggcaacagtgctctgtaaggactcttgaagtag

SEQ ID NO: 514 - RNA, 205 nt, Homo sapiens
  source 1..205: mol_type = other RNA
  ncRNA 1..205: ncRNA_class = lncRNA
ctccctgttgtgaagtgcaccattgataataccctggaccagtggctacaagtcgggggtgatatgtgtgtgcacgcctacctcagcgggcagcccttggaggctaccactctgtgccagctccacagcacctgccacctataggactagaagggagcacaagctttgctgaactgctcttcaaatttaagcagctaaaaatgcc

SEQ ID NO: 515 - RNA, 1979 nt, Homo sapiens
  source 1..1979: mol_type = other RNA
  ncRNA 1..1979: ncRNA_class = lncRNA
tgtgcttccagctcaagccggctctgtgttttaggccaaccaacacaattcatataacatccttgcacatttccaccatttcccgcctcaccatgcctcagaggactacaggctccccaagacgggacatggttgggcaggtaccggatagatgctgtcactgctgaaaaagaaaaaaatatccaaagtattcacgttgtacctgctgagtgccagtactgcatgctctggggattccattcctgcccctatggaagaggggtcaacttttccaaaacatcatcttctagaggaagaagctgaagcccagcaaggaaaagtaacataacgaagatctcagcagaatgacggttatctgagcatctatattctggggttgcaaaaagaacaagaacgtagggttcaatggacctgagttccaggcctcactctgtcaccagctgactgtaagaagcccacccacctccaccttctctagatcacatgttttatggccgcctcaaaggcgtgaagcaggtgagtcgtgctgcctctgccaacatctgctttgtgagcctgggagtatttcacggagatgtttctctcgacaggtttatggctggtcttggctcagtgccttgaaaacactgatcatgtgatgaaacgagaatgtcatgctgcgggccatcaataaaccacactggcattcccgctcccaccccgacaccactccacccatgcaatttccctccccttcagcctcctccctccccacctcccaaagatatccagaccaggccaactcattaggaagtgacatccaccaggattcttggccccagtcaaaagagtggccagagcttgtctgctgcttgcggatacttttctgttttgtgttaaacgtttgagtttggaaatgagcaagagctatgtctaagggaaaccagacagcttgtttgaaaatccactaaatggccatcggaggatcatgccagatcttagaacaaagtctcagttattgcctgaaagcataaactttcaggcaaggcatcttctccatgctcctcagcatctgcagagtagcagagacttgaaagagatgtgtcctctggatcccacgtcagatgatacatcttgggatttccctacctgggggaagagtgggttttcctgaggaaattgcaagtgggcctgagtttctagagtgtgaggtgaaatggatgccactgtactgaattctgaccaaaactctggagagatgctgcgaaggaactcagcttccactgaaagggtcagtgaaggcttcaaggacagtatcacagcactttcctatcacttctataaaaatcacatccactttttcccaccaacttgcccattctccttgtctgggacacaggccctggtactttacattcaggtcatttccaagggatagagagaaatagacagaggctgaaagagtgatggggccgggagacgctgagatcattcaactaccaatgccattctgaactaagactttttttttttttttttttttttttttttttgagatggagtctcgctctgtcacccaggctggagtgcagtggcatgatcttggctcactgcaagctctccctcctgggttcacaccattctcctgcctcagcctcccaagtagctgggactacaggcacctgccaccaaagactttcttaaaccacactctctccctgccaagaaaatgggtatcatgtccttcataacccatcctgaaaaaatgaaaccgacatgatggcagagtggtatgaaaaagactctgaatttggaattgagggacaagagctcaactgccaactctctttattccccctgggacattgggcaacttaatctacttctcagtgagttgatctgtcacttgggtctaataatacaacttcttacgactactttttaaagcaaataaaaagatatatttgagagaatgctttattaattgcaaaacactgccc

SEQ ID NO: 516 - RNA, 2034 nt, Homo sapiens
  source 1..2034: mol_type = other RNA
  ncRNA 1..2034: ncRNA_class = lncRNA
tttttgcgttctctttccctctcccaacatggcggcctcagcaaaaaagaagaataagaaggggaagactatctccctaacagactttctggctgaggatgggggtactggtggaggaagcacctatgtttccaaaccagtcagctgggctgatgaaacgatgacctggaaggagatgtttcgacaacttggcacagtaacaatgacgatgtgtatagggtgcctccaattgaccgttccatccttcccactgctccacgggctgctcgggaacccaatatcgaccggagccgtcttcccaaatcgccaccctacactgcttttctaggaaacctaccctatgatgttacagaagagtcaattaaggaattctttcgaggattaaatatcagtgcagtgcgtttaccacgtgaacccagcaatccagagaggttgaaaggttttggttatgctgaatttgaggacctggattccctgctcagtgccctgagtctcaatgaagagtctctaggtaacaggagaattcgagtggatgttgctgatcaagcacaggataaagacagggatgatcgttcttttggccgtgatagaaatcgggattctgacaaaacagatacggactggagggcttgtcctgctacagacagctttgatgactagaagaggtgatgatagctttggagacaagtatcgggatcgttatgattcagaccggtatcgggatgggtatcgggatggcccacgccgggatatggatggatatggtggccgggatcgctatgatgaccaaggcagcagagactatgatagaggctatgattcccggataggcagtggcagaagagcatttggcagcgggtatcgcagggatgatgactacagaggaggcggggaccgctatgaagactgatatgacagacgggatgatcggtcgtggagctccagagatgattactctcgggatgattataggtgtgatgatagacgtcccccccaaagacccaaactgaatctaaagccttggagtactcttaagcaagatgattcctctgctagtacctcccagtccactcgagctgcttctatctttggaggggcaaagcctgttgacacagctgctagagaaagagaagtagaagaacggctacagaaggaacaagagaagttgcagcgtcagctgagtgagccaaaactagaacgacggcctcgggagagacacccaagctggcgaagtgaagaaactcaggaacgggaacggtcaaggacaggaagtgagtcatcgcagactgggacctccaccacatctggcagaaatgcacgaaggagagagagtaagaagtctctagaaaatgaaacactcaataaggaggaagattgccactctccaacttctaaacctcccaaacctgatcagcccctaaaggtaatgccagcccctccaccaaaggagaatgcttgggtgaagcgaagttctaaccctcctgctcgatctcagagctcagacgcagagcagcaatcccctacaagtggtgggggaaaagtagctccagctcaaccatctgaggaaggaccagcaaggcaagatgaaaataaagtagatgggatgaatgtcccaaaaggccaaactgggaactctagccgtggtccaggagacggagggaacaaagaccactggaaggagtcagataggaaagatggcaaaaaggatcaagactccagatctgcacctgagccaaagaaacctgaggaaaatccagcttccaagttcagttctgcaagcaactatgctgctctctcttgatggtgaagatgaaaatgagggagaagattatgccgaatagacctctacatcctgtgcttttctcctagtttctctccaccctggaacattcgagagcaaatcaaaacctctatccagacaagacaaaataaaactcaccatctcctgaagatctttcttacctttttttaaaaacaaaaaatgaaactattttgcatgctgctgcagcctttaaagtattgaagtaactggag

SEQ ID NO: 517 - RNA, 788 nt, Homo sapiens
  source 1..788: mol_type = other RNA
  ncRNA 1..788: ncRNA_class = lncRNA
agcagtgaaggatccagtgagattttccaggttaacggtcataaccgcctactggtccaacgttcagaagtaacacaggcacctggagaatacacagtagatgtggaaggacacggttgtacatttatccaggccacccttaagtacaatgttctcctacctaagaaggcatctggattttctctttccttggaaatagtaaagaactactcttcgactgcttttgacctcacagtgaccctcaaatacactggaattcgcaataaatccagtatggtggttatagatgtaaaaatgctatcaggatttactctggacattgagttctgcagtggcgctgtcattttttctaatatctattctctatccttccctaacaatttcttcaaagcttgaaaacaagggccaagtgatgaagactgaagtcaagaatgaccatgttcttttctacttggaaaatggttttggtcgagcagacagtttccctttttctgttgagcagagcaaccttgtgttcaacattcagccagccccagccatggtctacgattattacgaaaaagaagaatatgccctagctttttacaacatcgacagtagttcagtttcccagtgagacaaagcaattactggaagaggtggaaaagcatttcttgtaacaaaccgattcttctgtatcaaacctggaaaaaaatcatgaaccatctgacatcgtgaacagtttgcagtgggctatggtttcttgtcaagtcttatttccttatcatcccattaaatgttgtcatttt

SEQ ID NO: 518 - RNA, 1044 nt, Homo sapiens
  source 1..1044: mol_type = other RNA
  ncRNA 1..1044: ncRNA_class = lncRNA
gccaggccccctcctgaaagaggctccttgaagataattctaacatctttgtcatcagtgttgacatctcttgattgtatcctgtcattccatttgagatcttcctggttctggctggcatcctctgacatcactccagcagaagaaagggaaggaacacgctgcattactgcgtgggatatcttctaaccataccgatgattctccgtatagctggtcttttccaccttatcatccttcctctgagaagtatgaaaacactaagacatgtcgagggccagatggagaactccatgccaacctgctttgccctgtggatgtgctggatgttcccgagggcaccttgcctgacaaacagagcactgagcaagccatacagttgttggaaaagatgaaaacgtcagccagtcctttcttcctggccgttgggtatcataagccacacatccccttcagataccccaaggaatttcagaagttgtatcccttggagaacatcaccctggcccccgatcccgaggtccctgatggcctaccccctgtggcctacaacccctggatggacatcaggcaacgggaagacgtccaagccttaaacatcagtgtgccgtatggtccaattcctgtggactttcagcggaaaatccgccagagctactttgcctctgtgtcatatttggatacacaggtcggccgcctcttgagtgctttggacgatcttcagctggccaacagcaccatcattgcatttacctcggatcatgggtgggctctaggtgaacatggagaatgggccaaatacagcaattttgatgttgctacccatgttcccctgatattctatgttcctggaaggacggcttcacttccggaggcaggcgagaagcttttcccttacctcgacccttttgattccgcctcacagttgatggagccaggcaggcaatccatggaccttgtggaacttgtgtctctttttcccacgctggctggacttgcaggactgcaggttccacctcgctgccccgttccttcatttcacgt

SEQ ID NO: 519 - protein, 103 aa, synthetic construct
MKTEVKNDHVLFYLENGFGRADSFPFSVEQSNLVFNIQPAPAMVYDYYEKEEYALAFYNIDSSSVSQGGGGSAYPYDVPDYAAYPYDVPDYAAYPYDVPDYAA

SEQ ID NO: 520 - protein, 101 aa, synthetic construct
MNVPKGQTGNSSRGPGDGGNKDHWKESDRKDGKKDQDSRSAPEPKKPEENPASKFSSASNYAALSGGGGSAYPYDVPDYAAYPYDVPDYAAYPYDVPDYAA

SEQ ID NO: 521 - protein, 75 aa, synthetic construct
MALCRCDPELCWISCEENSKEWMNAWKLTAGSHKAMTRKGGGGSAYPYDVPDYAAYPYDVPDYAAYPYDVPDYAA

SEQ ID NO: 522 - protein, 109 aa, synthetic construct
MDATVLNSDQNSGEMLRRNSASTERVSEGFKDSITALSYHFYKNHIHFFPPTCPFSLSGTQALVLYIQVISKGGGGGSAYPYDVPDYAAYPYDVPDYAAYPYDVPDYAA

SEQ ID NO: 523 - protein, 79 aa, synthetic construct
MYVSNREVQVTSIKETFLSMGPASGLMLGCSRQEMGKLEKRTVGGGGSAYPYDVPDYAAYPYDVPDYAAYPYDVPDYAA

SEQ ID NO: 524 - protein, 62 aa, synthetic construct
MCVHAYLSGQPLEATTLCQLHSTCHLGGGGSAYPYDVPDYAAYPYDVPDYAAYPYDVPDYAA

SEQ ID NO: 525 - protein, 53 aa, synthetic construct
MLGCSRQEMGKLEKRTVGGGGSAYPYDVPDYAAYPYDVPDYAAYPYDVPDYAA

SEQ ID NO: 526 - protein, 278 aa, synthetic construct
MACLGFQRHKAQLNLATRTWPCTLLFFLLFIPVFCGGGGSKTEVKNDHVLFYLENGFGRADSFPFSVEQSNLVFNIQPAPAMVYDYYEKEEYALAFYNIDSSSVSQGGGGSNVPKGQTGNSSRGPGDGGNKDHWKESDRKDGKKDQDSRSAPEPKKPEENPASKFSSASNYAALSGGGGSAYPYDVPDYAAYPYDVPDYAAYPYDVPDYAAGGGGSFLLWILAAVSSGLFFYSFLLTAVSLSKMLKKRSPLTTGVYVKMPPTEPECEKQFQPYFIPIN

SEQ ID NO: 527 - protein, 425 aa, synthetic construct
MACLGFQRHKAQLNLATRTWPCTLLFFLLFIPVFCGGGGSNVPKGQTGNSSRGPGDGGNKDHWKESDRKDGKKDQDSRSAPEPKKPEENPASKFSSASNYAALSGGGGSALCRCDPELCWISCEENSKEWMNAWKLTAGSHKAMTRKGGGGSYVSNREVQVTSIKETFLSMGPASGLMLGCSRQEMGKLEKRTVGGGGSCVHAYLSGQPLEATTLCQLHSTCHLGGGGSLGCSRQEMGKLEKRTVGGGGSDATVLNSDQNSGEMLRRNSASTERVSEGFKDSITALSYHFYKNHIHFFPPTCPFSLSGTQALVLYIQVISKGGGGGSAYPYDVPDYAAYPYDVPDYAAYPYDVPDYAAGGGGSFLLWILAAVSSGLFFYSFLLTAVSLSKMLKKRSPLTTGVYVKMPPTEPECEKQFQPYFIPIN

SEQ ID NO: 528 - RNA, 312 nt, synthetic construct
  source 1..312: mol_type = other RNA
atgaagaccgaggtgaagaacgaccacgtcctgttctacctcgagaacggcttcgggcgcgccgacagcttccccttctccgtggagcagagcaacctggtgttcaacatccagccggcccccgcgatggtctacgactactacgagaaggaggagtacgccctggccttctacaacatcgactccagctccgtgagccagggcggcggcggctccgcctatccttatgatgttcctgattatgcagcctacccctacgacgtgccggactacgccgcctacccctatgacgtgcctgattacgccgcgtga

SEQ ID NO: 529 - RNA, 306 nt, synthetic construct
  source 1..306: mol_type = other RNA
atgaacgtgcccaagggccagaccgggaacagctcccgcggcccgggggacggcgggaacaaggaccactggaaggagagcgaccggaaggacggcaagaaggaccaggactcccgcagcgcccccgagcccaagaagcccgaggagaacccggcctccaagttcagctccgcgagcaactacgccgccctgtccggcggcggcggctccgcctatccttatgatgttcctgattatgcagcctacccctacgacgtgccggactacgccgcctacccctatgacgtgcctgattacgccgcgtga

SEQ ID NO: 530 - RNA, 228 nt, synthetic construct
  source 1..228: mol_type = other RNA
atggccctgtgccgctgcgaccccgagctctgctggatcagctgcgaggagaactccaaggagtggatgaacgcctggaagctgaccgcgggcagccacaaggccatgacccggaagggcggcggcggctccgcctatccttatgatgttcctgattatgcagcctacccctacgacgtgccggactacgccgcctacccctatgacgtgcctgattacgccgcgtga

SEQ ID NO: 531 - RNA, 330 nt, synthetic construct
  source 1..330: mol_type = other RNA
atggacgccaccgtgctgaacagcgaccagaactccggcgagatgctccgccggaacagcgcctccaccgagcgcgtcagcgaggggttcaaggactccatcaccgcgctgagctaccacttctacaagaaccacatccacttcttcccgcccacgtgccccttctccctgagcggcacccaggccctcgtgctgtacatccaggtgatctccaagggcggcggcggcggctccgcctatccttatgatgttcctgattatgcagcctacccctacgacgtgccggactacgccgcctacccctatgacgtgcctgattacgccgcgtga

SEQ ID NO: 532 - RNA, 240 nt, synthetic construct
  source 1..240: mol_type = other RNA
atgtacgtgagcaaccgcgaggtccaggtgacctccatcaaggagaccttcctgagcatgggccccgcctccgggctcatgctgggctgcagccggcaggagatggggaagctggagaagcgcacggtgggcggcggcggctccgcctatccttatgatgttcctgattatgcagcctacccctacgacgtgccggactacgccgcctacccctatgacgtgcctgattacgccgcgtga

SEQ ID NO: 533 - RNA, 189 nt, synthetic construct
  source 1..189: mol_type = other RNA
atgtgcgtgcacgcctacctgagcggccagcccctcgaggccaccaccctgtgccagctgcactccacgtgccacctcggcggcggcggctccgcctatccttatgatgttcctgattatgcagcctacccctacgacgtgccggactacgccgcctacccctatgacgtgcctgattacgccgcgtga

SEQ ID NO: 534 - RNA, 162 nt, synthetic construct
  source 1..162: mol_type = other RNA
atgctgggctgctcccgccaggagatggggaagctggagaagcggaccgtcggcggcggcggctccgcctatccttatgatgttcctgattatgcagcctacccctacgacgtgccggactacgccgcctacccctatgacgtgcctgattacgccgcgtga

SEQ ID NO: 535 - RNA, 837 nt, synthetic construct
  source 1..837: mol_type = other RNA
atggcctgcctgggcttccagcgccacaaggcccagctcaacctggcgacccggacctggccctgcacgctcctgttcttcctgctgttcatcccggtgttctgcggcggcggcggctccaagaccgaggtgaagaacgaccacgtcctgttctacctcgagaacggcttcgggcgcgccgacagcttccccttctccgtggagcagagcaacctggtgttcaacatccagccggcccccgcgatggtctacgactactacgagaaggaggagtacgccctggccttctacaacatcgactccagctccgtgagccagggtggcgggggctccaacgtgcccaagggccagaccgggaacagctcccgcggcccgggggacggcgggaacaaggaccactggaaggagagcgaccggaaggacggcaagaaggaccaggactcccgcagcgcccccgagcccaagaagcccgaggagaacccggcctccaagttcagctccgcgagcaactacgccgccctgtccggcggcggcgggtctgcctatccttatgatgttcctgattatgcagcctacccctacgacgtgccggactacgccgcctacccctatgacgtgcctgattacgccgcgggcggaggcggctccttcctgctctggatcctggccgccgtgagctccggcctgttcttctacagcttcctcctgaccgcggtctccctgagcaagatgctcaagaagcgctcccccctgaccacgggggtgtacgtgaagatgccgcccaccgagcccgagtgcgagaagcagttccagccctacttcatcccgatcaactga

SEQ ID NO: 536 - RNA, 1278 nt, synthetic construct
  source 1..1278: mol_type = other RNA
atggcctgcctgggcttccagcgccacaaggcccagctcaacctggcgacccggacctggccctgcacgctcctgttcttcctgctgttcatcccggtgttctgcggcggcggcggctccaacgtgcccaagggccagaccgggaacagctcccgcggcccgggggacggcgggaacaaggaccactggaaggagagcgaccggaaggacggcaagaaggaccaggactcccgcagcgcccccgagcccaagaagcccgaggagaacccggcctccaagttcagctccgcgagcaactacgccgccctgtccggtggcgggggctccgccctgtgccgctgcgaccccgagctctgctggatcagctgcgaggagaactccaaggagtggatgaacgcctggaagctgaccgcgggcagccacaaggccatgacccggaagggcggcggcgggtcttacgtgagcaaccgcgaggtccaggtgacctccatcaaggagaccttcctgagcatgggccccgcctccgggctcatgctgggctgcagccggcaggagatggggaagctggagaagcgcacggtgggcggaggcggctcctgcgtgcacgcctacctgagcggccagcccctcgaggccaccaccctgtgccagctgcactccacgtgccacctcgggggaggcggatccctgggctgctcccgccaggagatggggaagctggagaagcggaccgtcggcggcggagggtctgacgccaccgtgctgaacagcgaccagaactccggcgagatgctccgccggaacagcgcctccaccgagcgcgtcagcgaggggttcaaggactccatcaccgcgctgagctaccacttctacaagaaccacatccacttcttcccgcccacgtgccccttctccctgagcggcacccaggccctcgtgctgtacatccaggtgatctccaagggcggcggcggcgggtctgcctatccttatgatgttcctgattatgcagcctacccctacgacgtgccggactacgccgcctacccctatgacgtgcctgattacgccgcgggcggaggcggctccttcctgctctggatcctggccgccgtgagctccggcctgttcttctacagcttcctcctgaccgcggtctccctgagcaagatgctcaagaagcgctcccccctgaccacgggggtgtacgtgaagatgccgcccaccgagcccgagtgcgagaagcagttccagccctacttcatcccgatcaactga

SEQ ID NO: 537 - RNA, 584 nt, synthetic construct
  source 1..584: mol_type = other RNA
aggagagtcccgcagtcggcgtccagcggctctgcttgttcgtgtgtgtgtcgttgcaggccttattcaagcttaccatgaagaccgaggtgaagaacgaccacgtcctgttctacctcgagaacggcttcgggcgcgccgacagcttccccttctccgtggagcagagcaacctggtgttcaacatccagccggcccccgcgatggtctacgactactacgagaaggaggagtacgccctggccttctacaacatcgactccagctccgtgagccagggcggcggcggctccgcctatccttatgatgttcctgattatgcagcctacccctacgacgtgccggactacgccgcctacccctatgacgtgcctgattacgccgcgtgaggactagtccctgttcccagagcccacttttttttctttttttgaaataaaatagcctgtctttcagatctcaaaggctcttttcagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 538 - RNA, 578 nt, synthetic construct
  source 1..578: mol_type = other RNA
aggagagtcccgcagtcggcgtccagcggctctgcttgttcgtgtgtgtgtcgttgcaggccttattcaagcttaccatgaacgtgcccaagggccagaccgggaacagctcccgcggcccgggggacggcgggaacaaggaccactggaaggagagcgaccggaaggacggcaagaaggaccaggactcccgcagcgcccccgagcccaagaagcccgaggagaacccggcctccaagttcagctccgcgagcaactacgccgccctgtccggcggcggcggctccgcctatccttatgatgttcctgattatgcagcctacccctacgacgtgccggactacgccgcctacccctatgacgtgcctgattacgccgcgtgaggactagtccctgttcccagagcccacttttttttctttttttgaaataaaatagcctgtctttcagatctcaaaggctcttttcagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 539 - RNA, 500 nt, synthetic construct
  source 1..500: mol_type = other RNA
aggagagtcccgcagtcggcgtccagcggctctgcttgttcgtgtgtgtgtcgttgcaggccttattcaagcttaccatggccctgtgccgctgcgaccccgagctctgctggatcagctgcgaggagaactccaaggagtggatgaacgcctggaagctgaccgcgggcagccacaaggccatgacccggaagggcggcggcggctccgcctatccttatgatgttcctgattatgcagcctacccctacgacgtgccggactacgccgcctacccctatgacgtgcctgattacgccgcgtgaggactagtccctgttcccagagcccacttttttttctttttttgaaataaaatagcctgtctttcagatctcaaaggctcttttcagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 540 - RNA, 602 nt, synthetic construct
  source 1..602: mol_type = other RNA
aggagagtcccgcagtcggcgtccagcggctctgcttgttcgtgtgtgtgtcgttgcaggccttattcaagcttaccatggacgccaccgtgctgaacagcgaccagaactccggcgagatgctccgccggaacagcgcctccaccgagcgcgtcagcgaggggttcaaggactccatcaccgcgctgagctaccacttctacaagaaccacatccacttcttcccgcccacgtgccccttctccctgagcggcacccaggccctcgtgctgtacatccaggtgatctccaagggcggcggcggcggctccgcctatccttatgatgttcctgattatgcagcctacccctacgacgtgccggactacgccgcctacccctatgacgtgcctgattacgccgcgtgaggactagtccctgttcccagagcccacttttttttctttttttgaaataaaatagcctgtctttcagatctcaaaggctcttttcagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 541 - RNA, 512 nt, synthetic construct
  source 1..512: mol_type = other RNA
aggagagtcccgcagtcggcgtccagcggctctgcttgttcgtgtgtgtgtcgttgcaggccttattcaagcttaccatgtacgtgagcaaccgcgaggtccaggtgacctccatcaaggagaccttcctgagcatgggccccgcctccgggctcatgctgggctgcagccggcaggagatggggaagctggagaagcgcacggtgggcggcggcggctccgcctatccttatgatgttcctgattatgcagcctacccctacgacgtgccggactacgccgcctacccctatgacgtgcctgattacgccgcgtgaggactagtccctgttcccagagcccacttttttttctttttttgaaataaaatagcctgtctttcagatctcaaaggctcttttcagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 542 - RNA, 461 nt, synthetic construct
  source 1..461: mol_type = other RNA
aggagagtcccgcagtcggcgtccagcggctctgcttgttcgtgtgtgtgtcgttgcaggccttattcaagcttaccatgtgcgtgcacgcctacctgagcggccagcccctcgaggccaccaccctgtgccagctgcactccacgtgccacctcggcggcggcggctccgcctatccttatgatgttcctgattatgcagcctacccctacgacgtgccggactacgccgcctacccctatgacgtgcctgattacgccgcgtgaggactagtccctgttcccagagcccacttttttttctttttttgaaataaaatagcctgtctttcagatctcaaaggctcttttcagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 543 - RNA, 434 nt, synthetic construct
  source 1..434: mol_type = other RNA
aggagagtcccgcagtcggcgtccagcggctctgcttgttcgtgtgtgtgtcgttgcaggccttattcaagcttaccatgctgggctgctcccgccaggagatggggaagctggagaagcggaccgtcggcggcggcggctccgcctatccttatgatgttcctgattatgcagcctacccctacgacgtgccggactacgccgcctacccctatgacgtgcctgattacgccgcgtgaggactagtccctgttcccagagcccacttttttttctttttttgaaataaaatagcctgtctttcagatctcaaaggctcttttcagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 544 - RNA, 1109 nt, synthetic construct
  source 1..1109: mol_type = other RNA
aggagagtcccgcagtcggcgtccagcggctctgcttgttcgtgtgtgtgtcgttgcaggccttattcaagcttaccatggcctgcctgggcttccagcgccacaaggcccagctcaacctggcgacccggacctggccctgcacgctcctgttcttcctgctgttcatcccggtgttctgcggcggcggcggctccaagaccgaggtgaagaacgaccacgtcctgttctacctcgagaacggcttcgggcgcgccgacagcttccccttctccgtggagcagagcaacctggtgttcaacatccagccggcccccgcgatggtctacgactactacgagaaggaggagtacgccctggccttctacaacatcgactccagctccgtgagccagggtggcgggggctccaacgtgcccaagggccagaccgggaacagctcccgcggcccgggggacggcgggaacaaggaccactggaaggagagcgaccggaaggacggcaagaaggaccaggactcccgcagcgcccccgagcccaagaagcccgaggagaacccggcctccaagttcagctccgcgagcaactacgccgccctgtccggcggcggcgggtctgcctatccttatgatgttcctgattatgcagcctacccctacgacgtgccggactacgccgcctacccctatgacgtgcctgattacgccgcgggcggaggcggctccttcctgctctggatcctggccgccgtgagctccggcctgttcttctacagcttcctcctgaccgcggtctccctgagcaagatgctcaagaagcgctcccccctgaccacgggggtgtacgtgaagatgccgcccaccgagcccgagtgcgagaagcagttccagccctacttcatcccgatcaactgaggactagtccctgttcccagagcccacttttttttctttttttgaaataaaatagcctgtctttcagatctcaaaggctcttttcagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 545 - RNA, 1550 nt, synthetic construct
  source 1..1550: mol_type = other RNA
aggagagtcccgcagtcggcgtccagcggctctgcttgttcgtgtgtgtgtcgttgcaggccttattcaagcttaccatggcctgcctgggcttccagcgccacaaggcccagctcaacctggcgacccggacctggccctgcacgctcctgttcttcctgctgttcatcccggtgttctgcggcggcggcggctccaacgtgcccaagggccagaccgggaacagctcccgcggcccgggggacggcgggaacaaggaccactggaaggagagcgaccggaaggacggcaagaaggaccaggactcccgcagcgcccccgagcccaagaagcccgaggagaacccggcctccaagttcagctccgcgagcaactacgccgccctgtccggtggcgggggctccgccctgtgccgctgcgaccccgagctctgctggatcagctgcgaggagaactccaaggagtggatgaacgcctggaagctgaccgcgggcagccacaaggccatgacccggaagggcggcggcgggtcttacgtgagcaaccgcgaggtccaggtgacctccatcaaggagaccttcctgagcatgggccccgcctccgggctcatgctgggctgcagccggcaggagatggggaagctggagaagcgcacggtgggcggaggcggctcctgcgtgcacgcctacctgagcggccagcccctcgaggccaccaccctgtgccagctgcactccacgtgccacctcgggggaggcggatccctgggctgctcccgccaggagatggggaagctggagaagcggaccgtcggcggcggagggtctgacgccaccgtgctgaacagcgaccagaactccggcgagatgctccgccggaacagcgcctccaccgagcgcgtcagcgaggggttcaaggactccatcaccgcgctgagctaccacttctacaagaaccacatccacttcttcccgcccacgtgccccttctccctgagcggcacccaggccctcgtgctgtacatccaggtgatctccaagggcggcggcggcgggtctgcctatccttatgatgttcctgattatgcagcctacccctacgacgtgccggactacgccgcctacccctatgacgtgcctgattacgccgcgggcggaggcggctccttcctgctctggatcctggccgccgtgagctccggcctgttcttctacagcttcctcctgaccgcggtctccctgagcaagatgctcaagaagcgctcccccctgaccacgggggtgtacgtgaagatgccgcccaccgagcccgagtgcgagaagcagttccagccctacttcatcccgatcaactgaggactagtccctgttcccagagcccacttttttttctttttttgaaataaaatagcctgtctttcagatctcaaaggctcttttcagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 546 - protein, 13 aa, Homo sapiens
FLSMGPASGLMLG

SEQ ID NO: 547 - protein, 11 aa, Homo sapiens
SRQEMGKLEKR

SEQ ID NO: 548 - protein, 11 aa, Homo sapiens
QEMGKLEKRTV

SEQ ID NO: 549 - protein, 11 aa, Homo sapiens
REVQVTSIKET

SEQ ID NO: 550 - protein, 10 aa, Homo sapiens
RQEMGKLEKR

SEQ ID NO: 551 - protein, 10 aa, Homo sapiens
SRQEMGKLEK

SEQ ID NO: 552 - protein, 10 aa, Homo sapiens
FLSMGPASGL

SEQ ID NO: 553 - protein, 10 aa, Homo sapiens
KETFLSMGPA

SEQ ID NO: 554 - protein, 10 aa, Homo sapiens
NREVQVTSIK

SEQ ID NO: 555 - protein, 10 aa, Homo sapiens
VQVTSIKETF

SEQ ID NO: 556 - protein, 10 aa, Homo sapiens
GPASGLMLGC

SEQ ID NO: 557 - protein, 9 aa, Homo sapiens
MGKLEKRTV

SEQ ID NO: 558 - protein, 9 aa, Homo sapiens
RQEMGKLEK

SEQ ID NO: 559 - protein, 9 aa, Homo sapiens
QEMGKLEKR

SEQ ID NO: 560 - protein, 9 aa, Homo sapiens
MGPASGLML

SEQ ID NO: 561 - protein, 9 aa, Homo sapiens
LSMGPASGL

SEQ ID NO: 562 - protein, 9 aa, Homo sapiens
KETFLSMGP

SEQ ID NO: 563 - protein, 9 aa, Homo sapiens
SMGPASGLM

SEQ ID NO: 564 - protein, 9 aa, Homo sapiens
ETFLSMGPA

SEQ ID NO: 565 - protein, 9 aa, Homo sapiens
NREVQVTSI

SEQ ID NO: 566 - protein, 9 aa, Homo sapiens
YVSNREVQV

SEQ ID NO: 567 - protein, 9 aa, Homo sapiens
REVQVTSIK

SEQ ID NO: 568 - protein, 9 aa, Homo sapiens
SIKETFLSM

SEQ ID NO: 569 - protein, 9 aa, Homo sapiens
QVTSIKETF

SEQ ID NO: 570 - protein, 9 aa, Homo sapiens
VTSIKETFL

SEQ ID NO: 571 - protein, 9 aa, Homo sapiens
VQVTSIKET

SEQ ID NO: 572 - protein, 11 aa, Homo sapiens
TAGSHKAMTRK

SEQ ID NO: 573 - protein, 11 aa, Homo sapiens
EENSKEWMNAW

SEQ ID NO: 574 - protein, 11 aa, Homo sapiens
WISCEENSKEW

SEQ ID NO: 575 - protein, 11 aa, Homo sapiens
ISCEENSKEWM

SEQ ID NO: 576 - protein, 10 aa, Homo sapiens
TAGSHKAMTR

SEQ ID NO: 577 - protein, 10 aa, Homo sapiens
AGSHKAMTRK

SEQ ID NO: 578 - protein, 10 aa, Homo sapiens
KLTAGSHKAM

SEQ ID NO: 579 - protein, 10 aa, Homo sapiens
KEWMNAWKLT

SEQ ID NO: 580 - protein, 10 aa, Homo sapiens
ISCEENSKEW

SEQ ID NO: 581 - protein, 10 aa, Homo sapiens
ENSKEWMNAW

SEQ ID NO: 582 - protein, 9 aa, Homo sapiens
GSHKAMTRK

SEQ ID NO: 583 - protein, 9 aa, Homo sapiens
LTAGSHKAM

SEQ ID NO: 584 - protein, 9 aa, Homo sapiens
AGSHKAMTR

SEQ ID NO: 585 - protein, 9 aa, Homo sapiens
KLTAGSHKA

SEQ ID NO: 586 - protein, 9 aa, Homo sapiens
KEWMNAWKL

SEQ ID NO: 587 - protein, 9 aa, Homo sapiens
NSKEWMNAW

SEQ ID NO: 588 - protein, 9 aa, Homo sapiens
SCEENSKEW

SEQ ID NO: 589 - protein, 9 aa, Homo sapiens
CEENSKEWM

SEQ ID NO: 590 - protein, 11 aa, Homo sapiens
QPLEATTLCQL

SEQ ID NO: 591 - protein, 11 aa, Homo sapiens
LSGQPLEATTL

SEQ ID NO: 592 - protein, 11 aa, Homo sapiens
SGQPLEATTLC

SEQ ID NO: 593 - protein, 11 aa, Homo sapiens
YLSGQPLEATT

SEQ ID NO: 594 - protein, 11 aa, Homo sapiens
HAYLSGQPLEA

SEQ ID NO: 595 - protein, 10 aa, Homo sapiens
SGQPLEATTL

SEQ ID NO: 596 - protein, 10 aa, Homo sapiens
AYLSGQPLEA

SEQ ID NO: 597 - protein, 10 aa, Homo sapiens
GQPLEATTLC

SEQ ID NO: 598 - protein, 10 aa, Homo sapiens
VHAYLSGQPL

SEQ ID NO: 599 - protein, 9 aa, Homo sapiens
YLSGQPLEA

SEQ ID NO: 600 - protein, 9 aa, Homo sapiens
LEATTLCQL

SEQ ID NO: 601 - protein, 9 aa, Homo sapiens
QPLEATTLC

SEQ ID NO: 602 - protein, 9 aa, Homo sapiens
GQPLEATTL

SEQ ID NO: 603 - protein, 9 aa, Homo sapiens
HAYLSGQPL

SEQ ID NO: 604 - protein, 12 aa, Homo sapiens
GTQALVLYIQVI

SEQ ID NO: 605 - protein, 11 aa, Homo sapiens
HFYKNHIHFFP

SEQ ID NO: 606 - protein, 11 aa, Homo sapiens
FYKNHIHFFPP

SEQ ID NO: 607 - protein, 11 aa, Homo sapiens
ERVSEGFKDSI

SEQ ID NO: 608 - protein, 11 aa, Homo sapiens
NSDQNSGEMLR

SEQ ID NO: 609 - protein, 10 aa, Homo sapiens
FYKNHIHFFP

SEQ ID NO: 610 - protein, 10 aa, Homo sapiens
ASTERVSEGF

SEQ ID NO: 611 - protein, 10 aa, Homo sapiens
RVSEGFKDSI

SEQ ID NO: 612 - protein, 10 aa, Homo sapiens
CPFSLSGTQA

SEQ ID NO: 613 - protein, 10 aa, Homo sapiens
FFPPTCPFSL

SEQ ID NO: 614 - protein, 10 aa, Homo sapiens
PFSLSGTQAL

SEQ ID NO: 615 - protein, 10 aa, Homo sapiens
DQNSGEMLRR

SEQ ID NO: 616 - protein, 10 aa, Homo sapiens
SDQNSGEMLR

SEQ ID NO: 617 - protein, 10 aa, Homo sapiens
LNSDQNSGEM

SEQ ID NO: 618 - protein, 9 aa, Homo sapiens
YHFYKNHIH

SEQ ID NO: 619 - protein, 9 aa, Homo sapiens
NHIHFFPPT

SEQ ID NO: 620 - protein, 9 aa, Homo sapiens
STERVSEGF

SEQ ID NO: 621 - protein, 9 aa, Homo sapiens
VSEGFKDSI

SEQ ID NO: 622 - protein, 9 aa, Homo sapiens
FSLSGTQAL

SEQ ID NO: 623 - protein, 9 aa, Homo sapiens
HFFPPTCPF

SEQ ID NO: 624 - protein, 9 aa, Homo sapiens
ITALSYHFY

SEQ ID NO: 625 - protein, 9 aa, Homo sapiens
SITALSYHF

SEQ ID NO: 626 - protein, 9 aa, Homo sapiens
GEMLRRNSA

SEQ ID NO: 627 - protein, 9 aa, Homo sapiens
DQNSGEMLR

SEQ ID NO: 628 - protein, 9 aa, Homo sapiens
SDQNSGEML

SEQ ID NO: 629 - protein, 9 aa, Homo sapiens
QNSGEMLRR

SEQ ID NO: 630 - protein, 9 aa, Homo sapiens
SASNYAALS

SEQ ID NO: 631 - protein, 11 aa, Homo sapiens
QDSRSAPEPKK

SEQ ID NO: 632 - protein, 11 aa, Homo sapiens
PASKFSSASNY

SEQ ID NO: 633 - protein, 10 aa, Homo sapiens
DSRSAPEPKK

SEQ ID NO: 634 - protein, 10 aa, Homo sapiens
QDSRSAPEPK

SEQ ID NO: 635 - protein, 10 aa, Homo sapiens
ASKFSSASNY

SEQ ID NO: 636 - protein, 10 aa, Homo sapiens
FSSASNYAAL

SEQ ID NO: 637 - protein, 10 aa, Homo sapiens
SKFSSASNYA

SEQ ID NO: 638 - protein, 9 aa, Homo sapiens
SRSAPEPKK

SEQ ID NO: 639 - protein, 9 aa, Homo sapiens
DSRSAPEPK

SEQ ID NO: 640 - protein, 9 aa, Homo sapiens
APEPKKPEE

SEQ ID NO: 641 - protein, 9 aa, Homo sapiens
SKFSSASNY

SEQ ID NO: 642 - protein, 9 aa, Homo sapiens
FSSASNYAA

SEQ ID NO: 643 - protein, 10 aa, Homo sapiens
FPFSVEQSNL

SEQ ID NO: 644 - protein, 10 aa, Homo sapiens
EEYALAFYNI

SEQ ID NO: 645 - protein, 9 aa, Homo sapiens
FSVEQSNLV

SEQ ID NO: 646 - protein, 9 aa, Homo sapiens
FPFSVEQSN

SEQ ID NO: 647 - protein, 9 aa, Homo sapiens
PFSVEQSNL

SEQ ID NO: 648 - protein, 9 aa, Homo sapiens
EYALAFYNI

SEQ ID NO: 649 - protein, 9 aa, Homo sapiens
KEEYALAFY

SEQ ID NO: 650 - protein, 9 aa, Homo sapiens
EEYALAFYN

SEQ ID NO: 651 - RNA, 39 nt, synthetic construct
  source 1..39: mol_type = other RNA
ttcctgagcatgggccccgcctccgggctcatgctgggc

SEQ ID NO: 652 - RNA, 33 nt, synthetic construct
  source 1..33: mol_type = other RNA
agccgccaggagatgggcaagctggagaagcgg

SEQ ID NO: 653 - RNA, 33 nt, synthetic construct
  source 1..33: mol_type = other RNA
caggagatgggcaagctggagaagcgcaccgtg

SEQ ID NO: 654 - RNA, 33 nt, synthetic construct
  source 1..33: mol_type = other RNA
cgcgaggtgcaggtcaccagcatcaaggagacc

SEQ ID NO: 655 - RNA, 30 nt, synthetic construct
  source 1..30: mol_type = other RNA
cgccaggagatgggcaagctggagaagcgg

SEQ ID NO: 656 - RNA, 30 nt, synthetic construct
  source 1..30: mol_type = other RNA
agccgccaggagatgggcaagctggagaag

SEQ ID NO: 657 - RNA, 30 nt, synthetic construct
  source 1..30: mol_type = other RNA
ttcctgagcatgggccccgcctccgggctc

SEQ ID NO: 658 - RNA, 30 nt, synthetic construct
  source 1..30: mol_type = other RNA
aaggagaccttcctgagcatgggccccgcc

SEQ ID NO: 659 - RNA, 30 nt, synthetic construct
  source 1..30: mol_type = other RNA
aaccgcgaggtgcaggtcaccagcatcaag

SEQ ID NO: 660 - RNA, 30 nt, synthetic construct
  source 1..30: mol_type = other RNA
gtgcaggtcaccagcatcaaggagaccttc

SEQ ID NO: 661 - RNA, 30 nt, synthetic construct
  source 1..30: mol_type = other RNA
ggccccgccagcgggctgatgctcggctgc

SEQ ID NO: 662 - RNA, 27 nt, synthetic construct
  source 1..27: mol_type = other RNA
atgggcaagctggagaagcgcaccgtg

SEQ ID NO: 663 - RNA, 27 nt, synthetic construct
  source 1..27: mol_type = other RNA
cgccaggagatgggcaagctggagaag

SEQ ID NO: 664 - RNA, 27 nt, synthetic construct
  source 1..27: mol_type = other RNA
caggagatgggcaagctggagaagcgc

SEQ ID NO: 665 - RNA, 27 nt, synthetic construct
  source 1..27: mol_type = other RNA
atgggccccgccagcgggctgatgctc

SEQ ID NO: 666 - RNA, 27 nt, synthetic construct
  source 1..27: mol_type = other RNA
ctgagcatgggccccgcctccgggctc

SEQ ID NO: 667 - RNA, 27 nt, synthetic construct
  source 1..27: mol_type = other RNA
aaggagaccttcctgagcatgggcccc

SEQ ID NO: 668 - RNA, 27 nt, synthetic construct
  source 1..27: mol_type = other RNA
agcatgggccccgcctccgggctgatg

SEQ ID NO: 669 - RNA, 27 nt, synthetic construct
  source 1..27: mol_type = other RNA
gagaccttcctgagcatgggccccgcc

SEQ ID NO: 670 - RNA, 27 nt, synthetic construct
  source 1..27: mol_type = other RNA
aaccgcgaggtgcaggtcaccagcatc

SEQ ID NO: 671 - RNA, 27 nt, synthetic construct
  source 1..27: mol_type = other RNA
tacgtgagcaaccgcgaggtccaggtg

SEQ ID NO: 672 - RNA, 27 nt, synthetic construct
  source 1..27: mol_type = other RNA
cgcgaggtgcaggtcaccagcatcaag

SEQ ID NO: 673 - RNA, 27 nt, synthetic construct
  source 1..27: mol_type = other RNA
agcatcaaggagaccttcctgtccatg

SEQ ID NO: 674 - RNA, 27 nt, synthetic construct
  source 1..27: mol_type = other RNA
caggtgaccagcatcaaggagaccttc

SEQ ID NO: 675 - RNA, 27 nt, synthetic construct
  source 1..27: mol_type = other RNA
gtgaccagcatcaaggagaccttcctg

SEQ ID NO: 676 - RNA, 27 nt, synthetic construct
  source 1..27: mol_type = other RNA
gtgcaggtcaccagcatcaaggagacc

SEQ ID NO: 677 - RNA, 33 nt, synthetic construct
  source 1..33: mol_type = other RNA
accgccggcagccacaaggccatgacccgcaag

SEQ ID NO: 678 - RNA, 33 nt, synthetic construct
  source 1..33: mol_type = other RNA
gaggagaacagcaaggagtggatgaacgcctgg

SEQ ID NO: 679 - RNA, 33 nt, synthetic construct
  source 1..33: mol_type = other RNA
tggatcagctgcgaggagaactccaaggagtgg

SEQ ID NO: 680 - RNA, 33 nt, synthetic construct
  source 1..33: mol_type = other RNA
atcagctgcgaggagaactccaaggagtggatg

SEQ ID NO: 681 - RNA, 30 nt, synthetic construct
  source 1..30: mol_type = other RNA
accgccggcagccacaaggccatgacccgc

SEQ ID NO: 682 - RNA, 30 nt, synthetic construct
  source 1..30: mol_type = other RNA
gccggcagccacaaggccatgacccgcaag

SEQ ID NO: 683 - RNA, 30 nt, synthetic construct
  source 1..30: mol_type = other RNA
aagctgaccgccggcagccacaaggccatg

SEQ ID NO: 684 - RNA, 30 nt, synthetic construct
  source 1..30: mol_type = other RNA
aaggagtggatgaacgcctggaagctgacc

SEQ ID NO: 685 - RNA, 30 nt, synthetic construct
  source 1..30: mol_type = other RNA
atcagctgcgaggagaactccaaggagtgg

SEQ ID NO: 686 - RNA, 30 nt, synthetic construct
  source 1..30: mol_type = other RNA
gagaacagcaaggagtggatgaacgcctgg

SEQ ID NO: 687 - RNA, 27 nt, synthetic construct
  source 1..27: mol_type = other RNA
ggcagccacaaggccatgacccgcaag

SEQ ID NO: 688 - RNA, 27 nt, synthetic construct
  source 1..27: mol_type = other RNA
ctgaccgccggcagccacaaggccatg

SEQ ID NO: 689 - RNA, 27 nt, synthetic construct
  source 1..27: mol_type = other RNA
gccggcagccacaaggccatgacccgc

SEQ ID NO: 690 - RNA, 27 nt, synthetic construct
  source 1..27: mol_type = other RNA
aagctgaccgccggcagccacaaggcc

SEQ ID NO: 691 - RNA, 27 nt, synthetic construct
  source 1..27: mol_type = other RNA
aaggagtggatgaacgcctggaagctg

SEQ ID NO: 692 - RNA, 27 nt, synthetic construct
  source 1..27: mol_type = other RNA
aacagcaaggagtggatgaacgcctgg

SEQ ID NO: 693 - RNA, 27 nt, synthetic construct
  source 1..27: mol_type = other RNA
agctgcgaggagaactccaaggagtgg

SEQ ID NO: 694 - RNA, 27 nt, synthetic construct
  source 1..27: mol_type = other RNA
tgcgaggagaacagcaaggagtggatg

SEQ ID NO: 695 - RNA, 33 nt, synthetic construct
  source 1..33: mol_type = other RNA
cagcccctggaggccaccaccctctgccagctg

SEQ ID NO: 696 - RNA, 33 nt, synthetic construct
  source 1..33: mol_type = other RNA
ctgagcggccagcccctcgaggccaccaccctg

SEQ ID NO: 697 - RNA, 33 nt, synthetic construct
  source 1..33: mol_type = other RNA
agcggccagcccctggaggccaccaccctctgc

SEQ ID NO: 698 - RNA, 33 nt, synthetic construct
  source 1..33: mol_type = other RNA
tacctgagcggccagcccctcgaggccaccacc

SEQ ID NO: 699 - RNA, 33 nt, synthetic construct
  source 1..33: mol_type = other RNA
cacgcctacctgagcggccagcccctcgaggcc

SEQ ID NO: 700 - RNA, 30 nt, synthetic construct
  source 1..30: mol_type = other RNA
agcggccagcccctggaggccaccaccctc

SEQ ID NO: 701 - RNA, 30 nt, synthetic construct
  source 1..30: mol_type = other RNA
gcctacctgagcggccagcccctcgaggcc

SEQ ID NO: 702 - RNA, 30 nt, synthetic construct
  source 1..30: mol_type = other RNA
ggccagcccctggaggccaccaccctctgc

SEQ ID NO: 703 - RNA, 30 nt, synthetic construct
  source 1..30: mol_type = other RNA
gtgcacgcctacctgagcggccagcccctc

SEQ ID NO: 704 - RNA, 27 nt, synthetic construct
  source 1..27: mol_type = other RNA
tacctgagcggccagcccctcgaggcc

SEQ ID NO: 705 - RNA, 27 nt, synthetic construct
  source 1..27: mol_type = other RNA
ctggaggccaccaccctctgccagctg

SEQ ID NO: 706 - RNA, 27 nt, synthetic construct
  source 1..27: mol_type = other RNA
cagcccctggaggccaccaccctctgc

SEQ ID NO: 707 - RNA, 27 nt, synthetic construct
  source 1..27: mol_type = other RNA
ggccagcccctggaggccaccaccctc

SEQ ID NO: 708 - RNA, 27 nt, synthetic construct
  source 1..27: mol_type = other RNA
cacgcctacctgagcggccagcccctc

SEQ ID NO: 709 - RNA, 36 nt, synthetic construct
  source 1..36: mol_type = other RNA
ggcacccaggccctggtgctctacatccaggtcatc

SEQ ID NO: 710 - RNA, 33 nt, synthetic construct
  source 1..33: mol_type = other RNA
cacttctacaagaaccacatccacttcttcccc

SEQ ID NO: 711 - RNA, 33 nt, synthetic construct
  source 1..33: mol_type = other RNA
ttctacaagaaccacatccacttcttcccgccc

SEQ ID NO: 712 - RNA, 33 nt, synthetic construct
  source 1..33: mol_type = other RNA
gagcgcgtgagcgagggcttcaaggactccatc

SEQ ID NO: 713 - RNA, 33 nt, synthetic construct
  source 1..33: mol_type = other RNA
aacagcgaccagaactccggcgagatgctgcgc

SEQ ID NO: 714 - RNA, 30 nt, synthetic construct
  source 1..30: mol_type = other RNA
ttctacaagaaccacatccacttcttcccc

SEQ ID NO: 715 - RNA, 30 nt, synthetic construct
  source 1..30: mol_type = other RNA
gccagcaccgagcgcgtgtccgagggcttc

SEQ ID NO: 716 - RNA, 30 nt, synthetic construct
  source 1..30: mol_type = other RNA
cgcgtgagcgagggcttcaaggactccatc

SEQ ID NO: 717 - RNA, 30 nt, synthetic construct
  source 1..30: mol_type = other RNA
tgccccttcagcctgtccggcacccaggcc

SEQ ID NO: 718 - RNA, 30 nt, synthetic construct
  source 1..30: mol_type = other RNA
ttcttccccccgacctgccccttcagcctg

SEQ ID NO: 719 - RNA, 30 nt, synthetic construct
  source 1..30: mol_type = other RNA
cccttcagcctgtccggcacccaggccctc

SEQ ID NO: 720 - RNA, 30 nt, synthetic construct
  source 1..30: mol_type = other RNA
gaccagaacagcggcgagatgctgcgccgg

SEQ ID NO: 721 - RNA, 30 nt, synthetic construct
  source 1..30: mol_type = other RNA
agcgaccagaactccggcgagatgctgcgc

SEQ ID NO: 722 - RNA, 30 nt, synthetic construct
  source 1..30: mol_type = other RNA
ctgaacagcgaccagaactccggcgagatg

SEQ ID NO: 723 - RNA, 27 nt, synthetic construct
  source 1..27: mol_type = other RNA
taccacttctacaagaaccacatccac

SEQ ID NO: 724 - RNA, 27 nt, synthetic construct
  source 1..27: mol_type = other RNA
aaccacatccacttcttcccgcccacc

SEQ ID NO: 725 - RNA, 27 nt, synthetic construct
  source 1..27: mol_type = other RNA
agcaccgagcgcgtgtccgagggcttc

SEQ ID NO: 726 - RNA, 27 nt, synthetic construct
  source 1..27: mol_type = other RNA
gtgagcgagggcttcaaggactccatc

SEQ ID NO: 727 - RNA, 27 nt, synthetic construct
  source 1..27: mol_type = other RNA
ttcagcctgtccggcacccaggccctc

SEQ ID NO: 728 - RNA, 27 nt, synthetic construct
  source 1..27: mol_type = other RNA
cacttcttccccccgacctgccccttc

SEQ ID NO: 729 - RNA, 27 nt, synthetic construct
  source 1..27: mol_type = other RNA
atcaccgccctgagctaccacttctac

SEQ ID NO: 730 - RNA, 27 nt, synthetic construct
  source 1..27: mol_type = other RNA
agcatcaccgccctgtcctaccacttc

SEQ ID NO: 731 - RNA, 27 nt, synthetic construct
  source 1..27: mol_type = other RNA
ggcgagatgctgcgccggaacagcgcc

SEQ ID NO: 732 - RNA, 27 nt, synthetic construct
  source 1..27: mol_type = other RNA
gaccagaacagcggcgagatgctgcgc

SEQ ID NO: 733 - RNA, 27 nt, synthetic construct
  source 1..27: mol_type = other RNA
agcgaccagaactccggcgagatgctg

SEQ ID NO: 734 - RNA, 27 nt, synthetic construct
  source 1..27: mol_type = other RNA
cagaacagcggcgagatgctgcgccgg

SEQ ID NO: 735 - RNA, 27 nt, synthetic construct
  source 1..27: mol_type = other RNA
agcgcctccaactacgccgcgctgagc

SEQ ID NO: 736 - RNA, 33 nt, synthetic construct
  source 1..33: mol_type = other RNA
caggacagccgctccgcccccgagccgaagaag

SEQ ID NO: 737 - RNA, 33 nt, synthetic construct
  source 1..33: mol_type = other RNA
cccgccagcaagttctccagcgcctccaactac

SEQ ID NO: 738 - RNA, 30 nt, synthetic construct
  source 1..30: mol_type = other RNA
gacagccgctccgcccccgagccgaagaag

SEQ ID NO: 739 - RNA, 30 nt, synthetic construct
  source 1..30: mol_type = other RNA
caggacagccgctccgcccccgagccgaag

SEQ ID NO: 740 - RNA, 30 nt, synthetic construct
  source 1..30: mol_type = other RNA
gccagcaagttctccagcgcctccaactac

SEQ ID NO: 741 - RNA, 30 nt, synthetic construct
  source 1..30: mol_type = other RNA
ttcagctccgccagcaactacgccgcgctg

SEQ ID NO: 742 - RNA, 30 nt, synthetic construct
  source 1..30: mol_type = other RNA
agcaagttctccagcgcctccaactacgcc

SEQ ID NO: 743 - RNA, 27 nt, synthetic construct
  source 1..27: mol_type = other RNA
agccgctccgcccccgagccgaagaag

SEQ ID NO: 744 - RNA, 27 nt, synthetic construct
  source 1..27: mol_type = other RNA
gacagccgctccgcccccgagccgaag

SEQ ID NO: 745 - RNA, 27 nt, synthetic construct
  source 1..27: mol_type = other RNA
gcccccgagccgaagaagcccgaggag

SEQ ID NO: 746 - RNA, 27 nt, synthetic construct
  source 1..27: mol_type = other RNA
agcaagttctccagcgcctccaactac

SEQ ID NO: 747 - RNA, 27 nt, synthetic construct
  source 1..27: mol_type = other RNA
ttcagctccgccagcaactacgccgcg

SEQ ID NO: 748 - RNA, 30 nt, synthetic construct
  source 1..30: mol_type = other RNA
ttccccttcagcgtggagcagtccaacctg

SEQ ID NO: 749 - RNA, 30 nt, synthetic construct
  source 1..30: mol_type = other RNA
gaggagtacgccctggccttctacaacatc

SEQ ID NO: 750 - RNA, 27 nt, synthetic construct
  source 1..27: mol_type = other RNA
ttcagcgtggagcagtccaacctggtc

SEQ ID NO: 751 - RNA, 27 nt, synthetic construct
  source 1..27: mol_type = other RNA
ttccccttcagcgtggagcagtccaac

SEQ ID NO: 752 - RNA, 27 nt, synthetic construct
  source 1..27: mol_type = other RNA
cccttcagcgtggagcagtccaacctg

SEQ ID NO: 753 - RNA, 27 nt, synthetic construct
  source 1..27: mol_type = other RNA
gagtacgccctggccttctacaacatc

SEQ ID NO: 754 - RNA, 27 nt, synthetic construct
  source 1..27: mol_type = other RNA
aaggaggagtacgccctggccttctac

SEQ ID NO: 755 - RNA, 27 nt, synthetic construct
  source 1..27: mol_type = other RNA
gaggagtacgccctggccttctacaac

SEQ ID NO: 756 - protein, 42 aa, Homo sapiens
YVSNREVQVTSIKETFLSMGPASGLMLGCSRQEMGKLEKRTV

SEQ ID NO: 757 - protein, 16 aa, Homo sapiens
LGCSRQEMGKLEKRTV

SEQ ID NO: 758 - protein, 38 aa, Homo sapiens
ALCRCDPELCWISCEENSKEWMNAWKLTAGSHKAMTRK

SEQ ID NO: 759 - protein, 25 aa, Homo sapiens
CVHAYLSGQPLEATTLCQLHSTCHL

SEQ ID NO: 760 - protein, 72 aa, Homo sapiens
DATVLNSDQNSGEMLRRNSASTERVSEGFKDSITALSYHFYKNHIHFFPPTCPFSLSGTQALVLYIQVISKG

SEQ ID NO: 761 - protein, 64 aa, Homo sapiens
NVPKGQTGNSSRGPGDGGNKDHWKESDRKDGKKDQDSRSAPEPKKPEENPASKFSSASNYAALS

SEQ ID NO: 762 - protein, 66 aa, Homo sapiens
KTEVKNDHVLFYLENGFGRADSFPFSVEQSNLVFNIQPAPAMVYDYYEKEEYALAFYNIDSSSVSQ

SEQ ID NO: 763 - protein, 64 aa, Homo sapiens
ILRIAGLFHLIILPLRSMKTLRHVEGQMENSMPTCFALWMCWMFPRAPCLTNRALSKPYSCWKR

SEQ ID NO: 764 - RNA, 126 nt, synthetic construct
  source 1..126: mol_type = other RNA
tacgtgagcaaccgcgaggtccaggtgacctccatcaaggagaccttcctgagcatgggccccgcctccgggctcatgctgggctgcagccggcaggagatggggaagctggagaagcgcacggtg

SEQ ID NO: 765 - RNA, 48 nt, synthetic construct
  source 1..48: mol_type = other RNA
ctgggctgctcccgccaggagatggggaagctggagaagcggaccgtc

SEQ ID NO: 766 - RNA, 114 nt, synthetic construct
  source 1..114: mol_type = other RNA
gccctgtgccgctgcgaccccgagctctgctggatcagctgcgaggagaactccaaggagtggatgaacgcctggaagctgaccgcgggcagccacaaggccatgacccggaag

SEQ ID NO: 767 - RNA, 75 nt, synthetic construct
  source 1..75: mol_type = other RNA
tgcgtgcacgcctacctgagcggccagcccctcgaggccaccaccctgtgccagctgcactccacgtgccacctc

SEQ ID NO: 768 - RNA, 216 nt, synthetic construct
  source 1..216: mol_type = other RNA
gacgccaccgtgctgaacagcgaccagaactccggcgagatgctccgccggaacagcgcctccaccgagcgcgtcagcgaggggttcaaggactccatcaccgcgctgagctaccacttctacaagaaccacatccacttcttcccgcccacgtgccccttctccctgagcggcacccaggccctcgtgctgtacatccaggtgatctccaagggc

SEQ ID NO: 769 - RNA, 192 nt, synthetic construct
  source 1..192: mol_type = other RNA
aacgtgcccaagggccagaccgggaacagctcccgcggcccgggggacggcgggaacaaggaccactggaaggagagcgaccggaaggacggcaagaaggaccaggactcccgcagcgcccccgagcccaagaagcccgaggagaacccggcctccaagttcagctccgcgagcaactacgccgccctgtcc

SEQ ID NO: 770 - RNA, 198 nt, synthetic construct
  source 1..198: mol_type = other RNA
aagaccgaggtgaagaacgaccacgtcctgttctacctcgagaacggcttcgggcgcgccgacagcttccccttctccgtggagcagagcaacctggtgttcaacatccagccggcccccgcgatggtctacgactactacgagaaggaggagtacgccctggccttctacaacatcgactccagctccgtgagccag

SEQ ID NO: 771 - RNA, 192 nt, synthetic construct
  source 1..192: mol_type = other RNA
atcctgcgcatcgccggcctcttccacctgatcatcctgcccctccggagcatgaagaccctgcgccacgtggaggggcagatggagaactccatgccgacctgcttcgccctgtggatgtgctggatgttcccccgggcgccctgcctcacgaaccgcgccctgagcaagccctactcctgctggaagcgg

(19) United States
(12) Patent Application Publication
(10) Pub. No.: US 2026/0335418 A1
(43) Pub. Date: Oct. 29, 2026

(54) CANCER ANTIGENS

(71) Applicant: CureVac SE, Tübingen (DE)

(72) Inventors: Esteban Peguero SÁNCHEZ, Tübingen (DE); Janna Frieda KRÜGER, Tübingen (DE); Bruno FANT, Gent (BE); Lore VAN OUDENHOVE, Gent (BE); Steve LEFEVER, Gent (BE); Cedric BOGAERT, Gent (BE)

(73) Assignee: CureVac SE, Tübingen (DE)

(21) Appl. No.: 19/487,349

(22) PCT Filed: May 23, 2024
(86) PCT No.: PCT/EP2024/064280

§ 371 Date: Nov. 24, 2025

(30) Foreign Application Priority Data
May 26, 2023 (WO) PCT/EP2023/064278

(51) Int. Cl. A61K 39/00; A61K 38/00; A61K 40/11; A61K 40/42; A61P 37/04; C07K 14/47; C07K 14/705; C07K 16/32

(52) U.S. Cl. CPC A61K 39/0011; A61K 40/11; A61K 40/42; A61P 37/04; C07K 14/4748; C07K 14/7051; C07K 16/32; A61K 38/00; A61K 2039/53; A61K 2039/70

(57) ABSTRACT

The present invention relates to tumour or cancer antigens, in particular amino acid sequences and nucleic acid sequences, that may be used in cancer immunotherapy. In particular, the invention is directed to an artificial nucleic acid, preferably RNA, comprising at least one coding sequence encoding at least one tumour or cancer antigen that comprises or consists of at least one antigenic peptide selected or derived from a peptide or protein encoded by a small open reading frame (smORF) of long non-coding RNA (lncRNA), or a fragment or variant thereof, and/or at least one antigenic peptide selected or derived from a peptide or protein of a tumour neoantigen, or a fragment or variant thereof. Further provided are pharmaceutical compositions comprising the artificial nucleic acid, preferably formulated in lipid-based carriers. Also provided are methods of treating or preventing disorders, diseases, or conditions, and medical uses, in particular for treating or preventing cancer, such as NSCLC, HNSCC, or melanoma.

### INTRODUCTION

Cancer is a major global health problem and is one of the leading causes of death worldwide. Traditional cancer treatments, such as surgery, chemotherapy, and radiation therapy, have limited efficacy and can cause significant side effects. Immunotherapy, which involves harnessing the power of the immune system to target cancer cells, has emerged as a promising new approach for the treatment of cancer. Cancer immunotherapy is the use of the immune system to treat cancer. Immunotherapies exploit the fact that cancer cells often have molecules on their surface that can be detected by the immune system, known as tumour antigens, which are often proteins or other macromolecules. Promising candidates for highly specific immunotherapy are neoantigens or antigens or tumour associated antigens.

In recent years, nucleic acid such as RNA has emerged as a promising therapeutic tool for the treatment of cancer and other diseases, as it can be used to direct the production of proteins that can inhibit or otherwise reduce the growth or survival of cancer cells. The advantages of using RNA include transient expression and a non-transforming character—RNA does not need to enter the nucleus to be expressed and moreover cannot integrate into the host genome, thereby eliminating the risk of oncogenesis. Accordingly, nucleic acid, e.g. RNA, may represent a promising class of molecules to provide the information for expressing tumour antigens such as neoantigens or tumour-associated antigens.

Accordingly, the object of the present invention is to provide neoantigens or tumour-associated antigens for cancer immunotherapy, in particular neoantigens or tumour-associated antigens provided by nucleic acid constructs.

The object mentioned above is solved by the underlying description and the accompanying claims. In particular, the object of the invention is solved by providing novel tumour antigens, nucleic acids encoding such tumour antigens, and the use of these tumour antigens/nucleic acids in treating cancer in a subject such as NSCLC, HNSCC, or melanoma. In particular, tumour antigens are provided that are unexpectedly originate from small open reading frames of long non-coding RNA (that, as the term suggests, have long been considered as being non-coding).

### SHORT DESCRIPTION OF THE INVENTION

The present invention inter alia relates to novel tumour or cancer antigens, in particular amino acid sequences and nucleic acid sequences, that may be used in cancer immunotherapy. In particular, the invention is directed to an artificial nucleic acid, preferably RNA, comprising at least one coding sequence encoding at least one tumour or cancer antigen that comprises or consists of at least one antigenic peptide selected or derived from a peptide or protein encoded by a small open reading frame (smORF) of lncRNA, or a fragment or variant thereof; and/or at least one antigenic peptide selected or derived from a peptide or protein of a tumour neoantigen, or a fragment or variant thereof. Further provided are pharmaceutical compositions comprising the artificial nucleic acid, preferably formulated in lipid-based carriers. Also provided are methods of treating or preventing disorders, diseases, or conditions, and medical uses, in particular cancer such as NSCLC, HNSCC, or melanoma.

As shown herein, the invention is inter alia based on the surprising finding that peptides encoded by small open reading frames (smORF) of long non-coding RNA (lncRNA) can serve as effective tumour associated antigens. Additionally, neoantigens have been identified that can serve as effective tumour specific antigens. These tumour antigens can be provided by an artificial nucleic acid, e.g. mRNA, for treating cancer, e.g. NSCLC, HNSCC, or melanoma.

Accordingly, in a first aspect, the present invention provides an artificial nucleic acid comprising at least one coding sequence encoding at least one tumour or cancer antigen that comprises (a) at least one antigenic peptide selected or derived from a peptide or protein encoded by a long non-coding RNA (lncRNA); and/or (b) at least one antigenic peptide selected or derived from a peptide or protein of a tumour neoantigen.

In preferred embodiments, the at least one antigenic peptide is selected or derived from a peptide or protein encoded by a long non-coding RNA (lncRNA) selected from lnc-WDR72-2:4, lnc-TRPC5-3:1, lnc-CLEC2D-9:1, KCNMB2-AS1:4, lnc-ZC3H8-6:1, lnc-NTF3-5:5, or LINC00893:25, or an immunogenic fragment or variant thereof.

In other preferred embodiments, the at least one antigenic peptide is selected from a peptide or protein of a tumour neoantigen is selected from NRAS, TYW1B, ECPAS, MAP2K1, TOMM22, GLB1, MAGE-A3, ATAD2, BRAF, EGFR, or TP53, or an immunogenic fragment or variant thereof.

In a second aspect, the present invention provides a pharmaceutical composition comprising at least one artificial nucleic acid as defined in the first aspect. The artificial nucleic acid, preferably an RNA, may be formulated in LNPs.

In a third aspect, the present invention provides a tumour antigen or a composition antigen.

In a fourth aspect, the present invention provides an antibody, a T-cell, or a TCR that has been raised against or stimulated with any of at least one nucleic acid encoding a tumour antigen of the invention.

In a fifth aspect, the present invention provides a combination comprising at least two or a plurality of therapeutic modalities of any of the foregoing aspects.

In a sixth aspect, the present invention provides a kit or kit of parts comprising at least one artificial nucleic acid of the first aspect; at least one pharmaceutical composition of the second aspect; at least one tumour antigen or composition of the third aspect; and/or at least one antibody, T-cell, or TCR of the fourth aspect.

In further aspects, the invention provides methods of treating or preventing disease, disorder or condition and medical uses of the artificial nucleic acid, the pharmaceutical composition, the tumour antigen or composition, the antibody, T-cell, or TCR, or the kit or kit of parts. Preferably, the disease, disorder or condition is cancer, or any disease, disorder, or condition related to cancer, such as NSCLC, HNSCC or melanoma.

### DEFINITIONS

For the sake of clarity and readability the following definitions are provided. Any technical feature mentioned for these definitions may be read on each and every embodiment of the invention. Additional definitions and explanations may be specifically provided in the context of these embodiments.

Percentages in the context of numbers should be understood as relative to the total number of the respective items. In other cases, and unless the context dictates otherwise, percentages should be understood as percentages by weight (wt.-%).

About: The term “about” is used when determinants or values do not need to be identical, i.e. 100% the same and that e.g. the respective values or determinants may diverge by 1% to 10%. Preferably, “about” means, that a determinant or values may diverge by +/−1%, +/−2%, +/−3%, +/−4%, +/−5%, +/−6%, +/−7%, +/−8%, +/−9%, +/−10%.

Antigenic peptide: The term “antigenic peptide” or “immunogenic peptide” as used herein refers to a peptide derived from a (antigenic or immunogenic) protein which stimulates the body's adaptive or cellular immune system to provide an adaptive or cellular immune response. Therefore an antigenic/immunogenic peptide comprises at least one epitope or antigen selected or derived from a tumour antigen as defined herein.

Cationic, cationizable: The term “cationic” means that the respective structure, compound, or group, or atom bears a positive charge, either permanently or not permanently, e.g. in response to certain conditions such as pH. The terms “cationic”, “cationisable”, and “permanently cationic” as used herein must be understood as defined in WO2023/031394 [p. 12, line 32 to p. 13, line 16].

Cationic compound, polycationic compound: Where a respective structure, compound, or group, or atom carries a plurality of positive charges, it may be referred to as polycationic. The terms as used herein must be understood as defined in WO2021/156267 [p. 88, line 12 to p. 89, line 22].

Coding sequence, coding region, cds: The terms “coding sequence” or “the corresponding abbreviation “cds” as used herein refers to a sequence of several nucleotide triplets that can be translated into a peptide or protein. A cds in the context of the present invention may be a DNA or RNA sequence consisting of several nucleotides that may be divided by three, which typically starts with a start codon and preferably terminates with a stop codon. In the context of the invention, the cds encodes at least one tumour antigen as defined herein.

Derived from: The term “derived from” as used herein in the context of a nucleic acid, i.e. for a nucleic acid derived from (another) nucleic acid, means that the nucleic acid, which is derived from (another) nucleic acid, shares at least 70%, 80%, 81%, 82%, 83%, 84%, 85%, 86%, 87%, 88%, 89%, 90%, 91%, 92%, 93%, 94%, 95%, 96%, 97%, 98%, or 99% sequence identity or is identical with the nucleic acid from which it is derived. In the context of amino acid sequences, the term “derived from” means that the amino acid sequence, which is derived from (another) amino acid sequence, shares at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity or is identical with the amino acid sequence from which it is derived.

Epitope: The term “epitope” as used herein refers to T cell epitopes and B cell epitopes. T cell epitopes or parts of the antigenic peptides or proteins and may comprise fragments preferably having a length of about 6 to about 20 or even more amino acids, e.g. fragments as processed and presented by MHC class I molecules, preferably having a length of about 8 to about 10 amino acids, e.g. 8, 9, or 10, (or even 11, or 12 amino acids), or fragments as processed and presented by MHC class II molecules, preferably having a length of about 13 to about 20 or even more amino acids. These fragments are typically recognized by T cells in form of a complex consisting of the peptide fragment and an MHC molecule, i.e. the fragments are typically not recognized in their native form. B cell epitopes are typically fragments located on the outer surface of (native) protein or peptide antigens, preferably having 5 to 15 amino acids, more preferably having 5 to 12 amino acids, even more preferably having 6 to 9 amino acids, which may be recognized by antibodies, i.e. in their native form. Such epitopes of proteins or peptides may furthermore be selected from any of the herein mentioned variants of such proteins or peptides. In this context epitopes can be conformational or discontinuous epitopes which are composed of segments of the proteins or peptides as defined herein that are discontinuous in the amino acid sequence of the proteins or peptides as defined herein but are brought together in the three-dimensional structure or continuous or linear epitopes which are composed of a single polypeptide chain.

Fragment: The term “fragment” as used herein in the context of a nucleic acid sequence (e.g. RNA or DNA) or an amino acid sequence may typically be a shorter portion of a reference sequence of e.g. a nucleic acid sequence or an amino acid sequence. Accordingly, a fragment typically consists of a sequence that is identical to the corresponding stretch within the reference sequence. A preferred fragment of a sequence in the context of the present invention, consists of a continuous stretch of entities, such as nucleotides or amino acids corresponding to a continuous stretch of entities in the molecule the fragment is derived from, which represents at least 50%, 60%, 65%, 70%, 75%, 80%, 85%, 90%, 95% of the total reference molecule from which the fragment is derived.

Identity (of a sequence); The term “identity” as used herein in the context of a nucleic acid sequence, or an amino acid sequence, refers to the percentage to which two sequences are identical. To determine the percentage to which two sequences are identical, the sequences can be aligned (by also introducing gaps, if necessary) to be subsequently compared to one another. In the context of the invention, the substitution of a nucleotide by a modified nucleotide (e.g. U substituted by with N1-methylpseudouridine (m1ψ) shall not be considered for calculating percent identity. The percentage to which two sequences are identical can e.g. be determined using an algorithm, e.g. an algorithm integrated in the BLAST program.

Immunogen, immunogenic: The terms “immunogen” or “immunogenic” as used herein refers to a compound that is able to stimulate/induce an immune response. Preferably, an immunogen is a peptide, polypeptide, or protein.

RNA in vitro transcription: The terms “RNA in vitro transcription” or “in vitro transcription” relate to a process where RNA is synthesized in a cell-free system in vitro. In RNA in vitro transcription (IVT), the RNA is obtained by DNA-dependent in vitro transcription of an appropriate DNA template in the presence of a DNA-dependent RNA polymerases (e.g. T7, T3, SP6, or Syn5), ribonucleotide triphosphates (NTPs, and optionally modified NTPs); optionally, a cap analogue, MgCl₂, and a buffer (comprising TRIS or HEPES, optionally DTT and/or spermidine).

Variant (of a sequence): The term “variant” as used herein in the context of a nucleic acid sequence will be recognized and understood by the person of ordinary skill in the art, and is e.g. intended to refer to a variant of a nucleic acid sequence derived from another nucleic acid sequence. E.g., a variant of a nucleic acid sequence may exhibit one or more nucleotide deletions, insertions, additions and/or substitutions compared to the nucleic acid sequence from which the variant is derived. A variant of a nucleic acid sequence may at least 50%, 60%, 70%, 80%, 90%, or 95% identical to the nucleic acid sequence the variant is derived from. A variant may be a functional variant in the sense that the variant has retained at least 50%, 60%, 70%, 80%, 90%, or 95% or more of the function of the sequence where it is derived from. A “variant” of a nucleic acid sequence may have at least 70%, 75%, 80%, 85%, 90%, 95%, or 99% nucleotide identity over a stretch of at least 30, 50, 75 or 100 nucleotides of such nucleic acid sequence.

The term “variant” as used herein in the context of proteins or peptides is e.g. intended to refer to a proteins or peptide variant having an amino acid sequence which differs from the original sequence in one or more mutation(s)/substitution(s), such as one or more substituted, inserted and/or deleted amino acid(s). Preferably, these fragments and/or variants have the same, or a comparable specific property. Insertions and substitutions are possible, in particular, at those sequence positions which cause no modification to the three-dimensional structure or do not affect the binding region. Modifications to a three-dimensional structure by insertion(s) or deletion(s) can easily be determined e.g. using CD spectra (circular dichroism spectra). A variant of a protein may be a functional variant of the protein, which means that the variant exerts essentially the same, or at least 40%, 50%, 60%, 70%, 80%, 90% of the function of the protein it is derived from. A “variant” of a protein or peptide may have at least 70%, 75%, 80%, 85%, 90%, 95%, 98% or 99% amino acid identity over a stretch of at least 30, 50, 75 or 100 amino acids of such protein or peptide.

NSCLC/Non-Small Cell Lung Cancer/lung cancer: Lung cancer, also known as lung carcinoma, is a malignant tumour that origin is based in cells of the lung. At diagnosis, lung cancer is classified based on the type of cells the tumour is derived from; tumours derived from different cells progress and respond to treatment differently. Around 85% of the lung cancers are non-small-cell lung cancers, like adenocarcinomas, squamous-cell carcinomas and large-cell carcinomas. NSCLC can be divided into different subgroups including lung adenocarcinomas (LUAD) and lung squamous cell carcinomas (LUSC)/squamous non-small-cell lung cancer (sqNSCLC).

Head and Neck cancer: Is a cancer that typically develops from tissues in the lip and oral cavity, larynx (throat), salivary glands, nose, sinuses, or skin of the face. The most common types of head and neck cancer occur in the lips, mouth, and larynx. About 90% are pathologically classified as squamous cell cancers (HNSCC).

Squamous cell carcinomas: Squamous-cell carcinoma is a type of cancer that typically comprises several different types of cancer that begin in squamous cells. These cells form on the surface of the skin, on the lining of hollow organs in the body, and on the lining of the respiratory and digestive tracts. Squamous-cell carcinoma comprise e.g. squamous head and neck cancer (e.g. cancer of the mouth, nasal cavity, nasopharynx, throat, and associated structures), cutaneous squamous-cell carcinoma, primary squamous-cell carcinoma of the thyroid, oesophageal squamous-cell carcinoma, lung squamous cell carcinomas, and squamous-cell carcinoma of penis, vagina, ovary, bladder and eye.

Melanoma: Is a type of skin cancer that develops from the pigment-producing cells known as melanocytes and is the most dangerous type of skin cancer. The outcome of melanoma depends on the stage at presentation. Types of melanomas include e.g. skin cutaneous melanoma (SKCM) or uveal melanoma. Typically, SKCM can be divided into different subgroups, e.g. a BRAF subgroup and a NRAS subgroup.

### DETAILED DESCRIPTION OF THE INVENTION

Where reference is made to “SEQ ID NOs” of other patent applications or patents, said sequences, e.g. amino acid sequences or nucleic acid sequences, are explicitly incorporated herein by reference. For “SEQ ID NOs” provided herein, information provided under “feature key”, i.e. “source” (for nucleic acids or proteins) or “misc_feature” (for nucleic acids) or “REGION” (for proteins), in the sequence listing according to WIPO ST.26 Standard is also explicitly included herein. Where reference is made to “SEQ ID NOs” in the context of RNA sequences, the skilled person will be able to derive RNA sequences from the referenced SEQ ID NOs also in cases where DNA sequences are provided. Where reference is made to “SEQ ID NOs” in the context of DNA sequences, the skilled person will be able to derive respective DNA sequences from the referenced SEQ ID NOs also in cases where RNA sequences are provided.

### 1: Nucleic Acid Encoding at Least One Tumour Antigen:

In a first aspect, the invention provides a nucleic acid encoding at least one tumour antigen.

Notably, specific features and embodiments that are described in the context of the first aspect of the invention, that is the nucleic acid of the invention, are likewise applicable to any other aspect of the invention including the pharmaceutical composition of the second aspect, the tumour antigen or a composition of tumour antigens of the third aspect, the antibody, a T-cell, or a TCR of the fourth aspect, the combination of the fifth aspect, the kit or kit of parts of the sixth aspect, or any of the aspects relating to medical uses or methods of treatment.

In preferred embodiments, the nucleic acid encoding the at least one tumour antigen is an artificial nucleic acid.

The term “artificial nucleic acid” as used herein refers to a nucleic acid that does not occur naturally. In other words, an artificial nucleic acid may be understood as a non-natural nucleic acid molecule. Such nucleic acid molecules may be non-natural due to its individual sequence (e.g. codon modified coding sequence, UTRs) and/or due to other modifications, e.g. structural modifications of nucleotides. Typically, an artificial nucleic acid may be designed and/or generated by genetic engineering to correspond to a desired artificial sequence of nucleotides. In this context, an artificial nucleic acid is a sequence that may not occur naturally, i.e. a sequence that differs from the wild type sequence or the naturally occurring sequence by at least one nucleotide. The term “artificial nucleic acid” is not restricted to mean “one single molecule” but is understood to comprise an ensemble of essentially identical nucleic acid molecules. The term “artificial nucleic acid” as used herein may relate to artificial DNA or artificial RNA. Preferably, the artificial nucleic acid is selected from an artificial RNA.

In preferred embodiments, the artificial nucleic acid comprises at least one coding sequence encoding at least one tumour antigen.

The term “tumour antigen” as used herein is intended to refer to tumour-associated antigens (TAAs), expressed by both normal and neoplastic tissue, and tumour-specific antigens (TSAs) including shared antigens, neoantigens, and unique antigens. Further tumour antigens can be loosely categorized as oncofetal (typically only expressed in fetal tissues and in cancerous somatic cells), oncoviral (encoded by tumorigenic transforming viruses), overexpressed/accumulated (expressed by both normal and neoplastic tissue, with the level of expression highly elevated in neoplasia), cancer-testis (expressed only by cancer cells and adult reproductive tissues such as testis and placenta), lineage-restricted (expressed largely by a single cancer histotype), mutated (only expressed by cancer as a result of genetic mutation or alteration in transcription), posttranslationally altered (tumour-associated alterations in glycosylation, etc.), or idiotypic (highly polymorphic genes where a tumour cell expresses a specific “clonotype”, i.e., as in B cell, T cell lymphoma/leukemia resulting from clonal aberrancies). It should be stressed that these categories are not mutually exclusive and tumour antigens may fall into more than one category.

In preferred embodiments, the tumour antigen (that is provided by the artificial nucleic acid) is produced in the cytosol upon administration of the artificial nucleic acid to a cell, tissue, or subject. The produced tumour antigen may be secreted or further processed.

Accordingly, the administration of the artificial nucleic acid (e.g. RNA) to a cell, tissue, or subject leads to a translation of the at least one coding sequence into at least one tumour antigen. Whenever reference is made to a “produced tumour antigen”, the term relates to the protein product that is generated from the artificial nucleic acid of the invention by translating the coding sequence of the nucleic acid into a protein. Accordingly, functional and structural features and embodiments that are described herein relating to the “tumour antigen” or relating to the “(produced) tumour antigen” should be understood to refer to tumour antigen amino acid sequences that are produced/translated in the cytosol upon administration of the artificial nucleic acid to a cell, tissue, or subject.

In embodiments, the artificial nucleic acid comprises at least one coding sequence encoding at least one tumour antigen that comprises or consists of at least one antigenic peptide selected or derived from a peptide or protein encoded by a long non-coding RNA (lncRNA), or an immunogenic fragment or variant of such a peptide or protein.

In embodiments, the artificial nucleic acid comprises at least one coding sequence encoding at least one tumour antigen that comprises or consists of at least one antigenic peptide selected or derived from a peptide or protein of a tumour neoantigen, or an immunogenic fragment or variant of such a peptide or protein.

The term “immunogenic fragment” or “immunogenic variant” has to be understood as any fragment/variant of the corresponding peptide or protein capable of raising an immune response in a subject, preferably a human subject.

In embodiments, the artificial nucleic acid comprises at least one coding sequence encoding at least one tumour antigen that comprises or consists of
a) at least one amino acid sequence encoded by a long non-coding RNA (lncRNA), or an immunogenic fragment or variant thereof; and/or
b) at least one amino acid sequence of a tumour neoantigen, or an immunogenic fragment or variant thereof.

In embodiments, the artificial nucleic acid comprises at least one coding sequence encoding at least one tumour antigen that comprises or consists of
a) at least one antigenic peptide selected or derived from a peptide or protein encoded by a long non-coding RNA (lncRNA), or an immunogenic fragment or variant thereof; and/or
b) at least one antigenic peptide selected or derived from a peptide or protein of a tumour neoantigen, or an immunogenic fragment or variant thereof.

In preferred embodiments, the artificial nucleic acid comprises at least one coding sequence encoding at least one tumour antigen that comprises or consists of at least one antigenic peptide selected or derived from a peptide or protein encoded by a long non-coding RNA (lncRNA) or an immunogenic fragment or variant thereof.

Tumour Antigens Encoded by Long Non-Coding RNA (lncRNA)

In preferred embodiments, the artificial nucleic acid comprises at least one coding sequence encoding at least one tumour antigen that comprises or consists of at least one antigenic peptide selected or derived from a peptide or protein encoded by a long non-coding RNA (lncRNA), or an immunogenic fragment or variant thereof.

The term “long non-coding RNA” and its abbreviation “lncRNA” relates to an RNA having a length of typically greater than 200 bases (up to e.g. 10.0000 bases). In the past, lncRNA has been considered to have no protein encoding function. Research on long non-coding RNA in recent years has revealed that these molecules participate in various regulatory aspects of cells. Compared with mRNA, long non-coding RNA has the characteristics of low expression abundance but has stronger tissue and cell expression specificity. Recently, several lncRNAs have been found to encode for peptides with biologically significant function, e.g. micropeptides or putative small open reading frames (also herein referred to as “mini ORF” or “small ORF” (smORF)). Accordingly, the term “long non-coding RNA” or its abbreviation “lncRNA” is used throughout the specification due to the nomenclature that is common in the art. Notably, lncRNA species in the context of the invention encode peptides or proteins that can serve as suitable tumour antigens.

Surprisingly, peptides have been identified by the inventors that are produced by certain lncRNA species. More surprisingly, these peptides, encoded by lncRNA, show specificity for certain tumours (e.g. melanoma, NSCLC, HNSCC) and can raise an antigen specific immune response, e.g. these peptides can be recognized by the immune cells as being foreign, can be presented, and can raise an antigen specific immune response (e.g. T-cell response). Thus, peptides selected or derived from smORFs of lncRNA are suitable for cancer immunotherapy.

In preferred embodiments, the at least one antigenic peptide is selected from an amino acid sequence such as a peptide or protein encoded by a long non-coding RNA (lncRNA), or a (immunogenic) fragment or variant thereof, wherein the lncRNA comprises at least one smORF (encoding said peptide or protein) and wherein the lncRNA is characterized by an increased expression in cancer cells (e.g. melanoma, NSCLC, HNSCC) compared to non-cancer cells. Moreover, the lncRNA comprises at least one smORF.

In preferred embodiments, the at least one antigenic peptide is selected from a peptide or protein encoded by a long non-coding RNA (lncRNA), or an immunogenic fragment or variant thereof, said lncRNA being selected from lnc-WDR72-2:4, lnc-TRPC5-3:1, lnc-CLEC2D-9:1, KCNMB2-AS1:4, lnc-ZC3H8-6:1, lnc-NTF3-5:5, or LINC00893:25, or a fragment or variant of any of these, preferably an immunogenic fragment or variant of any of these.

In preferred embodiments, the at least one antigenic peptide comprises an amino acid sequence encoded by a long non-coding RNA (lncRNA), or a fragment or variant thereof, said lncRNA being selected from lnc-WDR72-2:4, lnc-TRPC5-3:1, lnc-CLEC2D-9:1, KCNMB2-AS1:4, lnc-ZC3H8-6:1, lnc-NTF3-5:5, or LINC00893:25, or a fragment or variant of any of these, preferably an immunogenic fragment or variant of any of these.

The terms “amino acid sequence encoded by” or “peptide or protein encoded by” or “antigen encoded by” in the context of a certain lncRNA species (e.g. encoded by ZC3H8-6:1, WDR72-2:4, KCNMB2-AS1:4, or NTF3-5:5) defines that the origin or source of the respective amino acid sequence, peptide, protein, or antigen is a long non-coding RNA. Accordingly, in other words, the respective amino acid sequence, peptide, protein, or antigen originates from the respective lncRNA species. In the context of the invention, the respective amino acid sequence, peptide, protein, or antigen that is encoded by a certain lncRNA species (e.g. ZC3H8-6:1, WDR72-2:4, KCNMB2-AS1:4, or NTF3-5:5), or, in other words, that originates from a certain lncRNA species (e.g. ZC3H8-6:1, WDR72-2:4, KCNMB2-AS1:4, or NTF3-5:5) is provided nucleic acid of the invention (e.g. an mRNA).

Preferred in the context of the invention are amino acid sequences such as peptide or proteins encoded by lnc-NTF3-5:5, lnc-WDR72-2:4, KCNMB2-AS1:4, lnc-ZC3H8-6:1, or lnc-CLEC2D-9:1, or an immunogenic fragment or variant thereof, or nucleic acid sequences encoding amino acid sequences such as peptides or proteins encoded by lnc-NTF3-5:5, lnc-WDR72-2:4, KCNMB2-AS1:4, lnc-ZC3H8-6:1, or lnc-CLEC2D-9:1, or fragments or variants thereof.

Particularly preferred in the context of the invention are amino acid sequences such as peptides or proteins encoded by lnc-NTF3-5:5, or an immunogenic fragment or variant thereof, or nucleic acid sequences encoding amino acid sequences such as peptides or proteins encoded by lnc-NTF3-5:5, or fragments or variants thereof.

Importantly, lnc-WDR72-2:4, lnc-TRPC5-3:1, lnc-CLEC2D-9:1, KCNMB2-AS1:4, lnc-ZC3H8-6:1, lnc-NTF3-5:5, and LINC00893:25 are predominantly expressed in cancer cells as shown by RNA sequencing (see Example section) and peptides encoded by these lncRNAs have been identified by mass spectrometry (see Example section).

lnc-WDR72-2:4 is a long non-coding RNA that is transcribed from the following genomic location in humans: chr15,53513740,53540852,-. The naturally occurring (human) lncRNA sequence of lnc-WDR72-2:4 is according to SEQ ID NO: 512. The RNA sequence of lnc-WDR72-2:4 comprises at least two open reading frames (smORFs lncWDR722416421696 and lncWDR722415641696) that encode at least one amino acid sequence, e.g. at least one peptide that can serve as tumour antigen in the context of the invention. Suitably, any peptide or protein, or immunogenic fragment or variant thereof, produced by lnc-WDR72-2:4 can serve as a suitable tumour antigen in the context of the invention.

lnc-TRPC5-3:1 is a long non-coding RNA that is transcribed from the following genomic location in humans: chrX, 111619483,111621517,-. The naturally occurring (human) lncRNA sequence of lnc-TRPC5-3:1 is according to SEQ ID NO: 516. The RNA sequence of lnc-TRPC5-3:1 comprises at least one open reading frame (smORF lncTRPC53116031801) that encodes at least one amino acid sequence, e.g. at least one peptide that can serve as tumour antigen in the context of the invention. Suitably, any peptide or protein, or immunogenic fragment or variant thereof, produced by lnc-TRPC5-3:1 can serve as a suitable tumour antigen in the context of the invention.

lnc-CLEC2D-9:1 is a long non-coding RNA that is transcribed from the following genomic location in humans: chr12,9570760,9576267,+. The naturally occurring (human) lncRNA sequence of lnc-CLEC2D-9:1 is according to SEQ ID NO: 517. The RNA sequence of lnc-CLEC2D-9:1 comprises at least one open reading frame (smORF lncCLEC2D91412616) that encode at least one amino acid sequence, e.g. at least one peptide that can serve as tumour antigen in the context of the invention. Suitably, any peptide or protein, or immunogenic fragment or variant thereof, produced by lnc-CLEC2D-9:1 can serve as a suitable tumour antigen in the context of the invention.

KCNMB2-AS1:4 is a long non-coding RNA that is transcribed from the following genomic location in humans: chr3,178525486, 178860397,-. The naturally occurring (human) lncRNA sequence of KCNMB2-AS1:4 is according to SEQ ID NO: 513. The RNA sequence of KCNMB2-AS1:4 comprises at least one open reading frame (smORF KCNMB2AS14633753) that encodes at least one amino acid sequence, e.g. at least one peptide that can serve as tumour antigen in the context of the invention. Suitably, any peptide or protein, or immunogenic fragment or variant thereof, produced by KCNMB2-AS1:4 can serve as a suitable tumour antigen in the context of the invention.

lnc-ZC3H8-6:1 is a long non-coding RNA that is transcribed from the following genomic location in humans: chr2,111769354,111776910,-. The naturally occurring (human) lncRNA sequence of lnc-ZC3H8-6:1 is according to SEQ ID NO: 514. The RNA sequence of lnc-ZC3H8-6:1 comprises at least one open reading frame (smORF lncZC3H86163144) that encodes at least one amino acid sequence, e.g. at least one peptide that can serve as tumour antigen in the context of the invention. Suitably, any peptide or protein, or immunogenic fragment or variant thereof, produced by lnc-ZC3H8-6:1 can serve as a suitable tumour antigen in the context of the invention.

lnc-NTF3-5:5 is a long non-coding RNA that is transcribed from the following genomic location in humans: chr12,5367810,5379568,+. The naturally occurring (human) lncRNA sequence of lnc-NTF3-5:5 is according to SEQ ID NO: 515. The RNA sequence of lnc-NTF3-5:5 comprises at least one open reading frame (smORF lncNTF35511951417) that encodes at least one amino acid sequence, e.g. at least one peptide that can serve as tumour antigen in the context of the invention. Suitably, any peptide or protein, or immunogenic fragment or variant thereof, produced by lnc-NTF3-5:5 can serve as a suitable tumour antigen in the context of the invention.

LINC00893:25 (encoding smORF LINC00893) is a long non-coding RNA that is transcribed from the following genomic location in humans: chrX, 149483112,149532914,-. The naturally occurring (human) lncRNA sequence of LINC00893:25 is according to SEQ ID NO: 518. The RNA sequence of LINC00893:25 comprises at least one open reading frame (smORF LINC00893) that encodes at least one amino acid sequence, e.g. at least one peptide that can serve as tumour antigen in the context of the invention. Suitably, any peptide or protein, or immunogenic fragment or variant thereof, produced by LINC00893:25 can serve as a suitable tumour antigen in the context of the invention.

Preferably, the at least one antigenic peptide encoded by the long non-coding RNA (lncRNA), or immunogenic fragment or variant of any of these, comprises at least one T-cell epitope, preferably at least one CD8+ T cell epitope.

Preferably, the at least one antigenic peptide encoded by the long non-coding RNA lnc-WDR72-2:4, lnc-TRPC5-3:1, lnc-CLEC2D-9:1, KCNMB2-AS1:4, lnc-ZC3H8-6:1, lnc-NTF3-5:5, or LINC00893:25, or immunogenic fragment or variant of any of these, comprises at least one T-cell epitope, preferably at least one CD8+ T cell epitope.

In preferred embodiments, the at least one antigenic peptide encoded by the long non-coding RNA (lncRNA), or immunogenic fragment or variant of any of these, induce epitope-specific T cells in a subject, preferably epitope specific CD8+ T cells in a subject (in particular, a human subject).

In particularly preferred embodiments, the at least one antigenic peptide encoded by the long non-coding RNA lnc-WDR72-2:4, lnc-TRPC5-3:1, lnc-CLEC2D-9:1, KCNMB2-AS1:4, lnc-ZC3H8-6:1, lnc-NTF3-5:5, or LINC00893:25, or immunogenic fragment or variant of any of these, induce epitope-specific T cells in a subject, preferably epitope specific CD8+ T cells in a subject (in particular, a human subject).

The term “T-cell epitope” refers to a part or fragment of a protein or antigen that is recognized by a T cell when presented in the context of major histocompatibility complex (MHC) molecules, includes MHC class I and MHC class II molecules and relates to a complex of genes which is present in all vertebrates. MHC proteins or molecules are important for signalling between lymphocytes and antigen presenting cells or diseased cells in immune reactions, wherein the MHC proteins or molecules bind peptide epitopes and present them for recognition by T-cell receptors on T cells. The proteins encoded by the MHC are expressed on the surface of cells, and display both self-antigens (peptide fragments from the cell itself) and non-self-antigens (e.g., fragments of invading microorganisms) to a T cell. In the case of class I MHC/peptide complexes, the binding peptides (MHC class I ligands) are typically about 8 to about 10 amino acids long although longer or shorter peptides may be effective. In the case of class II MHC/peptide complexes, the binding peptides (MHC class II ligands) are typically about 10 to about 25 amino acids long and are in particular about 13 to about 18 amino acids long, whereas also longer and shorter peptides may be effective.

Preferably, the at least one antigenic peptide encoded by the lncRNA, or immunogenic fragment or variant thereof, in particular lnc-WDR72-2:4, lnc-TRPC5-3:1, lnc-CLEC2D-9:1, KCNMB2-AS1:4, lnc-ZC3H8-6:1, lnc-NTF3-5:5, or LINC00893:25, is an antigen or epitope displaying HLA binding affinity or MHC binding affinity.

The term “HLA” or “human leukocyte antigen” as used herein includes variants, isoforms, and species homologs of HLA, and analogues having at least one common epitope with an HLA. “Human leukocyte antigen (HLA),” as used herein, should preferably be understood as a gene complex encoding the “major histocompatibility complex (MHC)” proteins in humans. These cell surface proteins are responsible for the regulation of the immune system in humans. HLA genes are highly polymorphic, i.e. having may different alleles, which allows them to fine-tune the adaptive immune system of a subject. Within the context of this description, the term “HLA binding affinity” or “MHC binding affinity” is to be understood as affinity of binding between a specific antigen and a specific MHC allele. Within the context of this description, the term “HLA type” is to be understood as the complement of HLA gene alleles.

Preferably, the at least one antigenic peptide encoded by the lncRNA as specified herein is capable of binding to an antibody or a T-cell receptor. Preferably, the antigenic peptide comprises at least one MHCI or MHCII ligand.

In embodiments, the at least one antigenic peptide encoded by a lncRNA as defined herein is a shared tumour associated antigen, in particular, a shared tumour associated antigen in NSCLC and/or melanoma.

In preferred embodiments, the at least one antigenic peptide (provided by the artificial nucleic acid) is selected from a peptide or protein encoded by a long non-coding RNA (lncRNA) as defined herein and comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 124-254, 546-650, 756-763, or an immunogenic fragment or variant of any of these.

Accordingly, the at least one antigenic peptide preferably comprises at least one amino acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 124-254, 546-650, 756-763, or an immunogenic fragment or variant of any of these.

In that context, an immunogenic fragment of SEQ ID NOs: 124-254, 546-650, 756-763 has a shorter amino acid sequence as the respective reference sequence. Accordingly, an immunogenic fragment of SEQ ID NOs: 124-254, 546-650, 756-763 is N-terminally truncated by e.g. 1, 5, 10, or more amino acids and/or C-terminally truncated e.g. 1, 5, 10, or more amino acids. Notably, an immunogenic fragment still comprises at least one epitope, preferably at least one T-cell epitope, capable of raising an immune response. In embodiments, the at least one epitope has the size/length of an MHC class I or class II epitope. In preferred embodiments, the at least one epitope has the length of an MHC class I epitope. In some embodiments, the at least one epitope has the length of an MHC class II epitope but comprises at least one MHC class I epitope and could be processed during antigen processing during MHC: peptide complex loading. A typical immunogenic fragment in that context has the size of 5 to 20 amino acids, or 8 to 15 amino acids.

lnc-WDR72-2:4

In preferred embodiments, the at least one antigenic peptide comprises an amino acid sequence such as a peptide or protein encoded by lnc-WDR72-2:4 (smORF lncWDR722416421696 or smORF lncWDR722415641696), or an immunogenic fragment or variant thereof.

The full-length polypeptides that are encoded by the lncRNA lnc-WDR72-2:4 (SEQ ID NO: 124 and 144) comprises several predicted epitopes according to SEQ ID NOs 125-143, 145-146, 546-571. Accordingly, the full-length polypeptides (smORF lncWDR722416421696 or lncWDR722415641696 according to SEQ ID NO: 124 or 144) or any fragment or variant thereof comprising epitopes as defined herein are particularly useful in the context of the invention.

In preferred embodiments, the at least one antigenic peptide is selected from a peptide or protein encoded by lnc-WDR72-2:4 and comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 124-146, 546-571, 756, 757, or an immunogenic fragment or immunogenic variant of any of these.

Accordingly, the at least one antigenic peptide preferably comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 124-146, 546-571, 756, 757, or an immunogenic fragment or immunogenic variant of any of these.

A fragment in that context may be an N-terminal truncation of SEQ ID NOs: 124-146, 546-571, 756, 757, or a C-terminal truncation of SEQ ID NOs: 124-146, 546-571, 756, 757. In embodiments, the immunogenic fragment of SEQ ID NOs: 124-146, 546-571, 756, 757 is N-terminally truncated by 1, 2, 3, or 4 amino acids and/or C-terminally truncated by 1, 2, 3, or 4 amino acids. An immunogenic fragment of SEQ ID NOs: 124-146, 546-571, 756, 757 still comprises at least one epitope, preferably a T-cell epitope, capable of raising an immune response. A typical immunogenic fragment in that context has the size of 5 to 20 amino acids, or 8 to 15 amino acids.

In particularly preferred embodiments, the at least one antigenic peptide is selected from a peptide or protein encoded by lnc-WDR72-2:4 and comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to SEQ ID NO: 756, or an immunogenic fragment or immunogenic variant thereof.

lnc-TRPC5-3:1

In embodiments, the at least one antigenic peptide comprises an amino acid sequence such as a peptide or protein encoded by lnc-TRPC5-3:1 (smORF lncTRPC53116031801), or an immunogenic fragment or variant thereof.

The full-length polypeptides that are encoded by the lncRNA TRPC5-3:1 (SEQ ID NO: 227) comprises several predicted epitopes according to SEQ ID NOs 228-237, 630-642. Accordingly, the full-length polypeptide (smORF lncTRPC53116031801 according to SEQ ID NO: 227) or any fragment or variant thereof comprising epitopes as defined herein are particularly useful in the context of the invention.

In preferred embodiments, the at least one antigenic peptide is selected from a peptide or protein encoded by lnc-TRPC5-3:1 and comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 227-237, 630-642, 761, or an immunogenic fragment or immunogenic variant of any of these.

Accordingly, the at least one antigenic peptide preferably comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 227-237, 630-642, 761, or an immunogenic fragment or immunogenic variant of any of these.

A fragment in that context may be an N-terminal truncation SEQ ID NOs: 227-237, 630-642, 761, or a C-terminal truncation of SEQ ID NOs: 227-237, 630-642, 761. In embodiments, the immunogenic fragment of SEQ ID NOs: 227-237, 630-642, 761 is N-terminally truncated by 1, 2, 3, or 4 amino acids and/or C-terminally truncated by 1, 2, 3, or 4 amino acids. An immunogenic fragment of SEQ ID NOs: 227-237, 630-642, 761 still comprises at least one epitope, preferably a T-cell epitope, capable of raising an immune response. A typical immunogenic fragment in that context has the size of 5 to 20 amino acids, or 8 to 15 amino acids.

In preferred embodiments, the at least one antigenic peptide is selected from a peptide or protein encoded by lnc-TRPC5-3:1 and comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 761, or an immunogenic fragment or immunogenic variant thereof.

lnc-CLEC2D-9:1

In preferred embodiments, the at least one antigenic peptide comprises an amino acid sequence such as a peptide or protein encoded by lnc-CLEC2D-9:1 (smORF lncCLEC2D91412616), or an immunogenic fragment or variant thereof.

The full-length polypeptides that are encoded by the lncRNA lnc-CLEC2D-9:1 (SEQ ID NO: 238) comprises several predicted epitopes according to SEQ ID NOs 239-243, 643-650. Accordingly, the full-length polypeptide (smORF lncCLEC2D91412616 according to SEQ ID NO: 238) or any fragment or variant thereof comprising epitopes as defined herein are particularly useful in the context of the invention.

In preferred embodiments, the at least one antigenic peptide is selected from a peptide or protein encoded by lnc-CLEC2D-9:1 and comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 238-243, 643-650, 762, or an immunogenic fragment or immunogenic variant of any of these.

Accordingly, the at least one antigenic peptide preferably comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 238-243, 643-650, 762, or an immunogenic fragment or immunogenic variant of any of these.

An immunogenic fragment in that context may be an N-terminal truncation of SEQ ID NOs: 238-243, 643-650, 762, or a C-terminal truncation of SEQ ID NOs: 238-243, 643-650, 762. In embodiments, the immunogenic fragment of SEQ ID NOs: 238-243, 643-650, 762 is N-terminally truncated by 1, 2, 3, or 4 amino acids and/or C-terminally truncated by 1, 2, 3, or 4 amino acids. An immunogenic fragment of SEQ ID NOs: 238-243, 643-650, 762 still comprises at least one epitope, preferably a T-cell epitope, capable of raising an immune response. A typical immunogenic fragment in that context has the size of 5 to 20 amino acids, or 8 to 15 amino acids.

In preferred embodiments, the at least one antigenic peptide is selected from a peptide or protein encoded by lnc-CLEC2D-9:1 and comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 762, or an immunogenic fragment or immunogenic variant thereof.

### KCNMB2-AS1:4

In preferred embodiments, the at least one antigenic peptide comprises an amino acid sequence such as a peptide or protein encoded by KCNMB2-AS1:4 (smORF KCNMB2AS14633753), or an immunogenic fragment or variant thereof.

The full-length polypeptides that are encoded by the lncRNA KCNMB2-AS1:4 (SEQ ID NO: 147) comprises several predicted epitopes according to SEQ ID NOs 148-161, 572-589. Accordingly, the full-length polypeptide (smORF KCNMB2AS14633753 according to SEQ ID NO: 147) or any fragment or variant thereof comprising epitopes as defined herein are particularly useful in the context of the invention.

In preferred embodiments, the at least one antigenic peptide is selected from a peptide or protein encoded by KCNMB2-AS1:4 and comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 147-161, 572-589, 758, or an immunogenic fragment or immunogenic variant of any of these.

Accordingly, the at least one antigenic peptide preferably comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 147-161, 572-589, 758, or an immunogenic fragment or immunogenic variant of any of these.

A fragment in that context may be an N-terminal truncation of SEQ ID NOs: 147-161, 572-589, 758, or a C-terminal truncation of SEQ ID NOs: 147-161, 572-589, 758. In embodiments, the immunogenic fragment of SEQ ID NOs: 147-161, 572-589, 758 is N-terminally truncated by 1, 2, 3, or 4 amino acids and/or C-terminally truncated by 1, 2, 3, or 4 amino acids. An immunogenic fragment of SEQ ID NOs: 147-161, 572-589, 758 still comprises at least one epitope, preferably a T-cell epitope, capable of raising an immune response. A typical immunogenic fragment in that context has the size of 5 to 20 amino acids, or 8 to 15 amino acids.

In particularly preferred embodiments, the at least one antigenic peptide is selected from a peptide or protein encoded by KCNMB2-AS1:4 and comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 758, or an immunogenic fragment or immunogenic variant of any of these.

lnc-ZC3H8-6:1

In preferred embodiments, the at least one antigenic peptide comprises an amino acid sequence such as a peptide or protein encoded by lnc-ZC3H8-6:1 (smORF lncZC3H86163144), or an immunogenic fragment or variant thereof.

The full-length polypeptides that are encoded by the lncRNA lnc-ZC3H8-6:1 (SEQ ID NO: 162) comprises several predicted epitopes according to SEQ ID NOs 163-168, 590-603. Accordingly, the full-length polypeptide (smORF lncZC3H86163144 according to SEQ ID NO: 162) or any fragment or variant thereof comprising epitopes as defined herein are particularly useful in the context of the invention.

In preferred embodiments, the at least one antigenic peptide is selected from a peptide or protein encoded by lnc-ZC3H8-6:1 and comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 162-168, 590-603, 759, or an immunogenic fragment or immunogenic variant of any of these.

Accordingly, the at least one antigenic peptide preferably comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 162-168, 590-603, 759, or an immunogenic fragment or immunogenic variant of any of these.

A fragment in that context may be an N-terminal truncation of SEQ ID NOs: 162-168, 590-603, 759, or a C-terminal truncation of SEQ ID NOs: 162-168. In embodiments, the immunogenic fragment of SEQ ID NOs: 162-168, 590-603, 759 is N-terminally truncated by 1, 2, 3, or 4 amino acids and/or C-terminally truncated by 1, 2, 3, or 4 amino acids. An immunogenic fragment of SEQ ID NOs: 162-168, 590-603, 759 still comprises at least one epitope, preferably a T-cell epitope, capable of raising an immune response. A typical immunogenic fragment in that context has the size of 5 to 20 amino acids, or 8 to 15 amino acids.

In particularly preferred embodiments, the at least one antigenic peptide is selected from a peptide or protein encoded by lnc-ZC3H8-6:1 and comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 759, or an immunogenic fragment or immunogenic variant thereof.

lnc-NTF3-5:5

In preferred embodiments, the at least one antigenic peptide comprises an amino acid sequence such as a peptide or protein encoded by lnc-NTF3-5:5 (smORF lncNTF35511951417), or an immunogenic fragment or variant thereof.

The full-length polypeptides that are encoded by the lncRNA lnc-NTF3-5:5 (SEQ ID NO: 169) comprises several predicted epitopes according to SEQ ID NOs 170-226, 604-629. Accordingly, the full-length polypeptide (smORF lncNTF35511951417 according to SEQ ID NO: 169) or any fragment or variant thereof comprising epitopes as defined herein are particularly useful in the context of the invention.

In preferred embodiments, the at least one antigenic peptide is selected from a peptide or protein encoded by lnc-NTF3-5:5 and comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 169-226, 604-629, 760, or an immunogenic fragment or immunogenic variant of any of these.

Accordingly, the at least one antigenic peptide preferably comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 169-226, 604-629, 760, or an immunogenic fragment or immunogenic variant of any of these.

A fragment in that context may be an N-terminal truncation of SEQ ID NOs: 169-226, 604-629, 760, or a C-terminal truncation of SEQ ID NOs: 169-226. In embodiments, the immunogenic fragment of SEQ ID NOs: 169-226, 604-629, 760 is N-terminally truncated by 1, 2, 3, or 4 amino acids and/or C-terminally truncated by 1, 2, 3, or 4 amino acids. An immunogenic fragment of SEQ ID NOs: 169-226, 604-629, 760 still comprises at least one epitope, preferably a T-cell epitope, capable of raising an immune response. A typical immunogenic fragment in that context has the size of 5 to 20 amino acids, or 8 to 15 amino acids.

In particularly preferred embodiments, the at least one antigenic peptide is selected from a peptide or protein encoded by lnc-NTF3-5:5 and comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 760, or an immunogenic fragment or immunogenic variant thereof.

### LINC00893:25

In embodiments, the at least one antigenic peptide comprises an amino acid sequence such as a peptide or protein encoded by LINC00893:25 (LINC00893), or an immunogenic fragment or variant thereof.

The full-length polypeptide (smORF LINC00893 according to SEQ ID NO: 244) or any fragment or variant thereof comprising epitopes as defined herein are particularly useful in the context of the invention. Accordingly, the full-length polypeptides that are encoded by the lncRNA LINC00893 (SEQ ID NO: 244) comprises several predicted epitopes according to SEQ ID NOs 245-254.

In preferred embodiments, the at least one antigenic peptide is selected from a peptide or protein encoded by LINC00893:25 and comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 244-254, 763, or an immunogenic fragment or immunogenic variant of any of these.

Accordingly, the at least one antigenic peptide preferably comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 244-254, 763, or an immunogenic fragment or immunogenic variant of any of these.

A fragment in that context may be an N-terminal truncation of SEQ ID NOs: 244-254, 763, or a C-terminal truncation of SEQ ID NOs: 244-254, 763. In embodiments, the immunogenic fragment of SEQ ID NOs: 244-254, 763 is N-terminally truncated by 1, 2, 3, or 4 amino acids and/or C-terminally truncated by 1, 2, 3, or 4 amino acids. An immunogenic fragment of SEQ ID NOs: 244-254, 763 still comprises at least one epitope, preferably a T-cell epitope, capable of raising an immune response. A typical immunogenic fragment in that context has the size of 5 to 20 amino acids, or 8 to 15 amino acids.

In preferred embodiments, the at least one antigenic peptide is selected from a peptide or protein encoded by LINC00893:25 and comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 763, or an immunogenic fragment or immunogenic variant thereof.

Preferred Nucleic Acid Sequences Encoding lncRNA Encoded Antigens

In the following, nucleic acid coding sequences are described that encode the herein specified antigenic peptides (encoded by the respective lncRNAs) that may be comprised in the at least one cds of the artificial nucleic acid.

In preferred embodiments, the at least one coding sequence comprises a nucleic acid sequence encoding at least one antigenic peptide selected from a peptide or protein encoded by a lncRNA, wherein the nucleic acid sequence is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 318-448, 651-755, 764-771, or a fragment or a variant of any of these. Preferably in that context, a fragment has a length of at least 24 nucleotides.

Suitably, the nucleic acid sequence encoding at least one antigenic peptide selected from a peptide or protein encoded by a lncRNA is a codon modified cds, preferably wherein codon modified cds is selected from a C maximized cds (as further defined herein), a CAI maximized cds (as further defined herein), human codon usage adapted cds (as further defined herein), a G/C content modified cds (as further defined herein), and a G/C optimized cds (as further defined herein), or any combination thereof. In preferred embodiments in that context, the at least one codon modified coding sequence is a G/C optimized coding sequence.

Accordingly, in particularly preferred embodiments, the at least one coding sequence comprises a GC optimized nucleic acid sequence encoding at least one antigenic peptide selected from a peptide or protein encoded by a lncRNA, wherein the nucleic acid sequence is identical or at least 80%, 85%, 86%, 87%, 88%, 89%, 90%, 91%, 92%, 93%, 94%, 95%, 96%, 97%, 98%, or 99% identical to any one of SEQ ID NOs: 318-448, 651-755, 764-771, or a fragment of any of these. Preferably in that context, a fragment has a length of at least 24 nucleotides.

In particularly preferred embodiments, the at least one artificial nucleic acid comprises
at least one cds comprising a nucleic acid sequence encoding at least one tumour antigen that comprises at least one antigenic peptide selected from a peptide or protein encoded by lnc-NTF3-5:5, wherein the nucleic acid sequence is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 363-420, 709-734, 768, or a fragment or a variant of any of these, preferably SEQ ID NO 768, or a fragment or a variant thereof; or
at least one cds comprising a nucleic acid sequence encoding at least one tumour antigen that comprises at least one antigenic peptide selected from a peptide or protein encoded by lnc-WDR72-2:4, wherein the nucleic acid sequence is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 318-340, 651-676, 764, 765, or a fragment or a variant of any of these, preferably SEQ ID NO 765, or a fragment or a variant thereof; or
at least one cds comprising a nucleic acid sequence encoding at least one tumour antigen that comprises at least one antigenic peptide selected from a peptide or protein encoded by lnc-TRPC5-3:1, wherein the nucleic acid sequence is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 421-431, 735-747, 769, or a fragment or a variant of any of these, preferably SEQ ID NO 769, or a fragment or a variant thereof, or
at least one cds comprising a nucleic acid sequence encoding at least one tumour antigen that comprises at least one antigenic peptide selected from a peptide or protein encoded by lnc-CLEC2D-9:1, wherein the nucleic acid sequence is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 432-437, 748-755, 770, or a fragment or a variant of any of these, preferably SEQ ID NO 770, or a fragment or a variant thereof; or
at least one cds comprising a nucleic acid sequence encoding at least one tumour antigen that comprises at least one antigenic peptide selected from a peptide or protein encoded by KCNMB2-AS1:4, wherein the nucleic acid sequence is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 341-355, 677-694, 766, or a fragment or a variant of any of these, preferably SEQ ID NO 766, or a fragment or a variant thereof; or
at least one cds comprising a nucleic acid sequence encoding at least one tumour antigen that comprises at least one antigenic peptide selected from a peptide or protein encoded by lnc-ZC3H8-6:1, wherein the nucleic acid sequence is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 356-362, 695-708, 767, or a fragment or a variant of any of these, preferably SEQ ID NO 767, or a fragment or a variant thereof, or
at least one cds comprising a nucleic acid sequence encoding at least one tumour antigen that comprises at least one antigenic peptide selected from a peptide or protein encoded by LINC00893:25, wherein the nucleic acid sequence is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 438-448, 771, or a fragment or a variant of any of these, preferably SEQ ID NO 771, or a fragment or a variant thereof.

Preferred amino acid sequences and nucleic acid sequences in that context are provided in Table 1A. Therein, each row corresponds to suitable antigenic peptides or protein encoded by a long non-coding RNA (lncRNA). Column A provides a short description of the respective lncRNA that encodes the tumour antigens. Column B provides the SEQ ID NOs of respective full-length amino acid sequences encoded by the lncRNAs. Column C provides the amino acid SEQ ID NOs of respective fragments or epitopes. Column D provides SEQ ID NO of G/C optimized nucleic acid sequence encoding the full-length amino acid sequence of Column B. Column E provides SEQ ID NO of G/C optimized nucleic acid sequences encoding the respective fragments or epitopes of Column C. Column F indicates the preferred cancer indication of the antigen (N=NSCLC; M=Melanoma; H=HNSCC; see also aspect 7 (medical uses) that provides further cancer indications, in particular for lncRNA encoded antigens of row 1-4 and 6).

TABLE 1A
Preferred antigens encoded by lncRNA (amino acid sequences and coding sequences)

| | A | B | C | D | E | F |
|---|---|---|---|---|---|---|
| 1 | lnc-WDR72-2:4 | 124, | 125-143, 145-146, 546- | 318, | 319-337, 339-340, 651- | N, H |
| | | 144 | 571, 756, 757 | 338 | 676, 764, 765 | |
| 2 | KCNMB2-AS1:4 | 147 | 148-161, 572-589, 758 | 341 | 342-355, 677-694, 766 | N, H |
| 3 | lnc-ZC3H8-6:1 | 162 | 163-168, 590-603, 759 | 356 | 357-362, 695-708, 767 | N, H |
| 4 | lnc-NTF3-5:5 | 169 | 170-226, 604-629, 760 | 363 | 364-420, 709-734, 768 | N, H |
| 5 | lnc-TRPC5-3:1 | 227 | 228-237, 630-642, 761 | 421 | 422-431, 735-747, 769 | N, M, H |
| 6 | lnc-CLEC2D-9:1 | 238 | 239-243, 643-650, 762 | 432 | 433-437, 748-755, 770 | M |
| 7 | LINC00893:25 | 244 | 245-254, 763 | 438 | 439-448, 771 | M |

Particularly preferred amino acid sequences and nucleic acid sequences in that context are provided in Table 1B. Therein, each row corresponds to a particularly suitable antigenic peptides or protein encoded by a long non-coding RNA (lncRNA). Column A provides a short description of the respective lncRNA that encodes the tumour antigens. Column B provides the SEQ ID NOs of respective full-length amino acid sequence encoded by the lncRNA. Column C provides the amino acid SEQ ID NOs of preferred fragments or epitopes. Column D provides SEQ ID NO of G/C optimized nucleic acid sequence encoding the full-length amino acid sequence of Column B. Column E provides SEQ ID NO of G/C optimized nucleic acid sequences encoding the preferred fragments or epitopes of Column C.

TABLE 1B
Particularly preferred antigens encoded by lncRNA
(amino acid sequences and coding sequences)

| | A | B | C | D | E |
|---|---|---|---|---|---|
| 1 | lnc-WDR72-2:4 | 144 | 756 | 318 | 764 |
| 2 | KCNMB2-AS1:4 | 147 | 758 | 341 | 766 |
| 3 | lnc-ZC3H8-6:1 | 162 | 759 | 356 | 767 |
| 4 | lnc-NTF3-5:5 | 169 | 760 | 363 | 768 |
| 6 | lnc-CLEC2D-9:1 | 238 | 762 | 432 | 770 |

In particularly preferred embodiments, the at least one antigenic peptide comprises or consists of at least one amino acid sequence selected from Table 1A or 1B, columns B or C, or immunogenic fragments of any of these.

In particularly preferred embodiments, the at least one coding sequence of the artificial nucleic acid of the invention comprises or consists of at least one nucleic acid sequence as provided in Table 1A or 1B, columns D or E, or a fragment or variant of any of these.

Tumour Antigens Selected from Tumour Neoantigens

In preferred embodiments, the artificial nucleic acid comprises at least one coding sequence encoding at least one tumour antigen that comprises or consists of at least one antigenic peptide selected or derived from a peptide or protein or a tumour neoantigen, or an immunogenic fragment or variant thereof.

The term “tumour neoantigen” refers to a specific polypeptide generated by gene mutation of a tumour cell, which is presented to the cell surface by MHC molecules, in mammals HLA molecules, and thus recognized by a T lymphocyte to elicit an immune response. Neoantigens are therefore suitable to serve as markers for immunotherapy and further used to develop tumour neoantigen vaccines, because these neoantigens are generated by tumour cell mutation but not expressed in normal or healthy cells. Therefore, neoantigens may serve as promising targets for immunotherapy.

In preferred embodiments, the neoantigen is a shared neoantigen, preferably a shared neoantigen for NSCLC and/or melanoma cancer patients.

Surprisingly, peptides have been identified that are present in tumour cells. Even more surprisingly, these peptides show specificity in tumour cells and raise an immune response, preferably an antigen specific immune response.

In embodiments, the at least one antigenic peptide selected from a peptide or protein of a tumour neoantigen is selected from an NRAS neoantigen, TYW1B neoantigen, ECPAS neoantigen, MAP2K1 neoantigen, TOMM22 neoantigen, GLB1 neoantigen, MAGE-A3 neoantigen, ATAD2 neoantigen, BRAF neoantigen, EGFR neoantigen, TP53 neoantigen, DIP2B neoantigen, TRRAP neoantigen, RAC1 neoantigen, AP4B1 neoantigen, VWA5A neoantigen, CLTC neoantigen, RPL9 neoantigen, RPAP1 neoantigen, RPE65 neoantigen, PATZ1 neoantigen, PIK3CA neoantigen, NFE2L2 neoantigen, CNOT9 neoantigen, or an immunogenic fragment or variant of any of these.

For clarification, the terms “NRAS neoantigen”, “TYW1B neoantigen”, “ECPAS neoantigen”, “MAP2K1 neoantigen”, “TOMM22 neoantigen”, “GLB1 neoantigen”, “MAGE-A3 neoantigen”, “ATAD2 neoantigen”, “BRAF neoantigen”, “EGFR neoantigen”, “TP53 neoantigen”, “DIP2B neoantigen”, “TRRAP neoantigen”, “RAC1 neoantigen”, “AP4B1 neoantigen”, “VWA5A neoantigen”, “CLTC neoantigen”, “RPL9 neoantigen”, “RPAP1 neoantigen”, “RPE65 neoantigen”, “PATZ1 neoantigen”, “PIK3CA neoantigen”, “NFE2L2 neoantigen”, “CNOT9 neoantigen” relate to respective mutated proteins found in cancer cells and do not relate to the respective wild type or somatic protein.

In preferred embodiments, the at least one antigenic peptide selected from a peptide or protein of an NRAS neoantigen, TYW1B neoantigen, ECPAS neoantigen, MAP2K1 neoantigen, TOMM22 neoantigen, GLB1 neoantigen, MAGE-A3 neoantigen, ATAD2 neoantigen, BRAF neoantigen, EGFR neoantigen, TP53 neoantigen, or an immunogenic fragment or variant of any of these.

In preferred embodiments, the at least one antigenic peptide selected from a peptide or protein of an NRAS neoantigen, TYW1B neoantigen, ECPAS neoantigen, MAP2K1 neoantigen, TOMM22 neoantigen, GLB1 neoantigen, MAGE-A3 neoantigen, ATAD2 neoantigen, BRAF neoantigen, EGFR neoantigen, TP53 neoantigen, DIP2B neoantigen, TRRAP neoantigen, RAC1 neoantigen, AP4B1 neoantigen, VWA5A neoantigen, CLTC neoantigen, RPL9 neoantigen, RPAP1 neoantigen, RPE65 neoantigen, PATZ1 neoantigen, comprises at least one T-cell epitope, preferably at least one CD8+ T cell epitope.

In particularly preferred embodiments, the at least one antigenic peptide selected from a peptide or protein of a tumour neoantigen, or immunogenic fragment or variant thereof, in particular of an NRAS neoantigen, TYW1B neoantigen, ECPAS neoantigen, MAP2K1 neoantigen, TOMM22 neoantigen, GLB1 neoantigen, MAGE-A3 neoantigen, ATAD2 neoantigen, BRAF neoantigen, EGFR neoantigen, TP53 neoantigen, DIP2B neoantigen, TRRAP neoantigen, RAC1 neoantigen, AP4B1 neoantigen, VWA5A neoantigen, CLTC neoantigen, RPL9 neoantigen, RPAP1 neoantigen, RPE65 neoantigen, PATZ1 neoantigen, induce epitope-specific T cells in a subject, preferably epitope specific CD8+ T cells in a subject (in particular, a human subject).

In preferred embodiments, the at least one antigenic peptide selected from a peptide or protein of a tumour neoantigen, or immunogenic fragment or variant thereof, is an antigen or epitope displaying HLA binding or MHC binding affinity.

Preferably, the at least one antigenic peptide of a tumour neoantigen as specified herein bind to an antibody or a T-cell receptor. In preferred embodiments, the antigenic peptide comprise an MHCI or MHCII ligand/epitope.

In embodiments, the at least one antigenic peptide (provided by the artificial nucleic acid) is selected from a peptide or protein of a tumour neoantigen and comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 255-317, or an immunogenic fragment or variant of any of these.

In preferred embodiments, the at least one antigenic peptide (provided by the artificial nucleic acid) is selected from a peptide or protein of a tumour neoantigen and comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 255-286, or an immunogenic fragment or variant of any of these.

In that context, an immunogenic fragment of SEQ ID NOs: 255-317 has a shorter amino acid sequence as the respective reference sequence. An immunogenic fragment of SEQ ID NOs: 255-317 is N-terminally truncated by e.g. 1, 5, 10, or more amino acids and/or C-terminally truncated e.g. 1, 5, 10, or more amino acids. Notably, an immunogenic fragment still comprises at least one epitope, preferably at least one T-cell epitope, capable of raising an immune response. In some embodiments, the at least one epitope has the size/length of an MHC class I or class II epitope. In preferred embodiments, the at least one epitope has the length of an MHC class I epitope. In some embodiments, the at least one epitope has the length of an MHC class II epitope but comprises at least one MHC class I epitope and could be processed during antigen processing during MHC: peptide complex loading. A typical immunogenic fragment in that context has the size of 5 to 20 amino acids, or 8 to 15 amino acids.

### NRAS Amino Acid and Nucleic Acid Sequences

In preferred embodiments, the at least one antigenic peptide is selected from a peptide or protein of an NRAS tumour neoantigen and comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 267-272, or immunogenic fragments or variants of any of these. Accordingly, in preferred embodiments, the artificial nucleic acid comprises at least one cds encoding at least one tumour antigen that comprises or consists of at least one antigenic peptide selected from a peptide or protein of a NRAS tumour neoantigen, wherein the antigenic peptide comprises or consists of at least one of the amino acid sequences being identical or at least 80% identical to any one of SEQ ID NOs: 267-272, or an immunogenic fragment or variant thereof.

A fragment in that context may be an N-terminal truncation of SEQ ID NOs: 267-272 or a C-terminal truncation of SEQ ID NOs: 267-272. In embodiments, the immunogenic fragment of SEQ ID NOs: 267-272 is N-terminally truncated by 1, 2, 3, or 4 amino acids and/or C-terminally truncated by 1, 2, 3, or 4 amino acids. An immunogenic fragment of SEQ ID NOs: 267-272 still comprises at least one epitope, preferably a T-cell epitope, capable of raising an immune response. A typical immunogenic fragment in that context has the size of 5 to 20 amino acids, or 8 to 15 amino acids.

### TYW1B Amino Acid and Nucleic Acid Sequences

In preferred embodiments, the at least one antigenic peptide is selected from a peptide or protein of a TYW1B tumour neoantigen and comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 273, 274, or an immunogenic fragment or immunogenic variant of these. Accordingly, in preferred embodiments, the artificial nucleic acid comprises at least one cds encoding at least one tumour antigen that comprises or consists of at least one antigenic peptide selected from a peptide or protein of a TYW1B tumour neoantigen, wherein the antigenic peptide comprises or consists of at least one of the amino acid sequences being identical or at least 80% identical to any one of SEQ ID NOs: 273, 274, or an immunogenic fragment or variant thereof.

A fragment in that context may be an N-terminal truncation of SEQ ID NOs: 273, 274 or a C-terminal truncation of SEQ ID NOs: 273, 274. In embodiments, the immunogenic fragment of SEQ ID NOs: 273, 274 is N-terminally truncated by 1, 2, 3, or 4 amino acids and/or C-terminally truncated by 1, 2, 3, or 4 amino acids. An immunogenic fragment of SEQ ID NOs: 273, 274 still comprises at least one epitope, preferably a T-cell epitope, capable of raising an immune response. A typical immunogenic fragment in that context has the size of 5 to 20 amino acids, or 8 to 15 amino acids.

### ECPAS Amino Acid and Nucleic Acid Sequences

In preferred embodiments, the at least one antigenic peptide is selected from a peptide or protein of a ECPAS tumour neoantigen and comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 275, or an immunogenic fragment or immunogenic variant thereof. Accordingly, in preferred embodiments, the artificial nucleic acid comprises at least one cds encoding at least one tumour antigen that comprises or consists of at least one antigenic peptide selected from a peptide or protein of a ECPAS tumour neoantigen, wherein the antigenic peptide comprises or consists of at least one of the amino acid sequences being identical or at least 80% identical to any one of SEQ ID NOs: 275, or an immunogenic fragment or variant thereof.

A fragment in that context may be an N-terminal truncation of SEQ ID NOs: 275 or a C-terminal truncation of SEQ ID NOs: 275. In embodiments, the immunogenic fragment of SEQ ID NOs: 275 is N-terminally truncated by 1, 2, 3, or 4 amino acids and/or C-terminally truncated by 1, 2, 3, or 4 amino acids. An immunogenic fragment of SEQ ID NOs: 275 still comprises at least one epitope, preferably a T-cell epitope, capable of raising an immune response. A typical immunogenic fragment in that context has the size of 5 to 20 amino acids, or 8 to 15 amino acids.

### MAP2K1 Amino Acid and Nucleic Acid Sequences

In preferred embodiments, the at least one antigenic peptide is selected from a peptide or protein of a MAP2K1 tumour neoantigen and comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 276, 277, or an immunogenic fragment or immunogenic variant of these. Accordingly, in preferred embodiments, the artificial nucleic acid comprises at least one cds encoding at least one tumour antigen that comprises or consists at least one antigenic peptide selected from a peptide or protein of a MAP2K1 tumour neoantigen, wherein the antigenic peptide comprises or consists of at least one of the amino acid sequences being identical or at least 80% identical to any one of SEQ ID NOs: 276, 277, or an immunogenic fragment or variant of these.

A fragment in that context may be an N-terminal truncation of SEQ ID NOs: 276, 277 or a C-terminal truncation of SEQ ID NOs: 276, 277. In embodiments, the immunogenic fragment of SEQ ID NOs: 276, 277 is N-terminally truncated by 1, 2, 3, or 4 amino acids and/or C-terminally truncated by 1, 2, 3, or 4 amino acids. An immunogenic fragment of SEQ ID NOs: 276, 277 still comprises at least one epitope, preferably a T-cell epitope, capable of raising an immune response. A typical immunogenic fragment in that context has the size of 5 to 20 amino acids, or 8 to 15 amino acids.

### TOMM22 Amino Acid and Nucleic Acid Sequences

In preferred embodiments, the at least one antigenic peptide is selected from a peptide or protein of a TOMM22 tumour neoantigen and comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 278, 279, or an immunogenic fragment or immunogenic variant of these. Accordingly, in preferred embodiments, the artificial nucleic acid comprises at least one cds encoding at least one tumour antigen that comprises or consists of at least one antigenic peptide selected from a peptide or protein of a TOMM22 tumour neoantigen, wherein the antigenic peptide comprises or consists of at least one of the amino acid sequences being identical or at least 80% identical to any one of SEQ ID NOs: 278, 279, or an immunogenic fragment or variant of these.

A fragment in that context may be an N-terminal truncation of SEQ ID NOs: 278, 279 or a C-terminal truncation of SEQ ID NOs: 278, 279. In embodiments, the immunogenic fragment of SEQ ID NOs: 278, 279 is N-terminally truncated by 1, 2, 3, or 4 amino acids and/or C-terminally truncated by 1, 2, 3, or 4 amino acids. An immunogenic fragment of SEQ ID NOs: 278, 279 still comprises at least one epitope, preferably a T-cell epitope, capable of raising an immune response. A typical immunogenic fragment in that context has the size of 5 to 20 amino acids, or 8 to 15 amino acids.

### GLB1 Amino Acid and Nucleic Acid Sequences

In preferred embodiments, the at least one antigenic peptide is selected from a peptide or protein of a GLB1 tumour neoantigen and comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 280, 281, or an immunogenic fragment or immunogenic variant of any of these. Accordingly, in preferred embodiments, the artificial nucleic acid comprises at least one cds encoding at least one tumour antigen that comprises or consists of at least one antigenic peptide selected from a peptide or protein of a GLB1 tumour neoantigen, wherein the antigenic peptide comprises or consists of at least one of the amino acid sequences being identical or at least 80% identical to any one of SEQ ID NOs: 280, 281, or an immunogenic fragment or variant of these.

A fragment in that context may be an N-terminal truncation of SEQ ID NOs: 280, 281 or a C-terminal truncation of SEQ ID NOS: 280, 281. In embodiments, the immunogenic fragment of SEQ ID NOs: 280, 281 is N-terminally truncated by 1, 2, 3, or 4 amino acids and/or C-terminally truncated by 1, 2, 3, or 4 amino acids. An immunogenic fragment of SEQ ID NOs: 280, 281 still comprises at least one epitope, preferably a T-cell epitope, capable of raising an immune response. A typical immunogenic fragment in that context has the size of 5 to 20 amino acids, or 8 to 15 amino acids.

### MAGE-A3 Amino Acid and Nucleic Acid Sequences

In preferred embodiments, the at least one antigenic peptide is selected from a peptide or protein of a MAGE-A3 tumour neoantigen and comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 282, 283, or an immunogenic fragment or immunogenic variant of these. Accordingly, in preferred embodiments, the artificial nucleic acid comprises at least one cds encoding at least one tumour antigen that comprises or consists of at least one antigenic peptide selected from a peptide or protein of a MAGE-A3 tumour neoantigen, wherein the antigenic peptide comprises or consists of at least one of the amino acid sequences being identical or at least 80% identical to any one of SEQ ID NOs: 282, 283, or an immunogenic fragment or variant thereof.

A fragment in that context may be an N-terminal truncation of SEQ ID NOs: 282, 283 or a C-terminal truncation of SEQ ID NOs: 282, 283. In embodiments, the immunogenic fragment of SEQ ID NOs: 282, 283 is N-terminally truncated by 1, 2, 3, or 4 amino acids and/or C-terminally truncated by 1, 2, 3, or 4 amino acids. An immunogenic fragment of SEQ ID NOs: 282, 283 still comprises at least one epitope, preferably a T-cell epitope, capable of raising an immune response. A typical immunogenic fragment in that context has the size of 5 to 20 amino acids, or 8 to 15 amino acids.

### ATAD2 Amino Acid and Nucleic Acid Sequences

In preferred embodiments, the at least one antigenic peptide is selected from a peptide or protein of a ATAD2 tumour neoantigen and comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 284-286, or an immunogenic fragment or immunogenic variant of these. Accordingly, in preferred embodiments, the artificial nucleic acid comprises at least one cds encoding at least one tumour antigen that comprises or consists of at least one antigenic peptide selected from a peptide or protein of a ATAD2 tumour neoantigen, wherein the antigenic peptide comprises or consists of at least one of the amino acid sequences being identical or at least 80% identical to any one of SEQ ID NOs: 284-286, or an immunogenic fragment or variant thereof.

A fragment in that context may be an N-terminal truncation of SEQ ID NOs: 284-286 or a C-terminal truncation of SEQ ID NOs: 284-286. In embodiments, the immunogenic fragment of SEQ ID NOs: 284-286 is N-terminally truncated by 1, 2, 3, or 4 amino acids and/or C-terminally truncated by 1, 2, 3, or 4 amino acids. An immunogenic fragment of SEQ ID NOs: 284-286 still comprises at least one epitope, preferably a T-cell epitope, capable of raising an immune response. A typical immunogenic fragment in that context has the size of 5 to 20 amino acids, or 8 to 15 amino acids.

### BRAF Amino Acid and Nucleic Acid Sequences

In preferred embodiments, the at least one antigenic peptide is selected from a peptide or protein of a BRAF tumour neoantigen and comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 262-266, or an immunogenic fragment or immunogenic variant of any of these. Accordingly, in preferred embodiments, the artificial nucleic acid comprises at least one cds encoding at least one tumour antigen that comprises or consists of at least one antigenic peptide selected from a peptide or protein of a BRAF tumour neoantigen, wherein the antigenic peptide comprises or consists of at least one of the amino acid sequences being identical or at least 80% identical to any one of SEQ ID NOs: 262-266, or an immunogenic fragment or variant thereof.

A fragment in that context may be an N-terminal truncation of SEQ ID NOs: 262-266 or a C-terminal truncation of SEQ ID NOs: 262-266. In embodiments, the immunogenic fragment of SEQ ID NOs: 262-266 is N-terminally truncated by 1, 2, 3, or 4 amino acids and/or C-terminally truncated by 1, 2, 3, or 4 amino acids. An immunogenic fragment of SEQ ID NOs: 262-266 still comprises at least one epitope, preferably a T-cell epitope, capable of raising an immune response.

### EGFR Amino Acid and Nucleic Acid Sequences

In preferred embodiments, the at least one antigenic peptide is selected from a peptide or protein of a EGFR tumour neoantigen and comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 255-257, or an immunogenic fragment or immunogenic variant of any of these. Accordingly, in preferred embodiments, the artificial nucleic acid comprises at least one cds encoding at least one tumour antigen that comprises or consists of at least one antigenic peptide selected from a peptide or protein of a EGFR tumour neoantigen, wherein the antigenic peptide comprises or consists of at least one of the amino acid sequences being identical or at least 80% identical to any one of SEQ ID NOs: 255-257, or an immunogenic fragment or variant thereof.

A fragment in that context may be an N-terminal truncation of SEQ ID NOs: 255-257 or a C-terminal truncation of SEQ ID NOs: 255-257. In embodiments, the immunogenic fragment of SEQ ID NOs: 255-257 is N-terminally truncated by 1, 2, 3, or 4 amino acids and/or C-terminally truncated by 1, 2, 3, or 4 amino acids. An immunogenic fragment of SEQ ID NOs: 255-257 still comprises at least one epitope, preferably a T-cell epitope, capable of raising an immune response.

### TP53 Amino Acid and Nucleic Acid Sequences

In preferred embodiments, the at least one antigenic peptide is selected from a peptide or protein of a TP53 tumour neoantigen and comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 258-261, or an immunogenic fragment or immunogenic variant of any of these. Accordingly, in preferred embodiments, the artificial nucleic acid comprises at least one cds encoding at least one tumour antigen that comprises or consists of at least one antigenic peptide selected from a peptide or protein of a TP53 tumour neoantigen, wherein the antigenic peptide comprises or consists of at least one of the amino acid sequences being identical or at least 80% identical to any one of SEQ ID NOs: 258-261, or an immunogenic fragment or variant thereof.

A fragment in that context may be an N-terminal truncation of SEQ ID NOs: 258-261 or a C-terminal truncation of SEQ ID NOs: 258-261. In embodiments, the immunogenic fragment of SEQ ID NOs: 258-261 is N-terminally truncated by 1, 2, 3, or 4 amino acids and/or C-terminally truncated by 1, 2, 3, or 4 amino acids. An immunogenic fragment of SEQ ID NOs: 258-261 still comprises at least one epitope, preferably a T-cell epitope, capable of raising an immune response.

### DIP2B, TRRAP, RAC1, AP4B1, VWA5A, CLTC, RPL9, RPAP1, RPE65, PATZ1, PIK3CA, NFE2L2, CNOT9 Amino Acid and Nucleic Acid Sequences

In embodiments, the at least one antigenic peptide is selected from a peptide or protein of a DIP2B, TRRAP, RAC1, AP4B1, VWA5A, CLTC, RPL9, RPAP1, RPE65, PATZ1, PIK3CA, NFE2L2, or CNOT9 tumour neoantigen and comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 287-317, or an immunogenic fragment or immunogenic variant of any of these. Accordingly, in embodiments, the artificial nucleic acid comprises at least one cds encoding at least one tumour antigen that comprises or consists of at least one antigenic peptide selected from a peptide or protein of a DIP2B, TRRAP, RAC1, AP4B1, VWA5A, CLTC, RPL9, RPAP1, RPE65, PATZ1 tumour neoantigen, wherein the antigenic peptide comprises or consists of at least one of the amino acid sequences being identical or at least 80% identical to any one of SEQ ID NOs: 287-317, or an immunogenic fragment or variant thereof.

A fragment in that context may be an N-terminal truncation of SEQ ID NOs: 287-317 or a C-terminal truncation of SEQ ID NOs: 287-317. In embodiments, the immunogenic fragment of SEQ ID NOs: 287-317 is N-terminally truncated by 1, 2, 3, or 4 amino acids and/or C-terminally truncated by 1, 2, 3, or 4 amino acids. An immunogenic fragment of SEQ ID NOs: 287-317 still comprises at least one epitope, preferably a T-cell epitope, capable of raising an immune response.

In the following, preferred nucleic acid coding sequences are described that encode the herein specified antigenic peptides of the tumour neoantigen as defined herein that may be comprised in the at least one coding sequence of the artificial nucleic acid of the invention.

In preferred embodiments, the at least one coding sequence comprises a nucleic acid sequence encoding at least one antigenic peptide selected from a peptide or protein of a tumour neoantigen, wherein the nucleic acid sequence is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 449-511, or a fragment or a variant of any of these. Preferably in that context, a fragment has a length of at least 24 nucleotides.

In preferred embodiments, the at least one coding sequence comprises a nucleic acid sequence encoding at least one antigenic peptide selected from a peptide or protein of a tumour neoantigen, wherein the nucleic acid sequence is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 449-480, or a fragment or a variant of any of these. Preferably in that context, a fragment has a length of at least 24 nucleotides.

Suitably, the nucleic acid sequence encoding at least one antigenic peptide from a tumour neoantigen is a codon modified cds, preferably wherein codon modified cds is selected from a C maximized cds (as defined herein), a CAI maximized cds (as defined herein), human codon usage adapted cds (as defined herein), a G/C content modified cds (as defined herein), and a G/C optimized cds (as defined herein), or any combination thereof. In preferred embodiments in that context, the at least one codon modified cds is a G/C optimized cds.

Accordingly, in particularly preferred embodiments, the at least one coding sequence comprises a GC optimized nucleic acid sequence encoding at least one antigenic peptide selected from a peptide or protein of a tumour neoantigen, wherein the nucleic acid sequence is identical or at least 80%, 85%, 86%, 87%, 88%, 89%, 90%, 91%, 92%, 93%, 94%, 95%, 96%, 97%, 98%, or 99% identical to any one of SEQ ID NOs: 449-511, or a fragment of any of these. Preferably in that context, a fragment has a length of at least 24 nucleotides.

Preferred amino acid sequences and nucleic acid sequences in that context are provided in Table 2. Therein, each row corresponds to a suitable antigenic peptides of a tumour neoantigen. Column A provides a short description of the respective tumour neoantigen. Column B provides protein (amino acid sequence) SEQ ID NOs of preferred antigenic peptides of the tumour neoantigens. Column C provides SEQ ID NO of the corresponding G/C optimized nucleic acid sequences encoding the preferred antigenic peptides of the tumour neoantigens. Column F indicates the preferred cancer indication of the antigen (N=NSCLC; M=Melanoma).

TABLE 2
Preferred antigens of a tumour neoantigen
(amino acid sequences and coding sequences)

| row | A | B | C | D |
|---|---|---|---|---|
| 1 | EGFR | 255-257 | 449-451 | N |
| 2 | TP53 | 258-261 | 452-455 | N |
| 3 | BRAF | 262-266 | 456-460 | N, M |
| 4 | NRAS | 267-272 | 461-466 | M |
| 5 | TYW1B | 273, 274 | 467, 468 | M |
| 6 | ECPAS | 275 | 469 | M |
| 7 | MAP2K1 | 276, 277 | 470, 471 | M |
| 8 | TOMM22 | 278, 279 | 472, 473 | M |
| 9 | GLB1 | 280, 281 | 474, 475 | M |
| 10 | MAGE-A3 | 282, 283 | 476, 477 | M |
| 11 | ATAD2 | 284-286 | 478-480 | M |
| 12 | DIP2B | 287-289 | 481-483 | M |
| 13 | TRRAP | 290-295 | 484-489 | M |
| 14 | RAC1 | 296-299 | 490-493 | M |
| 15 | AP4B1 | 300, 301 | 494, 495 | M |
| 16 | VWA5A | 302 | 496 | M |
| 17 | CLTC | 303, 304 | 497, 498 | M |
| 18 | RPL9 | 305 | 499 | M |
| 19 | RPAP1 | 306-308 | 500-502 | M |
| 20 | RPE65 | 309-311 | 503-505 | M |
| 21 | PATZ1 | 312, 313 | 506, 507 | M |
| 22 | PIK3CA | 314 | 508 | N |
| 23 | NFE2L2 | 315 | 509 | N |
| 24 | CNOT9 | 316, 317 | 510, 511 | M |

In particularly preferred embodiments, the at least one antigenic peptide comprises or consists of at least one amino acid sequence selected from Table 2, column B, or immunogenic fragments of any of these. In even more preferred embodiments, the at least one antigenic peptide comprises or consists of at least one amino acid sequence selected from Table 2, column B, rows 1 to 11, or immunogenic fragments of any of these.

In particularly preferred embodiments, the at least one coding sequence of the artificial nucleic acid of the invention comprises or consists of at least one nucleic acid sequence as provided in Table 2, column C. In even more preferred embodiments, In particularly preferred embodiments, the at least one coding sequence of the artificial nucleic acid of the invention comprises or consists of at least one nucleic acid sequence as provided in Table 2, column C, rows 1 to 11, or immunogenic fragments thereof.

### Further Peptide or Protein Features

In embodiments, the at least one coding sequence encodes 2 to 10 different tumour antigens as specified herein, preferably 2 to 5 different tumour antigens as specified herein. Accordingly, the coding sequence may encode multiple tumour antigens that are provided in form of a fusion construct.

Suitably, the respective different tumour antigens are selected to obtain an increased coverage in a certain cancer disease, e.g. NSCLC, HNSCC, or melanoma. Suitably, the coverage of all the different tumour antigens is at a value of above 0.5, preferably above 0.6, more preferably above 0.7.

A “different tumour antigen” in the context of the invention relates to a difference on amino acid sequence level and has to be understood as a difference in at least one amino acid position or having a different amino acid sequence length. Accordingly, a “different tumour antigen” may be derived from the same tumour neoantigen but the “different tumour antigen” should differ in at least one amino acid position or length.

Accordingly, the at least one coding sequence may encode 2 to 10 different tumour antigens, preferably 2 to 5 different tumour antigens, wherein the different tumour antigens are selected from a peptide or protein encoded by a long non-coding RNA (lncRNA) as defined herein and/or from a peptide or protein of a tumour neoantigen as defined herein, or an immunogenic fragment or variant of any of these. Suitable amino acid and nucleic acid sequences in that context are defined above and may inter alia be taken from Table 1A and B and Table 2.

In preferred embodiments in that context, the different tumour antigens are selected to cover different HLA types. Accordingly, the different tumour antigen are selected from at least one tumour antigen as defined herein that comprises at least one epitope that binds to HLA-A type, at least one tumour antigen as defined herein that comprises at least one epitope binds to HLA-B type, at least one tumour antigen as defined herein that comprises at least one epitope binds to HLA-C type.

In embodiments, the different tumour antigens are separated by a linker element. Accordingly, the coding sequence may encode multiple different tumour antigens that are provided in form of a fusion construct, wherein the different tumour antigens in the fusion construct are separated by linker elements. Suitably, such linkers are non-immunogenic. Suitable linker elements may be selected from SEQ ID NOs: 2937, 76400-76418, 77018-77058 of published PCT patent application WO2019008001.

In embodiments, the at least one cds encodes at least one additional amino acid sequence selected from at least one immune response activating signal transduction protein, preferably at least one transmembrane domain and/or at least one cytoplasmic domain derived from said at least one immune response activating signal transduction protein.

Preferably, said immune response activating signal transduction protein is located in the external side of plasma membranes and readily and recurrently internalized to the endosomal pathways, preferably to target the encoded and translated antigens to desired intracellular pathways, which preferably intersect MHC class I and in particular also MHC class II pathways, for effective delivery of antigenic sequences to MHC class I and MHC class II processing compartments (as described in WO2019008001 and fully cooperated herein by reference).

In preferred embodiments, the immune response activating signal transduction protein is selected from CTLA4.

In preferred embodiments, the at least one immune response activating signal transduction protein comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 76636 of published PCT patent application WO2019008001 or a fragment or variant of any of these.

In embodiments, the at least one coding sequence encodes at least one additional amino acid sequence selected from at least one T-helper epitope. Suitable T-helper epitopes may be selected from SEQ ID NOs: 3083-3294 of published PCT patent application WO2019008001.

In embodiments, the at least one coding sequence encodes at least one additional amino acid sequence selected from at least one signal peptide. Suitable signal peptides may be selected from of SEQ ID NOs: 1-156, 76948-76951 of published PCT patent application WO2019008001. In preferred embodiments, the at least one signal peptide is selected from CTLA4.

### Nucleic Acid Sequence Features and Embodiments

In the following, suitable features and embodiments referring to artificial nucleic acid sequences of the invention are provided and described in detail (e.g. type of nucleic acid, structure of nucleic acid, elements of nucleic acid, modification of nucleic acid etc.). Notably, said features defining nucleic acid sequences may also apply to any nucleic acid sequence in any other aspect of the invention (e.g. composition, combination, kit, medical uses).

### Suitable Coding Sequences:

According to preferred embodiments, the artificial nucleic acid of the invention comprises at least one coding sequence encoding at least one tumour antigen as defined herein. In that context, any coding sequence encoding at least one tumour antigen as defined herein, or fragments and variants thereof may be understood as suitable coding sequence and may therefore be comprised in the nucleic acid of the invention.

In preferred embodiments, the artificial nucleic acid is a modified and/or stabilized nucleic acid.

According to preferred embodiments, the artificial nucleic acid may thus be provided as a “stabilized nucleic acid” that is to say a nucleic acid showing improved resistance to in vivo degradation and/or a nucleic acid showing improved stability in vivo, and/or a nucleic acid showing improved translatability in vivo. This is particularly important in embodiments where the nucleic acid is an RNA.

Preferably, the artificial nucleic of the present invention may be provided as a “stabilized nucleic acid”, preferably a “stabilized RNA”.

In the following, suitable modifications/adaptations are described that are capable of “stabilizing” the nucleic acid, preferably the RNA.

In particularly preferred embodiments, the artificial nucleic acid comprises at least one codon modified cds. Suitably, the amino acid sequence encoded by the at least one codon modified coding sequence is not being modified compared to the amino acid sequence encoded by the corresponding wild type or reference coding sequence.

The term “codon modified coding sequence” relates to coding sequences that differ in at least one codon (triplets of nucleotides coding for one amino acid) compared to the corresponding wild type or reference coding sequence. Suitably, a codon modified cds in the context of the invention may show improved resistance to in vivo degradation and/or improved stability in vivo, and/or improved translatability in vivo. Codon modifications in the broadest sense make use of the degeneracy of the genetic code wherein multiple codons may encode the same amino acid and may be used interchangeably to optimize/modify the coding sequence for in vivo applications.

In embodiments, the at least one cds is a codon modified cds, wherein the codon modified cds is selected from a C maximized cds (as further defined in WO2021239880 [p. 122, lines 33 to 39] which is hereby incorporated by reference); a CAI maximized cds (as further defined in WO2021239880 [p. 123, lines 33 to 44] which is hereby incorporated by reference); a human codon usage adapted cds (as further defined in WO2021239880 [p. 123, lines 7 to 17] which is hereby incorporated by reference); a G/C content modified cds (as further defined in WO2021239880 [p. 123, lines 19 to 31] which is hereby incorporated by reference); a G/C optimized cds; or any combination thereof.

When transfected into mammalian host cells, the nucleic acid comprising the codon modified coding sequence has a stability of between 12-18 hours, or greater than 18 hours, e.g., 24, 36, 48, 60, 72, or greater than 72 hours and is capable of being expressed by the mammalian host cell.

When transfected into mammalian host cells, the artificial nucleic acid comprising the codon modified coding sequence is translated into protein, wherein the amount of protein is at least comparable to, or preferably at least 10% more than, or at least 20% more than, or at least 30% more than, or at least 40% more than, or at least 50% more than, or at least 100% more than, or at least 200% or more than the amount of protein obtained by a naturally occurring or wild type or reference coding sequence transfected into mammalian host cells.

In particularly preferred embodiments, the artificial nucleic acid may be modified, wherein the G/C content of the at least one coding sequence may be optimized compared to the G/C content of the corresponding wild type or reference coding sequence (“G/C optimized coding sequence”). “Optimized” in that context refers to a coding sequence wherein the G/C content is preferably increased to the essentially highest possible G/C content. The generation of a G/C content optimized nucleic acid sequence may be carried out using a method according to WO2002098443. In this context, the disclosure of WO2002098443 is included in its full scope in the present invention.

In preferred embodiments, the at least one codon modified coding sequence is a G/C optimized coding sequence. Such G/C optimized nucleic acid sequences are provided in Table 1A or 1B or Table 2.

In preferred embodiments, the at least one cds of the artificial nucleic acid, preferably the RNA, has a G/C content of at least about 50%, 55%, or 60%. In particular embodiments, the at least one cds of the nucleic acid has a G/C content of at least about 50%, 51%, 52%, 53%, 54%, 55%, 56%, 57%, 58%, 59%, 60%, 61%, 62%, 63%, 64%, 65%, 66%, 67%, 68%, 69%, or 70%. In particularly preferred embodiments, the artificial nucleic acid may be modified, wherein the G/C content of the at least one cds may be optimized compared to the G/C content of the corresponding wild type or reference cds (“G/C optimized coding sequence”). “Optimized” in that context refers to a coding sequence wherein the G/C content is preferably increased to the essentially highest possible G/C content. The generation of a G/C content optimized nucleic acid sequence may be carried out using a method according to WO2002098443. In this context, the disclosure of WO2002098443 is included in its full scope in the present invention.

### UTRs:

In preferred embodiments, the artificial nucleic acid of the invention, preferably the RNA, comprises at least one untranslated region (UTR) or UTR element.

The terms “untranslated region” or “UTR” or “UTR element” refer to a part of a nucleic acid molecule typically located 5′ or 3′ of a coding sequence. An UTR is not translated into protein. An UTR may be part of the nucleic acid, e.g. an RNA. An UTR may comprise elements for controlling gene expression, also called regulatory elements. Such regulatory elements may be, e.g., ribosomal binding sites, miRNA binding sites, promotor elements etc.

In preferred embodiments, the artificial nucleic acid comprises a coding sequence and a 5′-UTR and/or 3′-UTR. Notably, UTRs may harbour regulatory sequence elements that determine RNA turnover, stability, and localization. Moreover, UTRs may harbour sequence elements that enhance translation. In medical applications, translation of the nucleic acid into at least one peptide or protein is of paramount importance to therapeutic efficacy. Certain combinations of 3′-UTRs and/or 5′-UTRs may enhance the expression of operably linked coding sequences encoding peptides or proteins as defined herein. Nucleic acid molecules harbouring said UTR combinations advantageously enable rapid and transient expression of encoded tumour antigens after administration to a subject.

Suitably, the artificial nucleic acid comprises at least one untranslated region (UTR) preferably selected from at least one 5′-UTR and/or at least one 3′-UTR. Said 5′-UTRs or 3′-UTRs may be derived from naturally occurring genes or may be synthetically engineered. In preferred embodiments, the artificial nucleic acid comprises at least one coding sequence as defined herein operably linked to at least one 3′-UTR and/or at least one 5′-UTR.

Preferably, the at least one untranslated region (UTR) is selected from at least one heterologous 5′-UTR and/or at least one heterologous 3′-UTR.

The term “heterologous” sequence or UTR as used herein is intended to refer to a nucleic acid sequence or UTR that is not from the same gene or the same genomic fusion or the same naturally occurring transcript (e.g. lncRNA). Accordingly, heterologous sequences or UTRs may be derivable from the same organism (e.g. human) or from a different organism. Heterologous sequences or UTRs do naturally (in nature) not occur in the same nucleic acid.

In preferred embodiments, the artificial nucleic acid of the invention comprises at least one 3′-UTR.

The terms “3′-untranslated region” or “3′-UTR” refer to a part of a nucleic acid located 3′ (i.e. downstream) of a cds and which is not translated into protein. A 3′-UTR may be part of a nucleic acid located between a cds and an (optional) terminal poly(A) sequence. A 3′-UTR may comprise elements for controlling gene expression, also called regulatory elements. Such regulatory elements may be, e.g., ribosomal binding sites, miRNA binding sites.

Preferably, the artificial nucleic acid comprises at least one 3′-UTR, which may be derivable from a gene that relates to an RNA with enhanced half-life (i.e. that provides a stable RNA).

In some embodiments, the 3′-UTR comprises one or more of a polyadenylation signal, a binding site for proteins that affect a nucleic acid stability of location in a cell, or one or more miRNA or binding sites for miRNAs.

In preferred embodiments, the artificial nucleic acid comprises at least one 3′-UTR, wherein the at least one 3′-UTR comprises or consists of a nucleic acid sequence derived or selected from a 3′-UTR of a gene selected from PSMB3, ALB7, alpha-globin, beta-globin, ANXA4, CASP1, COX6B1, FIG. 4, GNAS, NDUFA1, RPS9, SLC7A3 or TUBB4B, or from a homolog, a fragment, or variant of any one of these genes.

In preferred embodiments, the at least one 3′-UTR that is derived or selected from PSMB3, ALB7, alpha-globin, beta-globin, ANXA4, CASP1, COX6B1, FIG. 4, GNAS, NDUFA1, RPS9, SLC7A3 or TUBB4B comprises or consist of a nucleic acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 66-95, 112-123, or a fragment or a variant of these.

In other embodiments, the at least one 3′-UTR comprises or consist of a nucleic acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 96-111, or a fragment or a variant of any of these.

In particularly preferred embodiments, the artificial nucleic acid comprises a 3′-UTR derived or selected from a PSMB3 gene, wherein the at least one heterologous 3′-UTR comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 66, 67, 112-123, preferably SEQ ID NO: 67, or a fragment or a variant of any of these.

In preferred embodiments, the artificial nucleic acid of the invention comprises at least one 5′-UTR.

The terms “5′-untranslated region” or “5′-UTR” refer to a part of a nucleic acid located 5′ (i.e. “upstream”) of a cds and which is not translated into protein. A 5′-UTR may be part of a nucleic acid located 5′ of the cds. Typically, a 5′-UTR starts with the transcriptional start site and ends before the start codon of the cds. A 5′-UTR may comprise elements for controlling gene expression, also called regulatory elements. Such regulatory elements may be, e.g., ribosomal binding sites, miRNA binding sites etc. The 5′-UTR may be modified, e.g. by enzymatic or co-transcriptional addition of a 5′-cap structure (e.g. for mRNA as defined below).

Preferably, the artificial nucleic acid comprises at least one 5′-UTR, which may be derivable from a gene that relates to an RNA with enhanced half-life (i.e. that provides a stable RNA).

In some embodiments, the 5′-UTR comprises one or more of a binding site for proteins that affect a nucleic acid stability or nucleic acid location in a cell, or one or more miRNA or binding sites for miRNAs (as defined above).

Accordingly, miRNA or binding sites for miRNAs as defined above may be removed from the 5′-UTR or introduced into the 5′-UTR in order to tailor the expression of the nucleic acid to desired cell types or tissues (e.g. muscle cells).

In preferred embodiments, the artificial nucleic acid comprises at least one 5′-UTR, wherein the at least one 5′-UTR comprises a nucleic acid sequence derived or selected from a 5′-UTR of gene selected from HSD17B4, RPL32, AIG1, alpha-globin, ASAH1, ATP5A1, COX6C, DPYSL2, MDR, MP68, NDUFA4, NOSIP, RPL31, RPL35A, SLC7A3, TUBB4B, or UBQLN2, or from a homolog, a fragment or variant of any one of these genes,

In preferred embodiments, the at least one 5′-UTR derived or selected from HSD17B4, RPL32, AIG1, alpha-globin, ASAH1, ATP5A1, COX6C, DPYSL2, MDR, MP68, NDUFA4, NOSIP, RPL31, RPL35A, SLC7A3, TUBB4B, or UBQLN2 comprises or consist of a nucleic acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 12-45, 64, 65, or a fragment or a variant of any of these.

In other embodiments, the at least one 5′-UTR comprises or consist of a nucleic acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 46-63, or a fragment or a variant of any of these.

In particularly preferred embodiments, the nucleic acid comprises a 5′-UTR derived or selected from a HSD17B4 gene wherein the at least one heterologous 5′-UTR comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 12, 13, 64, 65, preferably SEQ ID NO: 13, or a fragment or a variant of any of these.

In embodiments, the at least one nucleic acid, preferably the RNA, comprises at least one cds operably linked to a 3′-UTR element and/or a 5′-UTR element selected from the 5′-UTR/3′-UTR combinations (5′UTR/3′UTR) provided in WO2021239880 [p. 127, line 35 to p. 128, line 2], which is hereby incorporated by reference.

In preferred embodiments, the at least one heterologous 5′-UTR is selected from HSD17B4 and the at least one heterologous 3′ UTR is selected from PSMB3.

Accordingly, in particularly preferred embodiments, the artificial nucleic acid, preferably the RNA comprises at least one cds as defined herein encoding at least one tumour antigen as defined herein, wherein said cds is operably linked to a HSD17B4 5′-UTR and a PSMB3 3′-UTR (HSD17B4/PSMB3 (a-1)). It has been shown by the inventors that this embodiment is particularly beneficial for expressing the tumour antigen in human cells.

In various embodiments, the nucleic acid, e.g. the RNA, is monocistronic, bicistronic, or multicistronic.

In preferred embodiments, the nucleic acid, preferably the RNA, is monocistronic.

Accordingly, in a particularly preferred embodiment, the artificial nucleic acid comprises a ribosome binding site, also referred to as “Kozak sequence”, identical to or at least 80%, 85%, 90%, 95% identical to any one of SEQ ID NOs: 1 or 2, or sequences GCCGCCACC (DNA), GCCGCCACC (RNA), GCCACC (DNA), GCCACC (RNA), ACC (DNA) or ACC (RNA), or fragments or variants of any of these. In preferred embodiments, the “Kozak sequence” comprises or consists of RNA sequence ACC.

### DNA and RNA Constructs:

In particularly preferred embodiments, the artificial nucleic acid of the invention is an isolated nucleic acid. The term “isolated nucleic acid” does not comprise a cell or a subject that comprises said nucleic acid but relates to the artificial nucleic acid as an isolated molecule or ensemble of isolated molecules. For example, the “isolated nucleic acid” can be an artificial nucleic acid isolated or purified from a cell (e.g. cell culture, bacterial culture), or can be an artificial nucleic acid (e.g. RNA) isolated from an RNA in vitro transcription.

In particularly preferred embodiments, the artificial nucleic acid of the invention is a therapeutic nucleic acid.

Accordingly, the artificial nucleic acid is suitably used in a therapeutic context, in particular to provide a therapeutic modality for providing at least one tumour antigen according to the invention.

In embodiments, the artificial nucleic acid of the invention is selected from a DNA or an RNA.

In embodiments, the artificial nucleic acid of the invention is selected from a DNA. The DNA may be any type of DNA that comprises a cds as defined herein including any type of single stranded DNA, any type of double stranded DNA, any type of linear DNA, and any type of circular DNA. A suitable DNA in the context of the invention may be selected from bacterial plasmid, an adenovirus, a poxvirus, a parapoxivirus (orf virus), a vaccinia virus, a fowlpox virus, a herpes virus, an adeno-associated virus (AAV), an alphavirus, a lentivirus, a lambda phage, a lymphocytic choriomeningitis virus and a Listeria sp, Salmonella sp. In preferred embodiments, the DNA a viral DNA, preferably an AAV DNA.

In particularly preferred embodiments, the artificial nucleic acid of the invention is an RNA. The RNA may be any type of RNA that comprises a cds as defined herein including any type of single stranded RNA, double stranded RNA, linear RNA, and circular RNA. In preferred embodiments, the RNA may be any type of RNA that comprises a cds as defined herein including any type of single stranded RNA, any type of double stranded RNA, any type of linear RNA, and any type of circular RNA, wherein the RNA is not a lncRNA. In preferred embodiments, the RNA is selected from mRNA, circular RNA, replicon RNA or self-replicating RNA, or viral RNA, preferably mRNA or a circular RNA.

In embodiments, the RNA is not a lncRNA. In particular, the artificial nucleic acid of the invention is not a lncRNA selected from lnc-WDR72-2:4, lnc-TRPC5-3:1, lnc-CLEC2D-9:1, KCNMB2-AS1:4, lnc-ZC3H8-6:1, lnc-NTF3-5:5, or LINC00893:25, which means that the RNA of the invention may of course comprise a coding sequence encoding a peptide encoded by lnc-WDR72-2:4, lnc-TRPC5-3:1, lnc-CLEC2D-9:1, KCNMB2-AS1:4, lnc-ZC3H8-6:1, lnc-NTF3-5:5, or LINC00893:25, but the RNA of the invention is not identical in sequence and length to lnc-WDR72-2:4, lnc-TRPC5-3:1, lnc-CLEC2D-9:1, KCNMB2-AS1:4, lnc-ZC3H8-6:1, lnc-NTF3-5:5, or LINC00893:25.

In embodiments, the RNA is a circular RNA. A “circular RNA” (circRNAs) is an RNA connected to form a circle and therefore does not comprise a 3′ or 5′ terminus. Said circRNA comprises at least one cds as defined herein. circRNA construct designs can be taken from WO2023073228, claims 1 to 51, hereby incorporated by reference.

In other embodiments, the RNA is a replicon RNA or self-replicating RNA. Such constructs may encode replicase elements derived from e.g. alphaviruses (e.g. SFV, SIN, VEE, or RRV) and a cds as defined herein.

In particularly preferred embodiments, the RNA is selected from an mRNA. Accordingly, the artificial nucleic acid of the invention is an mRNA, suitably an isolated mRNA.

An mRNA is preferred in the context of the invention to provide tumour antigens according to the invention because mRNA allows for regulated dosage, transient expression, complete degradation of the mRNA after protein synthesis, and do not pose the risk of insertional mutations.

Preferably, the artificial nucleic acid, preferably the RNA, comprises about 50 to about 20000 nucleotides, or about 500 to about 10000 nucleotides, or about 1000 to about 10000 nucleotides, or preferably about 1000 to about 5000 nucleotides or about 2000 to about 5000 nucleotides.

### Poly(N) Sequences, Histone Stem-Loops:

In preferred embodiments, the artificial nucleic acid comprises at least one poly(N) sequence, e.g. at least one poly(A) sequence, at least one poly(U) sequence, at least one poly(C) sequence, or combinations thereof.

In preferred embodiments, the artificial nucleic acid, e.g. the RNA, comprises at least one poly(A) sequence. In some embodiments, the artificial nucleic acid comprises least two, three, or more poly(A) sequences.

The terms “poly(A) sequence” or “poly(A) tail” refer to a sequence up to 1000 adenosines typically located at the 3′-end of a linear RNA. Typically, a poly(A) sequence is homopolymeric. Alternatively, a poly(A) sequence may be interrupted by at least one nucleotide different from an adenosine.

In embodiments, the at least one poly(A) sequence may comprise about 20 to about 500 adenosine nucleotides, about 40 to about 250 adenosine nucleotides, about 60 to about 250 adenosine nucleotides, preferably about 60 to about 150 adenosine nucleotides. Suitably, the length of the poly(A) sequence may be at least about or even more than about 50, 64, 75, 100, 200, 300, 400, or 500 adenosines, preferably consecutive adenosine nucleotides.

In particularly preferred embodiments, the at least one poly(A) sequence comprises about 100 adenosine nucleotides (A100), preferably about 100 consecutive adenosine nucleotides.

In alternative embodiments, the at least one nucleic acid comprises at least one interrupted poly(A) sequence, wherein the poly(A) sequence is interrupted by non-adenosine nucleotides, preferably by about 10 non-adenosine (N10) nucleotides. In that context, a poly(A) sequence A30-N10-A70 is preferred.

The poly(A) sequence as defined herein may be located directly at the 3′ terminus of the artificial nucleic acid, preferably the RNA. In preferred embodiments, the 3′-terminal nucleotide (that is the last 3′-terminal nucleotide in the polynucleotide chain) is the 3′-terminal A nucleotide of the at least one poly(A) sequence. The term “directly located at the 3′ terminus” has to be understood as being located exactly at the 3′ terminus—in other words, the 3′ terminus of the nucleic acid consists of a poly(A) sequence terminating with an A.

Advantageously, having a 3′ terminus ending on an adenosine nucleotide decreases the induction of interferons, e.g. IFNalpha, by the RNA of the invention if for example administered as a medicament to a human. This is important as the induction of interferons, e.g. IFNalpha, is thought to be one main factor for induction of side effects.

Accordingly, in particularly preferred embodiments, the artificial nucleic acid of the invention, e.g. the RNA, comprises a poly(A) sequence of about 100 consecutive adenosine nucleotides, wherein said poly(A) sequence is located directly at the 3′ terminus of the RNA, optionally wherein the 3′ terminal nucleotide is an adenosine

In preferred embodiments, the artificial nucleic acid, e.g. the RNA, comprises at least one histone stem-loop (hSL) or histone stem-loop structure. A hSL in the context of the invention may be located in the 3′ region. The term “histone stem-loop” (hSL) refers to a nucleic acid sequences that forms a stem-loop secondary structure predominantly found in histone mRNAs. Histone stem-loop sequences/structures may suitably be selected from hSL sequences as disclosed in WO2012019780. A hSL sequence that may be used herein may be derived from formulae (I) or (II) of WO2012019780. Accordingly, the artificial nucleic acid may comprise at least one hSL sequence derived from the specific formulae (Ia) or (IIa) of WO2012019780.

In preferred embodiments, the at least one histone stem-loop sequence comprises or consists of a nucleic acid sequence identical or at least 70%, 80%, 90%, 91%, 92%, 93%, 94%, 95%, 96%, 97%, 98%, or 99% identical to any one of SEQ ID NOs: 3 or 4, or a fragment or variant of any of these. Preferably, the histone stem-loop sequence comprises or consists of a nucleic acid sequence according to SEQ ID NO: 4, or a fragment or thereof.

In preferred embodiments, the artificial nucleic acid comprises a 3′-terminal sequence element. The 3′-terminal sequence element represents the 3′ terminus of the RNA. A 3′-terminal sequence element may comprise at least one poly(N) sequence as defined herein and, optionally, at least one hSL as defined herein.

In preferred embodiments, the artificial nucleic acid comprises at least one 3′-terminal sequence element comprising or consisting of an RNA sequence being identical or at least 70%, 80%, 90%, 91%, 92%, 93%, 94%, 95%, 96%, 97%, 98%, or 99% identical to any one of SEQ ID NOs: 5-11, or a fragment or variant of these sequences.

In preferred embodiments, the artificial nucleic acid comprises a 3′-terminal sequence element comprising a hSL as defined herein followed by a poly(A) sequence comprising about 100 consecutive adenosines.

In particularly preferred embodiments, the artificial nucleic acid comprises a 3′-terminal sequence element comprising or consisting of a nucleic acid sequence being identical or at least 70%, 80%, 90%, 91%, 92%, 93%, 94%, 95%, 96%, 97%, 98%, or 99% identical to SEQ ID NO: 5 or 6, or a fragment or variant thereof.

In some embodiments, the artificial nucleic acid comprises a 5′-terminal sequence element comprising or consisting of a nucleic acid sequence being identical or at least 70%, 80%, 90%, 91%, 92%, 93%, 94%, 95%, 96%, 97%, 98%, or 99% identical to any one of sequences AGGAGA, GGGAGA, GGGAAA, AGAAUA, AGAUUA, GAUGGG or GGGCG, or a fragment or variant of these sequences, preferably AGGAGA. Such a 5′-terminal sequence element may comprise e.g. a binding site for T7 RNA polymerase. Further, the first nucleotide of said 5′-terminal start sequence may preferably comprise a 2′O methylation, e.g. 2′O methylated guanosine or a 2′O methylated adenosine.

### Modified Nucleotides:

In various embodiments, the artificial nucleic acid, preferably the RNA, is modified, wherein the modification refers to chemical modifications comprising backbone modifications as well as sugar modifications or base modifications.

A modified nucleic acid or RNA may comprise nucleotide analogues/modifications, e.g. backbone modifications, sugar modifications or base modifications. A backbone modification in the context of the invention is a modification in which phosphates of the backbone of the nucleotides of the RNA are chemically modified. A sugar modification in the context of the invention is a chemical modification of the sugar of the nucleotides of the RNA. Furthermore, a base modification in the context of the invention is a chemical modification of the base moiety of the nucleotides of the RNA. In this context, nucleotide analogues or modifications are preferably selected from nucleotide analogues which are applicable for transcription and/or translation.

Accordingly, in preferred embodiments, the nucleic acid, preferably the RNA of the invention comprises at least one modified nucleotide.

In some embodiments, the at least one modified nucleotide is selected from pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine.

Particularly preferred in that context are pseudouridine (ψ) and N1-methylpseudouridine (m1ψ). Accordingly, in preferred embodiments, the nucleic acid is an RNA that comprises at least one modified nucleotide, preferably a modified nucleotide selected from pseudouridine (ψ) or N1-methylpseudouridine (m1ψ).

In some embodiments, essentially all, e.g. essentially 100% of the uracil in the coding sequence (or the full nucleic acid sequence) have a chemical modification, preferably a chemical modification in the 5-position of the uracil.

In preferred embodiments, 100% of the uracil in the full nucleic acid sequence, preferably the RNA sequence are substituted with a modified nucleotide, preferably N1-methylpseudouridine (m1ψ). Alternatively, 100% of the uracil in the full nucleic acid sequence, preferably the RNA sequence are substituted with pseudouridine (ψ).

In preferred embodiments, the artificial nucleic acid, preferably the RNA, does not comprise chemically modified nucleotides. Notably, a 5′-cap structure as defined below is typically not considered to be a chemically modified nucleotide. Accordingly, the artificial nucleic acid, preferably the RNA, comprises a sequence that consists only of G, C, A and U nucleotides and therefore does not comprise modified nucleotides, and optionally comprises a 5′-cap structure. RNA constructs that do not comprise chemically modified nucleotides may be beneficial in the context of cancer therapies as stronger T-cell responses can be induced by providing the cancer antigens via non-modified RNA (compared to m1ψ or ψ modified RNA).

In preferred embodiments, the artificial nucleic acid, preferably the RNA of the invention does not comprise N1-methylpseudouridine (m1ψ) substituted positions or pseudouridine (ψ) substituted positions.

### Cap Structures:

In preferred embodiments, the artificial nucleic acid is an RNA that comprises a 5′-cap structure.

Such a 5′-cap structure suitably stabilizes the nucleic acid and/or enhances expression of the encoded tumour antigen and/or reduces the stimulation of the innate immune system after administration.

Accordingly, in preferred embodiments, the artificial nucleic acid, preferably the RNA, comprises a 5′-cap structure, preferably m7G, cap0, cap1, cap2, a modified cap0 or a modified cap1 structure.

The term “5′-cap structure” as used herein will be recognized and understood by the person of ordinary skill in the art and is e.g. intended to refer to a 5′ modified nucleotide, particularly a guanine nucleotide, positioned at the 5′-end of an RNA. Preferably, the 5′-cap structure is connected via a 5′-5′-triphosphate linkage to the RNA.

5′-cap structures which may be suitable in the context of the present invention are cap0 (methylation of the first nucleobase, e.g. m7GpppN), cap1 (additional methylation of the ribose of the adjacent nucleotide of m7GpppN), cap2 (additional methylation of the ribose of the 2nd nucleotide downstream of the m7GpppN), cap3 (additional methylation of the ribose of the 3rd nucleotide downstream of the m7GpppN), cap4 (additional methylation of the ribose of the 4th nucleotide downstream of the m7GpppN), ARCA (anti-reverse cap analogue), modified ARCA (e.g. phosphothioate modified ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

Suitably, a 5′-cap (cap0 or cap1) structure may be formed in chemical RNA synthesis or in RNA in vitro transcription (co-transcriptional capping) using cap analogues.

The term “cap analogue” as used herein will be recognized and understood by the person of ordinary skill in the art and is e.g. intended to refer to a non-polymerizable di-nucleotide or tri-nucleotide that has cap functionality in that it facilitates translation or localization, and/or prevents degradation of an RNA molecule when incorporated at the 5′-end of the nucleic acid molecule. Non-polymerizable means that the cap analogue will be incorporated only at the 5′-terminus because it does not have a 5′ triphosphate and therefore cannot be extended in the 3′-direction by a template-dependent polymerase, particularly, by template-dependent RNA polymerase.

In embodiments, a cap1 structure is generated using a cap analogue as disclosed in WO2017053297, WO2017066793, WO2017066781, WO2017066791, WO2017066789, WO2017066782, WO2018075827, WO2017066797, and WO2023007019. Preferably, cap structures derivable from the structure disclosed in claim 1-5 of WO2017053297 may be suitably used to co-transcriptionally generate a cap1 structure. Further, any cap structures as defined in claim 1 to claim 37 of WO2023007019 may be suitably used to generate a cap1.

In preferred embodiments, the 5′-cap structure may suitably be added co-transcriptionally using tri-nucleotide cap analogue as defined herein, preferably in an RNA in vitro transcription reaction as defined herein.

In particularly preferred embodiments, the artificial nucleic acid, preferably the RNA of the invention comprises a cap1 structure or a modified cap1 structure.

In preferred embodiments, the cap1 structure is formed via co-transcriptional capping using tri-nucleotide cap analogues m7G(5′)ppp(5′)(2′OMeA)pG or m7G(5′)ppp(5′)(2′OMeG)pG. A particularly preferred cap1 analogue in that context is m7G(5′)ppp(5′)(2′OMeA)pG.

In other preferred embodiments, the cap1 structure is a modified cap1 structure and is formed using co-transcriptional capping using tri-nucleotide cap analogue 3′OMe-m7G(5′)ppp(5′)(2′OMeA)pG.

Alternatively, the 5′-cap structure may be formed via enzymatic capping using capping enzymes (e.g. vaccinia virus capping enzymes and/or 2′-O methyltransferases) to generate cap0, cap1 or cap2 structures.

It is preferred that at least 80%, 85%, 90%, 95% of the RNA species comprise a cap structure, preferably a cap1 structure, as determined by a capping assay (e.g. via an assay as described in cl. 27 to 46 of WO2015101416).

### Further RNA Features:

In various embodiments, the artificial nucleic acid is an RNA that provides at least one coding sequence encoding at least one tumour antigen as defined herein that is produced after administration to a cell or subject.

Suitable elements that the RNA of the invention preferably comprises are for example a 5′ Cap structure as defined herein, a 5′ UTR as defined herein, a 3′ UTR as defined herein, hSL as defined herein, poly(A) sequence as defined herein, and optional chemical modifications as defined herein.

In preferred embodiments, the RNA is preferably an in vitro transcribed RNA (e.g. an in vitro transcribed mRNA).

In preferred embodiments, the nucleotide mixture (i.e. the fraction of each nucleotide in the mixture) used for RNA in vitro transcription reactions is optimized for the given RNA sequence, preferably as described WO2015188933. Accordingly, in preferred embodiments, the nucleic acid of the invention is an in vitro transcribed RNA, preferably wherein RNA in vitro transcription has been performed in the presence of a sequence optimized nucleotide mixture.

In preferred embodiments, the artificial nucleic acid of the invention is a purified RNA, preferably a purified mRNA. Suitably, the RNA of the invention has been purified by at least one step of purification

The term “purified RNA” or refers to RNA which has a higher purity after certain purification steps than the starting material (e.g. the crude IVT RNA). Typical impurities comprise peptides, proteins, spermidine, BSA, abortive RNA fragments, dsRNA, free nucleotides, DNA, buffer components etc. It is desirable in this regard for the “degree of RNA purity” to be as close as possible to 100%. Preferably, “purified RNA” as used herein has a degree of purity of more than 75%, 80%, 85%, 90%, or 95%. The degree of purity may be determined by an analytical HPLC.

In embodiments, the nucleic acid is a purified RNA that has been purified by at least one step of (RP)-HPLC, AEX, size exclusion chromatography (SEC), hydroxyapatite chromatography, tangential flow filtration (TFF), filtration, precipitation, core-bead flow through chromatography, oligo (dT) purification, cellulose-based purification, or any combination thereof. Preferably, the RNA has been purified using RP-HPLC (preferably as described in WO2008077592) and/or TFF (preferably as described in WO2016193206) and/or oligo d (T) purification (preferably as described in WO2016180430) to e.g. to remove dsRNA, non-capped RNA and/or RNA fragments.

In embodiments, the RNA has an integrity of at least 60%, 70%, 80%, 90%. The term “RNA integrity” generally describes whether the complete RNA sequence is present. The RNA integrity can be determined by RP-HPLC and may be based on determining the area under the peak of the expected full-length RNA in a chromatogram. It is desirable in this regard for the “degree of RNA integrity” to be as close as possible to 100%.

In preferred embodiments, the nucleic acid, preferably the RNA, is suitable for use in treatment or prevention of a disease, disorder or condition, preferably a tumour or cancer disease, disorder, or condition or any disease, disorder, or condition related to tumour or cancer. The term “cancer” according to the disclosure also comprises cancer metastases.

### Preferred Nucleic Acid Constructs:

In various embodiments, the artificial nucleic acid comprises at least the following elements:
A) a 5′-cap structure, preferably as specified herein;
B) at least one cds encoding at least one tumour antigen as defined herein;
C) a 5′-UTR and/or a 3′-UTR, preferably as specified herein;
D) at least one poly(A) sequence, preferably as specified herein.

In preferred embodiments, the artificial nucleic acid, preferably the RNA, comprises the following sequence elements preferably in 5′- to 3′-direction:
A) a 5′-cap structure, preferably a cap1 structure;
B) a 5′-UTR preferably selected or derived from a 5′-UTR of a HSD17B4 gene, or a fragment thereof;
C) a coding sequence encoding at least one tumour antigen as defined herein;
D) a 3′-UTR preferably selected or derived from a 3′-UTR of a PSMB3 gene, or a fragment thereof;
E) optionally, a histone stem-loop; and
F) a poly(A) sequence preferably comprising about 100 A nucleotides.

In preferred embodiments, the artificial nucleic acid, preferably the RNA, comprises the following sequence elements preferably in 5′- to 3′-direction:
A) a 5′-cap structure, preferably a cap1 structure;
B) a 5′-UTR preferably selected or derived from a 5′-UTR of a HSD17B4 gene, or a fragment thereof;
C) a cds encoding at least one antigenic peptide selected from a peptide or protein encoded by a long non-coding RNA (lncRNA) comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 124-146, 546-571, 756, 757, or an immunogenic fragment or variant of any of these;
D) a 3′-UTR preferably selected or derived from a 3′-UTR of a PSMB3 gene, or a fragment thereof;
E) optionally, a histone stem-loop; and
F) a poly(A) sequence preferably comprising about 100 A nucleotides.

In particularly preferred embodiments, the nucleic acid is an mRNA that comprises the following sequence elements in 5′- to 3′-direction:
A) a 5′-cap structure, preferably a cap1 structure;
B) a 5′-UTR that comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 13, or a fragment or a variant of any of these;
C) a cds that comprises a nucleic acid sequence encoding at least one antigenic peptide selected or derived from a peptide or protein encoded by a long non-coding RNA (lncRNA), wherein the nucleic acid sequence is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 318-448, 651-755, 764-771, or a fragment or a variant of any of these;
D) a 3′-UTR that comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 67, or a fragment or a variant thereof;
E) optionally, a histone stem-loop; and
F) a poly(A) sequence, preferably comprising about 100 A nucleotides.

In particularly preferred embodiments, the nucleic acid is an mRNA that comprises the following sequence elements in 5′- to 3′-direction:
A) a 5′-cap structure, preferably a cap1 structure;
B) a 5′-UTR that comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 13, or a fragment or a variant of any of these;
C) a coding sequence that comprises a nucleic acid sequence encoding at least one antigenic peptide selected or derived from a peptide or protein of a neoantigen as defined herein, wherein the nucleic acid sequence is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 449-511, or a fragment or a variant of any of these;
D) a 3′-UTR that comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 67, or a fragment or a variant of any of these;
E) optionally, a histone stem-loop; and
F) a poly(A) sequence, preferably comprising about 100 A nucleotides.

### 2: Pharmaceutical Composition Comprising at Least One Nucleic Acid Encoding a Tumour Antigen:

In a second aspect, the invention provides a pharmaceutical composition comprising at least one artificial nucleic acid encoding at least one tumour antigen as defined in the first aspect.

Notably, features and embodiments described in the context of the first aspect (the nucleic acid of the invention) have to be read on and have to be understood as suitable embodiments of the pharmaceutical composition of the second aspect and vice versa.

In the context of the invention, a “composition” refers to any type of composition in which the specified ingredients (e.g. nucleic acid encoding at least one tumour antigen) may be incorporated, optionally along with any further constituents, usually with at least one pharmaceutically acceptable carrier or excipient. The composition may be a dry composition such as a powder, a granule, or a solid lyophilized form. Alternatively, the composition may be in liquid form, and each constituent may be independently incorporated in dissolved or dispersed form.

Preferably, the at least one artificial nucleic acid of the pharmaceutical composition is selected from an RNA as further defined in the first aspect. In particularly preferred embodiments, the at least one nucleic acid of the pharmaceutical composition is selected from an mRNA as further defined in first aspect.

In preferred embodiments, the pharmaceutical composition comprises a plurality or at least more than one nucleic acid species (e.g. RNA species), preferably wherein each nucleic acid species encodes a different tumour antigen.

Preferably, the pharmaceutical composition as defined herein may comprise 2 to 10 nucleic acid species each as defined in the first aspect, preferably 2 to 5 nucleic acid species each as defined in the first aspect, wherein each of the 2 to 10 nucleic acid species, preferably each of the 2 to 5 nucleic acid species, encode at least one different tumour antigen.

Suitably, the respective nucleic acid species encoding different tumour antigens are selected to obtain an increased coverage in a certain cancer disease, e.g. NSCLC or melanoma. Suitably, the coverage of all the different tumour antigens is at a value of above 0.5, preferably above 0.6, more preferably above 0.7.

In preferred embodiments in that context, the different tumour antigens (provided by the nucleic acid sequence) are selected to cover different HLA types. Accordingly, the different tumour antigen are selected from at least one tumour antigen as defined herein that comprises at least one epitope that binds to HLA-A type, at least one tumour antigen as defined herein that comprises at least one epitope binds to HLA-B type, at least one tumour antigen as defined herein that comprises at least one epitope binds to HLA-C type.

In various embodiments, the at least one artificial nucleic acid, preferably the at least one RNA of the pharmaceutical composition, is formulated with a pharmaceutically acceptable carrier or excipient.

### Formulation/Complexation:

In preferred embodiments, the at least one nucleic acid, preferably the at least one RNA is complexed or associated with at least one further compound to obtain a formulated composition. A formulation in that context may have the function of a transfection agent. A formulation in that context may also have the function of protecting the nucleic acid from degradation, e.g. to allow storage, shipment, etc.

In embodiments, the at least one nucleic acid, preferably the at least one RNA is formulated with at least one compound, e.g. peptides, proteins, lipids, polysaccharides, and/or polymers.

In embodiments, the at least one artificial nucleic acid, preferably the at least one RNA is formulated with at least one cationic (cationic or preferably ionizable) or polycationic compound (cationic or preferably ionizable). In preferred embodiments, the at least one artificial nucleic acid, preferably the at least one RNA is complexed or associated with or at least partially complexed or partially associated with one or more cationic (cationic or preferably ionizable) or polycationic compound.

The term “cationic or polycationic compound” refers to a charged molecule, which is positively charged at a pH value ranging from about 1 to 9, at a pH value ranging from about 3 to 8, at a pH value ranging from about 4 to 8, at a pH value ranging from about 5 to 8, more preferably at a pH value ranging from about 6 to 8, even more preferably at a pH value ranging from about 7 to 8, most preferably at a physiological pH, e.g. ranging from about 7.2 to about 7.5. Accordingly, a cationic component, e.g. a cationic peptide, cationic protein, cationic polymer, cationic polysaccharide, cationic lipid may be any positively charged compound or polymer which is positively charged under physiological conditions. A “cationic or polycationic peptide or protein” may contain at least one positively charged amino acid, or more than one positively charged amino acid, e.g. selected from Arg, His, Lys or Orn. Accordingly, “polycationic” components are also within the scope exhibiting more than one positive charge under the given conditions.

In preferred embodiments, the at least one cationic or polycationic compound is selected from a cationic or polycationic polymer, a cationic or polycationic polysaccharide, a cationic or polycationic lipid, a cationic or polycationic protein, a cationic or polycationic peptide, or any combinations thereof.

In particularly preferred embodiments, the at least one artificial nucleic acid, preferably the at least one RNA of the pharmaceutical composition is formulated in lipid-based carriers.

In the context of the invention, the term “lipid-based carriers” encompass lipid-based delivery systems for nucleic acid (e.g. RNA) that comprise a lipid component. A lipid-based carrier may additionally comprise other components suitable for encapsulating/incorporating/complexing a nucleic acid (e.g. RNA) including a cationic or polycationic polymer, a cationic or polycationic polysaccharide, a cationic or polycationic protein, a cationic or polycationic peptide, or any combinations thereof.

The at least one nucleic acid, preferably the RNA, may completely or partially be incorporated or encapsulated in a lipid-based carrier, wherein the at least one nucleic acid may be located in the interior space of the lipid-based carrier, within the lipid layer/membrane of the lipid-based carrier, or associated with the exterior of the lipid-based carrier. The incorporation of nucleic acid into lipid-based carriers may be referred to as “encapsulation”.

The term “encapsulated”, e.g. incorporated, complexed, encapsulated, partially encapsulated, associated, partially associated, refers to the essentially stable combination of nucleic acid, preferably RNA with one or more lipids into lipid-based carriers (e.g. larger complexes or assemblies) preferably without covalent binding of the nucleic acid. The lipid-based carriers-encapsulated nucleic acid (e.g. RNA) may be completely or partially located in the interior of the lipid-based carrier (e.g. the lipid portion and/or an interior space) and/or within the lipid layer/membrane of the lipid-based carriers. The encapsulation of a nucleic acid (e.g. RNA) into lipid-based carriers is also referred to herein as “incorporation” as the nucleic acid (e.g. RNA) is preferably contained within the interior of the lipid-based carriers. Without wishing to be bound to theory, the purpose of incorporating or encapsulating nucleic acid into lipid-based carriers may be to protect the nucleic acid from an environment which may contain enzymes, chemicals, or conditions that degrade the nucleic acid (e.g. RNA). Moreover, incorporating nucleic acid into lipid-based carriers may promote the uptake of the nucleic acid and their release from the endosomal compartment, and hence, may enhance the therapeutic effect of the nucleic acid (e.g. RNA) when administered to a cell or a subject.

In embodiments, the lipid-based carriers are selected from liposomes, lipid nanoparticles, lipoplexes, solid lipid nanoparticles, lipo-polyplexes, and/or nanoliposomes. In preferred embodiments, the lipid-based carriers are lipid nanoparticles (LNPs).

In particularly preferred embodiments, the lipid nanoparticles encapsulate the at least one nucleic acid, preferably the at least one RNA of the invention.

In preferred embodiments, the lipid-based carriers comprise at least one or more lipids selected from at least one aggregation-reducing lipid, at least one cationic lipid, at least one neutral lipid or phospholipid, or at least one steroid or steroid analogue, or any combinations thereof.

In preferred embodiments, the lipid-based carriers comprise (i) an aggregation-reducing lipid, (ii) a cationic lipid or ionizable lipid, and (iii) a neutral lipid/phospholipid or a steroid/steroid analogue.

In particularly preferred embodiments, the lipid-based carriers comprise an (i) aggregation-reducing lipid, (ii) a cationic lipid or ionizable lipid, (iii) a neutral lipid or phospholipid, (iv) and a steroid or steroid analogue.

### Aggregation Reducing Lipids/Polymer Conjugated Lipids:

In preferred embodiments, the lipid-based carriers comprise at least one aggregation reducing lipid or moiety.

The term “aggregation reducing moiety” refers to a molecule comprising a moiety suitable of reducing or preventing aggregation of the lipid-based carriers. The term “aggregation reducing lipid” refers to a molecule comprising both a lipid portion and a moiety suitable of reducing or preventing aggregation of the lipid-based carriers. Under storage conditions or during formulation, the lipid-based carriers may undergo charge-induced aggregation, a condition which can be undesirable for the stability of the lipid-based carriers. Therefore, it can be desirable to include a compound or moiety which can reduce aggregation, for example by sterically stabilizing the lipid-based carriers. Such a steric stabilization may occur when a compound having a sterically bulky but uncharged moiety that shields or screens the charged portions of a lipid-based carriers from close approach to other lipid-based carriers in the composition. In the context of the invention, stabilization of the lipid-based carriers is achieved by including lipids which may comprise a lipid bearing a sterically bulky group which, after formation of the lipid-based carrier, is preferably located on the exterior of the lipid-based carrier. Suitable aggregation reducing groups include hydrophilic groups, e.g. monosialoganglioside GM1, polyamide oligomers (PAO), or certain polymers, such as poly(oxyalkylenes), e.g., poly(ethylene glycol) or poly(propylene glycol).

Lipids comprising a polymer as aggregation reducing group are herein referred to as “polymer conjugated lipid”.

The term “polymer conjugated lipid” refers to a molecule comprising both a lipid portion and a polymer portion, wherein the polymer is suitable of reducing or preventing aggregation of lipid-based carriers comprising the RNA. A polymer has to be understood as a substance or material consisting of very large molecules, or macromolecules, composed of many repeating subunits. A suitable polymer in the context of the invention may be a hydrophilic polymer. An example of a polymer conjugated lipid is a PEGylated or PEG-conjugated lipid.

In preferred embodiments, the lipid-based carriers comprise an aggregation reducing lipid selected from a polymer conjugated lipid.

In preferred embodiments, the polymer conjugated lipid is a PEG-conjugated lipid (or PEGylated lipid, PEG lipid).

The average molecular weight of the PEG moiety in the PEG-conjugated lipid preferably ranges from about 500 to about 8,000 Daltons (e.g., from about 1,000 to about 4,000 Daltons). In one preferred embodiment, the average molecular weight of the PEG moiety is about 2,000 Daltons. A PEG-conjugated lipid may be selected or derived from 1,2-dimyristoyl-rac-glycero-3-methoxypolyethylene glycol-2000 (PEG2000 DMG or DMG-PEG 2000), C10-PEG2K, or Cer8-PEG2K. A PEG-conjugated lipid may also be selected or derived from formula (IVa) of WO2018078053. A suitable PEG-conjugated lipid of said formula (IVa) has the chemical term 2 [(polyethylene glycol)-2000]-N,N-ditetradecylacetamide, also referred to as ALC-0159.

In preferred embodiments, the lipid-based carriers comprise an aggregation reducing lipid, wherein the aggregation reducing lipid is not a PEG-conjugated lipid. Accordingly, the aggregation reducing lipid may suitably be selected from a PEG-less lipid, e.g. a PEG-less polymer conjugated lipid.

In preferred embodiments, the aggregation reducing lipid (or polymer conjugated lipid) is a PEG-free lipid that comprises a polymer different from PEG.

A PEG-free lipid in the context of the invention may be selected or derived from a POZ-lipid. In preferred embodiments, the POZ lipids or respectively preferred polymer conjugated lipids are described in WO2023031394, the full disclosure herewith incorporated by reference. In particular, the disclosure relating to polymer conjugated lipids as shown in any one of claims 1 to 8 of WO2023031394 are incorporated by reference.

In embodiments, the polymer conjugated lipid is a PEG-free lipid selected from a POZ-lipid.

Accordingly, in embodiments, the polymer conjugated lipid is a “POZ-lipid”, which preferably is defined as a compound according to formula (POZ): [H]-[linker]-[M], wherein
[H] is a homopolymer moiety comprising at least one polyoxazoline (POZ) monomer unit

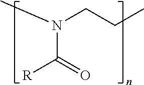

wherein R is C1-9 alkyl or C2-9 alkenyl, preferably C1, and n has a mean value ranging from 2 to 200, preferably from 20 to 100, more preferably from 24 to 26 or 45 to 50; [linker] is an optional linker group; [M] is a lipid moiety.

In preferred embodiment in the context of POZ-lipids, the aggregation-reducing lipid is selected or derived from PMOZ 1, PMOZ 2, PMOZ 3, PMOZ 4, or PMOZ 5 of WO2023031394.

In preferred embodiments, the aggregation-reducing lipid is selected or derived from PMOZ4 according to or derived from the following formula:

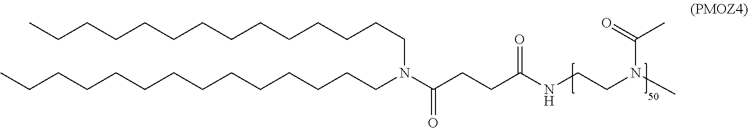

In preferred embodiments, the at least one aggregation-reducing lipid is selected from DMG-PEG 2000, C10-PEG2K, Cer8-PEG2K, or a POZ-lipid such as PMOZ4. In other preferred embodiments, the at least one aggregation-reducing lipid is selected from ALC-0159.

### Cationic Lipids:

In preferred embodiments, the lipid-based carriers comprise at least one cationic or ionizable lipid.

The cationic or ionizable lipid of the lipid-based carriers may be cationisable or ionizable, i.e. it becomes protonated as the pH is lowered below the pK of the ionizable group of the lipid, but is progressively more neutral at higher pH values. At pH values below the pK, the lipid is then able to associate with negatively charged nucleic acids. In certain embodiments, the cationic lipid comprises a zwitterionic lipid that assumes a positive charge on pH decrease.

In preferred embodiments, the lipid-based carriers comprise a cationic or ionizable lipid that preferably carries a net positive charge at physiological pH, more preferably the cationic or ionizable lipid comprises a tertiary nitrogen group or quaternary nitrogen group. Accordingly, in preferred embodiments, the lipid-based carriers comprise a cationic or ionizable lipid selected from an amino lipid, preferably wherein the amino lipid comprises a tertiary amine group.

In preferred embodiments, the at least one cationic or ionizable lipid is selected or derived from formula (III-1)

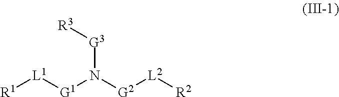

preferably, wherein one of L1 or L2 is —O(C═O)—, —(C═O)O—, —C(═O)—, —O—, —S(O)x-, —S—S—, —C(═O)S—, SC(═O)—, —NRaC(═O)—, —C(═O)NRa-, —NRaC(═O)NRa-, —OC(═O)NRa- or —NRaC(═O)O—, and the other of L1 or L2 is —O(C═O)—, —(C═O)O—, —C(═O)—, —O—, —S(O)x-, —S S—, —C(═O)S—, SC(═O)—, —NRaC(═O)—, —C(═O)NRa-, —NRaC(═O)NRa-, —OC(═O)NRa- or —NRaC(═O)O— or a direct bond; G1 and G2 are each independently unsubstituted C1-C12 alkylene or C1-C12 alkenylene; G3 is C1-C24 alkylene, C1-C24 alkenylene, C3-C8 cycloalkylene, C3-C8 cycloalkenylene; Ra is H or C1-C12 alkyl; R1 and R2 are each independently C6-C24 alkyl or C6-C24 alkenyl; R3 is H, OR5, CN, C(═O)OR4, OC(═O)R4 or —NR5C(═O)R4; R4 is C1-C12 alkyl; R5 is H or C1-C6 alkyl; and x is 0, 1 or 2.

In some embodiments, cationic or ionizable lipids may be selected from the lipids disclosed in WO2018078053 (i.e. lipids derived from formula I, II, and III of WO2018078053, or lipids as specified in claims 1 to 12 of WO2018078053), the disclosure of WO2018078053 hereby incorporated by reference in its entirety. In that context, lipids disclosed in Table 7 of WO2018078053 (e.g. lipids derived from formula I-1 to I-41) and lipids disclosed in Table 8 of WO2018078053 (e.g. lipids derived from formula II-1 to II-36) may be suitably used in the context of the invention. Accordingly, formula I-1 to formula I-41 and formula II-1 to formula II-36 of WO2018078053, and the specific disclosure relating thereto, are herewith incorporated by reference.

In embodiments, the lipid-based carriers of the pharmaceutical composition comprise a cationic lipid selected or derived from structures III-1 to III-36 of Table 9 of WO2018078053. Accordingly, formula III-1 to III-36 of WO2018078053, and the specific disclosure relating thereto, are herewith incorporated by reference.

In embodiments, the lipid-based carriers comprise a cationic lipid selected or derived from formula III-3 of WO2018078053. A preferred lipid of said formula III-3 has the chemical term ((4-hydroxybutyl) azanediyl)bis(hexane-6,1-diyl)bis(2-hexyldecanoate), also referred to as ALC-0315 i.e. CAS Number 2036272-55-4.

Further suitable cationic lipids may be selected or derived from cationic lipids according to PCT claims 1 to 14 of WO2021123332, or table 1 of WO2021123332, the disclosure relating to claims 1 to 14 or table 1 of WO2021123332 herewith incorporated by reference. Accordingly, suitable cationic lipids may be selected or derived from cationic lipids according Compound 1 to Compound 27 (C1-C27) of Table 1 of WO2021123332.

In embodiments, the lipid-based carriers comprise a cationic lipid selected or derived from (COATSOME® SS-EC)SS-33/4PE-15 (see C23 in Table 1 of WO2021123332). In other embodiments, the lipid-based carriers comprise a cationic lipid selected or derived from HEXA-C5DE-PipSS (see C2 in Table 1 of WO2021123332). In preferred embodiments, the lipid-based carriers comprise a cationic lipid selected or derived from compound C26 as disclosed in Table 1 of WO2021123332.

In other embodiments, the lipid-based carriers comprise a cationic lipid selected or derived from 9-Heptadecanyl 8-{(2-hydroxyethyl) [6-oxo-6-(undecyloxy) hexyl]amino}octanoate, also referred to as SM-102.

Accordingly, in preferred embodiments, the lipid-based carrier, preferably the LNP, comprises a cationic lipid selected or derived from above mentioned compound C26, SM-102, SS-33/4PE-15, or HEXA-C5DE-PipSS. In other preferred embodiments, the lipid-based carrier, preferably the LNP, comprise a cationic lipid selected or derived from ALC-0315.

### Neutral Lipids:

In preferred embodiments, the lipid-based carriers (e.g. LNPs) comprise at least one neutral lipid or phospholipid.

The term “neutral lipid” refers to any one of a number of lipid species that exist in either an uncharged or neutral zwitterionic form at physiological pH. Neutral lipids may be selected from DHPC, DHPC, DOPC, DPPC, DOPG, DPPG, DOPE, POPC, POPE, DOPE-mal, DPPE, DMPE, DSPE, 16-O-monomethyl PE, 16-O-dimethyl PE, 18-1-trans PE, SOPE, transDOPE, 1,2-diphytanoyl-sn-glycero-3-phosphoethanolamine (DPhyPE), DPhyPS (1,2-diphytanoyl-sn-glycero-3-phospho-L-serine), or mixtures thereof.

In preferred embodiments, the lipid-based carriers comprise a neutral lipid selected or derived from DSPC, DHPC, DPhyPE, or DPhyPS. Preferred in that context is DSPC.

### Steroids, Steroid Analogues or Sterols:

In preferred embodiments, the lipid-based carriers comprise a steroid, steroid analogue or sterol.

In embodiments, the steroid, steroid analogue or sterol is derived or selected from cholesterol, cholesteryl hemisuccinate (CHEMS), or any derivate of these. In preferred embodiments, the lipid-based carriers comprise cholesterol.

### Lipid-Based Carrier Compositions:

In embodiments, the lipid-based carriers, preferably the LNPs, comprising the at least one nucleic acid, preferably the at least one RNA, comprise
(i) at least one cationic lipid or ionizable lipid, preferably as defined herein;
(ii) at least one or two (e.g. two different) neutral lipids or phospholipids, preferably as defined herein;
(iii) at least one steroid or steroid analogue, preferably as defined herein; and
(iv) at least one aggregation reducing lipid, preferably as defined herein.

In embodiments, the lipid-based carriers, preferably the LNPs, comprising the at least one nucleic acid, preferably the at least one RNA, comprise
(i) at least one cationic lipid selected or derived from C26, SS-33/4PE-15, HEXA-C5DE-PipSS, SM-102;
(ii) at least one or two (e.g. two different) neutral lipid selected or derived from DSPC, DHPC, DPhyPE, DPhyPS;
(iii) at least one steroid or steroid analogue selected or derived from cholesterol; and
(iv) at least one aggregation reducing lipid selected or derived from DMG-PEG 2000, C10-PEG2K, Cer8-PEG2K, or ALC-0159 or “PMOZ 4”; and wherein the lipid-based carriers encapsulate the nucleic acid (e.g. the RNA).

In embodiments, the lipid-based carriers, preferably the LNPs, comprising the at least one nucleic acid, preferably the at least one RNA, comprise
(i) at least one cationic lipid selected or derived from ALC-0315;
(ii) at least one neutral lipid selected or derived from DSPC;
(iii) at least one steroid or steroid analogue selected or derived from cholesterol; and
(iv) at least one aggregation reducing lipid selected or derived from ALC-0159; and wherein the lipid-based carriers encapsulate the nucleic acid (e.g. the RNA).

In embodiments, the lipid-based carriers comprising the at least one nucleic acid, preferably the at least one RNA comprise
(i) a cationic lipid selected from C26;
(ii) a neutral lipid selected from DPhyPE and a neutral lipid selected from DPhyPS;
(iii) a steroid or steroid analogue selected from cholesterol; and
(iv) an aggregation reducing lipid selected from a POZ lipid as defined herein, preferably PMOZ 4; and wherein the lipid-based carriers encapsulate the nucleic acid (e.g. the RNA).

In embodiments, the cationic lipids (as defined herein), neutral lipid (as defined herein), steroid or steroid analogue (as defined herein), and/or aggregation reducing lipid (as defined herein) may be combined at various relative ratios.

In embodiments, the lipid-based carriers, preferably the LNPs, comprise (i) to (iv) in a molar ratio of about 20-60% cationic lipid or ionizable lipid, about 5-25% neutral lipid, about 25-55% steroid or steroid analogue, and about 0.5-15% aggregation reducing lipid e.g. polymer conjugated lipid, preferably wherein the lipid-based carriers encapsulate the at least one nucleic acid, preferably the at least one RNA.

In preferred embodiments, the lipid-based carriers, preferably the LNPs, comprise (i) to (iv) in a molar ratio of about 45-55% cationic lipid or ionizable lipid, about 5-15% neutral lipid, about 35-45% steroid or steroid analogue, and about 0.5-2.5% aggregation reducing lipid e.g. polymer conjugated lipid, preferably wherein the lipid-based carriers encapsulate the at least one nucleic acid, preferably the at least one RNA.

In more preferred embodiments, the lipid-based carriers, preferably the LNPs, comprise (i) to (iv) in a molar ratio of about 47-51% cationic lipid or ionizable lipid, about 8-12% neutral lipid, about 38-42% steroid or steroid analogue, and about 0.75-1.75% aggregation reducing lipid e.g. polymer conjugated lipid, preferably wherein the lipid-based carriers encapsulate the at least one nucleic acid, preferably the at least one RNA.

In embodiments, the lipid-based carriers, preferably the LNPs comprising the at least one nucleic acid, preferably the at least one RNA, comprise
(i) at least one cationic lipid selected from ALC-0315;
(ii) at least one neutral lipid selected from DSPC;
(iii) at least one steroid or steroid analogue selected from cholesterol; and
(iv) at least one aggregation reducing lipid selected from ALC-0159 and wherein the lipid-based carriers encapsulate the nucleic acid (e.g. the RNA), preferably wherein i) to (iv) are in a molar ratio of about 47.4% cationic lipid, about 10% neutral lipid, about 40.9% steroid or steroid analogue, and about 1.7% aggregation reducing lipid, preferably wherein the lipid-based carriers encapsulate the nucleic acid (e.g. the RNA).

In preferred embodiments, the lipid-based carrier, preferably the LNP comprising the at least one nucleic acid molecule, preferably RNA, comprises
(i) the cationic lipid C26 (VitE-C4DE-Pip-thioether);
(ii) the neutral lipid DPhyPE and the neutral lipid DPhyPS;
(iii) the steroid or steroid analog cholesterol; and
(iv) the aggregation reducing lipid PMOZ4, preferably wherein i) to (iv) are in a molar ratio of about 49% cationic lipid, about 10% neutral lipid, about 40% steroid or steroid analogue, and about 1% aggregation reducing lipid, preferably wherein the lipid-based carrier encapsulates the at least one nucleic acid molecule, preferably the RNA.

The amount of lipid comprised in the lipid-based carriers may be selected taking the amount of the nucleic acid cargo into account. In one embodiment, these amounts are selected such as to result in an N/P ratio of the lipid-based carriers encapsulating the nucleic acid in the range of about 0.1 to about 50. The N/P ratio is defined as the mole ratio of the nitrogen atoms (“N”) of the basic nitrogen-containing groups of the lipid to the phosphate groups (“P”) of the nucleic acid which is used as cargo. The N/P ratio may be calculated on the basis that, for example, 1 μg nucleic acid typically contains about 3 nmol phosphate residues, provided that the nucleic acid exhibits a statistical distribution of bases. The “N”-value of the lipid or lipidoid may be calculated on the basis of its molecular weight and the relative content of permanently cationic and—if present—cationisable groups.

In embodiments, the N/P ratio can be in the range of about 1 to about 50. In preferred embodiments, the range is from about 5 to about 20. In some embodiments, the N/P ratio is at about 17. In some embodiments, the N/P ratio is at about 14. In some embodiments, the N/P ratio is at about 6.

In various embodiments, the pharmaceutical composition comprises lipid-based carriers (encapsulating nucleic acid, preferably RNA as defined herein) that have a defined size (particle size, homogeneous size distribution).

The size of the lipid-based carrier is typically described herein as Z-average size. The term “Z-average size” refers to the mean diameter of particles as measured by dynamic light scattering (DLS) with data analysis using the so-called cumulant algorithm, which provides as results the so-called Z-average with the dimension of a length, and the polydispersity index (PDI), which is dimensionless. The terms “dynamic light scattering” or “DLS” refer to a method for analyzing particles in a liquid, wherein the liquid is typically illuminated with a monochromatic light source and wherein the light scattered by particles in the liquid is detected. DLS protocols and instruments are known in the art.

In preferred embodiments, the lipid-based carriers, preferably the LNPs, have a Z-average size of less than 400 nm, preferably less than 300 nm, more preferably less than 200 nm.

In embodiments, the lipid-based carriers, preferably the LNPs, have a Z-average size ranging from about 50 nm to about 200 nm, preferably about 50 nm to about 150 nm, more preferably about 50 nm to about 120 nm.

Preferably, the composition comprises less than about 10%, 9%, 8%, 7%, 6%, 5%, 4%, 3%, 2%, 1% lipid-based carriers that have a particle size exceeding about 500 nm. Preferably, the composition comprises less than about 10%, 9%, 8%, 7%, 6%, 5%, 4%, 3%, 2%, 1% LNPs that have a particle size smaller than about 20 nm.

In embodiments, the lipid-based carriers exhibit a zeta potential in the range of −50 mV to +50 mV, preferably −25 mV to +25 mV, more preferably −10 mV to +10 mV, most preferably −5 mV to +5 mV.

In embodiments, the polydispersity index (PDI) of the lipid-based carriers, preferably the LNPs, is in the range of 0.1 to 0.5. In preferred embodiments, the polydispersity index (PDI) value is less than about 0.3, preferably of less than about 0.2. Typically, the PDI is determined by dynamic light scattering.

In embodiments, at least 70%, 80%, 90%, 95% of the nucleic acid (e.g. RNA) is encapsulated in lipid-based carriers such as LNPs. The percentage of encapsulation may be determined by a RiboGreen assay as known in the art.

In embodiments, the plurality of lipid-based carriers have a lamellar morphology and/or a bilayer morphology. In embodiments, at least 80%, 85%, 90%, 95% of the lipid-based carriers have a spherical morphology.

In preferred embodiments, the surface of the lipid-based carriers, preferably the LNPs, is uncharged at pH 7.

### Administration of the Pharmaceutical Composition or the Nucleic Acid

Suitably, upon administration of the pharmaceutical composition or nucleic acid to a cell, tissue, or subject, the at least one tumour antigen is produced in an amount sufficient for inducing an antigen specific immune response in said cell, tissue, or subject.

In preferred embodiments, the administration is an intramuscular, intratumoral, or intravenous administration, preferably an intramuscular, intratumoral, or intravenous injection.

In preferred embodiments, upon intramuscular, intratumoral, or intravenous administration of the pharmaceutical composition or nucleic acid to a cell, tissue, or subject, the encoded tumour antigens are produced, preferably in an amount sufficient for inducing an antigen specific immune response in said cell, tissue, or subject.

In preferred embodiments, upon intramuscular, intratumoral, or intravenous administration of the pharmaceutical composition or nucleic acid to a cell, tissue, or subject, the encoded tumour antigens are produced and induce an increased immunogenicity in the subject.

In preferred embodiments, upon intramuscular, intratumoral, or intravenous administration of the pharmaceutical composition or nucleic acid to a cell, tissue, or subject, the encoded tumour antigens are produced and induce an epitope-specific or antigen-specific CD8+ T cells in the subject.

In preferred embodiments, upon intramuscular, intratumoral, or intravenous administration of the pharmaceutical composition or nucleic acid to a cell, tissue, or subject, the encoded tumour antigens are produced and induce an epitope-specific or antigen-specific CD4+ T cells in the subject.

In preferred embodiments, upon intramuscular, intratumoral, or intravenous administration of the pharmaceutical composition or nucleic acid to a cell, tissue, or subject, the encoded tumour antigens are produced and induce humoral immunity, e.g. antibody titers and/or increased variety of antibody species against the encoded tumour antigen in the subject.

In preferred embodiments, upon intramuscular, intratumoral, or intravenous administration of the pharmaceutical composition or nucleic acid to a cell, tissue, or subject, the encoded tumour antigens are produced and increase IFN-gamma production by CD8+ T cells upon exposure to the encoded tumour antigens in the subject.

In preferred embodiments, upon intramuscular, intratumoral, or intravenous administration of the pharmaceutical composition or nucleic acid to a cell, tissue, or subject, the encoded tumour antigens are produced and increase the presentation of the encoded tumour antigens on MHC molecules in the subject.

The term “presentation” of the encoded polypeptide or peptide relates to higher amount and/or variety of immunogenic and/or stable peptides presented via MHC class I and II molecules after protein degradation by the proteasomal machinery, and thus cellular immunity, e.g. T cell activation based thereon. The presentation of the encoded polypeptide or peptide on MHC class I and II molecules is increased on cells, comprising immune cells (e.g., T cells), antigen-presenting cells (e.g., dendritic cells, macrophages, engineered antigen-presenting cells), MHC class I-expressing cells, MHC class II-expressing cells, or any combination thereof.

For example, the administration of the pharmaceutical composition or nucleic acid, results in increased immunogenicity presentation on MHC molecules of the encoded tumour antigen that is increased by between about 0.1% and about 100% (e.g., about 0.5%, 1%, 2%, 3%, 4%, 5%, 6%, 7%, 8%, 9%, 10%, 15%, 20%, 25%, 30%, 35%, 40%, 45%, 50%, 60%, 70%, 80%, 90%, 95%, 98%, 99%, or about 100%) when compared to a subject without the administered nucleic acid/composition.

In some embodiments, the administration of the pharmaceutical composition or nucleic acid results in immunogenicity or presentation on MHC molecules of the encoded tumour antigen that is increased by about 2-fold to about 100-fold (e.g., about 3-fold, 4-fold, 5-fold, 6-fold, 7-fold, 8-fold, 9-fold, 10-fold, 12-fold, 14-fold, 16-fold, 18-fold, 20-fold, 25-fold, 30-fold, 40-fold, 50-fold, 60-fold, 70-fold, 80-fold, 90-fold, or about 100-fold) when compared to a subject without the administered nucleic acid/composition.

### 3: a Tumour Antigen/a Composition of Tumour Antigen:

In a third aspect, the invention provides a peptide tumour antigen or a composition comprising at least one peptide tumour antigen.

Notably, features and embodiments described in the context of the first aspect (the nucleic acid of the invention) or the second aspect (pharmaceutical composition of the invention) have to be read on and have to be understood as suitable embodiments of the tumour antigen or the composition of the present aspect.

In preferred embodiments, the tumour antigen comprises at least one antigenic peptide selected from a peptide or protein encoded by a long non-coding RNA (lncRNA) and/or at least one antigenic peptide selected from a peptide or protein of a tumour neoantigen.

In preferred embodiments in that context, the at least one antigenic peptide encoded by the long non-coding RNA (lncRNA), in particular lnc-WDR72-2:4, lnc-TRPC5-3:1, lnc-CLEC2D-9:1, KCNMB2-AS1:4, lnc-ZC3H8-6:1, lnc-NTF3-5:5, or LINC00893:25, or an immunogenic fragment or variant of any of these.

Preferred peptide tumour antigens are provided in Table 1A and 1B (column B or C).

In preferred embodiments, the at least one antigenic peptide is selected from a peptide or protein encoded by a long non-coding RNA (lncRNA) and comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 124-146, 546-571, 756, 757, or an immunogenic fragment or variant of any of these.

In preferred embodiments, the at least one antigenic peptide is selected from a peptide or protein of a tumour neoantigen, in particular selected from NRAS neoantigen, TYW1B neoantigen, ECPAS neoantigen, MAP2K1 neoantigen, TOMM22 neoantigen, GLB1 neoantigen, MAGE-A3 neoantigen, ATAD2 neoantigen, BRAF neoantigen, EGFR neoantigen, TP53 neoantigen, DIP2B neoantigen, TRRAP neoantigen, RAC1 neoantigen, AP4B1 neoantigen, VWA5A neoantigen, CLTC neoantigen, RPL9 neoantigen, RPAP1 neoantigen, RPE65 neoantigen, PATZ1 neoantigen, PIK3CA neoantigen, NFE2L2 neoantigen, or CNOT9 neoantigen or an immunogenic fragment or variant of any of these.

Preferred peptide tumour antigens are provided in Table 2 (column C), in particular column C, rows 1 to 11.

In embodiments, the at least one antigenic peptide selected from a peptide or protein of a tumour neoantigen comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 255-317, or an immunogenic fragment or immunogenic variant of any of these.

In preferred embodiments, the at least one antigenic peptide selected from a peptide or protein of a tumour neoantigen comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 255-286, or an immunogenic fragment or immunogenic variant of any of these.

Further provided herein is a composition comprising at least one peptide tumour antigen as defined herein.

A “composition comprising at least one tumour antigen” refers to any type of composition in which the specified peptide tumour antigens may be incorporated, optionally along with any further constituents, usually with at least one pharmaceutically acceptable carrier or excipient. The composition may be a dry composition such as a powder, a granule, or a solid lyophilized form. Alternatively, the composition may be in liquid form, and each constituent may be independently incorporated in dissolved or dispersed (e.g. suspended or emulsified) form.

In various embodiments, the at least one tumour antigen of the composition is formulated with a pharmaceutically acceptable carrier or excipient.

In embodiments, the composition comprises a plurality or at least more than one different peptide tumour antigen as defined herein. Preferably, the composition as defined herein may comprise 2 to 10 different peptide tumour antigens as defined herein, preferably 2 to 5 different peptide tumour antigens as defined herein.

### 4: An Antibody, a T-Cell, or a TCR:

In a fourth aspect, the invention provides an antibody, a T-cell, or a T-cell receptor (TCR) that has been raised against or stimulated with any tumour antigen or with any nucleic acid of any of the foregoing aspects.

Notably, features and embodiments described in the context of the first aspect (the nucleic acid of the invention) or the second aspect (pharmaceutical composition of the invention) or the third aspect (tumour antigen) have to be read on and have to be understood as suitable embodiments of the tumour antigens or nucleic acids of the present aspect.

In embodiments, the antibody is a therapeutic antibody that has been raised against any of the tumour antigens of the invention. Preferably, the antibody has been raised against any peptide tumour antigen provided in Table 1 A and B (columns B or C) or any peptide tumour antigens provided in Table 2 (column C).

In embodiments, the T-cell is a therapeutic T-cell that has been stimulated with any tumour antigen or with any nucleic acids of any of the foregoing aspects. Preferably, the T-cell has been stimulated with any tumour antigen or with any nucleic acid provided in Table 1A or 1B or Table 2.

In embodiments, the TCR has been raised against any of the tumour antigens of the invention (e.g. antigen peptide-loaded on MHC). Preferably, the TCR has been raised against any peptide tumour antigen provided in Table 1A or 1B (column B or C) or any peptide tumour antigens provided in Table 2 (column C).

### 5: A Combination:

In a fifth aspect, the invention provides a combination comprising at least two or a plurality of therapeutic modalities of the foregoing aspects.

Notably, features and embodiments described in the context of the first aspect (the nucleic acid of the invention) or the second aspect (pharmaceutical composition of the invention) or the third aspect (tumour antigen) or the fourth aspect (antibody, T-cell, TCR) have to be read on and have to be understood as suitable embodiments of the combination of the fifth aspect.

In preferred embodiments, the combination comprises at least two therapeutic modalities selected from
a) at least one or more artificial nucleic acids as defined in the first aspect; and/or
b) at least one or more pharmaceutical composition as defined in the second aspect; and/or
c) at least one or more tumour antigen or composition as defined in the third aspect; and/or
d) at least one or more antibody, T-cell, or TCR as defined in the fourth aspect;

In embodiments, the combination of the invention comprises at least one or more than one therapeutic modality as defined herein, preferably 2, 3, 4, 5, 6, 7, 8, or even more therapeutic modalities as defined herein.

The therapeutic modalities may be administered in a spatially separated and/or timely staggered manner.

### 6: a Kit or Kit of Parts:

In a sixth aspect, the invention provides a kit or kit of parts.

Notably, features and embodiments described in the context of the first aspect (the nucleic acid of the invention) or the second aspect (pharmaceutical composition of the invention) or the third aspect (tumour antigen) or the fourth aspect (antibody, T-cell, TCR) or the fifth aspect (combination) have to be read on and have to be understood as suitable embodiments of the kit or kit of parts of the sixth aspect.

In preferred embodiments, the kit or kit of parts comprises
i) at least one or more artificial nucleic acids as defined in the first aspect; and/or
ii) at least one or more pharmaceutical composition as defined in the second aspect; and/or
iii) at least one or more tumour antigen or composition as defined in the third aspect; and/or
iv) at least one or more antibody, T-cell, or TCR as defined in the fourth aspect;
In embodiments, the kit or kit of parts comprises a liquid vehicle for solubilising.

In embodiments, the kit or kit of parts comprises technical instructions providing information on administration and dosage of the components. The technical instructions of said kit may contain information about administration and dosage and patient groups. Such kits, preferably kits of parts, may be applied e.g. for any of the applications or uses mentioned herein, preferably for the use of the therapeutic agents i) to iv) for the treatment of cancer or a diseases, disorder, or condition related to cancer.

### 7: Medical Uses:

In a further aspect, the present invention relates to the medical use of the therapeutic agents of the foregoing aspects.

Notably, features and embodiments described in the context of the first aspect (the nucleic acid of the invention) or the second aspect (pharmaceutical composition of the invention) or the third aspect (tumour antigen) or the fourth aspect (antibody, T-cell, TCR) or the fifth aspect (combination) or the kit or kit of parts of the sixth aspect have to be read on and understood as suitable embodiments of any of the medical uses presented in the following aspects.

Accordingly, the invention provides an artificial nucleic acid of the first aspect, a pharmaceutical composition of the second aspect, a tumour antigen or a composition of tumour antigens of the third aspect, an antibody, T-cell, or TCR of the fourth aspect, a combination of the fifth aspect, or a kit or kit of parts of the sixth aspect for use as a medicament e.g. for treating or preventing a disease, disorder, or condition in a subject.

In preferred embodiments, the use may be for human medical purposes and for veterinary medical purposes, preferably for human medical purposes.

In preferred embodiments, the use may be for human medical purposes, in particular, for young infants, newborns, immunocompromised recipients, pregnant and breast-feeding women, and elderly people.

In preferred embodiments, the use may be for human medical purposes for subjects that have cancer.

### Medical Use in Treating or Preventing Cancer

In a further aspect, the invention provides an artificial nucleic acid of the first aspect, a pharmaceutical composition of the second aspect, a tumour antigen or a composition of tumour antigens of the third aspect, an antibody, T-cell, or TCR of the fourth aspect, a combination of the fifth aspect, or a kit or kit of parts of the sixth aspect for use as a medicament in treating or preventing cancer in a subject, or any disease, disorder, or condition related to cancer.

Notably, features and embodiments described in the context of any of the foregoing aspects may be read on and have to be understood as suitable embodiments of the present aspect.

As used herein, the term “cancer” refers to a neoplasm characterized by the uncontrolled and usually rapid proliferation of cells that tend to invade surrounding tissue and to metastasize to distant body sites. The term encompasses benign and malignant neoplasms. Malignancy in cancers is typically characterized by anaplasia, invasiveness, and metastasis; whereas benign malignancies typically have none of those properties. The terms includes neoplasms characterized by tumour growth as well as cancers of blood and lymphatic system. The term “cancer” according to the disclosure also comprises cancer metastases, e.g. cancer metastases associated with HNSCC, NSCLC, or melanoma.

In some embodiments, the treating or preventing cancer in a subject is a personalized treatment. Accordingly, prior to a treatment with the respective therapeutic agents, the cancer of the patient is analyzed by e.g. sequencing.

In various embodiments, the nucleic acid, the pharmaceutical composition, the tumour antigen or a composition of tumour antigens, the antibody, T-cell, or TCR, the combination, the kit or kit of parts is administered by intramuscular, intratumoral, or intravenous administration, in particular injection.

### Medical Use in Treating or Preventing Squamous Cell Carcinoma

In a further aspect, the invention provides an artificial nucleic acid of the first aspect, a pharmaceutical composition of the second aspect, a tumour antigen or a composition of tumour antigens of the third aspect, an antibody, T-cell, or TCR of the fourth aspect, a combination of the fifth aspect, or a kit or kit of parts of the sixth aspect for use as a medicament in treating or preventing squamous cell carcinoma.

Notably, features and embodiments described in the context of any of the foregoing aspects may be read on and have to be understood as suitable embodiments of the present aspect.

In a preferred embodiment, the squamous cell carcinoma is selected from squamous cell carcinomas (LUSC)/squamous NSCLC (sqNSCLC) or head and neck squamous cell carcinoma (HNSCC).

In some embodiments, the treating or preventing squamous cell carcinoma in a subject is a personalized treatment. Accordingly, prior to a treatment with the respective therapeutic agents, the squamous cell carcinoma of the patient is analyzed by e.g. sequencing.

### Medical Use in Treating or Preventing NSCLC

In a further aspect, the invention provides an artificial nucleic acid of the first aspect, a pharmaceutical composition of the second aspect, a tumour antigen or a composition of tumour antigens of the third aspect, an antibody, T-cell, or TCR of the fourth aspect, a combination of the fifth aspect, or a kit or kit of parts of the sixth aspect for use as a medicament in treating or preventing Non-small cell lung cancer (NSCLC) in a subject, or any disease, disorder, or condition related to NSCLC.

In embodiments, the use relates to treatment of NSCLC subgroups including lung adenocarcinomas (LUAD) and/or lung squamous cell carcinomas (LUSC). Preferably the use relates to treatment of squamous cell carcinomas (LUSC)/sqNSCLC.

Notably, features and embodiments described in the context of any of the foregoing aspects may be read on and have to be understood as suitable embodiments of the present aspect.

In preferred embodiments in that context, the at least one tumour antigen that is provided by the artificial nucleic acid, the pharmaceutical composition, the tumour antigen or a composition of tumour antigens, or the kit or kit of parts comprises
a) at least one antigenic peptide selected from a peptide or protein encoded by lnc-WDR72-2:4, lnc-TRPC5-3:1, KCNMB2-AS1:4, lnc-NTF3-5:5, or lnc-ZC3H8-6:1, or an immunogenic fragment or variant of any of these; and/or
b) at least one antigenic peptide selected from a peptide or protein of a tumour neoantigen selected from a BRAF neoantigen, EGFR neoantigen, TP53 neoantigen, PIK3CA neoantigen, or NFE2L2 neoantigen, or an immunogenic fragment or variant of any of these, preferably selected from an EGFR neoantigen or a TP53 neoantigen, or an immunogenic fragment or variant of any of these.

In particularly preferred embodiments in that context, the at least one tumour antigen that is provided by the artificial nucleic acid, the pharmaceutical composition, the tumour antigen or a composition of tumour antigens, or the kit or kit of parts comprises at least one antigenic peptide selected from a peptide or protein encoded by lnc-WDR72-2:4, lnc-TRPC5-3:1, KCNMB2-AS1:4, lnc-NTF3-5:5, or lnc-ZC3H8-6:1, or an immunogenic fragment or variant of any of these.

In various embodiments in that context, the nucleic acid, the pharmaceutical composition, the tumour antigen or a composition of tumour antigens, the antibody, T-cell, or TCR, the combination, the kit or kit of parts is administered by intramuscular, intratumoral, or intravenous administration, in particular intramuscular injection.

In some embodiments, the treating or preventing NSCLC in a subject is a personalized treatment. Accordingly, prior to a treatment with the respective therapeutic agents, the NSCLC cancer of the patient is analyzed by e.g. sequencing.

### Medical Use in Treating or Preventing HNSCC

In a further aspect, the invention provides an artificial nucleic acid of the first aspect, a pharmaceutical composition of the second aspect, a tumour antigen or a composition of tumour antigens of the third aspect, an antibody, T-cell, or TCR of the fourth aspect, a combination of the fifth aspect, or a kit or kit of parts of the sixth aspect for use as a medicament in treating or preventing head and neck squamous cell carcinoma (HNSCC) in a subject, or any disease, disorder, or condition related to HNSCC.

Notably, features and embodiments described in the context of any of the foregoing aspects may be read on and have to be understood as suitable embodiments of the present aspect.

In particularly preferred embodiments in that context, the at least one tumour antigen that is provided by the artificial nucleic acid, the pharmaceutical composition, the tumour antigen or a composition of tumour antigens, or the kit or kit of parts comprises at least one antigenic peptide selected from a peptide or protein encoded by lnc-WDR72-2:4, KCNMB2-AS1:4, lnc-NTF3-5:5, or lnc-ZC3H8-6:1, or an immunogenic fragment or variant of any of these

In various embodiments in that context, the nucleic acid, the pharmaceutical composition, the tumour antigen or a composition of tumour antigens, the antibody, T-cell, or TCR, the combination, the kit or kit of parts is administered by intramuscular, intratumoral, or intravenous administration, in particular intramuscular injection.

In some embodiments, the treating or preventing HNSCC in a subject is a personalized treatment. Accordingly, prior to a treatment with the respective therapeutic agents, the HNSCC cancer of the patient is analyzed by e.g. sequencing.

### Medical Use in Treating or Preventing Melanoma

In a further aspect, the invention provides an artificial nucleic acid of the first aspect, a pharmaceutical composition of the second aspect, a tumour antigen or a composition of tumour antigens of the third aspect, an antibody, T-cell, or TCR of the fourth aspect, a combination of the fifth aspect, or a kit or kit of parts of the sixth aspect for use as a medicament in treating or preventing melanoma in a subject, or any disease, disorder, or condition related to melanoma.

In embodiments, the use relates to treatment of skin cutaneous melanoma (SKCM) or uveal melanoma.

In embodiments, the use relates to treatment of melanoma BRAF subgroup and/or NRAS subgroup.

Notably, features and embodiments described in the context of any of the foregoing aspects may be read on and have to be understood as suitable embodiments of the present aspect.

In preferred embodiments in that context, the at least one tumour antigen that is provided by the artificial nucleic acid, the pharmaceutical composition, the tumour antigen or a composition of tumour antigens, or the kit or kit of parts comprises
a) at least one antigenic peptide selected from a peptide or protein encoded by lnc-TRPC5-3:1 or lnc-CLEC2D-9:1, or an immunogenic fragment or variant of any of these; and/or
b) at least one antigenic peptide selected from a peptide or protein of a tumour neoantigen selected from an NRAS neoantigen, TYW1B neoantigen, ECPAS neoantigen, MAP2K1 neoantigen, TOMM22 neoantigen, GLB1 neoantigen, MAGE-A3 neoantigen, ATAD2 neoantigen, BRAF neoantigen, DIP2B neoantigen, TRRAP neoantigen, RAC1 neoantigen, AP4B1 neoantigen, VWA5A neoantigen, CLTC neoantigen, RPL9 neoantigen, RPAP1 neoantigen, RPE65 neoantigen, PATZ1 neoantigen, CNOT9 neoantigen, preferably selected from an NRAS neoantigen, TYW1B neoantigen, ECPAS neoantigen, MAP2K1 neoantigen, TOMM22 neoantigen, GLB1 neoantigen, MAGE-A3 neoantigen, ATAD2 neoantigen, or BRAF neoantigen, or an immunogenic fragment or variant of any of these.

In particularly preferred embodiments in that context, the at least one tumour antigen that is provided by the artificial nucleic acid, the pharmaceutical composition, the tumour antigen or a composition of tumour antigens, or the kit or kit of parts comprises at least one antigenic peptide selected from a peptide or protein encoded by lnc-TRPC5-3:1 or lnc-CLEC2D-9:1, or an immunogenic fragment or variant of any of these.

In various embodiments in that context, the nucleic acid, the pharmaceutical composition, the tumour antigen or a composition of tumour antigens, the antibody, T-cell, or TCR, the combination, the kit or kit of parts is administered by intramuscular, intratumoral, or intravenous administration, in particular intramuscular injection.

In some embodiments, the treating or preventing melanoma in a subject is a personalized treatment. Accordingly, prior to a treatment with the respective therapeutic agents, the melanoma of the patient is analyzed by e.g., sequencing.

### Medical Use in Treating or Preventing Further Cancer Indications

In further aspects, the present invention relates to the medical use of the therapeutic agents of the foregoing aspects in treating or preventing further cancer indications.

Notably, features and embodiments described in the context of the first aspect (the nucleic acid of the invention) or the second aspect (pharmaceutical composition of the invention) or the third aspect (tumour antigen) or the fourth aspect (antibody, T-cell, TCR) or the fifth aspect (combination) or the kit or kit of parts of the sixth aspect have to be read on and understood as suitable embodiments of any of the medical uses presented in the following aspects.

In a further aspect, the invention provides an artificial nucleic acid of the first aspect, a pharmaceutical composition of the second aspect, a tumour antigen or a composition of tumour antigens of the third aspect, an antibody, T-cell, or TCR of the fourth aspect, a combination of the fifth aspect, or a kit or kit of parts of the sixth aspect for use as a medicament in treating or preventing Adrenocortical carcinoma, Bladder Urothelial Carcinoma, Breast invasive carcinoma, Cervical carcinoma (Cervical squamous cell carcinoma and endocervical adenocarcinoma), Colon adenocarcinoma, Kidney renal papillary cell carcinoma, Lung adenocarcinoma, Lymphoid Neoplasm Diffuse Large B-cell Lymphoma, Rectum adenocarcinoma, Sarcoma (in particular synoval Sarcoma), Skin Cutaneous Melanoma, Thymoma, Thyroid carcinoma, Hepatocellular carcinoma, Uterine Carcinosarcoma, or Uterine Corpus Endometrial Carcinoma in a subject, or any related disease, disorder, or condition, wherein the at least one tumour antigen that is provided by the artificial nucleic acid, the pharmaceutical composition, the tumour antigen or a composition of tumour antigens, the combination, or the kit or kit of parts comprises at least one antigenic peptide selected from a peptide or protein encoded by ZC3H8-6:1, or an immunogenic fragment or variant thereof.

In a further aspect, the invention provides an artificial nucleic acid of the first aspect, a pharmaceutical composition of the second aspect, a tumour antigen or a composition of tumour antigens of the third aspect, an antibody, T-cell, or TCR of the fourth aspect, a combination of the fifth aspect, or a kit or kit of parts of the sixth aspect for use as a medicament in treating or preventing Bladder Urothelial Carcinoma, Cervical carcinoma (Cervical squamous cell carcinoma and endocervical adenocarcinoma), Thymoma, or Uterine Corpus Endometrial Carcinoma in a subject, or any related disease, disorder, or condition related, wherein the at least one tumour antigen that is provided by the artificial nucleic acid, the pharmaceutical composition, the tumour antigen or a composition of tumour antigens, the combination, or the kit or kit of parts comprises at least one antigenic peptide selected from a peptide or protein encoded by KCNMB2-AS1:4, or an immunogenic fragment or variant thereof.

In a further aspect, the invention provides an artificial nucleic acid of the first aspect, a pharmaceutical composition of the second aspect, a tumour antigen or a composition of tumour antigens of the third aspect, an antibody, T-cell, or TCR of the fourth aspect, a combination of the fifth aspect, or a kit or kit of parts of the sixth aspect for use as a medicament in treating or preventing Ovarian serous cystadenocarcinoma, Testicular Germ Cell Tumors, Thymoma, or Uterine carcinosarcoma in a subject, or any related disease, disorder, or condition related, wherein the at least one tumour antigen that is provided by the artificial nucleic acid, the pharmaceutical composition, the tumour antigen or a composition of tumour antigens, the combination, or the kit or kit of parts comprises at least one antigenic peptide selected from a peptide or protein encoded by lnc-NTF3-5:5, or an immunogenic fragment or variant thereof.

In a further aspect, the invention provides an artificial nucleic acid of the first aspect, a pharmaceutical composition of the second aspect, a tumour antigen or a composition of tumour antigens of the third aspect, an antibody, T-cell, or TCR of the fourth aspect, a combination of the fifth aspect, or a kit or kit of parts of the sixth aspect for use as a medicament in treating or preventing Bladder Urothelial Carcinoma, Colon adenocarcinoma, Esophageal carcinoma, or Stomach adenocarcinoma in a subject, or any related disease, disorder, or condition, wherein the at least one tumour antigen that is provided by the artificial nucleic acid, the pharmaceutical composition, the tumour antigen or a composition of tumour antigens, the combination, or the kit or kit of parts comprises at least one antigenic peptide selected from a peptide or protein encoded by lnc-WDR72-2:4, or an immunogenic fragment or variant thereof.

In a further aspect, the invention provides an artificial nucleic acid of the first aspect, a pharmaceutical composition of the second aspect, a tumour antigen or a composition of tumour antigens of the third aspect, an antibody, T-cell, or TCR of the fourth aspect, a combination of the fifth aspect, or a kit or kit of parts of the sixth aspect for use as a medicament in treating or preventing Skin Cutaneous Melanoma, Testicular Germ Cell Tumors, or Uveal Melanoma in a subject, or any related disease, disorder, or condition, wherein the at least one tumour antigen that is provided by the artificial nucleic acid, the pharmaceutical composition, the tumour antigen or a composition of tumour antigens, the combination, or the kit or kit of parts comprises at least one antigenic peptide selected from a peptide or protein encoded by lnc-CLEC2D-9:1, or an immunogenic fragment or variant thereof.

### 8: Methods of Treatment:

In a further aspect, the present invention relates to a method of treating or preventing a disease, disorder or condition.

Notably, embodiments relating to the previous aspects may likewise be read on and be understood as suitable embodiments of method of treatments of the invention. In particular, specific features and embodiments relating to method of treatments as provided herein may also apply for medical uses of the invention and vice versa.

Preventing (inhibiting) or treating a disease relates to inhibiting the full development of a disease or condition, for example, in a subject who is at risk for a disease such as an infection or cancer. “Treatment” refers to a therapeutic intervention that ameliorates a sign or symptom of a disease or pathological condition after it has begun to develop. The term “ameliorating”, with reference to a disease or pathological condition, refers to any observable beneficial effect of the treatment. Inhibiting a disease can include preventing or reducing the risk of the disease. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters that are specific to the particular disease. A “prophylactic” treatment is a treatment administered to a subject who does not exhibit signs of a disease or exhibits only early signs for the purpose of decreasing the risk of developing pathology.

In preferred embodiments, the present invention relates to a method of treating or preventing a disease, disorder or condition, wherein the method comprises applying or administering to a subject in need thereof an effective amount of an artificial nucleic acid of the first aspect, a pharmaceutical composition of the second aspect, a tumour antigen or a composition of tumour antigens of the third aspect, an antibody, T-cell, or TCR of the fourth aspect, a combination of the fifth aspect, or a kit or kit of parts of the sixth aspect.

As used herein, “effective” when referring to an amount of a therapeutic compound refers to the quantity of the compound that is sufficient to yield a desired therapeutic response without undue adverse side effects (such as toxicity, irritation, or allergic response) commensurate with a reasonable benefit/risk ratio when used in the manner of this disclosure.

In preferred embodiments, the disease, disorder or condition is cancer, e.g. any disease, disorder or condition associated with cancer as defined herein. In preferred embodiments, the cancer is NSCLC. In preferred embodiments, the cancer is squamous cell carcinoma, preferably selected from squamous cell carcinomas (LUSC)/squamous non-small-cell lung cancer (sqNSCLC) or head and neck squamous cell carcinoma (HNSCC). In other preferred embodiments, the cancer is melanoma.

Preferred embodiments in that context are inter alia provided in the section “Medical use in treating or preventing squamous cell carcinoma” or “Medical use in treating or preventing NSCLC” or “Medical use in treating or preventing HNSCC” or “Medical use in treating or preventing melanoma” or “Medical use in treating or preventing further cancer indications” outlined in the context of the aspect “medical uses”.

In various embodiments, the nucleic acid, the pharmaceutical composition, the tumour antigen or a composition of tumour antigens, the antibody, T-cell, or TCR, the combination, the kit or kit of parts is administered by intramuscular, intratumoral, or intravenous administration, in particular injection.

In some embodiments, the treating or preventing cancer in a subject is a personalized treatment. Accordingly, prior to a treatment with the respective therapeutic agents, the cancer of the patient is analyzed by e.g. sequencing.

### ITEM LIST

Preferred embodiments of the present invention are provided in the following numbered list of items (Items 1-116):
1. An artificial nucleic acid comprising at least one coding sequence encoding at least one tumour antigen that comprises or consists of
   a) at least one antigenic peptide selected from a peptide or protein encoded by a long non-coding RNA (lncRNA), or an immunogenic fragment or variant thereof, and/or
   b) at least one antigenic peptide selected from a peptide or protein of a tumour neoantigen, or an immunogenic fragment or variant thereof.
2. The artificial nucleic acid of item 1, wherein the at least one antigenic peptide is selected from a peptide or protein encoded by a long non-coding RNA (lncRNA) selected from lnc-WDR72-2:4, lnc-TRPC5-3:1, lnc-CLEC2D-9:1, KCNMB2-AS1:4, lnc-ZC3H8-6:1, lnc-NTF3-5:5, or LINC00893:25, or an immunogenic fragment or variant of any of these.
3. The artificial nucleic acid of items 1 to 2, wherein the at least one antigenic peptide encoded by the long non-coding RNA (lncRNA) comprises at least one T-cell epitope, preferably at least one CD8+ T cells epitope.
4. The artificial nucleic acid of items 1 to 3, wherein the at least one antigenic peptide selected from a peptide or protein encoded by a long non-coding RNA (lncRNA) comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 124-254, 546-650, 756-763, or an immunogenic fragment or variant of any of these.
5. The artificial nucleic acid of items 1 to 4, comprising at least one coding sequence encoding at least one tumour antigen that comprises at least one antigenic peptide selected from a peptide or protein encoded by lnc-WDR72-2:4, wherein the antigenic peptide comprises or consists of at least one of the amino acid sequences being identical or at least 80% identical to any one of SEQ ID NOs: 124-146, 546-571, 756, 757, or an immunogenic fragment or variant of any of these.
6. The artificial nucleic acid of items 1 to 4, comprising at least one coding sequence encoding at least one tumour antigen that comprises at least one antigenic peptide selected from a peptide or protein encoded by lnc-TRPC5-3:1, wherein the antigenic peptide comprises or consists of at least one of the amino acid sequences being identical or at least 80% identical to any one of SEQ ID NOs: 227-237, 630-642, 761, or an immunogenic fragment or variant of any of these.
7. The artificial nucleic acid of items 1 to 4, comprising at least one coding sequence encoding at least one tumour antigen that comprises at least one antigenic peptide selected from a peptide or protein encoded by lnc-CLEC2D-9:1, wherein the antigenic peptide comprises or consists of at least one of the amino acid sequences being identical or at least 80% identical to any one of SEQ ID NOs: 238-243, 643-650, 762, or an immunogenic fragment or variant of any of these.
8. The artificial nucleic acid of items 1 to 4, comprising at least one coding sequence encoding at least one tumour antigen that comprises at least one antigenic peptide selected from a peptide or protein encoded by KCNMB2-AS1:4, wherein the antigenic peptide comprises or consists of at least one of the amino acid sequences being identical or at least 80% identical to any one of SEQ ID NOs: 147-161, 572-589, 758, or an immunogenic fragment or variant of any of these.
9. The artificial nucleic acid of items 1 to 4, comprising at least one coding sequence encoding at least one tumour antigen that comprises at least one antigenic peptide selected from a peptide or protein encoded by lnc-ZC3H8-6:1, wherein the antigenic peptide comprises or consists of at least one of the amino acid sequences being identical or at least 80% identical to any one of SEQ ID NOs: 162-168, 590-603, 759, or an immunogenic fragment or variant of any of these.
10. The artificial nucleic acid of items 1 to 4, comprising at least one coding sequence encoding at least one tumour antigen that comprises at least one antigenic peptide selected from a peptide or protein encoded by lnc-NTF3-5:5, wherein the antigenic peptide comprises or consists of at least one of the amino acid sequences being identical or at least 80% identical to any one of SEQ ID NOs: 169-226, 604-629, 760, or an immunogenic fragment or variant of any of these.
11. The artificial nucleic acid of items 1 to 4, comprising at least one coding sequence encoding at least one tumour antigen that comprises at least one antigenic peptide selected from a peptide or protein encoded by LINC00893:25, wherein the antigenic peptide comprises or consists of at least one of the amino acid sequences being identical or at least 80% identical to any one of SEQ ID NOs: 244-254, 763, or an immunogenic fragment or variant of any of these.
12. The artificial nucleic acid of items 1 to 11, wherein the at least one coding sequence comprises a nucleic acid sequence encoding at least one antigenic peptide selected from a peptide or protein encoded by a lncRNA, wherein the nucleic acid sequence is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 318-448, 651-755, 764-771, or a fragment or a variant of any of these.
13. The artificial nucleic acid of item 12, wherein the at least one coding sequence comprises a GC optimized nucleic acid sequence encoding at least one antigenic peptide selected from a peptide or protein encoded by a lncRNA, wherein the nucleic acid sequence is identical or at least 80% identical to any one of SEQ ID NOs: 318-448, 651-755, 764-771, or a fragment of any of these.
14. The artificial nucleic acid of item 1, wherein the tumour neoantigen is selected from an NRAS neoantigen, TYW1B neoantigen, ECPAS neoantigen, MAP2K1 neoantigen, TOMM22 neoantigen, GLB1 neoantigen, MAGE-A3 neoantigen, ATAD2 neoantigen, BRAF neoantigen, EGFR neoantigen, TP53 neoantigen, DIP2B neoantigen, TRRAP neoantigen, RAC1 neoantigen, AP4B1 neoantigen, VWA5A neoantigen, CLTC neoantigen, RPL9 neoantigen, RPAP1 neoantigen, RPE65 neoantigen, PATZ1 neoantigen, PIK3CA neoantigen, NFE2L2 neoantigen, or CNOT9 neoantigen, or an immunogenic fragment or variant of these.
15. The artificial nucleic acid of item 14, wherein the at least one antigenic peptide selected from a peptide or protein of a tumour neoantigen comprises at least one T-cell epitope, preferably at least one CD8+ T cells epitope.
16. The artificial nucleic acid of item 14 or 15, wherein the at least one antigenic peptide selected from a peptide or protein of a tumour neoantigen comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 255-317, or an immunogenic fragment or immunogenic variant of any of these.
17. The artificial nucleic acid of items 14 to 16, comprising at least one coding sequence encoding at least one antigenic peptide selected from a peptide or protein of a NRAS tumour neoantigen, wherein the antigenic peptide comprises or consists of at least one of the amino acid sequences being identical or at least 80% identical to any one of SEQ ID NOs: 267-272, or an immunogenic fragment or variant of any of these.
18. The artificial nucleic acid of items 14 to 16, comprising at least one coding sequence encoding at least one antigenic peptide selected from a peptide or protein of a TYW1B tumour neoantigen, wherein the antigenic peptide comprises or consists of at least one of the amino acid sequences being identical or at least 80% identical to any one of SEQ ID NOs: 273, 274, or an immunogenic fragment or variant of these.
19. The artificial nucleic acid of items 14 to 16, comprising at least one coding sequence encoding at least one antigenic peptide selected from a peptide or protein of a ECPAS tumour neoantigen, wherein the antigenic peptide comprises or consists of at least one of the amino acid sequences being identical or at least 80% identical to any one of SEQ ID NO: 275, or an immunogenic fragment or variant thereof.
20. The artificial nucleic acid of items 14 to 16, comprising at least one coding sequence encoding at least one antigenic peptide selected from a peptide or protein of a MAP2K1 tumour neoantigen, wherein the antigenic peptide comprises or consists of at least one of the amino acid sequences being identical or at least 80% identical to any one of SEQ ID NOs: 276, 277, or an immunogenic fragment or variant of these.
21. The artificial nucleic acid of items 14 to 16, comprising at least one coding sequence encoding at least one antigenic peptide selected from a peptide or protein of a TOMM22 tumour neoantigen, wherein the antigenic peptide comprises or consists of at least one of the amino acid sequences being identical or at least 80% identical to any one of SEQ ID NOs: 278, 279, or an immunogenic fragment or variant of these.
22. The artificial nucleic acid of items 14 to 16, comprising at least one coding sequence encoding at least one antigenic peptide selected from a peptide or protein of a GLB1 tumour neoantigen, wherein the antigenic peptide comprises or consists of at least one of the amino acid sequences being identical or at least 80% identical to any one of SEQ ID NOs: 280, 281, or an immunogenic fragment or variant of these.
23. The artificial nucleic acid of items 14 to 16, comprising at least one coding sequence encoding at least one antigenic peptide selected from a peptide or protein of a MAGE-A3 tumour neoantigen, wherein the antigenic peptide comprises or consists of at least one of the amino acid sequences being identical or at least 80% identical to any one of SEQ ID NOs: 282, 283, or an immunogenic fragment or variant of these.
24. The artificial nucleic acid of items 14 to 16, comprising at least one coding sequence encoding at least one antigenic peptide selected from a peptide or protein of a ATAD2 tumour neoantigen, wherein the antigenic peptide comprises or consists of at least one of the amino acid sequences being identical or at least 80% identical to any one of SEQ ID NOs: 284-286, or an immunogenic fragment or variant of any of these.
25. The artificial nucleic acid of items 14 to 16, comprising at least one coding sequence encoding at least one antigenic peptide selected from a peptide or protein of a BRAF tumour neoantigen, wherein the antigenic peptide comprises or consists of at least one of the amino acid sequences being identical or at least 80% identical to any one of SEQ ID NOs: 262-266, or an immunogenic fragment or variant of any of these.
26. The artificial nucleic acid of items 14 to 16, comprising at least one coding sequence encoding at least one antigenic peptide selected from a peptide or protein of a EGFR tumour neoantigen, wherein the antigenic peptide comprises or consists of at least one of the amino acid sequences being identical or at least 80% identical to any one of SEQ ID NOs: 255-257, or an immunogenic fragment or variant of any of these.
27. The artificial nucleic acid of items 14 to 16, comprising at least one coding sequence encoding at least one antigenic peptide selected from a peptide or protein of a TP53 tumour neoantigen, wherein the antigenic peptide comprises or consists of at least one of the amino acid sequences being identical or at least 80% identical to any one of SEQ ID NOs: 258-261, or an immunogenic fragment or variant of any of these.
28. The artificial nucleic acid of items 14 to 27, wherein the at least one coding sequence comprises a nucleic acid sequence encoding at least one antigenic peptide selected from a peptide or protein of a tumour neoantigen, wherein the nucleic acid sequence is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 449-511, or a fragment or a variant of any of these.
29. The artificial nucleic acid of item 28, wherein the at least one coding sequence comprises a GC optimized nucleic acid sequence encoding at least one antigenic peptide selected from a peptide or protein of a tumour neoantigen, wherein the nucleic acid sequence is identical or at least 80% identical to any one of SEQ ID NOs: 449-511, or a fragment of any of these.
30. The artificial nucleic acid of any one of the preceding items, wherein the at least one coding sequence encodes 2 to 10 different tumour antigens, preferably 2 to 5 different tumour antigens.
31. The artificial nucleic acid of item 30, wherein the different tumour antigens are separated by a linker element.
32. The artificial nucleic acid of any one of the preceding items, wherein the at least one coding sequence encodes at least one additional amino acid sequence selected from at least one immune response activating signal transduction protein.
33. The artificial nucleic acid of any one of the preceding items, wherein the at least one coding sequence encodes at least one additional amino acid sequence selected from at least one T-helper epitope.
34. The artificial nucleic acid of any one of the preceding items, wherein the at least one coding sequence encodes at least one additional amino acid sequence selected from at least one signal peptide.
35. The artificial nucleic acid of any one of the preceding items, wherein the at least one coding sequence is a codon modified coding sequence, preferably wherein codon modified coding sequence is selected from a C maximized coding sequence, a CAI maximized coding sequence, human codon usage adapted coding sequence, a G/C content modified coding sequence, and a G/C optimized coding sequence, or any combination thereof,
36. The artificial nucleic acid of item 35, wherein the at least one codon modified coding sequence is a G/C optimized coding sequence.
37. The artificial nucleic acid of any one of the preceding items, wherein the nucleic acid comprises at least one untranslated region (UTR) preferably selected from at least one heterologous 5′-UTR and/or at least one heterologous 3′-UTR.
38. The artificial nucleic acid of item 37, wherein the at least one heterologous 3′-UTR comprises or consists of a nucleic acid sequence selected or from a 3′-UTR of a gene selected from PSMB3, ALB7, alpha-globin, beta-globin, ANXA4, CASP1, COX6B1, FIG. 4, GNAS, NDUFA1, RPS9, SLC7A3 or TUBB4B, or from a homolog, a fragment or a variant of any one of these genes, preferably wherein the at least one heterologous 3′-UTR comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 66-95, 112-123, or a fragment or a variant of any of these.
39. The artificial nucleic acid of item 37 or 38, wherein the at least one heterologous 3′-UTR comprises or consists of a nucleic acid sequence selected or from a 3′-UTR of a PSMB3 gene, wherein the at least one heterologous 3′-UTR comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 66, 67, 112-123, preferably SEQ ID NO: 67, or a fragment or a variant of any of these.
40. The artificial nucleic acid of item 37, wherein the at least one heterologous 5′-UTR comprises or consists of a nucleic acid sequence selected or from a 5′-UTR of a gene selected from HSD17B4, RPL32, AIG1, alpha-globin, ASAH1, ATP5A1, COX6C, DPYSL2, MDR, MP68, NDUFA4, NOSIP, RPL31, RPL35A, SLC7A3, TUBB4B, or UBQLN2, or from a homolog, a fragment or variant of any one of these genes, preferably wherein the at least one heterologous 5′-UTR comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 12-45, 64, 65, or a fragment or a variant of any of these.
41. The artificial nucleic acid of item 37 or 40, wherein the at least one heterologous 5′-UTR comprises or consists of a nucleic acid sequence selected or from a 5′-UTR of a HSD17B4 gene, wherein the at least one heterologous 5′-UTR comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 12, 13, 64, 65, preferably SEQ ID NO: 13, or a fragment or a variant of any of these.
42. The artificial nucleic acid of items 37 to 41, wherein the at least one heterologous 5′-UTR is selected from HSD17B4 and the at least one heterologous 3′ UTR is selected from PSMB3.
43. The artificial nucleic acid of any one of the preceding items, wherein the artificial nucleic acid is selected from a DNA or an RNA, preferably an RNA.
44. The artificial nucleic acid of any one of the preceding items, wherein the artificial nucleic acid is an RNA selected from mRNA, circular RNA, replicon RNA, or viral RNA.
45. The artificial nucleic acid of any one of the preceding items, wherein the artificial nucleic acid is an mRNA.
46. The artificial nucleic acid of any one of the preceding items, wherein the artificial nucleic acid, preferably the RNA, comprises at least one poly(A) sequence, preferably wherein the at least one poly(A) sequence comprises about 40 to about 500 adenosine nucleotides.
47. The artificial nucleic acid of item 46, wherein the at least one poly(A) sequence comprises about 60 to about 150 adenosine nucleotides, preferably about 100 adenosine nucleotides.
48. The artificial nucleic acid of item 46 or 47, wherein the at least one poly(A) sequence is located at the 3′ terminus, optionally, wherein the 3′ terminal nucleotide is an adenosine.
49. The artificial nucleic acid of any one of the preceding items, wherein the artificial nucleic acid, preferably the RNA, comprises at least one poly(C) sequence and/or at least one miRNA binding site and/or at least one histone stem-loop sequence.
50. The artificial nucleic acid of any one of the preceding items, wherein the artificial nucleic acid, preferably the RNA, comprises at least one histone stem-loop sequence, wherein said histone stem-loop sequence comprises or consists of a nucleic acid sequence identical or at least 70%, 80%, 90%, 91%, 92%, 93%, 94%, 95%, 96%, 97%, 98%, or 99% identical to any one of SEQ ID NOs: 3 or 4, or a fragment or variant of these.
51. The artificial nucleic acid of any one of the preceding items, wherein the artificial nucleic acid is an RNA that comprises at least one modified nucleotide, preferably a modified nucleotide selected from pseudouridine (ψ) or N1-methylpseudouridine (m1ψ).
52. The artificial nucleic acid of any one of the preceding items, wherein the artificial nucleic acid is a modified RNA wherein each uracil is substituted by a modified nucleotide.
53. The artificial nucleic acid of item 51 or 52, wherein the modified nucleotide is N1-methylpseudouridine (m1ψ).
54. The artificial nucleic acid of items 1 to 50, wherein the nucleic acid is an RNA that does not comprise a modified nucleotide.
55. The artificial nucleic acid of any one of the preceding items, wherein the artificial nucleic acid is an RNA that comprises a 5′-cap structure.
56. The artificial nucleic acid of item 55, wherein the 5′-cap structure is selected from a cap1 structure or a modified cap1 structure.
57. The artificial nucleic acid of any one of the preceding items, wherein the artificial nucleic acid is an vitro transcribed RNA.
58. The artificial nucleic acid of any one of the preceding items, wherein the artificial nucleic acid is a purified RNA, preferably wherein the RNA has been purified by at least one step of RP-HPLC, AEX, SEC, hydroxyapatite chromatography, TFF, filtration, precipitation, core-bead flow through chromatography, oligo (dT) purification, cellulose-based purification, or any combination thereof.
59. The artificial nucleic acid of any one of the preceding items, wherein the artificial nucleic acid, preferably the RNA, has an integrity of at least 50%, preferably of at least 60%, more preferably of at least 70%, most preferably of at least 80%.
60. The artificial nucleic acid of any one of the preceding items, wherein the artificial nucleic acid, preferably the RNA, is suitable for use in treatment or prevention of a disease, disorder or condition, preferably a tumour disease, disorder or condition.
61. The artificial nucleic acid of any one of the preceding items, wherein the artificial nucleic acid, preferably the RNA, comprises the following sequence elements preferably in 5′- to 3′-direction:
   A) a 5′-cap structure;
   B) a 5′-UTR, preferably selected from a 5′-UTR of a HSD17B4 gene;
   C) a coding sequence encoding at least one tumour antigen;
   D) a 3′-UTR, preferably selected from a 3′-UTR of a PSMB3 gene;
   E) optionally, a histone stem-loop; and
   F) a poly(A) sequence, preferably comprising about 100 A nucleotides.
62. The artificial nucleic acid of items 1 to 61, wherein the artificial nucleic acid is an mRNA that comprises the following sequence elements in 5′- to 3′-direction:
   A) a 5′-cap structure;
   B) a 5′-UTR that comprises or consists of a nucleic acid sequence being identical or at least 80% identical to SEQ ID NO: 13, or a fragment or a variant thereof;
   C) a coding sequence that comprises a nucleic acid sequence encoding at least one antigenic peptide selected from a peptide or protein encoded by a long non-coding RNA (lncRNA), wherein the nucleic acid sequence is identical or at least 80% identical to any one of SEQ ID NOs: 318-448, 651-755, 764-771, or a fragment or a variant of any of these;
   D) a 3′-UTR that comprises or consists of a nucleic acid sequence being identical or at least 80% identical to SEQ ID NO: 67, or a fragment or a variant thereof;
   E) optionally, a histone stem-loop; and
   F) a poly(A) sequence, preferably comprising about 100 A nucleotides.
63. The artificial nucleic acid of items 1 to 61, wherein the artificial nucleic acid is an mRNA that comprises the following sequence elements in 5′- to 3′-direction:
   A) a 5′-cap structure;
   B) a 5′-UTR that comprises or consists of a nucleic acid sequence being identical or at least 80% identical to SEQ ID NO: 13, or a fragment or a variant thereof;
   C) a coding sequence that comprises a nucleic acid sequence encoding at least one antigenic peptide selected from a peptide or protein of a tumour neoantigen, wherein the nucleic acid sequence is identical or at least 80% identical to any one of SEQ ID NOs: 449-511, or a fragment or a variant of any of these;
   D) a 3′-UTR that comprises or consists of a nucleic acid sequence being identical or at least 80% identical to SEQ ID NO: 67, or a fragment or a variant thereof;
   E) optionally, a histone stem-loop; and
   F) a poly(A) sequence, preferably comprising about 100 A nucleotides.
64. A pharmaceutical composition comprising at least one artificial nucleic acid comprising at least one coding sequence encoding at least one tumour antigen as defined by any one of the items 1 to 63.
65. The pharmaceutical composition of item 64, comprising a plurality of artificial nucleic acids each encoding at least one different tumour antigen.
66. The pharmaceutical composition of item 64 or 65, wherein the at least one artificial nucleic acid, preferably the RNA, is formulated in at least one cationic or polycationic compound.
67. The pharmaceutical composition of item 66, wherein the at least one cationic or polycationic compound is selected from a cationic or polycationic polymer, a cationic or polycationic polysaccharide, a cationic or polycationic lipid, a cationic or polycationic protein, a cationic or polycationic peptide, or any combinations.
68. The pharmaceutical composition of items 64 to 67, wherein the at least one artificial nucleic acid, preferably the RNA, is formulated in lipid-based carriers.
69. The pharmaceutical composition of item 68, wherein the lipid-based carriers are selected from liposomes, lipid nanoparticles, lipoplexes, solid lipid nanoparticles, lipo-polyplexes, and/or nanoliposomes.
70. The pharmaceutical composition of item 68 or 69, wherein the lipid-based carriers are lipid nanoparticles.
71. The pharmaceutical composition of items 68 to 70, wherein the lipid-based carriers comprise at least one aggregation-reducing lipid, at least one cationic lipid or ionizable lipid, at least one neutral lipid or phospholipid, and at least one steroid or steroid analogue.
72. The pharmaceutical composition of item 71, wherein the aggregation reducing lipid is a polymer conjugated lipid selected from a PEG-conjugated lipid or a PEG-free lipid.
73. The pharmaceutical composition of item 72, wherein the polymer conjugated lipid is selected from DMG-PEG 2000, C10-PEG2K, Cer8-PEG2K, or a POZ-lipid.
74A. The pharmaceutical composition of items 71 to 73, wherein the cationic lipid or ionizable lipid is selected from an amino lipid, preferably wherein the amino lipid comprises a tertiary amine group.
74B. The pharmaceutical composition of items 71 to 74A, wherein cationic or ionizable lipid is selected or derived from formula (III-1, as defined herein), preferably, wherein one of L1 or L2 is —O(C═O)—, —(C═O)O—, —C(═O)—, —O—, —S(O)x-, —S—S—, —C(═O)S—, SC(═O)—, —NRaC(═O)—, —C(═O)NRa-, —NRaC(═O)NRa-, —OC(═O)NRa- or —NRaC(═O)O—, and the other of L1 or L2 is —O(C═O)—, —(C═O)O—, —C(═O)—, —O—, —S(O)x-, —S S—, —C(═O)S—, SC(═O)—, —NRaC(═O)—, —C(═O)NRa-, —NRaC(═O)NRa-, —OC(═O)NRa- or —NRaC(═O)O— or a direct bond; G1 and G2 are each independently unsubstituted C1-C12 alkylene or C1-C12 alkenylene; G3 is C1-C24 alkylene, C1-C24 alkenylene, C3-C8 cycloalkylene, C3-C8 cycloalkenylene; Ra is H or C1-C12 alkyl; R1 and R2 are each independently C6-C24 alkyl or C6-C24 alkenyl; R3 is H, OR5, CN, C(═O) OR4, OC(═O) R4 or —NR5C(═O)R4; R4 is C1-C12 alkyl; R5 is H or C1-C6 alkyl; and x is 0, 1 or 2.
74C. The pharmaceutical composition of items 71 to 74A or 74B, wherein the cationic lipid is selected or derived from formula III-3 of WO2018078053, preferably wherein the cationic lipid has the chemical term ((4-hydroxybutyl) azanediyl)bis(hexane-6,1-diyl)bis(2-hexyldecanoate), also referred to as ALC-0315.
75. The pharmaceutical composition of items 71 to 74A or 74B or 74C, wherein the cationic lipid or ionizable lipid is selected from ALC-0315, SM-102, SS-33/4PE-15, HEXA-C5DE-PipSS, or compound C26.
76. The pharmaceutical composition of items 71 to 75, wherein the neutral lipid or phospholipid is selected from DSPC, DHPC, or DPhyPE.
77. The pharmaceutical composition of items 71 to 76, wherein the steroid or steroid analogue is selected from cholesterol, cholesteryl hemisuccinate (CHEMS), preferably cholesterol.
78. The pharmaceutical composition of items 68 to 77, wherein the lipid-based carriers, preferably the LNPs, comprise
   (i) at least one cationic lipid, preferably as defined in items 74 or 75;
   (ii) at least one neutral lipid, preferably as defined in item 76;
   (iii) at least one steroid or steroid analogue, preferably as defined in item 77; and
   (iv) at least one aggregation reducing lipid, preferably as defined in items 72 to 73
79. The pharmaceutical composition of items 68 to 78, wherein the lipid-based carriers comprise about 20-60% cationic lipid, about 5-25% neutral lipid, about 25-55% steroid or steroid analogue, and about 0.5-15% aggregation reducing lipid.
80. The pharmaceutical composition of items 68 to 79, wherein the wt/wt ratio of lipid to nucleic acid (e.g. RNA) in the lipid-based carrier is from about 10:1 to about 60:1.
81. The pharmaceutical composition of items 68 to 80, wherein the N/P ratio of the lipid-based carriers encapsulating the nucleic acid, preferably the RNA, is in a range from about 1 to about 20.
82. The pharmaceutical composition of items 68 to 81, wherein the lipid-based carriers have a Z-average size in a range of about 50 nm to about 200 nm.
83. The pharmaceutical composition of items 64 to 82, additionally comprising at least one antagonist of at least one RNA sensing pattern recognition receptor selected from a Toll-like receptor antagonist, preferably a TLR7 antagonist and/or a TLR8 antagonist, most preferably an RNA according to SEQ ID NO: 85 of WO2021028439.
84. The pharmaceutical composition or the artificial nucleic acid of any one of the preceding items, wherein upon intramuscular, intratumoral, or intravenous administration of the composition or nucleic acid to a cell, tissue, or subject, the encoded tumour antigens are produced.
85. The pharmaceutical composition or the artificial nucleic acid of any one of the preceding items, wherein upon intramuscular, intratumoral, or intravenous administration of the composition or nucleic acid to a cell, tissue, or subject, the encoded tumour antigens are produced and induce an epitope-specific CD8+ T cells in the subject.
86. A tumour antigen or a composition of at least one tumour antigen, wherein the tumour antigen is characterized by any one of the preceding items, preferably by any one of the items 2 to 4 or 14 to 16.
87. The tumour antigen or a composition of item 86, wherein the tumour antigen comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 124-254, 546-650, 756-763, or an immunogenic fragment or immunogenic variant of any of these.
88. The tumour antigen or a composition of item 86, wherein the tumour antigen comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 255-317, or an immunogenic fragment or immunogenic variant of any of these.
89. The tumour antigen or a composition of items 86 to 88, wherein the composition comprises a plurality of tumour antigens and, optionally, wherein the antigens are formulated in a pharmaceutically acceptable carrier.
90. An antibody, a T-cell, or a TCR that has been raised against or stimulated with any of a tumour antigens or nucleic acids as defined in any one of the preceding items.
91. A combination comprising at least two or a plurality of therapeutic modalities selected from
   a) at least one artificial nucleic acid of items 1 to 62; and/or
   b) at least one pharmaceutical composition of items 63 to 85; and/or
   c) at least one tumour antigen or composition items 86 to 89; and/or
   d) at least one antibody, T-cell, or TCR of item 90;
92. A kit or kit of parts comprising at least one artificial nucleic acid of any one of items 1 to 62, and/or at least one pharmaceutical composition of any one of items 63 to 85, and/or at least one tumour antigen or a composition of tumour antigens of items 86 to 89, and/or at least one antibody, a T-cell, or a TCR of item 90, optionally comprising a liquid vehicle for solubilising, and optionally comprising technical instructions providing information on administration and dosage of the components.
93. An artificial nucleic acid of any one of items 1 to 62, a pharmaceutical composition of any one of items 63 to 85, a tumour antigen or a composition of tumour antigens of items 86 to 89, an antibody, T-cell, or TCR of item 90, a combination of item 91, or a kit or kit of parts of item 92 for use as a medicament.
94. An artificial nucleic acid of any one of items 1 to 62, a pharmaceutical composition of any one of items 63 to 85, a tumour antigen or a composition of tumour antigens of items 86 to 89, an antibody, T-cell, or TCR of item 90, a combination of item 91, or a kit or kit of parts of item 92 for use as a medicament in treating or preventing cancer in a subject, or any disease, disorder, or condition related to cancer.
95. An artificial nucleic acid of any one of items 1 to 62, a pharmaceutical composition of any one of items 63 to 85, a tumour antigen or a composition of tumour antigens of items 86 to 89, an antibody, T-cell, or TCR of item 90, a combination of item 91, or a kit or kit of parts of item 92, for use as a medicament in treating or preventing squamous cell carcinoma in a subject, or any disease, disorder, or condition related to squamous cell carcinoma, preferably wherein the squamous cell carcinoma is selected from LUSC/squamous non-small-cell lung cancer (sqNSCLC) or head and neck squamous cell carcinoma (HNSCC).
96. An artificial nucleic acid of any one of items 1 to 62, a pharmaceutical composition of any one of items 63 to 85, a tumour antigen or a composition of tumour antigens of items 86 to 89, an antibody, T-cell, or TCR of item 90, a combination of item 91, or a kit or kit of parts of item 92, for use as a medicament in treating or preventing NSCLC in a subject, or any disease, disorder, or condition related to NSCLC.
97. The use as a medicament in treating or preventing NSCLC of item 96, wherein the at least one tumour antigen that is provided by the artificial nucleic acid, the pharmaceutical composition, the tumour antigen or a composition of tumour antigens, the combination, or the kit or kit of parts comprises (a) at least one antigenic peptide selected from a peptide or protein encoded by lnc-WDR72-2:4, lnc-TRPC5-3:1, KCNMB2-AS1:4, lnc-NTF3-5:5, or lnc-ZC3H8-6:1, or an immunogenic fragment or variant of any of these; and/or (b) at least one antigenic peptide selected from a peptide or protein of a BRAF neoantigen, EGFR neoantigen, TP53 neoantigen, PIK3CA neoantigen, NFE2L2 neoantigen, preferably selected from EGFR neoantigen, or TP53 neoantigen, or an immunogenic fragment or variant of any of these.
96B. An artificial nucleic acid of any one of items 1 to 62, a pharmaceutical composition of any one of items 63 to 85, a tumour antigen or a composition of tumour antigens of items 86 to 89, an antibody, T-cell, or TCR of item 90, a combination of item 91, or a kit or kit of parts of item 92, for use as a medicament in treating or preventing HNSCC in a subject, or any disease, disorder, or condition related to HNSCC.
97B. The use as a medicament in treating or preventing HNSCC of item 96B, wherein the at least one tumour antigen that is provided by the artificial nucleic acid, the pharmaceutical composition, the tumour antigen or a composition of tumour antigens, the combination, or the kit or kit of parts comprises at least one antigenic peptide selected from a peptide or protein encoded by lnc-WDR72-2:4, KCNMB2-AS1:4, lnc-NTF3-5:5, or lnc-ZC3H8-6:1, or an immunogenic fragment or variant of any of these.
98. An artificial nucleic acid of any one of items 1 to 62, a pharmaceutical composition of any one of items 63 to 85, a tumour antigen or a composition of tumour antigens of items 86 to 89, an antibody, T-cell, or TCR of item 90, a combination of item 91, or a kit or kit of parts of item 92 for use as a medicament in treating or preventing melanoma in a subject, or any disease, disorder, or condition related to melanoma.
99. The use as a medicament in treating or preventing melanoma of item 98, wherein the at least one tumour antigen that is provided by the artificial nucleic acid, the pharmaceutical composition, the tumour antigen or a composition of tumour antigens, the combination, or the kit or kit of parts comprises (a) at least one antigenic peptide selected from a peptide or protein encoded by lnc-CLEC2D-9:1, lnc-TRPC5-3:1, or LINC00893:25, or an immunogenic fragment or variant of any of these; and/or (b) at least one antigenic peptide selected from a peptide or protein from an NRAS neoantigen, TYW1B neoantigen, ECPAS neoantigen, MAP2K1 neoantigen, TOMM22 neoantigen, GLB1 neoantigen, MAGE-A3 neoantigen, ATAD2 neoantigen, BRAF neoantigen, DIP2B neoantigen, TRRAP neoantigen, RAC1 neoantigen, AP4B1 neoantigen, VWA5A neoantigen, CLTC neoantigen, RPL9 neoantigen, RPAP1 neoantigen, RPE65 neoantigen, PATZ1 neoantigen, CNOT9 neoantigen, preferably selected from an NRAS neoantigen, TYW1B neoantigen, ECPAS neoantigen, MAP2K1 neoantigen, TOMM22 neoantigen, GLB1 neoantigen, MAGE-A3 neoantigen, ATAD2 neoantigen, or BRAF neoantigen, or an immunogenic fragment or variant of any of these.
99B. An artificial nucleic acid of any one of items 1 to 62, a pharmaceutical composition of any one of items 63 to 85, a tumour antigen or a composition of tumour antigens of items 86 to 89, an antibody, T-cell, or TCR of item 90, a combination of item 91, or a kit or kit of parts of item 92 for use as a medicament in treating or preventing Adrenocortical carcinoma, Bladder Urothelial Carcinoma, Breast invasive carcinoma, Cervical carcinoma (Cervical squamous cell carcinoma and endocervical adenocarcinoma), Colon adenocarcinoma, Kidney renal papillary cell carcinoma, Lung adenocarcinoma, Lymphoid Neoplasm Diffuse Large B-cell Lymphoma, Rectum adenocarcinoma, Sarcoma, Skin Cutaneous Melanoma, Thymoma, Thyroid carcinoma, Hepatocellular carcinoma, Uterine Carcinosarcoma, or Uterine Corpus Endometrial Carcinoma in a subject, or any related disease, disorder, or condition, wherein the at least one tumour antigen that is provided by the artificial nucleic acid, the pharmaceutical composition, the tumour antigen or a composition of tumour antigens, the combination, or the kit or kit of parts comprises at least one antigenic peptide selected from a peptide or protein encoded by lnc-ZC3H8-6:1, or an immunogenic fragment or variant thereof.
99C. An artificial nucleic acid of any one of items 1 to 62, a pharmaceutical composition of any one of items 63 to 85, a tumour antigen or a composition of tumour antigens of items 86 to 89, an antibody, T-cell, or TCR of item 90, a combination of item 91, or a kit or kit of parts of item 92 for use as a medicament in treating or preventing Bladder Urothelial Carcinoma, Cervical carcinoma (Cervical squamous cell carcinoma and endocervical adenocarcinoma), Thymoma, or Uterine Corpus Endometrial Carcinoma in a subject, or any related disease, wherein the at least one tumour antigen that is provided by the artificial nucleic acid, the pharmaceutical composition, the tumour antigen or a composition of tumour antigens, the combination, or the kit or kit of parts, comprises at least one antigenic peptide selected from a peptide or protein encoded by KCNMB2-AS1:4, or an immunogenic fragment or variant thereof.
99D. An artificial nucleic acid of any one of items 1 to 62, a pharmaceutical composition of any one of items 63 to 85, a tumour antigen or a composition of tumour antigens of items 86 to 89, an antibody, T-cell, or TCR of item 90, a combination of item 91, or a kit or kit of parts of item 92 for use as a medicament in treating or preventing Ovarian serous cystadenocarcinoma, Testicular Germ Cell Tumors, Thymoma, or Uterine carcinosarcoma in a subject, or any related disease, wherein the at least one tumour antigen that is provided by the artificial nucleic acid, the pharmaceutical composition, the tumour antigen or a composition of tumour antigens, the combination, or the kit or kit of parts comprises at least one antigenic peptide selected from a peptide or protein encoded by lnc-NTF3-5:5, or an immunogenic fragment or variant thereof.
99E. An artificial nucleic acid of any one of items 1 to 62, a pharmaceutical composition of any one of items 63 to 85, a tumour antigen or a composition of tumour antigens of items 86 to 89, an antibody, T-cell, or TCR of item 90, a combination of item 91, or a kit or kit of parts of item 92 for use as a medicament in treating or preventing Bladder Urothelial Carcinoma, Colon adenocarcinoma, Esophageal carcinoma, or Stomach adenocarcinoma in a subject, or any related disease, wherein the at least one tumour antigen that is provided by the artificial nucleic acid, the pharmaceutical composition, the tumour antigen or a composition of tumour antigens, the combination, or the kit or kit of parts comprises at least one antigenic peptide selected from a peptide or protein encoded by lnc-WDR72-2:4, or an immunogenic fragment or variant thereof.
99F. An artificial nucleic acid of any one of items 1 to 62, a pharmaceutical composition of any one of items 63 to 85, a tumour antigen or a composition of tumour antigens of items 86 to 89, an antibody, T-cell, or TCR of item 90, a combination of item 91, or a kit or kit of parts of item 92 for use as a medicament in treating or preventing Skin Cutaneous Melanoma, Testicular Germ Cell Tumors, or Uveal Melanoma in a subject, or any related disease, disorder, or condition, wherein the at least one tumour antigen that is provided by the artificial nucleic acid, the pharmaceutical composition, the tumour antigen or a composition of tumour antigens, the combination, or the kit or kit of parts comprises at least one antigenic peptide selected from a peptide or protein encoded by lnc-CLEC2D-9:1, or an immunogenic fragment thereof.
100. The use as a medicament of items 93 to 99F, wherein the medicament is administered to the subject by intramuscular, intratumoral, or intravenous administration.
101. A method of treating or preventing a disease, disorder or condition, the method comprising applying or administering to a subject in need thereof an effective amount of the artificial nucleic acid of items 1 to 62, pharmaceutical composition of items 63 to 85, tumour antigen or a composition of tumour antigens of items 86 to 89, antibody, T-cell, or TCR of item 90, combination of item 91, or kit or kit of parts of item 92.
102. The method of item 101, wherein the disease, disorder or condition is cancer.
103. The method of item 102, wherein cancer is squamous cell carcinoma, preferably selected from squamous non-small-cell lung cancer (sqNSCLC) or head and neck squamous cell carcinoma (HNSCC).
104 The method of items 102 or 103, wherein the cancer is NSCLC and wherein the at least one tumour antigen that is provided by the artificial nucleic acid, the pharmaceutical composition, the tumour antigen or a composition of tumour antigens, or the kit or kit of parts is comprises (a) at least one antigenic peptide selected from a peptide or protein encoded by lnc-WDR72-2:4, lnc-TRPC5-3:1, KCNMB2-AS1:4, lnc-NTF3-5:5, or lnc-ZC3H8-6:1, or an immunogenic fragment or variant of any of these; and/or (b) at least one antigenic peptide selected from a peptide or protein from a BRAF neoantigen, EGFR neoantigen, TP53 neoantigen, PIK3CA neoantigen, or NFE2L2 neoantigen, preferably selected from EGFR neoantigen or TP53 neoantigen, or an immunogenic fragment or variant of any of these.
105. The method of item 102, wherein the cancer is melanoma and wherein the at least one tumour antigen that is provided by the artificial nucleic acid, the pharmaceutical composition, the tumour antigen or a composition of tumour antigens, or the kit or kit of parts comprises (a) at least one antigenic peptide selected from a peptide or protein encoded by lnc-CLEC2D-9:1 or lnc-TRPC5-3:1, or an immunogenic fragment or variant of any of these; and/or (b) at least one antigenic peptide selected from a peptide or protein from an NRAS neoantigen, TYW1B neoantigen, ECPAS neoantigen, MAP2K1 neoantigen, TOMM22 neoantigen, GLB1 neoantigen, MAGE-A3 neoantigen, ATAD2 neoantigen, BRAF neoantigen, DIP2B neoantigen, TRRAP neoantigen, RAC1 neoantigen, AP4B1 neoantigen, VWA5A neoantigen, CLTC neoantigen, RPL9 neoantigen, RPAP1 neoantigen, RPE65 neoantigen, PATZ1 neoantigen, CNOT9 neoantigen, preferably selected from an NRAS neoantigen, TYW1B neoantigen, ECPAS neoantigen, MAP2K1 neoantigen, TOMM22 neoantigen, GLB1 neoantigen, MAGE-A3 neoantigen, ATAD2 neoantigen, or BRAF neoantigen, or an immunogenic fragment or variant of any of these.
106. The method of item 101, characterized by any one of the medical use features as defined in item 93 to 100.

### BRIEF DESCRIPTION OF THE FIGURES

FIG. 1 shows an exemplary neoantigen identification workflow. * Immunogenicity prediction: neolM; MHC presentation prediction: MHCnuggets & neoMS. ** Taking into account host gene expression, sample coverage, HLA coverage/distribution, MHC presentation ability and immunogenicity, etc.

FIG. 2. shows the high tumour specificity of the lncRNA transcript lnc-CLEC2D-9:1 (corresponding smORF: lncCLEC2D91412616). RNAseq data obtained from the TCGA (tumour tissue) and GTEX (healthy tissue) database shows high target expression in melanoma and low to absent expression in healthy tissues (except in the immune-privileged tissue testis).

FIG. 3 shows the high tumour specificity of the lncRNA transcript lnc-TRPC5-3:1 (corresponding smORF: lncTRPC53116031801). RNAseq data obtained from the TCGA (tumour tissue) and GTEX (healthy tissue) database shows high target expression in melanoma and low to absent expression in healthy tissues.

FIG. 4 shows the high tumour specificity of the lncRNA transcript lnc-TRPC5-3:1 (corresponding smORF: lncTRPC53116031801). RNAseq data obtained from the TCGA (tumour tissue) and GTEX (healthy tissue) database shows high target expression in NSCLC (both in lung adenocarcinoma, LUAD, and lung squamous cell carcinoma, LUSC) and low to absent expression in healthy tissues.

FIG. 5 shows the high tumour specificity of the lncRNA transcript lnc-WDR72-2:4 (corresponding smORFs: lncWDR722415641696 and lncWDR722416421696). RNAseq data obtained from the TCGA (tumour tissue) and GTEX (healthy tissue) database shows high target expression in NSCLC (lung squamous cell carcinoma, LUSC) and low to absent expression in healthy tissues.

FIG. 6 shows the high tumour specificity of the lncRNA transcript KCNMB2-AS1:4 (corresponding smORF: KCNMB2AS14633753). RNAseq data obtained from the TCGA (tumour tissue) and GTEX (healthy tissue) database shows high target expression in NSCLC (lung squamous cell carcinoma, LUSC) and low to absent expression in healthy tissues.

FIG. 7 shows the high tumour specificity of the lncRNA transcript lnc-NTF3-5:5 (corresponding smORF: lncNTF35511951417). RNAseq data obtained from the TCGA (tumour tissue) and GTEX (healthy tissue) database shows high target expression in NSCLC (lung squamous cell carcinoma, LUSC) and low to absent expression in healthy tissues.

FIG. 8 shows the high tumour specificity of the lncRNA transcript lnc-ZC3H8-6:1 (corresponding smORF: lncZC3H86163144). RNAseq data obtained from the TCGA (tumour tissue) and GTEX (healthy tissue) database shows high target expression in NSCLC (both in lung adenocarcinoma, LUAD, and lung squamous cell carcinoma, LUSC) and low to absent expression in healthy tissues.

FIG. 9 shows the near absence of the smORF lncTRPC53116031801's translated peptide in healthy tissues. Mass spectrometry data shows very low to absent levels (asterisks) in various healthy tissues in comparison to different highly abundant controls (e.g. housekeeping genes; dots).

FIG. 10 shows the in vitro antigen validation of melanoma neoantigens (peptide pool 1) in a DC-T cell assay (further details see Example 6.1, Table E3-E5). IFN-gamma readouts of pan T-cells after two stimulation rounds (R1 and R2) were performed for 10 PBMC donors (D1-D10). 7 donors showed an antigen specific T cell response to at least one of the peptides of pool 1. Recall and memory responses were observed in 1 and 1 donor, respectively.

FIG. 11 shows the in vitro antigen validation of melanoma neoantigens (peptide pool 2) in a DC-T cell assay (further details see Example 6.1, Table E3-E5). IFN-gamma readouts of pan T-cells after two stimulation rounds (R1 and R2) were performed for 10 PBMC donors (D1-D10). 7 donors showed an antigen specific T cell response to at least one of the peptides of pool 2. A memory response was observed in 2 donors.

FIG. 12 shows the in vitro antigen validation of melanoma cancer antigens (peptide pool 3) in a DC-T cell assay (further details see Example 6.1, Table E3-E5). IFN-gamma readouts of pan T-cells after two stimulation rounds (R1 and R2) were performed for 10 PBMC donors (D1-D10). All donors showed an antigen specific T cell response to at least one of the peptides of pool 3. Recall and memory responses were observed in 4 and 4 donors, respectively.

FIG. 13 shows the in vitro antigen validation of melanoma neoantigens (peptide pool 4) in a DC-T cell assay (further details see Example 6.1, Table E3-E5). IFN-gamma readouts of pan T-cells after two stimulation rounds (R1 and R2) were performed for 10 PBMC donors (D1-D10). 8 donors showed an antigen specific T cell response to at least one of the peptides of pool 4. Recall and memory responses were observed in 2 and 1 donors, respectively.

FIG. 14 shows the in vitro antigen validation of melanoma neoantigens (peptide pool 5) in a DC-T cell assay (further details see Example 6.1, Table E3-E5). IFN-gamma readouts of pan T-cells after two stimulation rounds (R1 and R2) were performed for 10 PBMC donors (D1-D10). 4 donors showed an antigen specific T cell response to peptide pool 5. Recall and memory responses were observed in 1 and 2 donors, respectively.

FIG. 15 shows an overview of all recall and memory responses observed in Example 6.1/FIGS. 10-14.

FIG. 16 shows the in vitro antigen validation of the indicated smORFs in a DC-T cell assay (further details see Example 6.2, Table E6-E8). IFN-gamma readouts of pan T-cells after two stimulation rounds (R1 and R2) were performed for various PBMC donors.

FIG. 17 shows the in vitro antigen validation of the indicated melanoma neoantigens in a DC-T cell assay (further details see Example 6.3, Table E9-E10A). IFN-gamma readouts of pan T-cells after two stimulation rounds (R1 and R2) were performed for various PBMC donors.

FIG. 18 shows the in vivo immunogenicity of an LNP-formulated mRNA vaccine R12300 encoding two smORFs (that are amongst others expressed in melanoma) in CB6F1 hybrid mice. Mice were vaccinated intramuscularly with 5 μg LNP-formulated R12300 at day 0, 7 and 14. Vaccination with irrelevant mRNA (PpLuc; R8730) served as control. At day 21, mice were sacrificed and splenocytes were isolated. Splenocytes were restimulated with a 15-mer peptide library for lncCLEC2D91412616 or DMSO as control and analyzed by flow cytometry. The magnitude of the CD8+ (top) and CD4+ (bottom) T cell responses against the encoded smORF peptide lncCLEC2D91412616 is shown as percentage of IFN-γ+ TNF+ cells of CD8+ or CD4+ T cells, respectively. Median values are plotted.

FIG. 19 shows the in vivo immunogenicity of an LNP-formulated mRNA vaccine R12301 encoding multiple smORFs (that are amongst others expressed in NSLSC) in CB6F1 hybrid mice. Mice were vaccinated intramuscularly with 5 μg LNP-formulated R12301 at day 0, 7 and 14. Vaccination with irrelevant mRNA (PpLuc; R8730) served as control. At day 21, mice were sacrificed and splenocytes were isolated. Splenocytes were individually restimulated with peptide libraries for each smORF (15-mer libraries covering the complete antigens) or DMSO as control and analyzed by flow cytometry. The magnitude of the CD8+ (top) and CD4+ (bottom) T cell responses against the 4 encoded smORF peptides KCNMB2AS14633753, lncNTF35511951417, lncWDR722415641696 and lncZC3H86163144 (NKZW-smORFs) is shown as percentage of IFN-γ+ TNF+ cells of CD8+ or CD4+ T cells, respectively. Median values are plotted.

### EXAMPLES

In the following, examples illustrating various embodiments of the invention are presented. However, the invention shall not to be limited in scope by the specific embodiments presented herein and should rather be understood as being applicable to other compositions or uses as e.g. defined in the specification. Accordingly, the following preparations and examples are given to enable those skilled in the art to more clearly understand and to practice the present invention. Indeed, various modifications of the invention in addition to those described herein will become readily apparent to those skilled in the art from the foregoing description, accompanying figures, and the examples below.

### Example 1: Antigen Discovery and Selection Methods

The goal of the example was to identify cancer antigens that produce epitopes in patients with NSCLC or melanoma. The analysis focused on two classes of targets: tumor-specifc antigens derived from somatic mutations (neoepitopes) and tumor-associated antigens encoded by small open reading frames (smORFs) identified in long non-coding RNAs (lncRNAs). The identified smORFs/lncRNAs showed a well defined tumour-associated expression and were only detected in low levels in normal tissue, using transcriptomics and proteomics. For both classes of antigens, major histocompatibility complex (MHC) binding and immunogenicity predictions were used to select the candidates.

### Discovery of Neoepitopes:

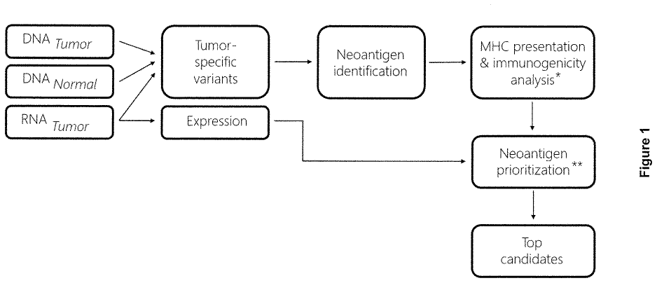

Sequence alignment map (BAM) files from the cancer genome atlas (TCGA) database were used to detect tumour-specific variants. A combination of parameters was used to select the most promising candidates. A threshold of 0.55 immunogenicity score (neoim_score, larger values reflect higher immunogenicity probability) was used. Additionally, only genes were considered that show expression of at least at 20 transcripts per million (TPM). Finally, the neoepitopes were prioritized based on their expected population coverage (fraction of patients having the mutation and the predicted binding MHC-I binding alleles). A neoantigen identification workflow is shown in FIG. 1.

Discovery of smORFs in lncRNAs:

To discover smORFs in lncRNAs, the coding potential in the lncRNA sequence was predicted as an initial step. Then, a transcriptomics differential expression analysis was conducted comparing tumour vs. normal expression; only transcripts with a log 2 (Fold change) greater than 2 and an adjusted p-value less than 1e−4 were considered for further analysis. Besides, the MHC-I binding and immunogenicity of the smORFs 8 to 11 nmers were predicted and only those smORFs predicted to bind MHC-I and having nmers with a neoim_score greater than 0.5 were kept. Further, only lncRNAs were considered with evidence of translation as determined by mass spectrometry (PXD009630—melanoma, and PXD002612—lung cancer). As a final filtering step, the lncRNA/smORFs were kept that show low expression in normal tissue, using transcriptomics (GTEx V7) and mass spectrometry (PXD010154).

The immunogenicity score (neoim_score) is generated using a machine learning algorithm and reflects the probability that an epitope is immunogenic. The RNAseq expression is given in TPMs; TPMs normalize RNAseq reads by the length of each gene and sequencing depth and are commonly used to compare expression levels in different samples. The expected coverage of the neoantigens was calculated by taking the product of the population frequency of the mutation and the HLA allele frequencies predicted to bind the epitope. The prediction of the HLA alleles was performed using a machine learning algorithm.

### Example 2: Antigen Discovery and Selection of Melanoma and NSCLC Neoantigens

The goal of the experiment was to discover melanoma and NSCLC cancer antigens derived from tumour-specific neoantigens. The resulting top hits are summarized in Table 3.

Table 3 shows identified melanoma and NSCLC neoantigens. For the respective neoantigens in the table, RNAseq expression levels (expr mean), predicted immunogenicity score (neoim_score), coverage in the respective cancer type (mean expected coverage), the HLA alleles expected to bind (HLA mupexi alleles), and cancer indication as well as subcohort (M=melanoma; M (BRAF)=BRAF-mutated melanoma; M (NRAS)=NRAS-mutated melanoma; N═NSCLC; LUSC=lung squamous cell carcinoma (sqNSCLC); LUAD=lung adenocarcinoma) are indicated. M/melanoma in this context furthermore specifically means skin cutananeous melanoma (SKCM).

TABLE 3
Identified suitable melanoma and NSCLC neoantigens

| | | | | | Mean | |
|---|---|---|---|---|---|---|
| SEQ | | | expr | | expected | |
| ID NO: | | neoim_{—} | mean | HLA alleles expected to | coverage | |
| Protein | Gene | score | (TPM) | bind (HLA mupexi alleles) | (fraction) | Indication |
| 262 | BRAF | 0.60 | 36.43 | A*03:01, A*11:01 | 0.239 | M (BRAF) |
| 265 | BRAF | 0.58 | 36.43 | A*03:01, A*11:01 | 0.055 | M (BRAF) |
| 276 | MAP2K1 | 0.72 | 24.50 | C*05:01, B*08:01, C*07:01 | 0.022 | M (BRAF) |
| 296 | RAC1 | 0.93 | 259.90 | C*07:01, A*02:10, C*05:01, | 0.029 | M (BRAF) |
| | | | | C*06:02 | | |
| 297 | RAC1 | 0.78 | 259.90 | B*51:01, C*07:01, A*02:10 | 0.025 | M (BRAF) |
| 290 | TRRAP | 0.56 | 28.93 | A*02:10, C*07:01, C*05:01, | 0.018 | M (BRAF) |
| | | | | C*06:02 | | |
| 291 | TRRAP | 0.69 | 28.93 | C*07:01, A*02:10, C*05:01 | 0.017 | M (BRAF) |
| 292 | TRRAP | 0.96 | 28.93 | A*02:10, C*07:01 | 0.015 | M (BRAF) |
| 294 | TRRAP | 0.69 | 28.93 | C*07:01, C*05:01 | 0.011 | M (BRAF) |
| 312 | PATZ1 | 0.74 | 32.09 | C*07:01, A*02:10, C*05:01 | 0.010 | M (BRAF) |
| 302 | VWA5A | 0.59 | 24.02 | A*02:10, C*07:01, C*05:01 | 0.010 | M (BRAF) |
| 293 | TRRAP | 0.64 | 28.93 | A*24:02, A*03:01 | 0.009 | M (BRAF) |
| 278 | TOMM22 | 0.70 | 89.51 | A*01:01, A*03:01, C*07:01, | 0.009 | M (BRAF) |
| | | | | C*05:01, A*02:10, C*06:02 | | |
| 307 | RPAP1 | 0.66 | 52.33 | A*01:01, A*02:10, A*03:01, | 0.008 | M (BRAF) |
| | | | | C*07:01, C*04:01 | | |
| 280 | GLB1 | 0.74 | 54.41 | C*07:01, C*05:01, A*02:10, | 0.008 | M (BRAF) |
| | | | | A*24:02, C*04:01, C*06:02 | | |
| 306 | RPAP1 | 0.61 | 52.33 | A*01:01, C*07:01, C*05:01, | 0.008 | M (BRAF) |
| | | | | A*02:10, C*04:01 | | |
| 273 | TYW1B | 0.60 | 28.79 | C*07:01, C*05:01, A*02:10, | 0.008 | M (BRAF) |
| | | | | A*01:01, C*04:01 | | |
| 275 | ECPAS | 0.70 | 63.21 | A*01:01, C*07:01, C*05:01, | 0.004 | M (BRAF) |
| | | | | C*06:02, A*02:10 | | |
| 316 | CNOT9 | 0.64 | 52.19 | A*02:10, A*03:01, B*07:02, | 0.024 | M (BRAF) |
| | | | | B*08:01, C*07:01 | | |
| 270 | NRAS | 0.56 | 52.41 | A*01:01 | 0.089 | M (NRAS) |
| 269 | NRAS | 0.59 | 52.41 | A*01:01 | 0.042 | M (NRAS) |
| 267 | NRAS | 0.59 | 52.41 | A*11:01 | 0.035 | M (NRAS) |
| 272 | NRAS | 0.87 | 52.4 | A*01:01 | 0.124 | M (NRAS) |
| 296 | RAC1 | 0.93 | 259.90 | C*07:01, A*02:10, C*05:01, | 0.056 | M (NRAS) |
| | | | | C*06:02 | | |
| 297 | RAC1 | 0.78 | 259.90 | B*51:01, C*07:01, A*02:10 | 0.048 | M (NRAS) |
| 316 | CNOT9 | 0.64 | 52.19 | A*02:10, A*03:01, B*07:02, | 0.023 | M (NRAS) |
| | | | | B*08:01, C*07:01 | | |
| 309 | RPE65 | 0.58 | 22.08 | C*07:01, A*02:10, A*01:01, | 0.016 | M (NRAS) |
| | | | | A*24:02, C*05:01, C*04:01 | | |
| 282 | MAGE- | 0.84 | 54.14 | C*07:01, C*05:01, A*02:10, | 0.016 | M (NRAS) |
| | A3 | | | A*03:01, C*04:01, C*06:02 | | |
| 303 | CLTC | 0.61 | 91.23 | C*06:02, A*02:10, A*01:01, | 0.016 | M (NRAS) |
| | | | | C*07:01, C*05:01 | | |
| 305 | RPL9 | 0.59 | 72.94 | A*01:01, A*02:10, A*03:01, | 0.015 | M (NRAS) |
| | | | | C*07:01 | | |
| 300 | AP4B1 | 0.74 | 35.93 | C*07:01, C*05:01, A*02:10, | 0.015 | M (NRAS) |
| | | | | A*03:01, C*04:01 | | |
| 284 | ATAD2 | 0.68 | 31.38 | C*07:01, A*02:10, A*03:01, | 0.015 | M (NRAS) |
| | | | | C*05:01, C*04:01 | | |
| 285 | ATAD2 | 0.60 | 31.38 | C*04:01, A*02:10, A*03:01, | 0.015 | M (NRAS) |
| | | | | C*07:01, C*05:01 | | |
| 288 | DIP2B | 0.68 | 44.37 | C*07:01, C*05:01, C*04:01, | 0.015 | M (NRAS) |
| | | | | C*06:02, A*02:10 | | |
| 310 | RPE65 | 0.56 | 22.08 | A*03:01, A*11:01, A*02:10 | 0.013 | M (NRAS) |
| 287 | DIP2B | 0.58 | 44.37 | C*07:01, C*06:02, A*02:10 | 0.013 | M (NRAS) |
| 260 | TP53 | 0.58 | 29.09 | B*08:01, B*07:02 | 0.010 | N (LUSC) |
| 259 | TP53 | 0.62 | 29.09 | B*08:01, B*07:02 | 0.010 | N (LUSC) |
| 258 | TP53 | 0.61 | 29.09 | C*07:02 | 0.007 | N (LUSC) |
| 314 | PIK3CA | 0.65 | 55.3 | A*03:01, A*01:01, A*11:01 | 0.013 | N (LUSC) |
| 315 | NFE2L2 | 0.60 | 222.9 | C*05:01, A*02:10 | 0.06 | N (LUSC) |
| 256 | EGFR | 0.65 | 103.15 | A*02:10, A*03:01 | 0.024 | N (LUAD) |
| 255 | EGFR | 0.60 | 103.15 | A*01:01 | 0.011 | N (LUAD) |
| 260 | TP53 | 0.58 | 39.70 | B*07:02, B*08:01 | 0.007 | N (LUAD) |
| 259 | TP53 | 0.62 | 39.70 | B*07:02, B*08:01 | 0.007 | N (LUAD) |
| 262 | BRAF | 0.60 | 89.02 | A*11:01, A*03:01 | 0.005 | N (LUAD) |
| 258 | TP53 | 0.61 | 39.70 | C*07:02 | 0.005 | N (LUAD) |

### Example 3: Antigen Discovery and Selection of Melanoma and NSCLC Antigens Encoded by lncRNAs

The goal of the experiment was to discover melanoma and NSCLC cancer antigens derived from lncRNA-smORFs. The resulting top hits are summarized in Table 4.

Table 4 shows identified melanoma and NSCLC antigens encoded by lncRNA transcripts. For the respective smORFs in the table, RNAseq expression levels (expr mean), predicted immunogenicity score (neoim_score), and cancer indication as well as subcohort (M=melanoma; N═NSCLC; LUSC=lung squamous cell carcinoma (sqNSCLC); LUAD=lung adenocarcinoma) are indicated. M/melanoma in this context furthermore specifically means skin cutananeous melanoma (SKCM).

TABLE 4
Identified suitable melanoma and NSCLC
antigens encoded by lncRNA transcripts

| | | expr | Indication |
|---|---|---|---|
| smORF | neoim_score | mean(TPM) | (subcohort) |
| lncTRPC53116031801 | 0.595 | 3.79 | N (LUSC) |
| lncTRPC53116031801 | 0.595 | 3.79 | N (LUAD) |
| lncZC3H86163144 | 0.520 | 1.09 | N (LUSC) |
| lncTRPC53116031801 | 0.595 | 4.43 | M |
| lncWDR722415641696 | 0.571 | 2.01 | N (LUSC) |
| lncWDR722416421696 | 0.566 | 2.01 | N (LUSC) |
| KCNMB2AS14633753 | 0.550 | 1.91 | N (LUSC) |
| lncNTF35511951417 | 0.612 | 2.73 | N (LUSC) |
| lncCLEC2D91412616 | 0.692 | 8.01 | M |

Additionally, data from mass-spectrometry studies including 50 melanoma patients (PXD009630—melanoma) and 57 lung cancer patients (PXD002612—lung adenocarcinoma) confirmed that the preferred smORFs are indeed translated. In this context, the frequency of patients where the smORFs' translated peptides could be detected in the tumor samples by mass spectrometry are given in Table 4A.

TABLE 4A
Frequency of the identified lncRNA-
smORFs in melanoma and NSCLC

| smORF | Frequency* | Indication |
|---|---|---|
| lncTRPC53116031801 | 75% | N (LUAD) |
| lncZC3H86163144 | 30% | N (LUAD) |
| lncTRPC53116031801 | 30% | M |
| lncWDR722415641696 | 13% | N (LUAD) |
| lncWDR722416421696 | 13% | N (LUAD) |
| KCNMB2AS14633753 | 9% | N (LUAD) |
| lncNTF35511951417 | 9% | N (LUAD) |
| lncCLEC2D91412616 | 6% | M |

*Tumor MS sample frequency: for NSCLC(N) out of 57 samples; for Melanoma(M) out of 50 samples

### Example 4: High Tumour Specificity of Preferred lncRNAs

The goal of the experiment was to comparatively analyze the expression of preferred lncRNA transcripts (Table E1) in tumour and healthy tissue. To this end, RNAseq expression data of the TCGA (tumour tissue) and GTEX (healthy tissue) database was analyzed. Furthermore, mass-spectrometry data was analyzed.

### Results

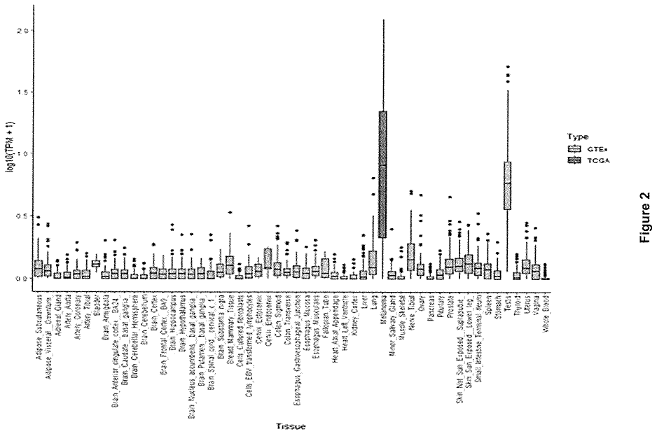

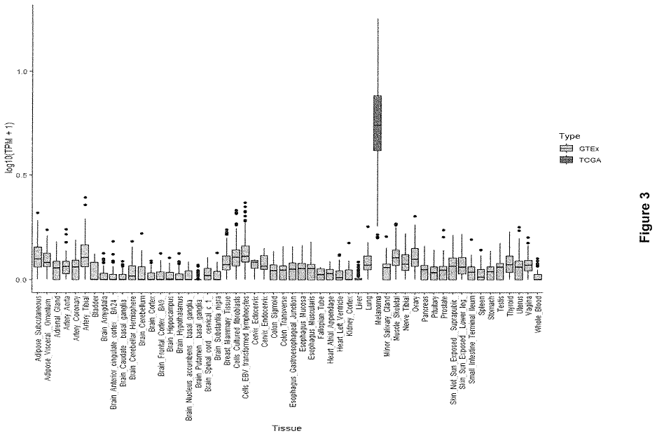

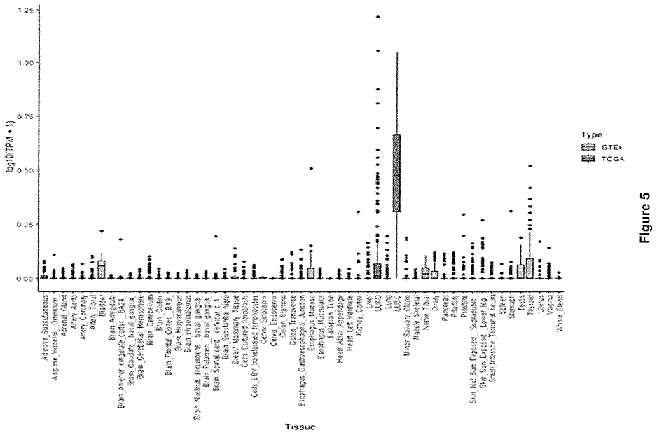

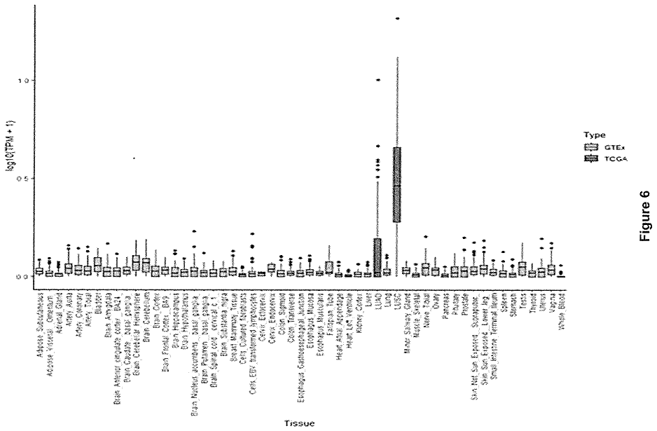

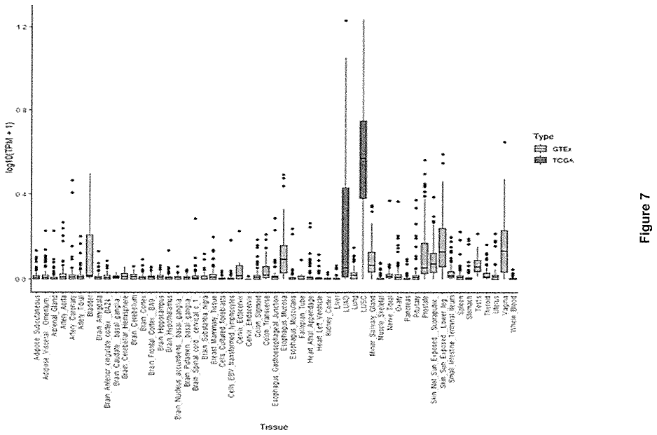

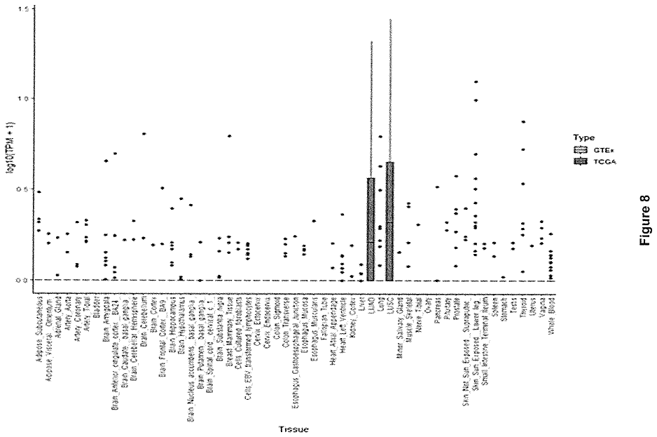

LncRNA transcripts lnc-CLEC2D-9:1, lnc-TRPC5-3:1 were highly expressed in melanoma and lnc-TRPC5-3:1, lnc-WDR72-2:4, KCNMB2-AS1:4, lnc-NTF3-5:5, lnc-ZC3H8-6:1 were highly expressed in NSCLC. All lncRNA transcripts had only low to absent expression in healthy tissue. One exception was the observed expression of lnc-CLEC2D-9:1 in the testis, which, however, was considered neglectable due the testis being an immune-privileged site. The results are shown in FIGS. 2-8 and summarized in Table E1.

TABLE E1
Expression of preferred lncRNA transcripts in melanoma/NSCLC and healthy tissues

| lncRNA | Preferred derived | Expression in | Expression in | Expression in |
|---|---|---|---|---|
| transcript | smORF | melanoma | NSCLC | healthy tissue |
| lnc-CLEC2D-9:1 | lncCLEC2D91412616 | High | n.a. | Low to absent |
| | | | | (except testis) |
| lnc-TRPC5-3:1 | lncTRPC53116031801 | High | High (LUSC/LUAD) | Low to absent |
| lnc-WDR72-2:4 | lncWDR722415641696 | n.a. | High (LUSC) | Low to absent |
| | lncWDR722416421696 | | | |
| KCNMB2-AS1:4 | KCNMB2AS14633753 | n.a. | High (LUSC) | Low to absent |
| lnc-NTF3-5:5 | lncNTF35511951417 | n.a. | High (LUSC) | Low to absent |
| lnc-ZC3H8-6:1 | lncZC3H86163144 | n.a. | High (LUSC/LUAD) | Low to absent |

n.a., not available; LUSC, lung squamous cell carcinoma; LUAD, lung adenocarcinoma

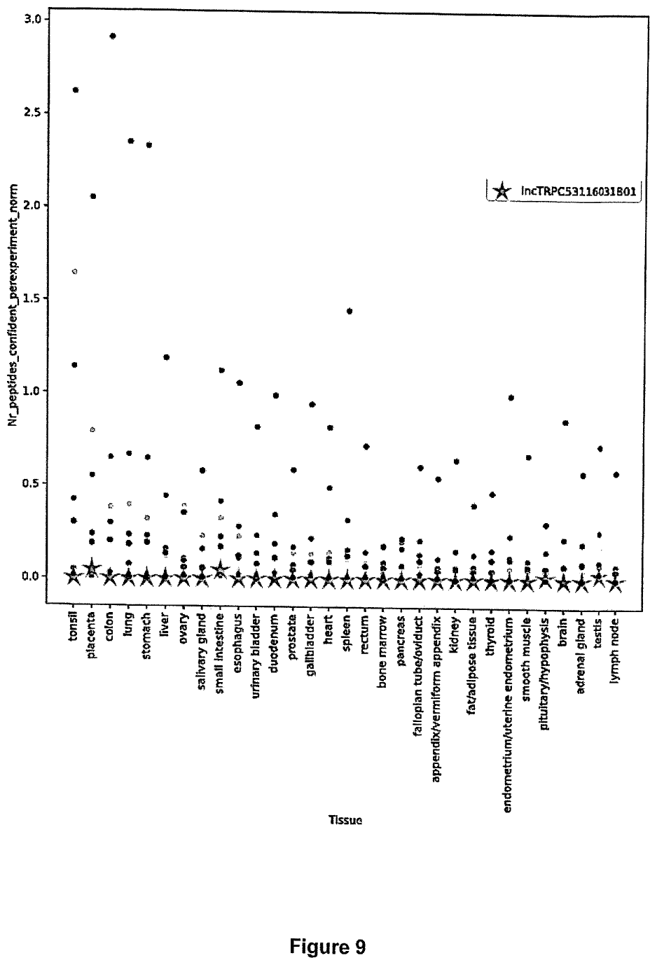

Furthermore, low to absent expression in healthy tissues of the preferred smORFs' translated peptides was confirmed in mass spectrometry analyses. Exemplary data is shown for lncTRPC53116031801 in FIG. 9.

Overall, the data suggests high and frequent tumour expression and low/absent healthy tissue expression for the preferred lncRNA and derived smORFs.

### Example 5: Expression of lncRNA Transcripts in the Thymus

The goal of the experiment was to analyze the expression of the preferred lncRNA transcripts (Table E1) in the thymus. In general, antigen expression in the thymus is associated with central tolerance and thus ideally should be low to absent for target antigens employed in vaccines. To this end, transcriptomics analyses were performed on publicly available datasets.

### Results

Exemplary data from three datasets depicted in Table E2 show only low to absent expression of the lncRNA transcripts in the thymus. This suggests a low likelihood for central tolerance of smORFs derived from these lncRNA transcripts, such as the preferred smORFs stated in Table E1.

TABLE E2
Expression of preferred lncRNA transcripts in the thymus

| | Dataset | Dataset | Dataset |
|---|---|---|---|
| lncRNA | SRR18012442 | SRR18012429 | SRR5576274 |
| transcript | [TPM] | [TPM] | [TPM] |
| lnc-TRPC5-3:1 | 0 | 0.417935 | 0 |
| lnc-WDR72-2:4 | 0.171821 | 0 | 0 |
| KCNMB2-AS1:4 | 0 | 0 | 0 |
| lnc-NTF3-5:5 | 0 | 0 | 0 |
| lnc-ZC3H8-6:1 | 0 | 0 | 0 |
| lnc-CLEC2D-9:1 | 0 | 0 | 0.497972 |

### Example 6: Antigen Validation—In Vitro Immunogenicity

The goal of the experiment was to analyse the in vitro immunogenicity of different melanoma and NSCLC cancer antigens derived from tumour-specific neoantigens and lncRNA-derived smORFs described by Examples 1-5.

Peptides comprising the epitopes of interest were synthesized according to standard procedures. The amino acid sequences of the peptides are given in the respective Examples. Peptide pools (Examples 6.1 and 6.2) or single peptides (Example 6.3 and 6.4) were used.

Immunogenicity of test peptides was assessed using an in vitro DC-T cell assay. In this assay, cryopreserved PBMCs from multiple (healthy) donors of a series of HLA types were retrieved from cryo-storage and monocytes were isolated. Cultured monocytes were differentiated into immature dendritic cells (iDCs) using a cytokine cocktail (GM-CSF and IL-4). The iDCs were loaded with the test peptides and further matured with CD40L to mature dendritic cells (mDC). Subsequently, the mDCs were co-cultured with previously isolated autologous pan T cells for 8 days in the presence of a cytokine cocktail. During co-culture, the medium and cytokines were regularly refreshed. After 8 days, the pan T cells were harvested (round 1=R1) and re-stimulated with peptide loaded monocytes and co-cultured for another 8 days in the presence of a cytokine cocktail (round 2=R2). After round 1 and round 2, the pan T cells were harvested and re-stimulated with peptide loaded monocytes on IFN-γ FluoroSpot plates. After overnight stimulation, the FluoroSpot plates were developed according to the Manufacturer's protocol. The number of IFN-γ secreting pan T cells were measured using the Mabtech IRIS™ FluoroSpot Reader. Furthermore, moDC quality control was performed using flow cytometry. Besides the peptides of interest, appropriate negative (DMSO or a control pool of 29 peptides derived from Myelin-oligodendrocyte glycoprotein (MOG) and positive controls (MART, CEF/CEFBA) were used. Materials need to be sterile, endotoxin levels should be below 0.1EU/mg and protein concentration ≥1 mg/ml.

For analysis, IFNγ+ spot forming units (SFU) were measured with a Fluorospot assay for each R1 and R2 and the delta SFU (dSFU) was calculated as the absolute difference in spots between the tested smORF-derived peptides and the respective DMSO control condition after normalizing the SFU/well to SFU/million.

In Example 6.1 and 6.2 (pooled peptides), a response was considered a “recall response” if the following criteria was met: i) dSFU for R1 was at least 30, and ii) dSFU R2 was at least 2-fold higher compared to dSFU R1.

In Example 6.3 (single peptides), a response was considered a “recall response” if the following criteria was met: i) dSFU for R1 was at least 5, and ii) dSFU R2 was at least 2-fold higher compared to dSFU R1.

A response was considered a “memory response” if the following criteria was met: i) dSFU for R1 was at least 100, and ii) dSFU R2 was 0.1-2.0 fold compared to dSFU R1.

It should be noted that failure to meet the above criteria in a given condition does not necessarily mean that there was no recall or memory response at all. Rather, it may be that a recall or memory response was of a lesser magnitude than the above thresholds or incompatible with the above criteria that were chosen for uniform batch analysis.

### Example 6.1: Antigen Validation—In Vitro Immunogenicity of Melanoma Cancer Antigens—Pooled Peptides

The goal of the experiment was to analyse the in vitro immunogenicity of different melanoma cancer antigens derived from tumour-specific neoantigens and encoded by lncRNA-derived smORFs.

In this assay, cryopreserved PBMCs from 10 multiple (healthy) donors with a series of different HLA types (see Table E3) were tested with 5 different peptide pools (see Table E4) derived from melanoma cancer antigens.

TABLE E3
Overview of PBMC donors used in Example
6.1 and corresponding HLA alleles

| | | | |
|---|---|---|---|
| Donor ID | HLA-A | HLA-B | HLA-C |

| | | | | | | |
|---|---|---|---|---|---|---|
| D1 | 01:01 | 02:01 | 08:01 | 08:01 | 07:01 | 07:01 |
| D2 | 02:01 | 32:01 | 39:01 | 44:02 | 05:01 | 07:02 |
| D3 | 02:01 | 32:01 | 15:01 | 44:02 | 03:03 | 05:01 |
| D4 | 01:01 | 11:01 | 52:01 | 53:01 | 04:01 | 12:02 |
| D5 | 01:01 | 03:01 | 08:01 | 35:01 | 04:01 | 07:01 |
| D6 | 02:05 | 03:01 | 35:01 | 50:01 | 04:01 | 06:02 |
| D7 | 01:01 | 23:01 | 41:01 | 44:02 | 05:01 | 17:01 |
| D8 | 02:01 | 02:01 | 08:01 | 35:01 | 04:01 | 07:01 |
| D9 | 03:01 | 03:01 | 07:02 | 27:05 | 07:02 | 01:02 |
| D10 | 01:02 | 02:05 | 49:01 | 49:01 | 07:01 | 07:01 |

TABLE E4
Overview of melanoma peptide pools used in Example 6.1

| Pool | | SEQ ID NO: | | Predicted HLA binding |
|---|---|---|---|---|
| ID | Sequence | Protein | Source | e.g. to [HLA-] |
| 1 | KIGDFGLATEKSRWSG | 263 | BRAF | A*02:01 |
| | KIGDFGLATMK | 266 | BRAF | A*03:01 |
| | ILDTAGLEEYS | 269 | NRAS | A*01:01 |
| 2 | KVSLEAFFV | 302 | VWA5A | C*07:01 |
| | MFPRAPCLTNRALSK | 248 | LINC0089325197395 | B*07:02 |
| | RLDVLYLHL | 305 | RPL9 | A*01:01 |
| | EPFYLFDGQALLHKF | 311 | RPE65 | C*07:01 |
| | CLLDILDTAGKEEYS | 268 | NRAS | A*11:01 |
| 3 | HIAALKEMVVFLLLY | 286 | ATAD2 | C*07:01 |
| | VTFLDLPVAIINLKE | 274 | TYW1B | C*07:01 |
| | DSFLADQLDLIYVAY | 289 | DIP2B | C*07:01 |
| | FHLIILPLRSMKTLR | 247 | LINC0089325197395 | B*07:02 |
| | RRPLIDQVVQTVLSE | 304 | CLTC | C*06:02 |
| 4 | TTNAFSGEYIPTVFD | 298 | RAC1 | C*07:01 |
| | FLFAAENEQML | 291 | TRRAP | C*07:01 |
| | RIAGLFHLIILPLRS | 246 | LINC0089325197395 | A*02:01 |
| | YQDPAAVFFV | 275 | ECPAS | A*01:01 |
| | LQVLHECNSSYIVGF | 277 | MAP2K1 | C*05:01 |
| 5 | KVAELVYFL | 282 | MAGE-A3 | C*07:01 |
| | DRGLFYYRLLLVVID | 301 | AP4B1 | C*07:01 |
| | AGATFDLFLFVAQKM | 279 | TOMM22 | A*01:01 |
| | NMFLLESFGFQIVQS | 313 | PATZ1 | C*07:01 |
| | SSNILTDWTIFLLDT | 281 | GLB1 | C*07:01 |

### Results:

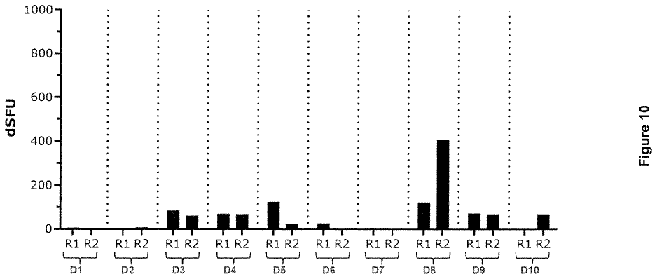

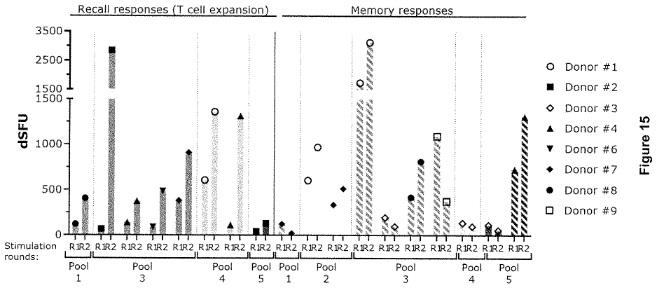

The in vitro immunogenicity of melanoma cancer antigens using the peptide pools stated in Table E4 is shown in FIGS. 10-14. The graphs show IFN-gamma readouts of the assay described in the present example. Summaries of the recall and memory responses defined according to the above-described criteria for uniform batch analysis are shown in FIG. 15 and Table E5.

TABLE E5
Summary of recall and memory responses shown in FIGS. 15

| | Amount of | Recall | Amount of | Memory |
|---|---|---|---|---|
| Peptide | recall | response - | memory | response - |
| pool | responses | PBMC donors | responses | PBMC donors |
| 1 | 1 | D8 | 1 | D5 |
| 2 | 0 | | 2 | D1, D7 |
| 3 | 4 | D2, D4, D6, D7 | 4 | D1, D3, D8, D9 |
| 4 | 2 | D1, D4 | 1 | D3 |
| 5 | 1 | D2 | 2 | D3, D4 |

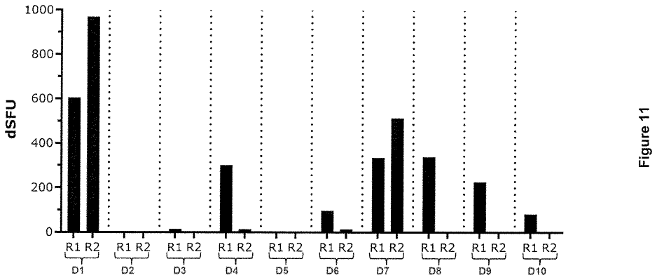

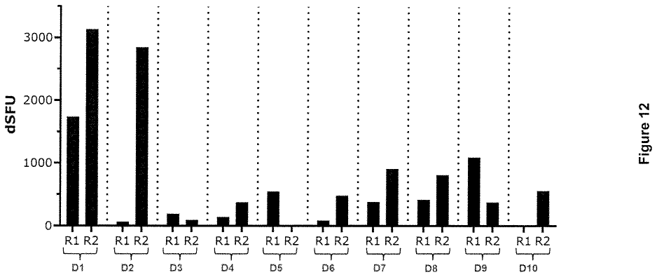

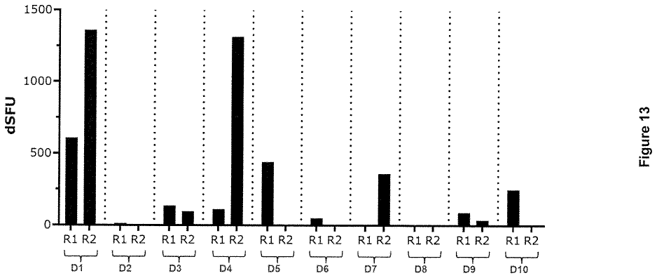

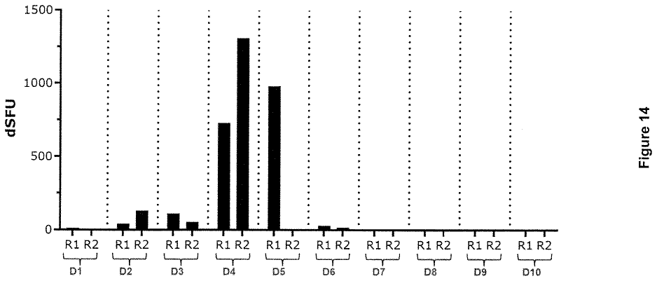

As shown in FIG. 10, 7 PBMC donors showed an antigen specific T cell response to at least one of the peptides of pool 1. 1 PBMC donor showed a recall response to at least one of the peptides of pool 1. 1 PBMC donor showed a memory response to at least one of the peptides of pool 1 (see also FIG. 15/Table E5). As shown in FIG. 11, 7 PBMC donors showed an antigen specific T cell response to at least one of the peptides of pool 2. 2 PBMC donor showed a memory response to at least one of the peptides of pool 2 (see also FIG. 15/Table E5). As shown in FIG. 12, 10 PBMC donors showed an antigen specific T cell response to at least one of the peptides of pool 3. 4 PBMC donors showed a recall response to at least one of the peptides of pool 3. 4 PBMC donors showed a memory response to at least one of the peptides of pool 3 (see also FIG. 15/Table E5). As shown in FIG. 13, 8 PBMC donors showed an antigen specific T cell response to at least one of the peptides of pool 4. 2 PBMC donors showed a recall response to at least one of the peptides of pool 4. 1 PBMC donor showed a memory response to at least one of the peptides of pool 4 (see also FIG. 15/Table E5). As shown in FIG. 14, 4 PBMC donors showed an antigen specific T cell response to at least one of the peptides of pool 5. 1 PBMC donor showed a recall response to at least one of the peptides of pool 5. 2 PBMC donors showed a memory response to at least one of the peptides of pool 5 (see also FIG. 15/Table E5).

In summary, every peptide pool comprises at least one cancer antigen that could lead to an immune response in human T cells. Both recall and memory responses could be observed in this experimental setup.

### Example 6.2: Antigen Validation—In Vitro Immunogenicity of NSCLC and Melanoma Cancer Antigens-Pooled Peptides

The goal of the experiment was to analyse the in vitro immunogenicity of different NSCLC and melanoma cancer antigens derived from tumour-specific neoantigens and encoded by lncRNA-derived smORFs.

In this assay, cryopreserved PBMCs from 10 multiple (healthy) donors with a series of different HLA types (see Table E6) and 8 different peptide pools (see Table E7) derived from NSCLC and melanoma cancer antigens were tested.

TABLE E6
Overview of PBMC donors used in Example
6.2 and corresponding HLA alleles

| | | | |
|---|---|---|---|
| Donor ID | HLA-A | HLA-B | HLA-C |

| | | | | | | |
|---|---|---|---|---|---|---|
| D1 | 24:02 | 68:02 | 07:02 | 14:02 | 07:02 | 08:02 |
| D2 | 02:01 | 03:01 | 35:01 | 51:01 | 04:01 | 14:02 |
| D3 | 01:01 | 11:01 | 52:01 | 53:01 | 04:01 | 12:02 |
| D4 | 02:01 | 03:01 | 27:05 | 51:01 | 01:02 | 01:02 |
| D5 | 02:05 | 03:01 | 35:01 | 50:01 | 04:01 | 06:02 |
| D6 | 01:01 | 23:01 | 41:01 | 44:02 | 05:01 | 17:01 |
| D7 | 11:01 | 32:01 | 14:01 | 35:01 | 04:01 | 08:02 |
| D8 | 01:02 | 24:02 | 18:01 | 49:01 | 07:01 | 12:03 |
| D9 | 02:01 | 02:01 | 08:01 | 35:01 | 04:01 | 07:01 |
| D10 | 03:01 | 03:01 | 07:02 | 27:05 | 07:02 | 01:02 |

TABLE E7
Overview of NSCLC/melanoma peptide pools used in Example 6.2

| | | SEQ ID | | Predicted | |
|---|---|---|---|---|---|
| | | NO: | | HLA binding | Expression |
| Pool | Sequence | Protein | Source | e.g. to [HLA-] | in |
| 1 | FPFSVEQSNLV | 243 | IncCLEC2D91412616 | B*51:01 | Melanoma |
| | KEEYALAFYNIDSSSV | 240 | IncCLEC2D91412616 | A*24:02 | |
| 2 | DQDSRSAPEPKKPEE | 229 | IncTRPC53116031801 | B*27:05 | Melanoma/ |
| | PASKFSSASNYAALS | 230 | IncTRPC53116031801 | A*24:02 | NSCLC |
| 3 | KEWMNAWKLTAGSHK | 148 | KCNMB2AS14633753 | B*50:01 | NSCLC |
| | AWKLTAGSHKAMTRK | 149 | KCNMB2AS14633753 | A*68:01 | |
| | RCDPELCWISCEENS | 150 | KCNMB2AS14633753 | A*01:01 | |
| | WISCEENSKEWMNAW | 151 | KCNMB2AS14633753 | B*40:02 | |
| 4 | SITALSYHFYKNHIHF | 170 | IncNTF35511951417 | A*24:02 | NSCLC |
| | VLNSDQNSGEMLRRNSA | 172 | IncNTF35511951417 | A*01:01 | |
| | ASTERVSEGFKDSIT | 173 | IncNTF35511951417 | | |
| | ALSYHFYKNHIHFFPPT | 174 | IncNTF35511951417 | A*24:02 | |
| | HFFPPTCPFSLSGTQAL | 175 | IncNTF35511951417 | | |
| 5 | REVQVTSIKETFLSM | 125 | IncWDR722415641696 | A*25:01 | NSCLC |
| | MYVSNREVQVTSIKE | 126 | IncWDR722415641696 | A*01:01 | |
| | TFLSMGPASGLMLGC | 127 | IncWDR722415641696 | | |
| | IKETFLSMGPASGLMLG | 128 | IncWDR722415641696 | B*57:01 | |
| 6 | AYLSGQPLEATTLCQL | 163 | IncZC3H86163144 | A*24:02 | NSCLC |
| | CVHAYLSGQPLEATT | 164 | IncZC3H86163144 | A*02:10 | |
| 7 | MLGCSRQEMGKLEKR | 129, | IncWDR722416421696 | A*01:01 | NSCLC |
| | | 145 | | | |
| | GCSRQEMGKLEKRTV | 130, | IncWDR722416421696 | | |
| | | 146 | | | |
| 8 | KITDFGRAKLLG | 257 | EGFR | A*01:01 | NSCLC |
| | VDSTPPPGTRFRAMAI | 261 | TP53 | A*03:01 | NSCLC |
| | KIGDFGLATKK | 265 | BRAF | A*02:01 | NSCLC |

### Results:

Exemplary in vitro immunogenicity results for the NSCLC/melanoma cancer antigens and the peptide pools stated in Table E7 are shown in FIG. 15. The graph shows IFN-gamma readouts of the assay described in the present example. Recall and memory responses shown in FIG. 15 are furthermore summarized in Table E8.

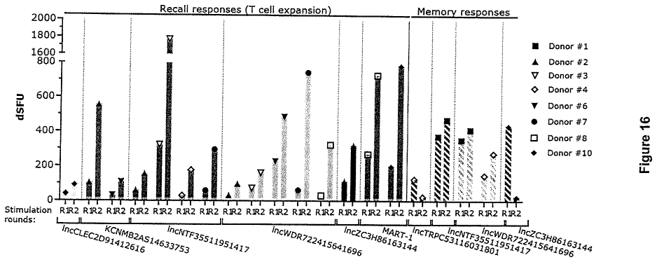

TABLE E8
Summary of recall and memory responses shown in FIG. 16

| | | Amount of | | Amount of | |
|---|---|---|---|---|---|
| | | recall | Recall response - | memory | Memory response - |
| Antigen | Pool | responses | PBMC donors | responses | PBMC donors |
| lncCLEC2D91412616 | 1 | 1 | D10 | | |
| lncTRPC53116031801 | 2 | | | 1 | D4 |
| KCNMB2AS14633753 | 3 | 2 | D2, D6 | | |
| lncNTF35511951417 | 4 | 4 | D2, D3, D4, D7 | 1 | D1 |
| lncWDR722415641696 | 5 | 5 | D2, D3, D6, D7, D8 | 2 | D1, D4 |
| lncZC3H86163144 | 6 | 1 | D2 | 1 | D10 |
| MART1 | | 2 | D8, D10 | | |

The condition Pool 8/Donor D9 in Example 6.2 showed an immune response characterized by a dSFU R1 and a dSFU R2 of 21.25 and 236, respectively.

In summary, lncRNA encoded peptides (lncCLEC2D91412616, KCNMB2AS14633753, lncZC3H86163144, lncNTF35511951417 and lncWDR722415641696) were immunogenic. Notably, the observed in vitro immunogenicity of lncRNA encoded peptides was comparable or even superior (lncNTF35511951417 and lncWDR722415641696) to the well-known immunogenic TAA MART-1, further underscoring their promise as cancer targets.

### Example 6.3: Antigen Validation—In Vitro Immunogenicity of Melanoma Cancer Antigens—Single Peptides

The goal of the experiment was to analyse the in vitro immunogenicity of different melanoma cancer antigens derived from tumour-specific neoantigens and encoded by lncRNAs.

Cryopreserved PBMCs from 5 (healthy) donors with a series of different HLA types (see Table E9) and 9 single peptides derived from melanoma cancer antigens (see Table E10) were tested.

TABLE E9
Overview of PBMC donors used in Example
6.3 and corresponding HLA alleles

| | | | |
|---|---|---|---|
| Donor ID | HLA-A | HLA-B | HLA-C |

| | | | | | | |
|---|---|---|---|---|---|---|
| D1 | 01:01 | 02:01 | 08:01 | 08:01 | 07:01 | 07:01 |
| D2 | 02:01 | 32:01 | 39:01 | 44:02 | 05:01 | 07:02 |
| D3 | 01:01 | 11:01 | 52:01 | 53:01 | 04:01 | 12:02 |
| D4 | 01:01 | 23:01 | 41:01 | 44:02 | 05:01 | 17:01 |
| D5 | 01:02 | 02:05 | 49:01 | 49:01 | 07:01 | 07:01 |

TABLE E10
Overview of melanoma single peptides

| | | | | Predicted HLA |
|---|---|---|---|---|
| Peptide | | SEQ ID NO: | | binding e.g. |
| identifier | Sequence | Protein | Source | to [HLA-] |
| 1 | ILDTAGLEEYS | 269 | NRAS | A*01:01 |
| 2 | HIAALKEMVVFLLLY | 286 | ATAD2 | C*07:01 |
| 3 | VTFLDLPVAIINLKE | 274 | TYW1B | C*07:01 |
| 4 | RIAGLFHLIILPLRS | 246 | LINC0089325197395 | A*02:01 |
| 5 | LQVLHECNSSYIVGF | 277 | MAP2K1 | C*05:01 |
| 6 | AGATFDLFLFVAQKM | 279 | TOMM22 | A*01:01 |
| 7 | YQDPAAVFFV | 275 | ECPAS | A*01:01 |
| 8 | SSNILTDWTIFLLDT | 281 | GLB1 | C*07:01 |
| 9 | KVAELVYFL | 282 | MAGE-A3 | C*07:01 |

In future experiments further single peptides from pools 1 and 2 of Table E7 are tested.

### Results:

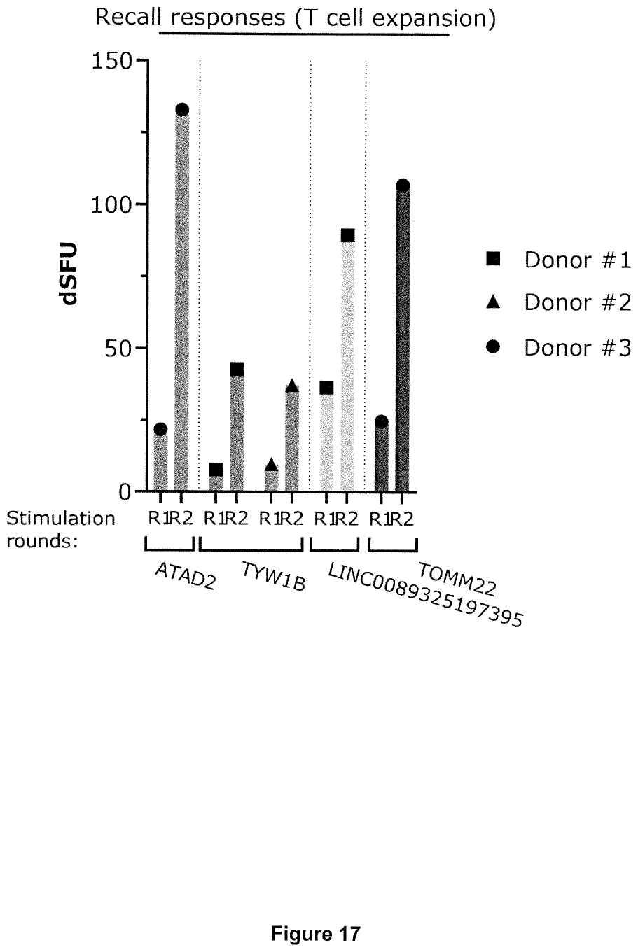

Exemplary in vitro immunogenicity results for the melanoma cancer antigens using the single peptides stated in Table E10 are shown in FIG. 17. The graph shows IFN-gamma readouts of the assay described in the present example. Recall responses shown in FIG. 17 are furthermore summarized in Table E10A.

TABLE E10A
Summary of recall responses shown in FIG. 17

| Peptide | | Amount of recall | Recall response - |
|---|---|---|---|
| ID | Source | responses | donors |
| 2 | ATAD2 | 1 | D3 |
| 3 | TYW1B | 2 | D1, D2 |
| 4 | LINC0089325197395 | 1 | D1 |
| 7 | TOMM22 | 1 | D3 |

### Example 6.4: Antigen Validation—In Vitro Immunogenicity of NSCLC and Melanoma Cancer Antigens-Single Peptides

The goal of the experiment is to analyse the in vitro immunogenicity of different NSCLC cancer antigens derived from tumour-specific neoantigens and encoded by lncRNAs. In this assay, cryopreserved PBMCs from multiple (healthy) donors with a series of different HLA types are tested with selected single NSCLC peptides from Table E7.

### Example 7: Antigen Validation—Immunopeptidomics

To further study and confirm epitope presentation on tumour cell MHC-I/II, immunopeptidomics analyses are performed. To this end, non-targeted mass spectrometry with primary samples from 15 melanoma (including BRAF mutated) and 15 NSCLC (10 LUSC+5 LUAD) patients is carried out according to standard procedures of the art. In addition, whole genome sequencing, RNA-seq and Ribo-seq on respective samples are carried out.

### Example 8: Preparation of Nucleic Acid Encoding Cancer Antigens

The present example provides methods of obtaining RNAs of the invention as well as methods of generating compositions of the invention comprising nucleic acid(s), in particular RNA, formulated in lipid-based carriers.

### Example 8.1. Preparation of DNA Templates for RNA In Vitro Transcription

DNA sequences encoding cancer antigens of the invention were prepared and used for subsequent RNA in vitro transcription reactions. Some DNA sequences were prepared by modifying the wild type or reference encoding DNA sequences by introducing a G/C optimized coding sequence for stabilization and expression optimization. Sequences were introduced into a pUC derived DNA vector to comprise a stabilizing UTR sequences and a stretch of adenosines and an optional histone stem-loop (hSL). The obtained plasmid DNA templates were transformed and propagated in bacteria using common protocols known in the art. Eventually, the plasmid DNA templates were extracted, purified, and linearized using a type II restriction enzyme. The herein used RNA constructs are provided in Table E11.

TABLE E11
RNA constructs used in the Examples (smORF nomenclature see in Table E12)

| | | SEQ ID | SEQ ID | SEQ ID |
|---|---|---|---|---|
| | | NO: | NO: | NO: |
| RNA ID | Name | Protein | CDS | RNA |
| R12293 | 5′UTR-smORF1-Linker(GS4)-3xHAtag-3′UTR-PolyA | 519 | 528 | 537 |
| R12294 | 5′UTR-smORF2-Linker(GS4)-3xHAtag-3′UTR-PolyA | 520 | 529 | 538 |
| R12295 | 5′UTR-smORF3-Linker(GS4)-3xHAtag-3′UTR-PolyA | 521 | 530 | 539 |
| R12296 | 5′UTR-smORF4-Linker(GS4)-3xHAtag-3′UTR-PolyA | 522 | 531 | 540 |
| R12297 | 5′UTR-smORF5-Linker(GS4)-3xHAtag-3′UTR-PolyA | 523 | 532 | 541 |
| R12298 | 5′UTR-smORF6-Linker(GS4)-3xHAtag-3′UTR-PolyA | 524 | 533 | 542 |
| R12299 | 5′UTR-smORF7-Linker(GS4)-3xHAtag-3′UTR-PolyA | 525 | 534 | 543 |
| R12300 | 5′UTR-SP(CTLA4)-Linker(GS4)-smORF1-Linker(GS4)-smORF2- | 526 | 535 | 544 |
| | Linker(GS4)-3xHAtag-Linker(GS4)-TMD/TMCD(CTLA4)-3′UTR-PolyA | | | |
| R12301 | 5′UTR-SP(CTLA4)-Linker(GS4)-smORF2-Linker(GS4)-smORF3- | 527 | 536 | 545 |
| | Linker(GS4)-smORF5-Linker(GS4)-smORF6-Linker(GS4)-smORF7- | | | |
| | Linker(GS4)-smORF4-Linker(GS4)-3xHAtag-Linker(GS4)- | | | |
| | TMD/TMCD(CTLA4)-3′UTR-PolyA | | | |

TABLE E12
smORF nomenclature used in Table E11

| Abbreviation | smORF | SEQ ID NO: Protein |
|---|---|---|
| smORF_1 | lncCLEC2D91412616 | 238 |
| smORF_2 | lncTRPC53116031801 | 227 |
| smORF_3 | KCNMB2AS14633753 | 147 |
| smORF_4 | lncNTF35511951417 | 169 |
| smORF_5 | lncWDR722415641696 | 124 |
| smORF_6 | lncZC3H86163144 | 162 |
| smORF_7 | lncWDR722416421696 | 144 |

### Example 8.2. RNA In Vitro Transcription from Plasmid DNA Templates

Linearized DNA templates were used for DNA dependent RNA in vitro transcription (IVT) using T7 RNA polymerase in the presence of a sequence optimized nucleotide mixture (ATP/GTP/CTP/UTP) and cap analog (for Cap1: m7G(5′)ppp(5′)(2′OMeA)pG; TriLink) under suitable buffer conditions. Other constructs are produced in the presence of a nucleotide mixture comprising (ATP/GTP/CTP/pseudouridine (ψ) or (ATP/GTP/CTP/N1-methylpseudouridine (m1ψ)) and a cap analog. After RNA in vitro transcription, the obtained RNA IVT reaction was subjected to purification steps comprising RP-HPLC.

### Example 8.3 Preparation of Lipid-Based Carriers Encapsulating the mRNA

LNPs used in the working examples were prepared using the NanoAssemblr™ microfluidic system (Precision NanoSystems lnc., Vancouver, BC) according to standard protocols which enables controlled, bottom-up, molecular self-assembly of nanoparticles via custom-engineered microfluidic mixing chips that enable millisecond mixing of nanoparticle components at a nanolitre scale. For preparation of lipid nanoparticle compositions the following excipients/lipids were used: (i) ionizable lipid: VitE-C4DE-Pip-thioether as described herein and in Table 1 of published patent application WO2021123332 (compound C26); (ii) cholesterol (Avanti Polar Lipids; Alabaster, AL) as described herein; (iii) neutral lipid/phospholipid “DPhyPE” (1,2-diphytanoyl-sn-glycero-3-phosphoethanolamine; Avanti Polar Lipids; Alabaster, AL) as described herein; (iv) phosphatidylserine DPhyPS (1,2-diphytanoyl-sn-glycero-3-phospho-L-serine; Avanti Polar Lipids; Alabaster, AL, 850408P) as described herein; and (v) a polymer conjugated lipid, PMOZ-lipid “PMOZ 4”, as described herein. The LNP compositions were furthermore characterized as described in Table E13.

TABLE E13
LNP compositions

| | Ionizable | | | | PMOz- | | | | |
|---|---|---|---|---|---|---|---|---|---|
| | lipid | Cholesterol | DPhyPE | DPhyPS | dm- | N:P | (lipid:RNA) | mRNA | mRNA |
| | (mol %) | (mol %) | (mol %) | (mol %) | amide | ratio | m/m ratio | Type | Length |
| 1 | 49 | 40 | 7.5 | 2.5 | 1 | 14 | 32.1 | R8730 | 1925 |
| 2 | 49 | 40 | 7.5 | 2.5 | 1 | 14 | 32.3 | R12300 | 1109 |
| 3 | 49 | 40 | 7.5 | 2.5 | 1 | 14 | 32.2 | R12301 | 1550 |

The lipids were solubilized in alcoholic solution (ethanol) according to standard procedures. In detail, LNPs were prepared by mixing appropriate volumes of lipid stock solutions for cholesterol, phospholipids, and polymer conjugated lipid in ethanol buffer (20 mg/ml in EtOH); the cationic lipid C26 was solubilized in 30 mg/ml t-butanol and added to the ethanol premix of lipids. Subsequently, this ethanol mix of lipids was combined with an aqueous phase (50 mM sodium acetate, pH 4.0) containing 1 g/L of mRNA. Briefly, mRNA as indicated in the working examples, was diluted to 0.05 to 0.2 mg/ml in 50 mM acetate buffer, pH 4. Syringe pumps were installed into inlet parts of the NanoAssemblr™ (Precision NanoSystems lnc., Vancouver, BC) and used to mix the ethanolic lipid solution with the mRNA aqueous solution at a ratio of about 1:5 to 1:3 (vol/vol) with total flow rates from about 14 ml/min to about 18 ml/min. The ethanol was then removed and the external buffer replaced with PBS/sucrose buffer (pH 7.4, 75 mM NaCl, 10 mM phosphate, 150 mM sucrose) by dialysis (Slide-A-Lyzer™ Dialysis Cassettes, ThermoFisher). Finally, the lipid nanoparticles were filtered through a 0.2 μm pore sterile filter. Lipid nanoparticle particle diameter size was from about 90 nm to about 140 nm as determined by quasi-elastic light scattering using a Malvern Zetasizer Nano (Malvern Instruments Ltd.; UK).

### Example 9: Translation of smORF Encoding mRNA and Stability of the Translated Protein

Translation of smORF encoding mRNA (mRNA constructs described in Table E11) and stability of the expressed protein was measured according to standard procedures known in the art using western blot and flow cytometry. HEK293T cells (400000 cells/well, 200000 cells/ml) were transfected with 1 μg/ml mRNA using Lipofectamin and the amount of the expressed protein was—after 4 h and 16-18 h incubation with and without Carfilzomib—measured by FACS using antibodies to the CTLA4 domains or HA-tag. Additionally, HeLa cells were transfected with mRNA, supplemented or not with Carfilzomib 2 h post-treatment, and harvested and lysed 18 h post-treatment. Subsequently, the amount of the expressed protein was assessed by Western blot using an antibody to the HA-tag. Using FACS and Western Blot, translation of mRNA constructs encoding single smORFs or several smORFs as well as the stability of their encoded fusion proteins could be confirmed via CTLA-4 or HA staining.

### Example 10: In Vitro Immunogenicity of mRNA Constructs Encoding Multiple smORFs and/or TSAs

mRNA constructs encoding for multiple smORFs and/or TSA are produced as described before (see R12300 and R12301 in Example 8). Alternatively, e.g. variants with helper epitope(s) replacing the HA tag are produced as follows: Variant R12300 (mRNA encoding for two smORFs expressed in melanoma): 5′UTR-SP(CTLA4)-Linker(GS4)-smORF1-Linker(GS4)-smORF2-Linker(GS4)-Helper epitope(s)-Linker(GS4)-TMD/TMCD (CTLA4)-3′UTR-PolyA Variant R12301 (mRNA encoding for six smORFs expressed in NSCLC): 5′UTR-SP(CTLA4)-Linker(GS4)-smORF2-Linker(GS4)-smORF3-Linker(GS4)-smORF5-Linker(GS4)-smORF6-Linker(GS4)-smORF7-Linker(GS4)-smORF4-Linker(GS4)-Helper epitope(s)-Linker(GS4)-TMD/TMCD (CTLA4)-3′UTR-PolyA

In vitro immunogenicity of mRNA constructs is analyzed by a similar assay as described in Example 6. The first stimulation cycle is performed with mRNA and for the second stimulation cycle the corresponding peptides are used.

### Example 11: In Vivo Immunogenicity of mRNA Constructs Encoding for smORFs

To analyze the in vivo immunogenicity of mRNA constructs encoding for smORFs, R12300 and R12301 constructs were produced and formulated as described in Example 8 and tested as follows.

For vaccination, the constructs were applied to female CB6F1 hybrid mice on days 0, 7 and day 14 intramuscularly (i.m.; Musculus tibialis) with doses of 5 μg LNP-formulated mRNA (R12300, R12301 or PpLuc control mRNA R8730).

21 days after the first mRNA administration, mice were sacrificed, and spleens were collected for further analysis. Cancer antigen-specific cellular responses in splenocyte samples obtained in this step were measured as antigen-specific T cell activation. This was analyzed by intracellular cytokine staining and subsequent analysis by flow cytometry according to standard protocols as follows: isolated splenocytes were individually restimulated with peptide libraries for each smORF (15-mer libraries covering the complete antigens) at 1 μg/ml final peptide concentration in the presence of anti-CD28 (BD Biosciences, San Jose, USA) for 6 hours with the addition of GolgiPlug (BD Biosciences) after 1h. Unstimulated splenocytes were treated the same way, but supplemented with DMSO instead of the peptide cocktail. Additional controls were splenocytes stimulated with PMA/ionomycin (no anti-CD28; PMA and ionomycin from Sigma-Aldrich; Merck KGaA, Darmstadt, Germany) (positive control) and splenocytes which were left unstained by fluorophore-conjugated antibodies (negative control). After stimulation, splenocytes were stained with surface and intracellular, fluorophore-conjugated antibodies and analysed by flow cytometry. CD8 and CD4 responses are shown as IFN-gamma/TNF-alpha double-positive CD8 and CD4 T cells.

### Results:

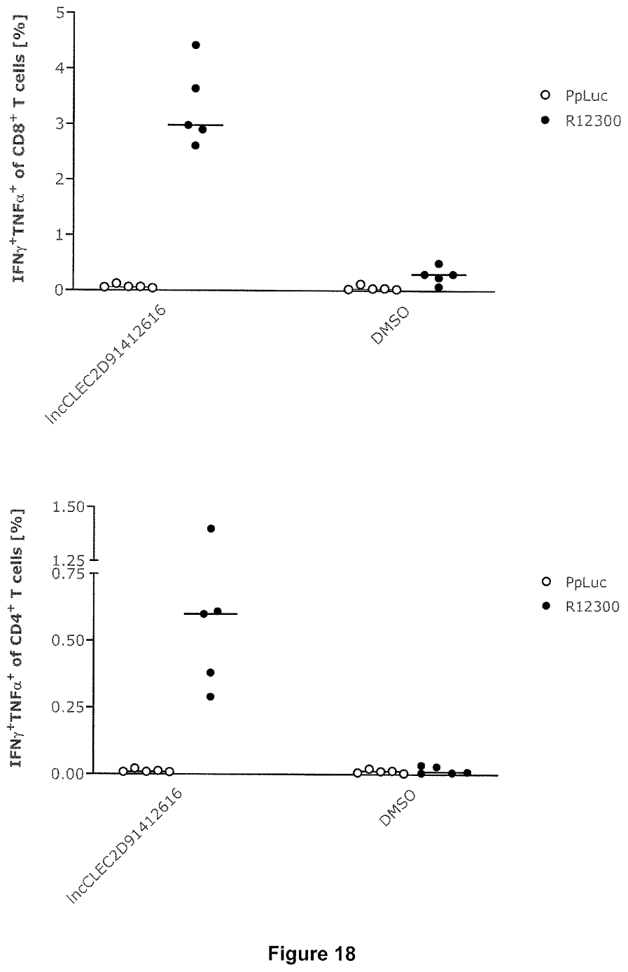

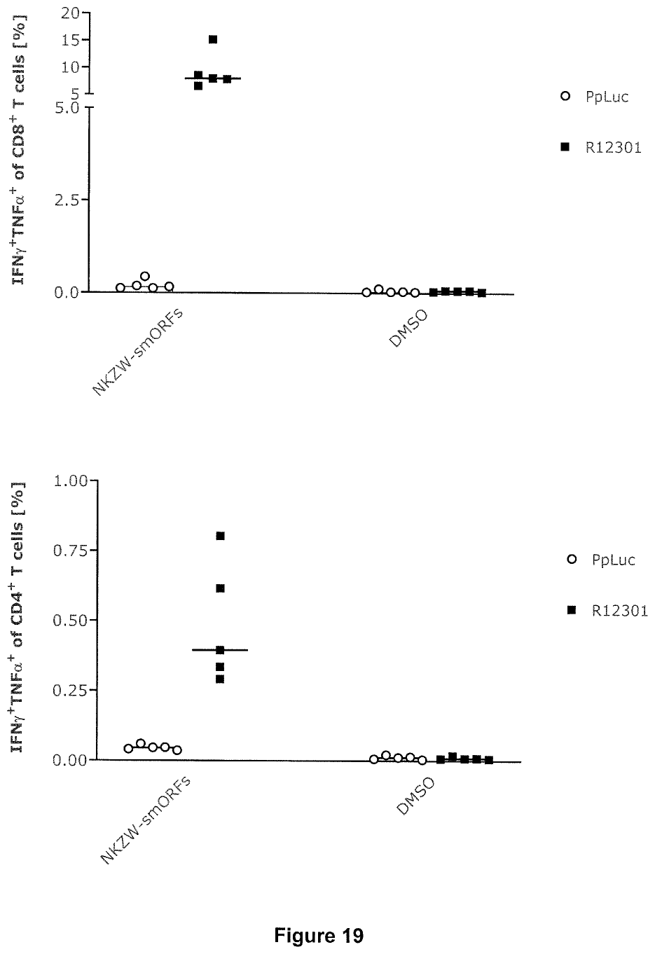

Exemplary data for the construct R12300 are shown in FIG. 18. The data shows that the mRNA R12300 raised potent CD8 (top) and CD4 (bottom) responses against the lncCLEC2D91412616 smORF. Exemplary data for the construct R12301 and the T-cell responses against the NKZW-smORFs (KCNMB2AS14633753, lncNTF35511951417, lncWDR722415641696, lncZC3H86163144) are shown in FIG. 19. The data shows that R12301 raised potent CD8 (top) and CD4 (bottom) responses. In summary, these data underline that mRNA encoding for smORFs can elicit potent immuneresponses against these encoded targets in vivo.

### Example 12: Broad lncRNA Indication Screening

The goal of the experiment was to analyze whether the lncRNAs KCNMB2-AS1:4, lnc-NTF3-5:5, lnc-WDR72-2:4, lnc-ZC3H8-6:1 and lnc-CLEC2D-9:1 are (over) expressed in indications beyond melanoma and NSCLC.

To this end, all indications in the TCGA tumor database were analyzed. For each indication in the TCGA tumour database and lncRNA, the ratio of expression in tumour (TCGA) to expression in normal tissue (GTEX) was calculated; only indications with strong overexpression in tumour compared to healthy tissue were further considered. Subsequently, it was determined what proportion of patients in a given indication express the lncRNA in the tumour.

In this context, for a lncRNA to be considered expressed in the tumour of an individual patient, the tumour expression had to exceed a certain threshold value, which was a multiple of the expression in healthy tissue for said lncRNA.

### Results:

Table E14 shows examples of tumour expression values (TCGA mean) as well as determined expression thresholds that were used to calculate the proportion of patients who (over) express the lncRNA in a specific indication.

TABLE E14
Examples of tumor expression values
and applied expression thresholds

| | | TCGA | Expression |
|---|---|---|---|
| | | mean | threshold |
| TCGA study/indication | Transcript ID | [TPM] | [TPM] |
| Bladder Urothelial Carcinoma | lnc-WDR72-2:4 | 3.37 | 2.04 |
| Colon adenocarcinoma | lnc-WDR72-2:4 | 1.75 | 2.04 |
| Esophageal carcinoma | lnc-WDR72-2:4 | 4.17 | 2.04 |
| Head and Neck squamous cell | lnc-NTF3-5:5 | 2.08 | 2.60 |
| carcinoma | | | |
| Head and Neck squamous cell | lnc-WDR72-2:4 | 2.73 | 2.04 |
| carcinoma | | | |
| Ovarian serous | lnc-NTF3-5:5 | 3.18 | 2.60 |
| cystadenocarcinoma | | | |
| Stomach adenocarcinoma | lnc-WDR72-2:4 | 1.72 | 2.04 |
| Testicular Germ Cell Tumors | lnc-CLEC2D-9:1 | 7.43 | 2.19 |
| Testicular Germ Cell Tumors | lnc-NTF3-5:5 | 3.91 | 2.60 |
| Thymoma | lnc-NTF3-5:5 | 3.09 | 2.60 |
| Uterine Carcinosarcoma | lnc-NTF3-5:5 | 3.22 | 2.60 |
| Uveal Melanoma | lnc-CLEC2D-9:1 | 6.72 | 2.19 |

Table E15 summarizes indications in the TCGA database that (over) expressed at least one of the five studied lncRNAs as well as the proportion of patients in a given indication that (over) expressed the one or more lncRNAs. As can be seen in this table, the five lncRNAs are widely expressed across a multitude of indications and to high extents within those indications (ranging from 26-94% of patients in a given study/indication). Notably, the four lncRNAs lnc-KCNMB2-AS1:4, KCNMB2-AS1:4, WDR72-2:4, lnc-ZC3H8-6:1 are particularly (over) expressed in squamous cell carcinomas, namely LUSC, HSNCC, cervical carcinoma and esophageal carcinoma. In the context of sarcomas, it was observed that lnc-ZC3H8-6:1 is expressed in synovial sarcomas.

TABLE E15
Broad lncRNA indication screening

| | | lnc- | lnc- | lnc- | lnc- |
|---|---|---|---|---|---|
| | KCNMB2- | NTF3- | WDR72- | ZC3H8- | CLEC2D- |
| TCGA study/indication | AS1:4 | 5:5 | 2:4 | 6:1 | 9:1 |
| Adrenocortical carcinoma | | | | 76 | |
| Bladder Urothelial Carcinoma | 46 | | 41 | 75 | |
| Breast invasive carcinoma | | | | 48 | |
| Cervical carcinoma (Cervical | 31 | | | 67 | |
| squamous cell carcinoma and | | | | | |
| endocervical adenocarcinoma) | | | | | |
| Colon adenocarcinoma | | | 40 | 53 | |
| Esophageal carcinoma | | | 48 | | |
| Head and Neck squamous cell | 46 | 26 | 47 | 62 | |
| carcinoma (HSNCC) | | | | | |
| Kidney renal papillary cell carcinoma | | | | 60 | |
| Lung adenocarcinoma | | | | 58 | |
| Lung squamous cell carcinoma | 81 | 57 | 56 | 63 | |
| Lymphoid Neoplasm Diffuse Large B- | | | | 76 | |
| cell Lymphoma | | | | | |
| Ovarian serous cystadenocarcinoma | | 38 | | | |
| Rectum adenocarcinoma | | | | 62 | |
| Sarcoma | | | | 60 | |
| Skin Cutaneous Melanoma | | | | 70 | 52 |
| Stomach adenocarcinoma | | | 29 | | |
| Testicular Germ Cell Tumors | | 42 | | | 41 |
| Thymoma | 39 | 38 | | 83 | |
| Thyroid carcinoma | | | | 59 | |
| Hepatocellular carcinoma | | | | 61 | |
| Uterine Carcinosarcoma | | 40 | | 94 | |
| Uterine Corpus Endometrial Carcinoma | 46 | | | 89 | |
| Uveal Melanoma | | | | | 31 |

Numbers show the proportion of patients in a given indication considered to express the indicated lncRNA.

1. An artificial nucleic acid comprising at least one coding sequence encoding at least one tumour antigen that comprises or consists of at least one antigenic peptide selected from a peptide or protein encoded by a long non-coding RNA (lncRNA), or an immunogenic fragment or variant thereof.

2. The artificial nucleic acid of claim 1, wherein the at least one antigenic peptide is selected from a peptide or protein encoded by a long non-coding RNA (lncRNA) selected from lnc-NTF3-5:5, lnc-WDR72-2:4, KCNMB2-AS1:4, lnc-ZC3H8-6:1, lnc-CLEC2D-9:1, lnc-TRPC5-3:1, or LINC00893:25, or an immunogenic fragment or variant of any of these.

3. The artificial nucleic acid of claims 1 to 2, wherein the at least one antigenic peptide encoded by the long non-coding RNA (lncRNA) comprises at least one T-cell epitope, preferably at least one CD8+ T cells epitope.

4. The artificial nucleic acid of claims 1 to 3, wherein the at least one antigenic peptide selected from a peptide or protein encoded by a long non-coding RNA (lncRNA) comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 124-254, 546-650, 756-763, or an immunogenic fragment or variant of any of these.

5. The artificial nucleic acid of claims 1 to 4, comprising
at least one coding sequence encoding at least one tumour antigen that comprises at least one antigenic peptide selected from a peptide or protein encoded by lnc-NTF3-5:5, wherein the antigenic peptide comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 169-226, 604-629, 760, or an immunogenic fragment or variant of any of these; or
at least one coding sequence encoding at least one tumour antigen that comprises at least one antigenic peptide selected from a peptide or protein encoded by lnc-WDR72-2:4, wherein the antigenic peptide comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 124-146, 546-571, 756, 757, or an immunogenic fragment or variant of any of these; or
at least one coding sequence encoding at least one tumour antigen that comprises at least one antigenic peptide selected from a peptide or protein encoded by lnc-TRPC5-3:1, wherein the antigenic peptide comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 227-237, 630-642, 761, or an immunogenic fragment or variant of any of these; or
at least one coding sequence encoding at least one tumour antigen that comprises at least one antigenic peptide selected from a peptide or protein encoded by lnc-CLEC2D-9:1, wherein the antigenic peptide comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 238-243, 643-650, 762, or an immunogenic fragment or variant of any of these; or
at least one antigenic peptide selected from a peptide or protein encoded by KCNMB2-AS1:4, wherein the antigenic peptide comprises or consists of at least one of the amino acid sequences being identical or at least 80% identical to any one of SEQ ID NOs: 147-161, 572-589, 758, or an immunogenic fragment or variant of any of these; or
at least one coding sequence encoding at least one tumour antigen that comprises at least one antigenic peptide selected from a peptide or protein encoded by lnc-ZC3H8-6:1, wherein the antigenic peptide comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 162-168, 590-603, 759, or an immunogenic fragment or variant of any of these; or
at least one coding sequence encoding at least one tumour antigen that comprises at least one antigenic peptide selected from a peptide or protein encoded by LINC00893:25, wherein the antigenic peptide comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 244-254, 763, or an immunogenic fragment or variant of any of these.

6. The artificial nucleic acid of claims 1 to 5, comprising
at least one coding sequence encoding at least one tumour antigen that comprises at least one antigenic peptide selected from a peptide or protein encoded by lnc-NTF3-5:5, wherein the antigenic peptide comprises or consists of at least one of the amino acid sequences being identical or at least 80% identical to SEQ ID NOs: 760, or an immunogenic fragment or variant thereof, or
at least one coding sequence encoding at least one tumour antigen that comprises at least one antigenic peptide selected from a peptide or protein encoded by lnc-WDR72-2:4, wherein the antigenic peptide comprises or consists of at least one of the amino acid sequences being identical or at least 80% identical to SEQ ID NOs: 756, or an immunogenic fragment or variant thereof; or
at least one antigenic peptide selected from a peptide or protein encoded by KCNMB2-AS1:4, wherein the antigenic peptide comprises or consists of at least one of the amino acid sequences being identical or at least 80% identical to SEQ ID NOs: 758, or an immunogenic fragment or variant thereof; or
at least one coding sequence encoding at least one tumour antigen that comprises at least one antigenic peptide selected from a peptide or protein encoded by lnc-ZC3H8-6:1, wherein the antigenic peptide comprises or consists of at least one of the amino acid sequences being identical or at least 80% identical to SEQ ID NOs: 759, or an immunogenic fragment or variant thereof, or
at least one coding sequence encoding at least one tumour antigen that comprises at least one antigenic peptide selected from a peptide or protein encoded by lnc-CLEC2D-9:1, wherein the antigenic peptide comprises or consists of at least one of the amino acid sequences being identical or at least 80% identical to SEQ ID NOs: 762, or an immunogenic fragment or variant thereof.

7. The artificial nucleic acid of claims 1 to 6, wherein the at least one coding sequence comprises a nucleic acid sequence, preferably a GC optimized nucleic acid sequence, encoding at least one antigenic peptide selected from a peptide or protein encoded by a lncRNA, wherein the nucleic acid sequence is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 318-448, 651-755, 764-771, or a fragment or a variant of any of these.

8. The artificial nucleic acid of claims 1 to 7, comprising
at least one coding sequence comprising a nucleic acid sequence encoding at least one tumour antigen that comprises at least one antigenic peptide selected from a peptide or protein encoded by lnc-NTF3-5:5, wherein the nucleic acid sequence is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 363-420, 709-734, 768, or a fragment or a variant of any of these; or
at least one coding sequence comprising a nucleic acid sequence encoding at least one tumour antigen that comprises at least one antigenic peptide selected from a peptide or protein encoded by lnc-WDR72-2:4, wherein the nucleic acid sequence is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 318-340, 651-676, 764, 765, or a fragment or a variant of any of these; or
at least one coding sequence comprising a nucleic acid sequence encoding at least one tumour antigen that comprises at least one antigenic peptide selected from a peptide or protein encoded by lnc-TRPC5-3:1, wherein the nucleic acid sequence is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 421-431, 735-747, 769, or a fragment or a variant of any of these; or
at least one coding sequence comprising a nucleic acid sequence encoding at least one tumour antigen that comprises at least one antigenic peptide selected from a peptide or protein encoded by lnc-CLEC2D-9:1, wherein the nucleic acid sequence is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 432-437, 748-755, 770, or a fragment or a variant of any of these; or
at least one coding sequence comprising a nucleic acid sequence encoding at least one tumour antigen that comprises at least one antigenic peptide selected from a peptide or protein encoded by KCNMB2-AS1:4, wherein the nucleic acid sequence is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 341-355, 677-694, 766, or a fragment or a variant of any of these; or
at least one coding sequence comprising a nucleic acid sequence encoding at least one tumour antigen that comprises at least one antigenic peptide selected from a peptide or protein encoded by lnc-ZC3H8-6:1, wherein the nucleic acid sequence is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 356-362, 695-708, 767, or a fragment or a variant of any of these; or
at least one coding sequence comprising a nucleic acid sequence encoding at least one tumour antigen that comprises at least one antigenic peptide selected from a peptide or protein encoded by LINC00893:25, wherein the nucleic acid sequence is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 438-448, 771, or a fragment or a variant of any of these;

9. The artificial nucleic acid of any one of the preceding claims, wherein the at least one coding sequence encodes 2 to 10 different tumour antigens, preferably 2 to 5 different tumour antigens.

10. The artificial nucleic acid of any one of the preceding claims, wherein the at least one coding sequence encodes at least one additional amino acid sequence selected from at least one immune response activating signal transduction protein, at least one T-helper epitope, and/or at least one signal peptide.

11. The artificial nucleic acid of any one of the preceding claims, wherein the at least one coding sequence is a codon modified coding sequence, preferably wherein codon modified coding sequence is selected from a C maximized coding sequence, a CAI maximized coding sequence, human codon usage adapted coding sequence, a G/C content modified coding sequence, and a G/C optimized coding sequence, or any combination thereof.

12. The artificial nucleic acid of claim 11, wherein the at least one codon modified coding sequence is a G/C optimized coding sequence.

13. The artificial nucleic acid of any one of the preceding claims, wherein the nucleic acid comprises at least one untranslated region (UTR) preferably selected from at least one heterologous 5′-UTR and/or at least one heterologous 3′-UTR.

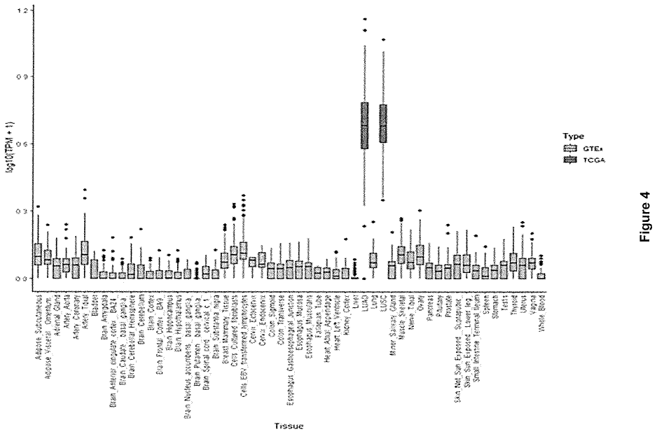

14. The artificial nucleic acid of claim 13, wherein the at least one heterologous 3′-UTR comprises or consists of a nucleic acid sequence from a 3′-UTR of a gene selected from PSMB3, ALB7, alpha-globin, beta-globin, ANXA4, CASP1, COX6B1, FIG. 4, GNAS, NDUFA1, RPS9, SLC7A3 or TUBB4B, or from a homolog, a fragment or a variant of any one of these genes, preferably wherein the at least one heterologous 3′-UTR comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 66-95, 112-123, or a fragment or a variant of any of these.

15. The artificial nucleic acid of claim 13, wherein the at least one heterologous 5′-UTR comprises or consists of a nucleic acid sequence from a 5′-UTR of a gene selected from HSD17B4, RPL32, AIG1, alpha-globin, ASAH1, ATP5A1, COX6C, DPYSL2, MDR, MP68, NDUFA4, NOSIP, RPL31, RPL35A, SLC7A3, TUBB4B, or UBQLN2, or from a homolog, a fragment or variant of any one of these genes, preferably wherein the at least one heterologous 5′-UTR comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 12-45, 64, 65, or a fragment or a variant of any of these.

16. The artificial nucleic acid of claims 13 to 15, wherein the at least one heterologous 5′-UTR is selected from HSD17B4 and the at least one heterologous 3′ UTR is selected from PSMB3.

17. The artificial nucleic acid of any one of the preceding claims, wherein the artificial nucleic acid is selected from a DNA or an RNA, preferably an RNA.

18. The artificial nucleic acid of any one of the preceding claims, wherein the artificial nucleic acid is an RNA selected from mRNA, circular RNA, replicon RNA, or viral RNA, preferably an mRNA.

19. The artificial nucleic acid of any one of the preceding claims, wherein the artificial nucleic acid, preferably the RNA, comprises at least one poly(A) sequence, preferably wherein the at least one poly(A) sequence comprises about 40 to about 500 adenosine nucleotides.

20. The artificial nucleic acid of claim 19, wherein the at least one poly(A) sequence comprises about 60 to about 150 adenosine nucleotides, preferably about 100 adenosine nucleotides.

21. The artificial nucleic acid of claim 19 or 20, wherein the at least one poly(A) sequence is located at the 3′ terminus, optionally, wherein the 3′ terminal nucleotide is an adenosine.

22. The artificial nucleic acid of any one of the preceding claims, wherein the artificial nucleic acid, preferably the RNA, comprises at least one histone stem-loop sequence, wherein said histone stem-loop sequence preferably comprises or consists of a nucleic acid sequence identical or at least 70%, 80%, 90%, 91%, 92%, 93%, 94%, 95%, 96%, 97%, 98%, or 99% identical to any one of SEQ ID NOs: 3 or 4, or a fragment or variant of any of these.

23. The artificial nucleic acid of any one of the preceding claims, wherein the artificial nucleic acid is an RNA that comprises at least one modified nucleotide, preferably a modified nucleotide selected from pseudouridine (ψ) or N1-methylpseudouridine (m1ψ), more preferably N1-methylpseudouridine (m1ψ).

24. The artificial nucleic acid of claims 1 to 22, wherein the nucleic acid is an RNA that does not comprise a modified nucleotide.

25. The artificial nucleic acid of any one of the preceding claims, wherein the artificial nucleic acid is an RNA that comprises a 5′-cap structure, preferably a cap1 structure or a modified cap1 structure.

26. The artificial nucleic acid of any one of the preceding claims, wherein the artificial nucleic acid is an in vitro transcribed RNA.

27. The artificial nucleic acid of any one of the preceding claims, wherein the artificial nucleic acid, preferably the RNA, comprises the following sequence elements preferably in 5′- to 3′-direction:
A) a 5′-cap structure;
B) a 5′-UTR, preferably selected from a 5′-UTR of a HSD17B4 gene;
C) a coding sequence encoding at least one tumour antigen;
D) a 3′-UTR, preferably selected from a 3′-UTR of a PSMB3 gene;
E) optionally, a histone stem-loop; and
F) a poly(A) sequence, preferably comprising about 100 A nucleotides.

28. The artificial nucleic acid of any one of the preceding claims, wherein the artificial nucleic acid is an mRNA that comprises the following sequence elements in 5′- to 3′-direction:
A) a 5′-cap structure;
B) a 5′-UTR that comprises or consists of a nucleic acid sequence being identical or at least 80% identical to SEQ ID NO: 13, or a fragment or a variant thereof;
C) a coding sequence that comprises a nucleic acid sequence encoding at least one antigenic peptide selected from a peptide or protein encoded by a long non-coding RNA (lncRNA), wherein the nucleic acid sequence is identical or at least 80% identical to any one of SEQ ID NOs: 318-448, 651-755, 764-771, or a fragment or a variant of any of these;
D) a 3′-UTR that comprises or consists of a nucleic acid sequence being identical or at least 80% identical to SEQ ID NO: 67, or a fragment or a variant thereof;
E) optionally, a histone stem-loop; and
F) a poly(A) sequence, preferably comprising about 100 A nucleotides.

29. A pharmaceutical composition comprising at least one artificial nucleic acid comprising at least one coding sequence encoding at least one tumour antigen as defined by any one of the claims 1 to 28.

30. The pharmaceutical composition of claim 29, comprising a plurality of artificial nucleic acids each encoding at least one different tumour antigen.

31. The pharmaceutical composition of claim 29 or 30, wherein the at least one artificial nucleic acid, preferably the RNA, is formulated in at least one cationic or polycationic compound, wherein the at least one cationic or polycationic compound is optionally selected from a cationic or polycationic polymer, a cationic or polycationic polysaccharide, a cationic or polycationic lipid, a cationic or polycationic protein, a cationic or polycationic peptide, or any combinations thereof.

32. The pharmaceutical composition of claims 29 to 31, wherein the at least one artificial nucleic acid, preferably the RNA, is formulated in lipid-based carriers.

33. The pharmaceutical composition of claim 32, wherein the lipid-based carriers are selected from liposomes, lipid nanoparticles, lipoplexes, solid lipid nanoparticles, lipo-polyplexes, and/or nanoliposomes.

34. The pharmaceutical composition of claim 32 or 33, wherein the lipid-based carriers are lipid nanoparticles.

35. The pharmaceutical composition of claims 32 to 34, wherein the lipid-based carriers, preferably the LNPs, comprise
(i) at least one cationic lipid;
(ii) at least one neutral lipid;
(iii) at least one steroid or steroid analogue; and
(iv) at least one aggregation reducing lipid.

36. The pharmaceutical composition of claims 32 to 37, wherein the lipid-based carriers have a Z-average size in a range of about 50 nm to about 200 nm, preferably from about 50 nm to about 150 nm.

37. The pharmaceutical composition or the artificial nucleic acid of any one of the preceding claims, wherein upon intramuscular, intratumoral, or intravenous administration of the composition or nucleic acid to a cell, tissue, or subject, the encoded tumour antigens are produced.

38. The pharmaceutical composition or the artificial nucleic acid of any one of the preceding claims, wherein upon intramuscular, intratumoral, or intravenous administration of the composition or nucleic acid to a cell, tissue, or subject, the encoded tumour antigens are produced and induce an epitope-specific CD8+ T cells in the subject.

39. A tumour antigen or a composition of at least one tumour antigen, wherein the tumour antigen is characterized by any one of the preceding claims, preferably by any one of the claims 2 to 6, more preferably wherein the tumour antigen comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 124-254, 546-650, 756-763, or an immunogenic fragment or immunogenic variant of any of these.

40. An antibody, a T-cell, or a TCR that has been raised against or stimulated with any one of the tumour antigens or nucleic acids as defined in any one of the preceding claims, preferably raised against or stimulated with a tumour antigen of claim 39.

41. A combination comprising at least two or a plurality of therapeutic modalities selected from
a) at least one artificial nucleic acid as defined in claims 1 to 28; and/or
b) at least one pharmaceutical composition as defined in claims 29 to 38; and/or
c) at least one tumour antigen or composition as defined in claim 39; and/or
d) at least one antibody, T-cell, or TCR as defined in claim 40;

42. A kit or kit of parts comprising at least one artificial nucleic acid of any one of claims 1 to 28, and/or at least one pharmaceutical composition of any one of claims 29 to 38, and/or at least one tumour antigen or a composition of tumour antigens of claim 39, and/or at least one antibody, a T-cell, or a TCR of claim 40, optionally comprising a liquid vehicle for solubilising, and optionally comprising technical instructions providing information on administration and dosage of the components.

43. An artificial nucleic acid of any one of claims 1 to 28, a pharmaceutical composition of any one of claims 29 to 38, a tumour antigen or a composition of tumour antigens of claim 39, an antibody, T-cell, or a TCR of claim 40, a combination of claim 41, or a kit or kit of parts of claim 42, for use as a medicament.

44. An artificial nucleic acid of any one of claims 1 to 28, a pharmaceutical composition of any one of claims 29 to 38, a tumour antigen or a composition of tumour antigens of claim 39, an antibody, T-cell, or TCR of claim 40, a combination of claim 41, or a kit or kit of parts of claim 42, for use as a medicament in treating or preventing cancer in a subject, or any disease, disorder, or condition related to cancer.

45. An artificial nucleic acid of any one of claims 1 to 28, a pharmaceutical composition of any one of claims 29 to 38, a tumour antigen or a composition of tumour antigens of claim 39, an antibody, T-cell, or TCR of claim 40, a combination of claim 41, or a kit or kit of parts of claim 42, for use as a medicament in treating or preventing squamous cell carcinoma in a subject, or any disease, disorder, or condition related to squamous cell carcinoma.

46. An artificial nucleic acid of any one of claims 1 to 28, a pharmaceutical composition of any one of claims 29 to 38, a tumour antigen or a composition of tumour antigens of claim 39, an antibody, T-cell, or TCR of claim 40, a combination of claim 41, or a kit or kit of parts of claim 42, for use as a medicament in treating or preventing NSCLC in a subject, or any disease, disorder, or condition related to NSCLC, in particular squamous cell carcinomas/squamous non-small-cell lung cancer.

47. The use as a medicament in treating or preventing NSCLC of claim 46, wherein the at least one tumour antigen that is provided by the artificial nucleic acid, the pharmaceutical composition, the tumour antigen or a composition of tumour antigens, the combination, or the kit or kit of parts, comprises at least one antigenic peptide selected from a peptide or protein encoded by a long non-coding RNA (lncRNA) selected from lnc-WDR72-2:4, lnc-TRPC5-3:1, KCNMB2-AS1:4, lnc-NTF3-5:5, or lnc-ZC3H8-6:1, or a fragment or variant of any of these.

48. An artificial nucleic acid of any one of claims 1 to 28, a pharmaceutical composition of any one of claims 29 to 38, a tumour antigen or a composition of tumour antigens of claim 39, an antibody, T-cell, or TCR of claim 40, a combination of claim 41, or a kit or kit of parts of claim 42 for use as a medicament in treating or preventing HNSCC in a subject, or any disease, disorder, or condition related to HNSCC.

49. The use as a medicament in treating or preventing HNSCC of claim 48, wherein the at least one tumour antigen that is provided by the artificial nucleic acid, the pharmaceutical composition, the tumour antigen or a composition of tumour antigens, the combination, or the kit or kit of parts, comprises at least one antigenic peptide selected from a peptide or protein encoded by a long non-coding RNA (lncRNA) selected from lnc-WDR72-2:4, KCNMB2-AS1:4, lnc-NTF3-5:5, or lnc-ZC3H8-6:1, or a fragment or variant of any of these.

50. An artificial nucleic acid of any one of claims 1 to 28, a pharmaceutical composition of any one of claims 29 to 38, a tumour antigen or a composition of tumour antigens of claim 39, an antibody, T-cell, or TCR of claim 40, a combination of claim 41, or a kit or kit of parts of claim 42 for use as a medicament in treating or preventing melanoma in a subject, or any disease, disorder, or condition related to melanoma.

51. The use as a medicament in treating or preventing melanoma of claim 50, wherein the at least one tumour antigen that is provided by the artificial nucleic acid, the pharmaceutical composition, the tumour antigen or a composition of tumour antigens, the combination, or the kit or kit of parts, comprises at least one antigenic peptide selected from a peptide or protein encoded by a long non-coding RNA (lncRNA) selected from lnc-CLEC2D-9:1, lnc-TRPC5-3:1, or LINC00893:25, or a fragment or variant of any of these.

52. An artificial nucleic acid of any one of claims 1 to 28, a pharmaceutical composition of any one of claims 29 to 38, a tumour antigen or a composition of tumour antigens of claim 39, an antibody, T-cell, or TCR of claim 40, a combination of claim 41, or a kit or kit of parts of claim 42, for use as a medicament in treating or preventing Adrenocortical carcinoma, Bladder Urothelial Carcinoma, Breast invasive carcinoma, Cervical carcinoma (Cervical squamous cell carcinoma and endocervical adenocarcinoma), Colon adenocarcinoma, Kidney renal papillary cell carcinoma, Lung adenocarcinoma, Lymphoid Neoplasm Diffuse Large B-cell Lymphoma, Rectum adenocarcinoma, Sarcoma, Skin Cutaneous Melanoma, Thymoma, Thyroid carcinoma, Hepatocellular carcinoma, Uterine Carcinosarcoma, or Uterine Corpus Endometrial Carcinoma in a subject, or any related disease, disorder, or condition, wherein the at least one tumour antigen that is provided by the artificial nucleic acid, the pharmaceutical composition, the tumour antigen or a composition of tumour antigens, the combination, or the kit or kit of parts, comprises at least one antigenic peptide selected from a peptide or protein encoded by lnc-ZC3H8-6:1, or a fragment or variant thereof.

53. An artificial nucleic acid of any one of claims 1 to 28, a pharmaceutical composition of any one of claims 29 to 38, a tumour antigen or a composition of tumour antigens of claim 39, an antibody, T-cell, or TCR of claim 40, a combination of claim 41, or a kit or kit of parts of claim 42 for use as a medicament in treating or preventing Bladder Urothelial Carcinoma, Cervical carcinoma (Cervical squamous cell carcinoma and endocervical adenocarcinoma), Thymoma, or Uterine Corpus Endometrial Carcinoma in a subject, or any related disease, disorder, or condition related, wherein the at least one tumour antigen that is provided by the artificial nucleic acid, the pharmaceutical composition, the tumour antigen or a composition of tumour antigens, the combination, or the kit or kit of parts comprises at least one antigenic peptide selected from a peptide or protein encoded by KCNMB2-AS1:4, or a fragment or variant thereof.

54. An artificial nucleic acid of any one of claims 1 to 28, a pharmaceutical composition of any one of claims 29 to 38, a tumour antigen or a composition of tumour antigens of claim 39, an antibody, T-cell, or TCR of claim 40, a combination of claim 41, or a kit or kit of parts of claim 42 for use as a medicament in treating or preventing Ovarian serous cystadenocarcinoma, Testicular Germ Cell Tumors, Thymoma, or Uterine carcinosarcoma in a subject, or any related disease, disorder, or condition related, wherein the at least one tumour antigen that is provided by the artificial nucleic acid, the pharmaceutical composition, the tumour antigen or a composition of tumour antigens, the combination, or the kit or kit of parts, comprises at least one antigenic peptide selected from a peptide or protein encoded by lnc-NTF3-5:5, or a fragment or variant thereof.

55. An artificial nucleic acid of any one of claims 1 to 28, a pharmaceutical composition of any one of claims 29 to 38, a tumour antigen or a composition of tumour antigens of claim 39, an antibody, T-cell, or TCR of claim 40, a combination of claim 41, or a kit or kit of parts of claim 42 for use as a medicament in treating or preventing Bladder Urothelial Carcinoma, Colon adenocarcinoma, Esophageal carcinoma, or Stomach adenocarcinoma in a subject, or any related disease, disorder, or condition, wherein the at least one tumour antigen that is provided by the artificial nucleic acid, the pharmaceutical composition, the tumour antigen or a composition of tumour antigens, the combination, or the kit or kit of parts, comprises at least one antigenic peptide selected from a peptide or protein encoded by lnc-WDR72-2:4, or a fragment or variant thereof.

56. An artificial nucleic acid of any one of claims 1 to 28, a pharmaceutical composition of any one of claims 29 to 38, a tumour antigen or a composition of tumour antigens of claim 39, an antibody, T-cell, or TCR of claim 40, a combination of claim 41, or a kit or kit of parts of claim 42 for use as a medicament in treating or preventing Skin Cutaneous Melanoma, Testicular Germ Cell Tumors, or Uveal Melanoma in a subject, or any related disease, disorder, or condition, wherein the at least one tumour antigen that is provided by the artificial nucleic acid, the pharmaceutical composition, the tumour antigen or a composition of tumour antigens, the combination, or the kit or kit of parts, comprises at least one antigenic peptide selected from a peptide or protein encoded by lnc-CLEC2D-9:1, or a fragment or variant thereof.

57. A method of treating or preventing a disease, disorder or condition, wherein the method comprises applying or administering to a subject in need thereof an effective amount of the artificial nucleic acid of claims 1 to 28, a composition of claims 29 to 38, a tumour antigen or a composition of tumour antigens of claim 39, an antibody, T-cell, or TCR of claim 40, a combination of claim 41, or a kit or kit of parts of claim 42.

58. The method of claim 57, wherein the method is further characterized by any one of the medical use features as defined in claims 43 to 56.
